(19) United States
(12) Patent Application Publication
(10) Pub. No.: US 2018/0344735 A1
(43) Pub. Date: Dec. 6, 2018

(54) USE OF sGC STIMULATORS FOR THE TREATMENT OF GASTROINTESTINAL SPHINCTER DYSFUNCTION

(71) Applicant: Ironwood Pharmaceuticals, Inc., Cambridge, MA (US)

(72) Inventors: Kenneth E. Carlson, Somerville, MA (US); Joon Jung, Newton, MA (US); Mark G. Currie, Boston, MA (US)

(21) Appl. No.: 16/061,992

(22) PCT Filed: Dec. 13, 2016
(86) PCT No.: PCT/US2016/066357

§ 371 Date: Jun. 13, 2018

Related U.S. Application Data
(60) Provisional application No. 62/266,988, filed on Dec. 14, 2015.

(51) Int. Cl. A61K 31/506; A61K 31/4422; A61K 35/74; A61K 38/48; A61P 1/00; A61K 9/00

(52) U.S. Cl. CPC A61K 31/506; A61K 31/4422; A61K 9/006; A61K 38/4893; A61P 1/00; A61K 35/74

(57) ABSTRACT

The present disclosure relates to methods, uses, pharmaceutical compositions and kits comprising an sGC stimulator or a pharmaceutically acceptable salt thereof, alone or in combination with one or more additional therapeutic agents, for the treatment of a gastrointestinal sphincter disorder. Gastrointestinal sphincter disorders include, but are not limited to, an achalasia of a sphincter of the gastrointestinal tract, a spastic sphincter of the gastrointestinal tract or sphincter spasms, or a hypertensive sphincter of the gastrointestinal tract.

### CROSS REFERENCE TO RELATED APPLICATION

This application claims priority of U.S. provisional application 62/266,988, filed Dec. 14, 2015, the entire disclosure of which is hereby incorporated herein by reference.

### TECHNICAL FIELD

The present disclosure relates to methods of using soluble guanylate cyclase (sGC) stimulators and pharmaceutically acceptable salts thereof, alone or in combination with one or more additional therapeutic agents, for the treatment of gastrointestinal sphincter dysfunction or disorders, such as achalasias of a sphincter of the gastrointestinal tract, hypertensive sphincter disorders of the gastrointestinal tract and spastic sphincter disorders of the gastrointestinal tract.

### BACKGROUND

### Gastrointestinal Sphincter Dysfunction

The gastrointestinal tract is commonly divided into several parts: mouth, throat, esophagus, stomach, small intestine and large intestine. These parts are separated from each other by special muscles called sphincters which normally stay tightly closed and regulate the movement of food from one part to another, and mostly unidirectionally from mouth to anus.

The human body contains more than 60 sphincters in different body systems. The gastrointestinal tract contains several important sphincters: upper and lower esophageal sphincters (UES and LES, respectively), the pyloric sphincter or pylorus (at the lower end of the stomach), the ileocecal sphincter or valve at the junction of the latest part of the small intestine (ileum) and the large intestine, which functions to limit the reflux of colonic contents back into the ileum, the sphincter of Oddi (also named Glisson's sphincter), controlling secretions from the liver, pancreas and gall bladder into the duodenum and, at the anus, two sphincters are present, which control the exit of feces from the body (internal anal sphincter and external anal sphincter). The control of the inner anal sphincter is involuntary and the control of the outer sphincter is voluntary.

An achalasia refers to the failure of circular smooth muscle fibers to relax, which can cause a sphincter to remain closed and fail to open when needed and frequently results in the widening of the structure above the muscular constriction. Achalasias of a sphincter of the gastrointestinal tract are characterized by exceedingly high values of manometric pressure at the sphincter.

A sphincter is considered hypertensive when its resting pressure after swallow (for example, as measured by manometry) is higher than normal but not as high as in achalasias. The sphincter still partially opens and thus the symptoms associated are less severe.

For example, in achalasia of the esophagus, or simply, achalasia, the lower esophageal sphincter (LES) fails to relax upon wet swallow (<75% relaxation observed) and a value higher than 100 mm Hg is obtained by manometry (less than 26 mm Hg is considered normal). Values between 26 mm Hg and 100 mm Hg fall under hypertensive LES (HTLES). HTLES is usually defined by a resting pressure measured at the respiratory inversion point on stationary manometry of greater than 26 mm Hg (ninety-fifth percentile of normal). The most common symptoms in patients with HTLES are regurgitation (75%), heartburn (71%), dysphagia (71%), and chest pain (49%). The most common primary presenting symptoms are heartburn and dysphagia.

A spastic sphincter is one that is able to relax to a normal extent, but it may do it at the wrong times or for the wrong duration of time (resulting in spasms). A spastic sphincter usually becomes inflamed, resulting in pain. A spastic sphincter may also allow leakage of gastrointestinal contents into the wrong part of the gastrointestinal tract (for instance, contents of the colon into the small intestine).

Without a modifier or qualifier, the term “achalasia” usually refers to achalasia of the esophagus, due to a dysfunction of the LES. It is also called “esophageal achalasia”, “achalasia cardiae”, “cardiospasm” or, sometimes, “esophageal aperistalsis” (as dysfunction of the LES is frequently, although not always, associated with esophageal body peristalsis dysfunction). However, achalasias can happen at various points along the gastrointestinal tract; achalasia of the internal anal sphincter, for instance, is Hirschsprung's disease.

Throughout this disclosure, the terms achalasia (used in singular) and esophageal achalasia are used interchangeably. When referring to an achalasia not affecting the LES, a modifier or qualifier will be used. For instance, other types of achalasias contemplated in this disclosure are: achalasia of the pyloric sphincter (pyloric or pylorus achalasia), achalasia of the ileocecal sphincter (ileocecal achalasia), achalasia of the sphincter of Oddi or Glisson's sphincter (sphincter of Oddi achalasia or dysfunction, SOD) and achalasia of the internal anal sphincter (Hirschsprung's disease).

Esophageal achalasia is a form of dysphagia (difficulty swallowing). It is a rare disease characterized by failure of the LES to relax and aperistalsis of the esophageal body in response to deglution. It is a motility disorder involving the smooth muscle layer of the esophagus and the LES. It has an annual incidence of approximately 2 in 100,000 and a prevalence rate of 10 in 100,000. There is no gender predominance for the occurrence of disease.

Characteristic clinical manifestations of achalasia are difficulty swallowing solids or liquids, regurgitation of undigested food, and sometimes chest pain (cardiospasm) or heartburn. In many instances this set of symptoms results in weight loss. Some people may also experience coughing when lying in a horizontal position. Food and liquids are usually retained in the esophagus and may be inhaled into the lungs (aspiration). In addition, 40% of patients with achalasia report occurrence of at least one respiratory symptom, including cough, hoarseness, wheezing, shortness of breath and sore throat.

Clinical symptoms can initially manifest at any age, but usually manifest between the ages of 25 and 60. Diagnosis is reached with esophageal manometry (esophageal motility measurement) and barium swallow radiographic studies.

Various treatments are available, although they are all palliative and none cures the condition. Sublingual nifedipine (a Calcium channel blocker) significantly improves outcomes in 75% of people with mild or moderate disease. Certain medications or Botox (for chemical denervation) may be used in some cases, but more permanent relief is brought by esophageal pneumatic dilatation (balloon dilatation) and surgical cleaving of the muscle (Laparoscopic Heller's Myotomy). All the current treatment modalities suffer from either low effectiveness or being initially effective but having efficacy that diminishes over time or high levels of relapse. In most cases, subsequent treatments involve cumulative risks.

Manometry is the gold standard for establishing the diagnosis of achalasia. Some characteristic manometric findings of achalasia are the following: LES fails to relax upon wet swallow (<75% relaxation observed); pressure of LES <26 mm Hg is normal, whereas a value >100 is considered achalasia; aperistalsis in esophageal body is observed and relative increase in intra-esophageal pressure can be measured as compared with intra-gastric pressure. All patients with suspected achalasia also undergo upper gastrointestinal endoscopy to rule out other causes, such as mechanical obstruction due to a tumor. Values between 26 mm Hg and 100 mm Hg fall under hypertensive LES (HTLES). The most common form of achalasia is primary achalasia, also named idiopathic achalasia, which has no known underlying cause. It is believed to be due to the loss of distal esophageal inhibitory neurons. However, a small proportion occurs secondary to other conditions, such as esophageal cancer or Chagas disease (an infectious disease common in South America).

Several types of hereditary achalasia are also known. These extremely rare forms have infantile onset, usually displaying initial symptoms within 2-22 months of birth. They are associated with mutations in a single gene, for instance involving loss of function of neuronal nitric oxide synthase (nNOS) or soluble guanylate cyclase (sGC).

Although achalasia is a relatively rare condition, it carries a risk of complications, including aspiration pneumonia and esophageal cancer.

Other achalasias similarly carry risk of complications.

### Sphincter Dysfunction and the NO/cGMP Pathway

Postganglionic myenteric neurons of the myenteric plexus are responsible for controlling esophageal contractility. There are two populations of neurons involved in this process: excitatory neurons (using acetylcholine or Ach as the neurotransmitter) and inhibitory neurons (using nitric oxide (NO) or vasoactive intestinal peptide (VIP) as the neurotransmitter). Both types of neurons innervate the muscle of the muscularis propia and the LES. The myenteric plexus is a layer of nervous tissue situated between the two layers of smooth muscle that form the muscularis propia. Both circular and striatal smooth muscle tissue form the muscularis propia of the esophageal body. The LES is formed by circular smooth muscle. LES pressure at any moment reflects the balance between excitatory and inhibitory neurotransmission. At the LES, inhibitory neurons mainly use NO as the neurotransmitter.

Achalasia is believed to be due to the loss of inhibitory myenteric neurons. In the early disease stages, myenteric neurons have been found (through tissues obtained from autopsy and myotomy) to be surrounded by inflammatory cells. The presence of antibodies has also been considered to suggest an autoimmune mechanism. In the end stages of the disease, there is a marked depletion of myenteric ganglia and development of fibrosis. In severe cases, the myenteric nerves have been found to be almost completely replaced by collagen. Whereas at the LES, loss of inhibitory myenteric neurons is responsible for failure to relax, in the peristaltic esophageal body, achalasia is characterized by a loss of intrinsic acetylcholine-containing nerves, which leads to excessive relaxation and lack of peristalsis. However, usually resolving the LES issue alone already results in major symptomatic relief for the patient.

Similarly, most of the muscle along the walls and sphincters of the digestive system is smooth muscle, except for the first section of the esophagus, the UES and the external anal sphincter. Motility of the gastrointestinal tract at the smooth muscle level is controlled by the enteric nervous system through the myenteric plexus. Thus, relaxation of the sphincters situated along the gastrointestinal tracts is controlled by the tissue concentrations of nitric oxide synthesized by the neurons of the inhibitory cells of the myenteric plexus.

In cells, NO is synthesized from arginine and oxygen by various nitric oxide synthase (NOS) enzymes and by sequential reduction of inorganic nitrate. Three distinct isoforms of NOS have been identified: inducible NOS (iNOS or NOS II) found in activated macrophage cells; constitutive neuronal NOS (nNOS or NOS I), involved in neurotransmission, long term potentiation and gastrointestinal motility among other things; and constitutive endothelial NOS (eNOS or NOS III) which regulates smooth muscle relaxation in the vasculature and blood pressure.

Soluble guanylate cyclase (sGC) is the primary receptor or target for NO in vivo. sGC is expressed in the smooth muscle as well as other cells of the gastrointestinal tract. sGC can be activated via both NO-dependent and NO-independent mechanisms. In response to this activation, sGC converts guanosine triphosphate (GTP) into the secondary messenger cyclic guanosine monophosphate (cGMP). The increased level of cGMP, in turn, modulates the activity of downstream effectors including protein kinases, phosphodiesterases (PDEs) and ion channels.

Experimental and clinical evidence suggest the notion that reduced availability of endogenously produced NO by inhibitory myenteric neurons contributes to the development of achalasia. For example, mice lacking neuronal NO synthase (nNOS) show achalasia-like symptoms including LES hypertension with impaired relaxation. Consistent with this animal model, some achalasia patients have polymorphisms of genes encoding NO synthase (NOS). Low nNOS activity has also been observed in biopsies of the muscularis externa of the esophagus from achalasia patients. In addition, in a recent genetic study, nine individuals shown to have mutations leading to a loss of function of the sGC enzyme developed severe moyamoya and early-onset achalasia. The reported benefit of treatment (off-label) with nitrate donors and phosphodiesterase 5 (PDE5) inhibitors provides further evidence supporting the potential of the NO-sGC-cGMP pathway in achalasia. Both nitrates, which increase NO concentration, and the PDE5 inhibitor sildenafil, which blocks the degradation of cGMP, have been shown to reduce LES pressure in achalasia patients.

NO-independent, heme-dependent, sGC stimulators, such as the ones presented in this disclosure, have several important differentiating characteristics, when compared to other types of sGC modulators, including crucial dependency on the presence of the reduced prosthetic heme moiety for their activity, strong synergistic enzyme activation when combined with NO and stimulation of the synthesis of cGMP by direct stimulation of sGC, independent of NO. The benzylindazole compound YC-1 was the first sGC stimulator to be identified. Additional sGC stimulators with improved potency and specificity for sGC have since been developed.

Thus, in patients suffering from achalasias of the GI, the augmentation of cGMP production by sGC stimulators in response to impaired NO signaling can ameliorate excessive pressure in the LES and potentially elsewhere in the esophageal body, and consequently may improve the symptoms of achalasia.

Similarly, experimental and clinical evidence supports the notion that a dysfunctional NO-sGC-cGMP pathway is the cause of many sphincter dysfunctions affecting sphincters along the GI tract, including other achalasias of gastrointestinal tract sphincters, hypertensive sphincters of the gastrointestinal tract and spastic sphincters of the gastrointestinal tract, or spasms.

Depending on the disease, the dysfunctional NO-sGC-cGMP pathway affecting different sections of the gastrointestinal tract may be the result of damage to the myenteric inhibitory neurons (thus reducing NOS expression and NO synthesis) or damage to the smooth muscle (thus reducing expression of the target of NO, the sGC enzyme) or both. In some cases, both tissues may be relatively intact but NO availability may become reduced due, for instance, to oxidative stress. In spastic sphincters, relaxation still takes place, but the pattern of contractions is affected, probably due to un-coordinated or disorganized signaling among the various tissues involved.

Sphincter dysfunction is considered primary when it is not associated to another systemic disease.

Sphincter dysfunction can also be secondary to other diseases. For instance, the metabolic/endocrine conditions of diabetes may result in damage to the nerves of the enteric nervous system, giving rise to diabetic sphincter dysfunction in the stomach, esophagus or the intestines. In systemic sclerosis, or other connective tissue diseases, for instance, smooth muscle is replaced by fibrotic tissue, making the muscles rigid and unable to relax.

Similarly, the role played by the enteric nervous system (ENS) in neurological or neurodegenerative disorders, as well as neuronal injury, has also become increasingly evident. Pathogenic mechanisms that give rise to CNS disorders might also lead to ENS dysfunction, and in particular sphincter dysfunction, and nerves that interconnect the ENS and CNS can be conduits for disease spread. ENS dysfunction has been shown in the etiopathogenesis of autism spectrum disorder, motor neuron diseases such as amyotrophic lateral sclerosis (ALS), transmissible spongiform encephalopathies, Parkinson disease (PD) and Alzheimer disease (AD). Animal models suggest that common pathophysiological mechanisms account for the frequency of gastrointestinal comorbidity in these conditions. Other neuronal, neurodegenerative diseases that are accompanied by a component of GI dysfunction are dementias, synucleinopathies, multiple system atrophy (MSA), Lewy bodies dementia, prion diseases, multiple sclerosis, frontotemporal lobar degeneration, Huntington's disease, and spinocerebellar ataxia (spinal muscular atrophy).

Dysfunction of the ENS, and in particular of the sphincters, may also develop as a result of cerebrovascular injury, stroke, brain surgery, head or neck trauma.

Dysfunction of the ENS, and in particular of the sphincters, may also develop as a result of paraneoplastic syndrome, an autoimmune disease that attacks neurons of the enteric nervous system and is associated with different cancers, such as small cell lung cancer, breast or ovarian cancer, multiple myeloma and Hodgkin's lymphoma.

Nitrate-type NO donors, such as sublingual isosorbide dinitrate have been used as a treatment of achalasia. However, the effect of nitrates is of short duration. In addition, nitrates are known to possess limitations that preclude their long term use, such as the development of tolerance. This therapy rarely yields satisfactory long term relief.

There are also reports of the use of PDE5 inhibitors (e.g., sildenafil) for the treatment of achalasia. According to a report from 2000, sildenafil was able to reduce LES pressure but clinical symptoms were not improved. In addition, patients reported side effects such as dizziness and headaches.

### SUMMARY

Since compounds that stimulate sGC synergistically with NO and in an NO-independent manner offer considerable advantages over other current alternative therapies that target the dysfunctional NO-sGC-cGMP pathway, there is a need to develop methods of treating disorders involving dysfunction of the gastrointestinal sphincters, such as achalasias, spastic sphincters and hypertensive sphincters by administering stimulators of sGC.

There remains a need for novel treatments for these diseases. Targeting the aberrant NO pathway by using an sGC stimulator of the disclosure is a novel useful therapeutic approach for treating the symptoms that are associated with impaired NO function in these diseases.

In one aspect, the invention provides a method of treating a gastrointestinal sphincter disorder, comprising administering a therapeutically or prophylactically effective amount of an sGC stimulator, or pharmaceutically acceptable salt thereof, alone or in combination with a therapeutically or prophylactically effective amount of one or more additional therapeutic agents to a patient in need thereof.

In another aspect, the invention provides pharmaceutical compositions comprising an sGC stimulator or a pharmaceutically acceptable salt thereof, for use in the treatment of a gastrointestinal sphincter disorder in a patient in need thereof.

In another aspect, the invention provides pharmaceutical compositions comprising an sGC stimulator, or a pharmaceutically acceptable salt thereof, in combination with one or more additional therapeutic agents, for use in the treatment of a gastrointestinal sphincter disorder in a patient in need thereof.

In some embodiments, the gastrointestinal sphincter disorder is selected from an achalasia of a sphincter of the gastrointestinal tract, a spastic sphincter disorder of the gastrointestinal tract or a hypertensive sphincter disorder of the gastrointestinal tract.

In another aspect, the invention provides a method of treating an achalasia of a sphincter of the gastrointestinal tract, comprising administering a therapeutically or prophylactically effective amount of an sGC stimulator, or pharmaceutically acceptable salt thereof, alone or in combination with a therapeutically or prophylactically effective amount of one or more additional therapeutic agents to a patient in need thereof.

In another aspect, the invention provides pharmaceutical compositions comprising an sGC stimulator or a pharmaceutically acceptable salt thereof, for use in the treatment of an achalasia of a sphincter of the gastrointestinal tract in a patient in need thereof.

In another aspect, the invention provides pharmaceutical compositions comprising an sGC stimulator, or a pharmaceutically acceptable salt thereof, in combination with one or more additional therapeutic agents, for use in the treatment of an achalasia of a sphincter of the gastrointestinal tract in a patient in need thereof.

In another aspect, the invention provides a method of treating spastic sphincter disorder of the gastrointestinal tract, comprising administering a therapeutically or prophylactically effective amount of an sGC stimulator, or pharmaceutically acceptable salt thereof, alone or in combination with a therapeutically or prophylactically effective amount of one or more additional therapeutic agents to a patient in need thereof.

In another aspect, the invention provides pharmaceutical compositions comprising an sGC stimulator or a pharmaceutically acceptable salt thereof, for use in the treatment of spastic sphincter disorder of the gastrointestinal tract in a patient in need thereof.

In another aspect, the invention provides pharmaceutical compositions comprising an sGC stimulator, or a pharmaceutically acceptable salt thereof, in combination with one or more additional therapeutic agents, for use in the treatment of spastic sphincter disorder of the gastrointestinal tract in a patient in need thereof.

In another aspect, the invention provides a method of treating a hypertensive sphincter disorder of the gastrointestinal tract, comprising administering a therapeutically or prophylactically effective amount of an sGC stimulator, or pharmaceutically acceptable salt thereof, alone or in combination with a therapeutically or prophylactically effective amount of one or more additional therapeutic agents to a patient in need thereof.

In another aspect, the invention provides pharmaceutical compositions comprising an sGC stimulator or a pharmaceutically acceptable salt thereof, for use in the treatment of a hypertensive sphincter disorder of the gastrointestinal tract in a patient in need thereof.

In another aspect, the invention provides pharmaceutical compositions comprising an sGC stimulator, or a pharmaceutically acceptable salt thereof, in combination with one or more additional therapeutic agents, for use in the treatment of a hypertensive sphincter disorder of the gastrointestinal tract in a patient in need thereof.

In still a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is an sGC stimulator, a pharmaceutically acceptable salt thereof, or a pharmaceutical composition comprising an sGC stimulator or a pharmaceutically acceptable salt thereof for use in the treatment of a gastrointestinal sphincter disorder in a patient in need thereof.

In some embodiments of the above aspects, the gastrointestinal sphincter is selected from: lower esophageal sphincter (LES), pyloric sphincter (pylorus), ileocecal sphincter or valve (ICV), the sphincter of Oddi (SO, also named Glisson's sphincter) and internal anal sphincter (IAS).

In some embodiments of the above aspects, the gastrointestinal sphincter dysfunction or disease is selected from: lower esophageal sphincter (LES) achalasia, esophageal achalasia, spastic LES, hypertensive LES (HTNLES), pyloric sphincter (pylorus) achalasia, pyloric spasm (pylorospasm), hypertensive pylori, ileocecal sphincter or valve (ICV) achalasia, hypertensive ICV, spastic ICV or ICV spasm, sphincter of Oddi dysfunction (SOD), sphincter of Oddi achalasia, spastic sphincter of Oddi, hypertensive sphincter of Oddi, internal anal sphincter (IAS) achalasia, hypertensive IAS, spastic IAS or IAS spasm.

In some embodiments of the above aspects, the achalasia of a sphincter of the gastrointestinal tract is selected from lower esophageal sphincter (LES) achalasia, pyloric sphincter (pylorus) achalasia, ileocecal sphincter or valve (ICV) achalasia, sphincter of Oddi achalasia, and internal anal sphincter (IAS) achalasia.

In some embodiments of the above aspects, the spastic sphincter disorder of the gastrointestinal tract is selected from spastic LES, pyloric spasm (pylorospasm), spastic ICV or ICV spam, spastic sphincter of Oddi, and spastic IAS or IAS spasm.

In some embodiments of the above aspects, the hypertensive sphincter disorder of the gastrointestinal tract is selected from hypertensive LES (HTNLES), hypertensive pylori, hypertensive ICV, hypertensive sphincter of Oddi, and hypertensive IAS.

In some embodiments of the above aspects, the gastrointestinal sphincter disorder is associated with a metabolic or endocrine disorder.

In some embodiments, the metabolic or endocrine disorder is diabetes.

In some embodiments of the above aspects, the gastrointestinal sphincter disorder is associated with a connective tissue disease. In some embodiments, the connective tissue disease is systemic sclerosis.

In some embodiments of the above aspects, the gastrointestinal sphincter disorder is associated with a neurological or neurodegenerative disease.

In some embodiments, the neurological or neurodegenerative disease is selected from an autism spectrum disorder, a motor neuron disease, amyotrophic lateral sclerosis (ALS), a transmissible spongiform encephalopathy, Parkinson disease (PD), Alzheimer disease (AD) a dementia, a synucleinopathy, multiple system atrophy (MSA), Lewy bodies dementia, a prion disease, multiple sclerosis (MS), frontotemporal lobar degeneration, Huntington's disease (HD) or spinocerebellar ataxia (spinal muscular atrophy).

In some embodiments of the above aspects, the gastrointestinal sphincter dysfunction or disease is associated with cerebrovascular injury, stroke, brain surgery, head or neck trauma.

In some embodiments of the above aspects, the gastrointestinal sphincter dysfunction or disease is associated with paraneoplastic syndrome.

In some embodiments of the above aspects, the gastrointestinal sphincter dysfunction is associated with diabetes, systemic sclerosis, Chagas disease, a neurodegenerative or neurological disease, brain, head or neck injury or trauma or a paraneoplastic syndrome.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls. The compounds described herein may be defined by their chemical structures and/or chemical names. Where a compound is referred to by both a chemical structure and a chemical name, and the chemical structure and chemical name conflict, the chemical structure is determinative of the compound's identity.

### Therapeutic Methods

The terms “disease”, “disorder” and “condition” may be used interchangeably here to refer to an sGC, cGMP and/or NO mediated medical or pathological condition.

As used herein, the terms “subject” and “patient” are used interchangeably to refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject or patient is a human.

As used herein, the term a “patient in need thereof” is used to refer to a patient suffering from one of the gastrointestinal sphincter disorders here described, for example gastrointestinal sphincter achalasias, spastic sphincters or hypertensive sphincters.

In some embodiments, the “patient in need thereof” is a patient with achalasia (for example, idiopathic achalasia) or who has been diagnosed with achalasia or who is genetically predisposed to the development of achalasia. In still other embodiments a patient in need thereof is a person (usually a child, sometimes an infant) that has been genetically tested and found to have a mutation in a gene that predisposes him or her to the development of an achalasia, even though he or she may not show any physical symptoms of achalasia yet. In some instances, a “patient in need thereof” displays symptoms of achalasia even though a diagnosis has not been made yet.

As used herein, the term “treat”, “treating” or “treatment” with regard to a disorder or disease refers to alleviating or abrogating the cause and/or effects or symptoms or clinical manifestations of the disorder or disease. As used herein, the terms “treat”, “treatment” and “treating” refer to the reduction or amelioration or slowing down of the progression, severity and/or duration of gastrointestinal sphincter dysfunction, for example, an achalasia of a sphincter of the gastrointestinal tract, a spastic sphincter of the gastrointestinal tract or a hypertensive sphincter of the gastrointestinal tract.

In some embodiments, the terms “treat”, “treatment” and “treating” refer or the reduction, amelioration or slowing down of the progression, the severity and/or the duration of one or more symptoms or clinical manifestations (preferably, one or more measurable symptoms or clinical manifestations) of the condition, as a result of the administration of one or more therapies (e.g., an sGC stimulator or a pharmaceutically acceptable salt thereof, either alone or in combination therapy).

In some embodiments, the terms “treat,” “treatment” and “treating” refer to delaying the onset of a symptom or set of symptoms or clinical manifestations or to delaying the onset of a loss in certain physical function (e.g., ability of the LES or another gastrointestinal sphincter to relax).

In some embodiments, the terms “treat,” “treatment” and “treating” refer to the amelioration of at least one measurable physical parameter of an achalasia of a gastrointestinal tract sphincter or achalasia of the LES (e.g., aperistalsis). In other embodiments the terms “treat”, “treatment” and “treating” refer to the reduction, inhibition or slowing down of the progression of said condition, either physically by, e.g., stabilization of a measurable symptom or set of symptoms (e.g., regurgitation or pain), or physiologically by, e.g., stabilization of a measurable parameter (increased LES or other sphincter's manometric pressure), or both. As used herein, the term “treating”, “treat” or “treatment” also refer to averting the cause and/or effects or clinical manifestation of a disease or disorder or one of the symptoms developed as a result of the disease or disorder prior to the disease or disorder fully manifesting itself.

“Treatment” can involve administering a compound described herein to a patient diagnosed with a gastrointestinal sphincter dysfunction here described and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms fully develop.

In one aspect, the invention provides a method of treating achalasia, comprising administering a therapeutically or prophylactically effective amount of an sGC stimulator, or pharmaceutically acceptable salt thereof, alone or in combination with a therapeutically or prophylactically effective amount of one or more additional therapeutic agents to a patient in need thereof patient.

In a further aspect, the invention provides a use of an sGC stimulator or a pharmaceutically acceptable salt thereof, in the manufacture of a medicament for the treatment of achalasia in a patient in need thereof.

In another aspect, the invention provides pharmaceutical compositions comprising a sGC stimulator or a pharmaceutically acceptable salt thereof, for use in the treatment of achalasia in a patient in need thereof. In another aspect, the invention provides pharmaceutical compositions comprising an sGC stimulator, or a pharmaceutically acceptable salt thereof, in combination with one or more additional therapeutic agents, for use in the treatment of achalasia in a patient in need thereof.

In still a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is an sGC stimulator, a pharmaceutically acceptable salt thereof, or a pharmaceutical composition comprising an sGC stimulator or a pharmaceutically acceptable salt thereof for use in the treatment of achalasia in a patient in need thereof.

In some embodiments of the above methods, uses, compositions and kits, the patient in need thereof is an adult. In other embodiments the patient is a child. In still other embodiments the patient in need thereof is an infant.

In some embodiments of the above methods, uses, compositions and kits, the administration of an sGC stimulator or pharmaceutically acceptable salt thereof, alone or in combination with another therapeutic agent, results in an observable or measurable decrease in the degree of failure of the esophageal smooth muscle to relax after swallowing. In other embodiments, it results in an observable or measurable decrease in the degree of failure of the LES to relax after swallowing. In other embodiments, it results in an observable or measurable decrease in the degree of aperistalsis of the esophageal body in response to swallowing. In other embodiments, it results in an observable or measurable decrease in the degree of dysphagia. In other embodiments, it results in an observable or measurable reduction in regurgitation of undigested food. In still other embodiments, it results in an observable or measurable decrease in the progression of esophageal fibrosis. In other embodiments, it results in an observable or measurable reduction in inflammation around the myenteric plexus.

In some embodiments of the above methods, uses, compositions and kits, the administration of an sGC stimulator or pharmaceutically acceptable salt thereof, alone or in combination with another therapeutic agent, results in an observable or measurable reduction in heartburn. In other embodiments, it results in a measurable or observable reduction in chest pain. In other embodiments, it results in an observable or measurable reduction of wheezing. In other embodiments, it results in an observable or measurable reduction of coughing. In other embodiments, it results in an observable or measurable reduction of hoarseness. In other embodiments, it results in an observable or measurable reduction of sore throat. In other embodiments, it results in an observable or measurable reduction of coughing when lying in a horizontal position. In other embodiments, it results in an observable or measurable reduction in the degree of retention of food in the esophagus. In other embodiments, it results in an observable or measurable reduction of aspiration of food into the lungs. In other embodiments, it results in an observable or measurable reduction of cardiospasm.

In some embodiments of the above methods, uses, compositions and kits, the administration of an sGC stimulator or pharmaceutically acceptable salt thereof, alone or in combination with another therapeutic agent, results in an observable or measurable inhibition of weight loss.

In some embodiments of the above methods, uses, compositions and kits, the administration of an sGC stimulator or a pharmaceutically acceptable salt thereof, alone or in combination with another therapeutic agent, results in an observable or measurable improvement in the ability of esophageal smooth muscles fibers to relax after swallowing. In other embodiments, it results in an observable or measurable improvement in the ability of the LES to relax after swallowing. In other embodiments, it results in an observable or measurable improvement in peristalsis of the esophagus.

In other embodiments, it results in an observable or measurable improvement in the ability to swallow liquids or solids. In other embodiments, it results in an observable or measurable improvement in chest pain. In still other embodiments, it results in an observable or measurable improvement in heartburn.

In some embodiments of the above methods, uses, compositions and kits, the administration of an sGC stimulator or a pharmaceutically acceptable salt thereof, alone or in combination with another therapeutic agent, results in a measurable reduction in the LES pressure after swallowing as measured by manometry.

In some embodiments of the above methods, uses, compositions and kits, the administration of an sGC stimulator or a pharmaceutically acceptable salt thereof, alone or in combination with another therapeutic agent, results in a measurable increase in the percentage of relaxation of the LES after swallowing as measured by manometry.

In some embodiments of the above methods, uses, compositions and kits, the administration of an sGC stimulator or a pharmaceutically acceptable salt thereof, alone or in combination with another therapeutic agent, results in a measurable decrease in intra-esophageal pressure compared to intragastric pressure after swallowing as measured by manometry.

In some embodiments of the above methods, uses, compositions and kits, the administration of an sGC stimulator, or a pharmaceutically acceptable salt thereof, or a pharmaceutical composition comprising an sGC stimulator or a pharmaceutically acceptable salt thereof, alone or in combination with another therapeutic agent, results in the improvement or reduction, or slowing down in the development of one or more symptoms selected from: dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus, aspiration of food into the lungs, vomiting, projectile vomiting, constipation, abdominal pain, bloating, fullness, nausea.

In some embodiments of the above methods, uses, compositions and kits, the administration of an sGC stimulator or a pharmaceutically acceptable salt thereof, or a pharmaceutical composition comprising an sGC stimulator or a pharmaceutically acceptable salt thereof, alone or in combination with another therapeutic agent, to a patient in need thereof, is aimed at treating one or more symptoms selected from: dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus, aspiration of food into the lungs, vomiting, projectile vomiting, constipation, abdominal pain, bloating, fullness, nausea.

The pyloric valve is a sphincter-type valve that controls the opening between the bottom end of the stomach and the beginning of the small intestine. It is located about 2 inches above the navel.

The pyloric valve's principal function is to control the flow of partially digested material from the stomach into the duodenum, the topmost section of the small intestine, where most of the nutrients get extracted from what is eaten. When the valve is working well, it opens slightly a few times a minute to allow a small amount of food to move into the duodenum. Its secondary function is to prevent bile from flowing back from the small intestine into the stomach (bile reflux).

When the pyloric valve is malfunctioning, it creates discomfort and many serious medical problems. Malfunctioning of this valve results in disorders such as achalasia (or failure to relax), hypertension or spasms (relaxation that occurs inappropriately, e.g., at the wrong times or for the wrong duration of time).

When the valve spasms, it becomes inflamed, resulting in pain as food tries passing from the stomach into the small intestine. If the spasms are severe, it may result in nausea and violent vomiting as the stomach attempts to clear itself. The usual symptoms of a spastic pyloric valve that is not opening properly are bloating and a sharp pain after eating.

Pyloric spasm or pylorospasm may be associated with other diseases, for example diabetes or systemic sclerosis.

When the valve completely fails to open as it occurs in pyloric achalasia, the most common symptom is projectile or severe vomiting, accompanied by distension of the stomach and pain, as partially undigested food accumulates and is unable to pass into the intestines. This occurs, for example, in pyloric stenosis or pylorostenosis and infantile hypertrophic pyloric stenosis. The latter may be familial or idiopathic.

The ileocecal valve (ICV) is a sphincter located at the junction of the end of the small intestine and beginning of the large intestine. Its purpose is twofold: 1) To retain the contents of the small intestine long enough for the digestive process to be completed, and 2) As a barrier to prevent bacteria laden material in the large intestine from ‘back flowing’ into the small intestine and contaminating it.

When the ileocecal valve is closed, the partially digested food stays in the small intestine, where the body renders and absorbs nutrients. Once material has been allowed to pass through the ileocecal valve to enter the large intestine, the valve closes again to prevent back flow from the large intestine. If the ileocecal valve fails to relax and stays in the closed position, it can cause tightness in the bowel movements or constipation. If it becomes spastic, it may allow leakage of the contents of the large intestine into the small intestine with all the downstream consequences of this. One complication is for example small intestinal bacterial overgrowth (SIBO).

The sphincter of Oddi is a muscular valve that controls the flow of digestive juices (bile and pancreatic juice) through ducts from the liver and pancreas into the first part of the small intestine (duodenum). Sphincter of Oddi dysfunction (SOD) describes the situation when the sphincter does not relax at the appropriate time (due to scarring or spasm). The back-up of juices causes episodes of severe abdominal pain. Sphincter of Oddi dysfunction may also include complete failure to relax (achalasia) or hypertensive sphincter.

Sphincter of Oddi manometry (SOM), involves passing a catheter into the bile and/or pancreatic duct during endoscopic retrograde cholangiopancreatography (ERCP) to measure the pressure of the biliary and/or pancreatic sphincter. It is considered the gold standard diagnostic modality for SOD.

Patients with a similar pain problem, but who have little or no abnormalities on blood tests and standard scans (including MRCP), are categorized as having SOD Type III. The episodes of pain are assumed due to intermittent spasm of the sphincter. It is very difficult to effectively evaluate and manage patients with Type III SOD.

Hirschsprung's disease (HD) is a form of megacolon that occurs when part or all of the large intestine or antecedent parts of the gastrointestinal tract have no ganglion cells and therefore cannot function. During normal prenatal development, cells from the neural crest migrate into the large intestine (colon) to form the networks of nerves called the myenteric plexus (Auerbach plexus) (between the smooth muscle layers of the gastrointestinal tract wall) and the submucosal plexus (Meissner plexus) (within the submucosa of the gastrointestinal tract wall). In Hirschsprung's disease, the migration is not complete and part of the colon lacks these nerve bodies that regulate the activity of the colon. The affected segment of the colon cannot relax and pass stool through the colon, creating an obstruction. In most affected people, the disorder affects the part of the colon that is nearest the anus, i.e., the anal sphincters and related area. In rare cases, the lack of nerve bodies involves more of the colon. In five percent of cases, the entire colon is affected. The stomach and esophagus may be affected too.

Hirschsprung's disease occurs in about one in 5,000 of live births. It is usually diagnosed in children, and affects boys more often than girls. About 10% of cases are familial.

Typically, Hirschsprung's disease is diagnosed shortly after birth, although it may develop well into adulthood, because of the presence of megacolon, or because the baby fails to pass the first stool (meconium) within 48 hours of delivery. Normally, 90% of babies pass their first meconium within 24 hours, and 99% within 48 hours. Other symptoms include green or brown vomit, explosive stools after a doctor inserts a finger into the rectum, swelling of the abdomen, lots of gas and bloody diarrhea.

Some cases are diagnosed later, into childhood, but usually before age 10. The child may experience fecal retention, constipation, or abdominal distention. With an incidence of one in 5,000 births, the most cited feature is absence of ganglion cells: notably in males, 75 percent have none in the end of the colon (recto-sigmoid) and eight percent lack ganglion cells in the entire colon. The enlarged section of the bowel is found proximally, while the narrowed, aganglionic section is found distally, closer to the end of the bowel, in the sphincter area. The absence of ganglion cells results in a persistent over-stimulation of nerves in the affected region, resulting in contraction.

The lack of ganglion cells in the myenteric and submucosal plexus is well-documented in Hirschsprung's disease. The segment lacking neurons (aganglionic) becomes constricted, causing the normal, proximal section of bowel to become distended with feces. Definitive diagnosis is made by suction biopsy of the distally narrowed segment. A histologic examination of the tissue would show a lack of ganglionic nerve cells. Diagnostic techniques involve anorectal manometry, barium enema, and rectal biopsy. The suction rectal biopsy is considered the current international gold standard in the diagnosis of Hirschsprung's disease.

Radiologic findings may also assist with diagnosis. Cineanography (fluoroscopy of contrast medium passing anorectal region) assists in determining the level of the affected intestines. Treatment of Hirschsprung's disease consists of surgical removal (resection) of the abnormal section of the colon, followed by reanastomosis.

### sGC Stimulators: Definitions and General Terminology

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

Compounds herein disclosed may be optionally substituted with one or more substituents, such as illustrated generally below, or as exemplified by particular classes, subclasses and species of the invention. The phrase “optionally substituted” is used interchangeably with the phrase “substituted or unsubstituted.” In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position unless otherwise specified. As will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal to or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound is one that is not substantially altered when kept at a temperature of 25° C. or less, in the absence of moisture or other chemically reactive conditions, for at least a week. A chemically feasible compound is a compound that can be prepared by a person skilled in the art based on the disclosures herein supplemented, if necessary, relevant knowledge of the art.

A compound, such as those herein disclosed, may be present in its free form (e.g. an amorphous form, or a crystalline form or a polymorph). Under certain conditions, compounds may also form co-forms. As used herein, the term co-form is synonymous with the term multi-component crystalline form. When one of the components in the co-form has clearly transferred a proton to the other component, the resulting co-form is referred to as a “salt”. The formation of a salt is determined by how large the difference is in the pKas between the partners that form the mixture. For purposes of this disclosure, compounds include pharmaceutically acceptable salts, even if the term “pharmaceutically acceptable salts” is not explicitly noted.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are also within the scope of the invention. As an example, a substituent drawn as below:

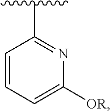

wherein R may be hydrogen, would include both compounds shown below:

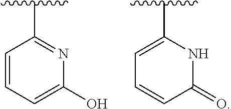

One embodiment of this invention includes isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like. To be perfectly clear, the term “aliphatic chain” may be used interchangeably with the term “aliphatic” or “aliphatic group”.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Cycloalkyl”, as used herein, refers to a ring system in which is completely saturated and which has a single point of attachment to the rest of the molecule. Unless otherwise specified, a cycloalkyl group may be monocyclic, bicyclic, tricyclic, fused, spiro or bridged. In one embodiment, the term “cycloalkyl” refers to a monocyclic C₃-C₁₂ saturated hydrocarbon or a bicyclic C₇-C₁₂ saturated hydrocarbon. In some embodiments, any individual ring in a bicyclic or tricyclic ring system has 3-7 members. Suitable cycloalkyl groups include, but are not limited to cyclopropyl, cyclobutyl, cyclopentyl, cyclohexyl, cycloheptyl, cycloheptenyl, norbornyl, cyclooctyl, cyclononyl, cyclodecyl, cycloundecyl, cyclododecyl, and the like.

“Heterocycle” (or “heterocyclyl” or “heterocyclic), as used herein, refers to a ring system in which one or more ring members are an independently selected heteroatom, which is completely saturated or that contains one or more units of” unsaturation but which is not aromatic, and which has a single point of attachment to the rest of the molecule. Unless otherwise specified, through this disclosure, heterocycle is used as a synonym of “non-aromatic heterocycle”. In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is on the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule.

Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “aralkyl” refers to a radical having an aryl ring substituted with an alkylene group, wherein the open end of the alkylene group allows the aralkyl radical to bond to another part of the compound. The alkylene group is a bivalent, straight-chain or branched, saturated hydrocarbon group. As used herein, the term “C₇₋₁₂ aralkyl” means an aralkyl radical wherein the total number of carbon atoms in the aryl ring and the alkylene group combined is 7 to 12. Examples of “aralkyl” include, but not limited to, a phenyl ring substituted by a C₁₋₆ alkylene group, e.g., benzyl and phenylethyl, and a naphthyl group substituted by a C₁₋₂ alkylene group.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6, 5 heteroaromatic system, as used herein, for example, is a six membered heteroaromatic ring fused to a second five membered ring wherein the radical or point of attachment is on the six-membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tricyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to an atom or a chain of atoms connecting two different parts of a molecule. The two atoms that are connected through the bridge (usually but not always, two tertiary carbon atoms) are referred to as “bridgeheads”. In addition to the bridge, the two bridgeheads are connected by at least two individual atoms or chains of atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. “Spiro” bicyclic ring systems share only one ring atom (usually a quaternary carbon atom) between the two rings.

The term “ring atom” refers to an atom such as C, N, O or S that is part of the ring of an aromatic ring, a cycloaliphatic ring, a heterocyclic or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR+(as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloaliphatic ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, cycloaliphatic or aryl ring, wherein the group is attached to the rest of the molecule by a single point of attachment; and b) two independent occurrences of a substituent that are bound to different atoms and are taken together with both of those atoms to form a heterocyclyl, heteroaryl, cycloaliphatic or aryl ring, wherein the ring that is formed has two points of attachment with the rest of the molecule. For example, where a phenyl group is substituted with two occurrences of —OR^{o} as in Formula D1:

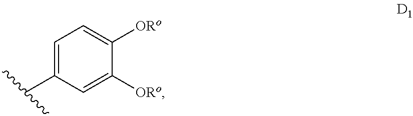

these two occurrences of —OR^{o} are taken together with the carbon atoms to which they are bound to form a fused 6-membered oxygen containing ring as in Formula D2:

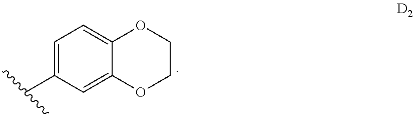 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. If this is the case, this will clearly be indicated in the definition of the specific alkyl or aliphatic chain (for instance, a certain variable will be described as being a C₁₋₆ alkyl group, wherein said alkyl group is optionally interrupted by a certain group). Unless otherwise indicated, alkyl and aliphatic chains will be considered to be formed by carbon atoms only without interruptions. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to an H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R′)—, —C(O)—, and —N(R′)— to form —N(R′)C(O)N(R′)— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

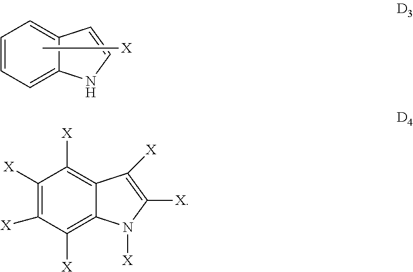

This also applies to multiple ring systems fused to optional ring systems (which would be represented by dotted lines). For example, in Formula D5, X is an optional substituent both for ring A and ring B.

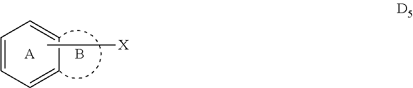

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

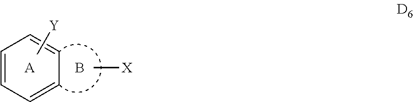

As used herein, the terms “alkoxy” or “alkylthio” refer to an alkyl group, as previously defined, attached to the molecule, or to another chain or ring, through an oxygen (“alkoxy” i.e., —O-alkyl) or a sulfur (“alkylthio” i.e., —S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)O CH₂CH₂CH₃; a C₅ alkoxyalkenyl could be, for instance, —CH═CHO CH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —CEN.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, a “carbonyl”, used alone or in connection with another group refers to —C(O)- or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom (e.g., it can also be attached to a sulfur atom). An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

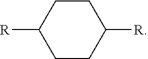

A linker can additionally be optionally substituted in any portion or position.

Divalent groups of the type R—CH═ or R₂C═, wherein both free valences are in the same atom and are attached to the same substituent, are also possible. In this case, they will be referred to by their IUPAC accepted names. For instance an alkylidene (such as, for example, a methylidene (═CH₂) or an ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W. et al., “Protective Groups in Organic Synthesis”, Third Edition, John Wiley & Sons, New York: 1999, the entire contents of which is hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In some embodiments of the above methods, uses, pharmaceutical compositions and kits, the sGC stimulator is selected from those described in patent application publications WO2013101830 (e.g., any one of compounds 1 to 122), WO2012064559 (e.g., any one of compounds I-1 to 1-68), WO2012003405 (e.g., any one of compounds I-1 to I-312), WO2011115804 (e.g., any one of compounds I-1 to 1-63), WO2014047111 (e.g., any one of compounds I-1 to 1-5), WO2014047325 (e.g., any one of compounds I-1 to I-10); WO2014144100 (e.g., any one of compounds I-1 to 1-634); WO2015089182 (e.g., any one of compounds I-1 to I-72), WO2016044447 (e.g., any one of compounds 1 to 217), WO2016044446 (e.g., any one of compounds I-1 to 1-94), WO2016044445 (e.g., any one of compound I-1 to 1-39), WO2016044441 (e.g., any one of compound I-1 to 1-20) or is a pharmaceutically acceptable salt thereof.

In other embodiments of the above methods, uses, pharmaceutical compositions and kits, the sGC stimulator is a compound described in one or more of the following publications: US20140088080 (WO2012165399), WO2014084312, U.S. Pat. No. 6,414,009, U.S. Pat. No. 6,462,068, U.S. Pat. No. 6,387,940, U.S. Pat. No. 6,410,740 (WO 98 16507), U.S. Pat. No. 6,451,805 (WO 98 23619), U.S. Pat. No. 6,180,656 (WO 98 16223), US20040235863 (WO2003004503), US 20060052397, U.S. Pat. No. 7,173,037 (WO2003095451), US 20060167016, U.S. Pat. No. 7,091,198 (WO2004009589), US 20060014951, U.S. Pat. No. 7,410,973 (WO2004009590), US 20100004235 (WO2007124854, e.g., Examples 1, 2, 3, 6, 7, 18 or 19), US20100029653 (WO 2008031513, e.g., Examples 1, 2, 3, 4 or 7), US20100113507 (WO2007128454, e.g., Example 1, 4 or 7), US 20110038857, U.S. Pat. No. 8,114,400 (WO2008061657), US20110218202 (WO 2010065275, e.g., Examples 1, 3, 59, 60 or 111), US20110245273 (WO 2010078900, e.g., Examples 1 or 5), US2012029002 (WO 2010079120), US20120022084, US 20130237551, U.S. Pat. No. 8,420,656 (WO 2011147809, WO 2011147810), US20130210824 (WO2013104598), US20130172372 (WO2012004259, e.g., Examples 2, 3 or 4), US20130267548 (WO2012059549, e.g., Examples 1, 2, 7, 8 or 13), WO 2012143510 (e.g., Examples 1, 2, 3, 4, 5, 6, 7, 8, 9 or 10), WO2012004258 (e.g., Examples 1, 18, 19 or 27), WO2012152629 (e.g., Examples 11 or 12), WO2012152630 (e.g., Examples 1, 5, 8, 11, 15 or 19), WO2012010577 (e.g., Examples 3-1, 4, 5 or 6), WO2012028647 (e.g., Examples 1, 2 or 3), WO2013104597 (e.g., Examples 16, 18, 22 or 23), WO2013131923 (e.g., Examples 1, 2, 7, 8 or 9), WO2013104703, WO2013004785 (e.g., Examples 1, 3 or 6), WO2013030288, US20090209556, U.S. Pat. No. 8,455,638, US20110118282 (WO2009032249), US20100292192, US20110201621, U.S. Pat. No. 7,947,664, U.S. Pat. No. 8,053,455 (WO2009094242), US20100216764, U.S. Pat. No. 8,507,512, (WO2010099054), US20110218202 (WO2010065275), US20130012511 (WO2011119518), US20130072492 (WO2011149921, e.g., Example #160, Example #164 and Example #181), US20130210798 (WO2012058132), U.S. Pat. No. 8,796,305 (WO2014068095), US20140128372 and US20140179672 (WO2014068099), U.S. Pat. No. 8,778,964 (US20140128386, US20140128424, WO2014068104), WO2014131741, US20140249168 (WO2014131760), WO2011064156, WO2011073118, WO1998023619, WO2000006567, WO2000006569, WO2000021954, WO2000066582, WO2001083490, WO2002042299, WO2002042300, WO2002042301, WO2002042302, WO2002092596, WO2003097063, WO2004031186, WO2004031187, WO2014195333, WO2015018814, WO2015082411, WO2015124544, U.S. Pat. No. 6,833,364 (DE19834047), WO2001017998 (DE19942809), WO2001047494 (DE19962926), WO2002036120 (DE10054278), WO2011064171, WO2013086935, WO2014128109, WO2012010578, WO2013076168, WO2000006568, WO2015124544, WO2015150366, WO2015150364, WO2015150363, WO2015150362, WO2015140199, WO2015150350, WO2015140254, WO2015088885 and WO2015088886.

In some further embodiments of the above methods, uses, pharmaceutical compositions and kits, the sGC stimulator is a compound described in one or more of the following publications: WO2000006568, WO2001017998, WO2001047494 and WO2002036120.

In some further embodiments of the above methods, uses, pharmaceutical compositions and kits, the sGC stimulator is a compound described in one or more of the following publications: US20110131411, WO2011064156 and WO2011073118.

In some further embodiments of the above methods, uses, pharmaceutical compositions and kits, the sGC stimulator is a compound described in one or more of the following publications: US20140315926, WO2003095451, WO2011064171, WO2013086935 and WO2014128109.

In some further embodiments of the above methods, uses, pharmaceutical compositions and kits, the sGC stimulator is a compound described in one or more of the following publications: WO2011147809, WO2012010578, WO2012059549 and WO2013076168.

In some embodiments of the above methods, uses, pharmaceutical compositions and kits, the sGC stimulator is a compound depicted below:

riociguat (BAY 63-2521, Adempas®, FDA approved drug, described in DE19834044)

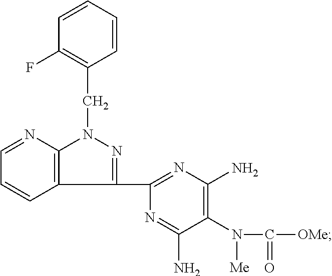 [SMILES: COC(=O)N(C)c1c(N)nc(-c2nn(Cc3ccccc3F)c3ncccc23)nc1N]

neliciguat (BAY 60-4552, described in WO 2003095451):

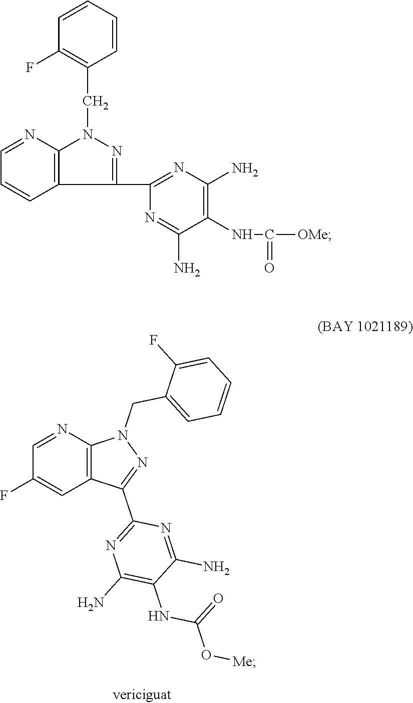 [SMILES: COC(=O)Nc1c(N)nc(-c2nn(Cc3ccccc3F)c3ncccc23)nc1N ; COC(=O)Nc1c(N)nc(-c2nn(Cc3ccccc3F)c3ncc(F)cc23)nc1N]

BAY 41-2272 (described in DE19834047 and DE19942809)

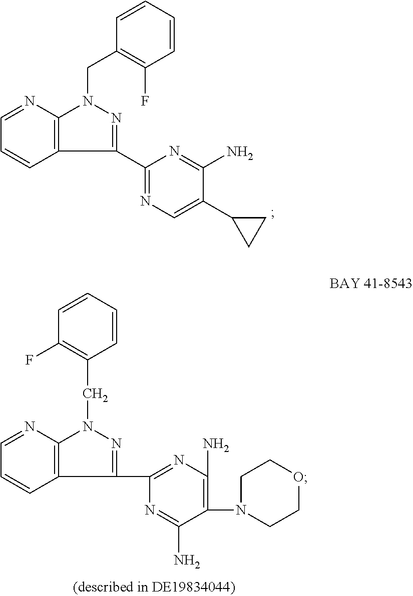 [SMILES: Nc1nc(-c2nn(Cc3ccccc3F)c3ncccc23)ncc1C1CC1 ; Nc1nc(-c2nn(Cc3ccccc3F)c3ncccc23)nc(N)c1N1CCOCC1]

etriciguat (described in WO 2003086407):

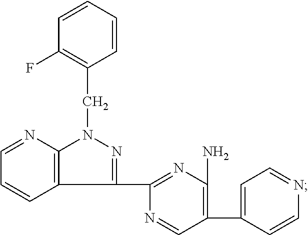 [SMILES: Nc1nc(-c2nn(Cc3ccccc3F)c3ncccc23)ncc1-c1ccncc1]

or

one of the compounds depicted below and described in US20130072492 (WO 2011149921):

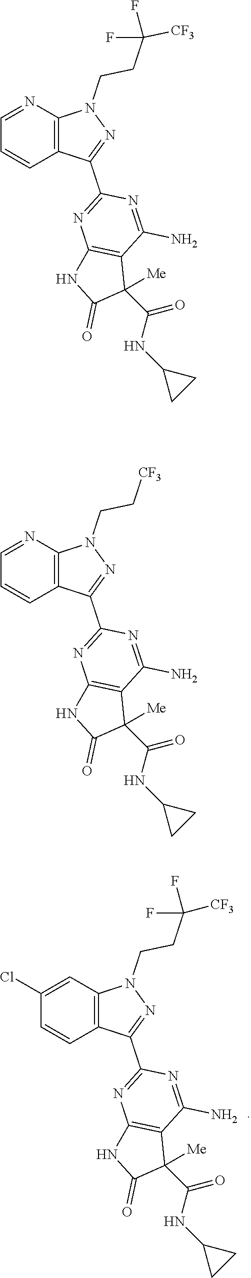

In another aspect, the invention is directed to a compound according to Formula I′, or a pharmaceutically acceptable salt thereof.

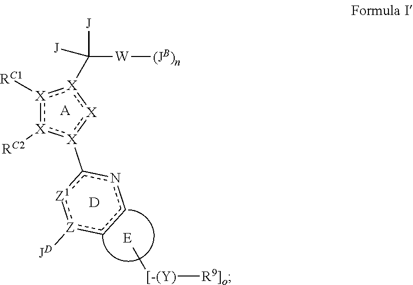

wherein:
ring A is a 5-membered heteroaryl ring; each instance of X is independently selected from C or N and the bond between each two instances of X is either a single or a double bond so as to make ring A an aromatic heterocycle; wherein a minimum of 2 instances of X and a maximum of 3 instances of X in ring A can simultaneously be N;
W is either
i) absent, and J^{B} is connected directly to the carbon atom bearing two J groups; each J is independently selected from hydrogen or methyl, n is 1 and J^{B} is a C₂₋₇ alkyl chain optionally substituted by between 2 and up to 9 instances of fluorine; wherein, optionally, one —CH₂— unit of said C₂₋₇ alkyl chain can be replaced by —O— or —S—.
ii) a ring B selected from phenyl, a 5 or 6-membered heteroaryl ring, containing 1 or 2 ring heteroatoms independently selected from N, O or S, a C₃₋₇ cycloalkyl ring and a 4 to 7-membered heterocyclic ring, containing up to 3 heteroatoms independently selected from O, N or S;
wherein when W is ring B
   each J is hydrogen;
   n is 0 or an integer selected from 1, 2 or 3;
   each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³;
   each R^{B} is independently selected from hydrogen, a C₁₋₆ aliphatic or a C₃₋₈ cycloaliphatic; wherein each of said R^{B} that is a C₁₋₆ aliphatic and each of said R^{B} that is a C₃₋₈ cycloaliphatic ring is optionally and independently substituted with up to 3 instances of R^{3a};
   each R³ is independently selected from halogen, —CN, C₁₋₄ alkyl, C₁₋₄ haloalkyl, —O(C₁₋₄ alkyl) or —O(C₁₋₄ haloalkyl);
   each R^{3a} is independently selected from halogen, —CN, C₁₋₄ alkyl, C₁₋₄ haloalkyl, —O(C₁₋₄ alkyl) or —O(C₁₋₄ haloalkyl);
Z¹ in ring D is selected from CH, CF or N; Z is selected from C or N; wherein if Z¹ is CH or CF, then Z must be C; and if Z is N, then Z may be C or N;
each J^{D} is independently selected from J^{A}, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, —N(R^{d})SO₂NHR^{D}, —N(R^{d})SO₂NHC(O)OR^{D}, —N(R^{d})SO₂NHC(O)R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocyclic ring and each said 5 to 10-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{D} moiety, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R^{5d};
J^{A} is selected from a lone pair on nitrogen, hydrogen, halogen, oxo, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
each R^{D} is independently selected from hydrogen, a C₁₋₆ aliphatic, —(C₁₋₆ aliphatic)-R^{f}, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 10-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—;
each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b}; wherein when any R^{d} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{dd})—, —CO— or —O—;
each R^{dd} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b};
each R^{f} is independently selected from a C₁₋₃ alkyl, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 4 heteroatoms independently selected from O, N or S; and wherein each said C₃₋₈ cycloaliphatic ring, each said 4 to 10-membered heterocyclic ring, each said phenyl and each said 5 to 6-membered heteroaryl ring is optionally and independently substituted by up to 5 instances of R^{5c};
when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R⁵;
when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R⁵;
when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})SO₂R^{D}, the R^{D} group together with the sulfur atom attached to the R^{D} group, with the nitrogen atom attached to the R^{a} group, and with said R^{a} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
each R⁵ is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of the —(C₁₋₆ alkyl)-R⁶ moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
each R^{5a} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
each R^{5b} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5a} or two instances of R^{5b} attached to the same or different atoms of R^{D} or R^{d}, respectively, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
each R^{5c} is independently selected from halogen, —CN, C₁₋₆ alkyl, —(C₁₋₆ alkyl)-R^{6b}, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R^{6b} moiety, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5c} attached to the same or different atoms of R^{f}, together with said atom or atoms to which it is attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
each R^{5d} is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl, C₁₋₆ alkyl portion of the —(C₁₋₆ alkyl)-R⁶moiety, C₇₋₁₂ aralkyl, C₃₋₈ cycloalkyl ring, 4 to 7-membered heterocyclic ring, 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5d} attached to the same or different atoms of J^{D}, together with said atom or atoms of J^{D} to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —C(O)NH₂, —OH or halogen; wherein R is hydrogen or a C₁₋₂ alkyl;
each R⁶ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6b} linked to a nitrogen atom of R^{5c}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
ring E is a 5 to 7-membered heterocycle or a 5-membered heteroaryl ring; said heterocycle or heteroaryl ring containing up to 4 heteroatoms independently selected from N, O and S;
o is 0 or an integer selected from 1, 2, 3 or 4;
Y is either absent or is a C₁₋₆ alkyl chain, optionally substituted by up to 6 instances of fluoro; and wherein in said Y that is a C₁₋₆ alkyl chain, up to 3 methylene units of this alkyl chain, can be replaced by a group selected from —O—, —C(O)— or —N((Y′)—R⁹⁰)—, wherein
Y¹ is either absent or is a C₁₋₆ alkyl chain, optionally substituted by up to 6 instances of fluoro; and:
when Y¹ is absent, each R⁹⁰ is independently selected from hydrogen, —COR¹⁰, —C(O)OR¹⁰, —C(O)N(R¹⁰)₂, —C(O)N(R¹⁰)SO₂R¹⁰, —SO₂R¹⁰, —SO₂N(R¹⁰)₂, —SO₂N(R¹⁰)COOR¹⁰, —SO₂N(R¹⁰)C(O)R¹⁰, —(C═O)NHOR¹⁰ a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹; and
when Y¹ is present, each R⁹⁰ is independently selected from hydrogen, halogen, —CN, —OR, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R⁹ is independently selected from hydrogen, halogen, a C₁₋₆ alkyl, —CN, —OR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₁₋₆ alkyl, each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹; each R¹⁰ is independently selected from hydrogen, a C₁₋₆ alkyl, —(C₁₋₆ alkyl)-R¹³, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R¹³ moiety, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11a};
each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11b};
each R¹¹ is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R^{11a} is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹; and
each R^{11b} is independently selected from halogen, C₁₋₆ alkyl, oxo, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R¹² is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
each R¹²¹ is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
R^{C1} is either
i) a ring C; or
ii) is selected from a lone pair on a nitrogen atom, hydrogen, halogen, oxo, —CN, C₁₋₆ aliphatic, —(C₁₋₆ aliphatic)-R^{N}, —OR⁷, —OC(O)R⁷, —O(R⁷)C(O)N(R⁷)₂, —COR⁷, —C(O)OR⁷, —C(O)N(R⁷)₂, —N(R⁷)C(O)R⁷, —N(R⁷)C(O)OR⁷, —N(R⁷)C(O)N(R⁷)₂, —N(R⁷)₂, —SR⁷, —S(O)R⁷, —SO₂R⁷, —SO₂N(R⁷)₂, —C(O)N(R⁷)SO₂R⁷, —SO₂N(R⁷)COOR⁷, —SO₂N(R⁷)C(O)R⁷ or —N(R⁷)SO₂R⁷; wherein each said C₁₋₆ aliphatic, each C₁₋₆ aliphatic portion of said —(C₁₋₆ aliphatic)-R^{N}, is optionally and independently substituted with up to 6 instances of fluoro and up to 2 instances of —CN, —OR, oxo, —N(R)₂, —N(R⁸)C(O)R⁸, —N(R⁸)C(O)OR⁸, —N(R)C(O)N(R)₂, —SO₂R⁸, —SO₂N(R⁸)₂, —NHOR⁸, —SO₂N(R⁸)COOR⁸, —SO₂N(R⁸)C(O)R⁸, —N(R)SO₂R⁸;
wherein each R⁷ is independently selected from hydrogen, C₁₋₆ alkyl, C₁₋₆ fluoroalkyl, a C₃₋₈ cycloalkyl ring, phenyl, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁸ is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{N} is independently selected from a phenyl ring, a monocyclic 5 or 6-membered heteroaryl ring, a monocyclic C₃₋₆ cycloaliphatic ring, or a monocyclic 4 to 6-membered heterocycle; wherein said monocyclic 5 or 6-membered heteroaryl ring or said monocyclic 4 to 6-membered heterocycle contain between 1 and 4 heteroatoms selected from N, O or S; wherein said monocyclic 5 or 6-membered heteroaryl ring is not a 1,3,5-triazinyl ring; and wherein said phenyl, said monocyclic 5 to 6-membered heteroaryl ring, said monocyclic C₃₋₆ cycloaliphatic ring, or said monocyclic 4 to 6-membered heterocycle is optionally and independently substituted with up to 6 instances of fluoro and/or up to 3 instances of J^{M};
each J^{M} is independently selected from —CN, a C₁₋₆ aliphatic, —OR^{M}, —SR^{M}, —N(R^{M})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7c};
each R^{M} is independently selected from hydrogen, a C₁₋₆ aliphatic, a C₃₋₈ cycloaliphatic ring or a 4 to 8-membered heterocyclic ring; wherein each said 4 to 8-membered heterocyclic ring contains between 1 and 3 heteroatoms independently selected from O, N or S; and wherein
ring C is a phenyl ring, a monocyclic 5 or 6-membered heteroaryl ring, a bicyclic 8 to 10-membered heteroaryl ring, a monocyclic 3 to 10-membered cycloaliphatic ring, or a monocyclic 4 to 10-membered heterocycle; wherein said monocyclic 5 or 6-membered heteroaryl ring, said bicyclic 8 to 10-membered heteroaryl ring, or said monocyclic 4 to 10-membered heterocycle contain between 1 and 4 heteroatoms selected from N, O or S; wherein said monocyclic 5 or 6-membered heteroaryl ring is not a 1,3,5-triazinyl ring; and wherein said phenyl, monocyclic 5 to 6-membered heteroaryl ring, bicyclic 8 to 10-membered heteroaryl ring, monocyclic 3 to 10-membered cycloaliphatic ring, or monocyclic 4 to 10-membered heterocycle is optionally and independently substituted with up to p instances of J^{C}; wherein p is 0 or an integer selected from 1, 2 or 3.
each J^{C} is independently selected from halogen, —CN, —NO₂, a C₁₋₆ aliphatic, —OR^{H}, —SR^{H}, —N(R^{H})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7d}; or
alternatively, two J^{C} groups attached to two vicinal ring C atoms, taken together with said two vicinal ring C atoms, form a 5 to 7-membered heterocycle that is a new ring fused to ring C; wherein said 5 to 7-membered heterocycle contains from 1 to 2 heteroatoms independently selected from N, O or S;
each R^{H} is independently selected from hydrogen, a C₁₋₆ aliphatic, a C₃₋₈ cycloaliphatic ring or a 4 to 8-membered heterocyclic ring; wherein each said 4 to 8-membered heterocyclic ring contains between 1 and 3 heteroatoms independently selected from O, N or S; alternatively, two instances of R^{H} linked to the same nitrogen atom of —N(R^{H})₂, together with said nitrogen atom of —N(R^{H})₂, form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S;
each R^{7c} is independently selected from halogen, —CN, —NO₂, C₁₋₄ alkyl, C₁₋₄ haloalkyl, C₃₋₈ cycloalkyl ring, —OR^{8b}, —SR^{8b}, —N(R^{8b})₂, —C(O)O(C₁₋₄ alkyl), —C(O)OH, —NR(CO)CO(C₁₋₄ alkyl) or an oxo group; wherein each said cycloalkyl group is optionally and independently substituted with up to 3 instances of halogen;
each R^{7d} is independently selected from halogen, —CN, —NO₂, C₁₋₄ alkyl, C₁₋₄ haloalkyl, C₃₋₈ cycloalkyl ring, —C(O)O(C₁₋₄ alkyl), —C(O)OH, —OR^{8c}, —SR^{8c}, —N(R^{8c})₂, or an oxo group; wherein each said cycloalkyl group is optionally and independently substituted with up to 3 instances of halogen;
each R^{8b} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{8c} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; and
R^{C2} is selected from a lone pair on a nitrogen atom, hydrogen, halogen, —OH, —O(C₁₋₆ alkyl), —O(haloC₁₋₆ alkyl), —O(C₁₋₆ haloalkyl), —O(cyclopropyl), cyclopropyl, C₁₋₆ alkyl, C₁₋₆ haloalkyl and —CN;

In some embodiments of the compounds of Formula I′, the compound is of Formula I, or a pharmaceutically acceptable salt thereof:

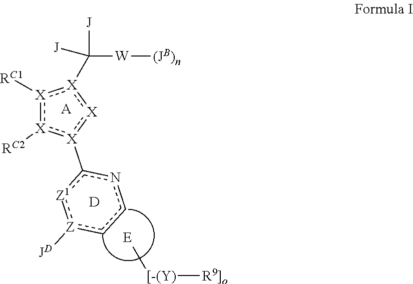

wherein:
ring A is a 5-membered heteroaryl ring; each instance of X is independently selected from C or N and the bond between each two instances of X is either a single or a double bond so as to make ring A an aromatic heterocycle; wherein a minimum of 2 instances of X and a maximum of 3 instances of X in ring A can simultaneously be N;
W is either
i) absent, and J^{B} is connected directly to the carbon atom bearing two J groups; each J is independently selected from hydrogen or methyl, n is 1 and J^{B} is a C₂₋₇ alkyl chain optionally substituted by between 2 and 9 instances of fluorine; wherein, optionally, one —CH₂— unit of said C₂₋₇ alkyl chain can be replaced by —O— or —S—.
ii) a ring B selected from phenyl, a 5 or 6-membered heteroaryl ring, containing 1 or 2 ring heteroatoms independently selected from N, O or S, a C₃₋₇ cycloalkyl ring and a 4 to 7-membered heterocyclic compound, containing up to 3 heteroatoms independently selected from O, N or S;
wherein when W is ring B
   each J is hydrogen;
   n is 0 or an integer selected from 1, 2 or 3;
   each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³;
   each R^{B} is independently selected from hydrogen, a C₁₋₆ aliphatic or a C₃₋₈ cycloaliphatic;
   wherein each of said R^{B} that is a C₁₋₆ aliphatic and each of said R^{B} that is a C₃₋₈ cycloaliphatic ring is optionally and independently substituted with up to 3 instances of R^{3a};
   each R³ is independently selected from halogen, —CN, C₁₋₄ alkyl, C₁₋₄ haloalkyl, —O(C₁₋₄ alkyl) or —O(C₁₋₄ haloalkyl);
   each R^{3a} is independently selected from halogen, —CN, C₁₋₄ alkyl, C₁₋₄ haloalkyl, —O(C₁₋₄ alkyl) or —O(C₁₋₄ haloalkyl);
Z¹ in ring D is selected from CH or N; Z is selected from C or N; wherein if Z¹ is CH, then Z must be C; and if Z1 is N, then Z may be C or N;
each J^{D} is independently selected from J^{A}, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, —N(R^{d})SO₂NHR^{D}, —N(R^{d})SO₂NHC(O)OR^{D}, —N(R^{d})SO₂NHC(O)R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocyclic ring and each said 5 to 10-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{D} moiety, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R^{5d};
J^{A} is selected from a lone pair on nitrogen, hydrogen, halogen, oxo, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
each R^{D} is independently selected from hydrogen, a C₁₋₆ aliphatic, —(C₁₋₆ aliphatic)-R^{f}, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 10-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—;
each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b}; wherein when any R^{d} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{dd})—, —CO— or —O—;
each R^{dd} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b};
each R^{f} is independently selected from a C₁₋₃ alkyl, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 4 heteroatoms independently selected from O, N or S; and wherein each said C₃₋₈ cycloaliphatic ring, each said 4 to 10-membered heterocyclic ring, each said phenyl and each said 5 to 6-membered heteroaryl ring is optionally and independently substituted by up to 5 instances of R^{5c};
when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R⁵;
when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R⁵;
when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})SO₂R^{D}, the R^{D} group together with the sulfur atom attached to the R^{D} group, with the nitrogen atom attached to the R^{d} group, and with said R^{d} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
each R⁵ is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of the —(C₁₋₆ alkyl)-R⁶ moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
each R^{5a} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
each R^{5b} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5a} or two instances of R^{5b} attached to the same or different atoms of R^{D} or R^{d}, respectively, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
each R^{5c} is independently selected from halogen, —CN, C₁₋₆ alkyl, —(C₁₋₆ alkyl)-R^{6b}, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR⁶b a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R^{6b} moiety, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵ attached to the same or different atoms of R^{f}, together with said atom or atoms to which it is attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
each R^{5d} is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl, C₁₋₆ alkyl portion of the —(C₁₋₆ alkyl)-R⁶moiety, C₇₋₁₂ aralkyl, C₃₋₈ cycloalkyl ring, 4 to 7-membered heterocyclic ring, 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5d} attached to the same or different atoms of J^{D}, together with said atom or atoms of J^{D} to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —C(O)NH₂, —OH or halogen; wherein R is hydrogen or a C₁₋₂ alkyl;
each R⁶ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6b} linked to a nitrogen atom of R^{5C}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
ring E is a 5 to 7-membered heterocycle or a 5-membered heteroaryl ring; said heterocycle or heteroaryl ring containing up to 4 heteroatoms independently selected from N, O and S;
o is 0 or an integer selected from 1, 2, 3 or 4;
Y is either absent or is a C₁₋₆ alkyl chain, optionally substituted by up to 6 instances of fluoro; and wherein in said Y that is a C₁₋₆ alkyl chain, up to 3 methylene units of this alkyl chain, can be replaced by a group selected from —O—, —C(O)— or —N((Y′)—R⁹⁰)—, wherein
Y¹ is either absent or is a C₁₋₆ alkyl chain, optionally substituted by up to 6 instances of fluoro; and:
when Y¹ is absent, each R⁹⁰ is independently selected from hydrogen, —COR¹⁰, —C(O)OR, —C(O)N(R¹⁰)₂, —C(O)N(R¹⁰)SO₂R¹⁰, —SO₂R¹⁰, —SO₂N(R¹⁰)₂, —SO₂N(R¹⁰)COOR¹⁰, —SO₂N(R¹⁰)C(O)R¹⁰, —(C═O)NHOR¹⁰ a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹; and
when Y¹ is present, each R⁹⁰ is independently selected from hydrogen, halogen, —CN, —OR, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R⁹ is independently selected from hydrogen, halogen, a C₁₋₆ alkyl, —CN, —OR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₁₋₆ alkyl, each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R¹⁰ is independently selected from hydrogen, a C₁₋₆ alkyl, —(C₁₋₆ alkyl)-R¹³, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R¹³ moiety, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11a};
each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11b};
each R¹¹ is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R^{11a} is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹; and
each R^{11b} is independently selected from halogen, C₁₋₆ alkyl, oxo, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R¹² is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
each R¹²¹ is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo.
R^{C1} is either
i) a ring C; or
ii) is selected from hydrogen, halogen, oxo, —CN, C₁₋₆ aliphatic, —(C₁₋₆ aliphatic)-R^{N}, —OR⁷, —OC(O)R⁷, —O(R)C(O)N(R)₂, —COR⁷, —C(O)OR⁷, —C(O)N(R)₂, —N(R)C(O)R⁷, —N(R)C(O)OR⁷, —N(R)C(O)N(R)₂, —N(R)₂, —SR⁷, —S(O)R⁷, —SO₂R⁷, —SO₂N(R)₂, —C(O)N(R)SO₂R⁷, —SO₂N(R)COOR⁷, —SO₂N(R)C(O)R⁷ or —N(R)SO₂R; wherein each said C₁₋₆ aliphatic, each C₁₋₆ aliphatic portion of said —(C₁₋₆ aliphatic)-R^{N}, is optionally and independently substituted with up to 6 instances of fluoro and up to 2 instances of —CN, —OR⁸, oxo, —N(R)₂, —N(R⁸)C(O)R⁸, —N(R⁸)C(O)OR⁸, —N(R)C(O)N(R)₂, —SO₂R⁸, —SO₂N(R)₂, —NHOR⁸, —SO₂N(R⁸)COOR⁸, —SO₂N(R⁸)C(O)R⁸, —N(R)SO₂R⁸;
wherein each R⁷ is independently selected from hydrogen, C₁₋₆ alkyl, C₁₋₆ fluoroalkyl, a C₃₋₈ cycloalkyl ring, phenyl, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁸ is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{N} is independently selected from a phenyl ring, a monocyclic 5 or 6-membered heteroaryl ring, a monocyclic C₃₋₆ cycloaliphatic ring, or a monocyclic 4 to 6-membered heterocycle; wherein said monocyclic 5 or 6-membered heteroaryl ring or said monocyclic 4 to 6-membered heterocycle contain between 1 and 4 heteroatoms selected from N, O or S; wherein said monocyclic 5 or 6-membered heteroaryl ring is not a 1,3,5-triazinyl ring; and wherein said phenyl, said monocyclic 5 to 6-membered heteroaryl ring, said monocyclic C₃₋₆ cycloaliphatic ring, or said monocyclic 4 to 6-membered heterocycle is optionally and independently substituted with up to 6 instances of fluoro and/or up to 3 instances of J^{M};
each J^{M} is independently selected from —CN, a C₁₋₆ aliphatic, —OR^{M}, —SR^{M}, —N(R^{M})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7c};
each R^{M} is independently selected from hydrogen, a C₁₋₆ aliphatic, a C₃₋₈ cycloaliphatic ring or a 4 to 8-membered heterocyclic ring; wherein each said 4 to 8-membered heterocyclic ring contains between 1 and 3 heteroatoms independently selected from O, N or S; and wherein
ring C is a phenyl ring, a monocyclic 5 or 6-membered heteroaryl ring, a bicyclic 8 to 10-membered heteroaryl ring, a monocyclic 3 to 10-membered cycloaliphatic ring, or a monocyclic 4 to 10-membered heterocycle; wherein said monocyclic 5 or 6-membered heteroaryl ring, said bicyclic 8 to 10-membered heteroaryl ring, or said monocyclic 4 to 10-membered heterocycle contain between 1 and 4 heteroatoms selected from N, O or S; wherein said monocyclic 5 or 6-membered heteroaryl ring is not a 1,3,5-triazinyl ring; and wherein said phenyl, monocyclic 5 to 6-membered heteroaryl ring, bicyclic 8 to 10-membered heteroaryl ring, monocyclic 3 to 10-membered cycloaliphatic ring, or monocyclic 4 to 10-membered heterocycle is optionally and independently substituted with up to p instances of J^{C}; wherein p is 0 or an integer selected from 1, 2 or 3.
each J^{C} is independently selected from halogen, —CN, —NO₂, a C₁₋₆ aliphatic, —OR^{H}, —SR^{H}, —N(R^{H})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7d}; or
alternatively, two J^{C} groups attached to two vicinal ring C atoms, taken together with said two vicinal ring C atoms, form a 5 to 7-membered heterocycle that is a new ring fused to ring C; wherein said 5 to 7-membered heterocycle contains from 1 to 2 heteroatoms independently selected from N, O or S;
each R^{H} is independently selected from hydrogen, a C₁₋₆ aliphatic, a C₃₋₈ cycloaliphatic ring or a 4 to 8-membered heterocyclic ring; wherein each said 4 to 8-membered heterocyclic ring contains between 1 and 3 heteroatoms independently selected from O, N or S; alternatively, two instances of R^{H} linked to the same nitrogen atom of —N(R^{H})₂, together with said nitrogen atom of —N(R^{H})₂, form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S;
each R^{7c} is independently selected from halogen, —CN, —NO₂, C₁₋₄ alkyl, C₁₋₄ haloalkyl, C₃₋₈ cycloalkyl ring, —OR^{8b}, —SR^{8b}, —N(R^{8b})₂, —C(O)O(C₁₋₄ alkyl), —C(O)OH, —NR(CO)CO(C₁₋₄ alkyl) or an oxo group; wherein each said cycloalkyl group is optionally and independently substituted with up to 3 instances of halogen;
each R^{7d} is independently selected from halogen, —CN, —NO₂, C₁₋₄ alkyl, C₁₋₄ haloalkyl, C₃₋₈ cycloalkyl ring, —C(O)O(C₁₋₄ alkyl), —C(O)OH, —OR^{8c}, —SR^{8c}, —N(R^{8c})₂, or an oxo group; wherein each said cycloalkyl group is optionally and independently substituted with up to 3 instances of halogen;
each R^{8b} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{8c} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; and
R^{C2} is selected from hydrogen, halogen, —OH, —O(C₁₋₆ alkyl), —O(haloC₁₋₆ alkyl), —O(C₁₋₆ haloalkyl), —O(cyclopropyl), cyclopropyl, C₁₋₆ alkyl, C₁₋₆ haloalkyl and —CN.

In some embodiments of the compounds of Formula I′ or Formula I the compound is one of Formula I′B or a pharmaceutically acceptable salt thereof:

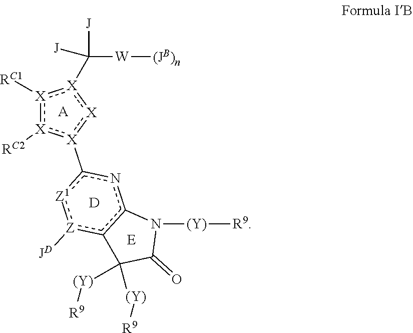

In some embodiments of the compounds of Formula I′, Formula I or Formula I′B, the compound is one of Formula I′C or a pharmaceutically acceptable salt thereof:

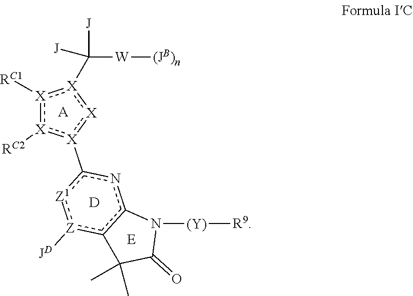

In some of the above embodiments, the compound is one selected from the Table X, below, or a pharmaceutically acceptable salt thereof:

TABLE X

| | |
|---|---|
| 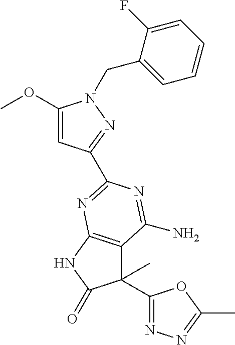 | I-1 |
| 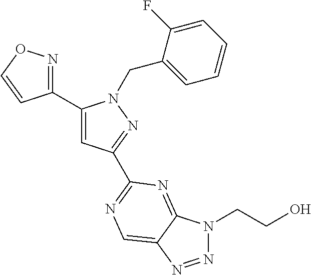 [SMILES: OCCn1nnc2cnc(-c3cc(-c4ccon4)n(Cc4ccccc4F)n3)nc21] | I-2 |
| 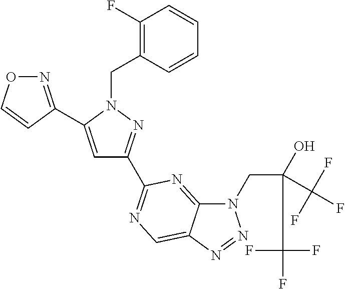 [SMILES: OC(Cn1nnc2cnc(-c3cc(-c4ccon4)n(Cc4ccccc4F)n3)nc21)(C(F)(F)F)C(F)(F)F] | I-3 |
| 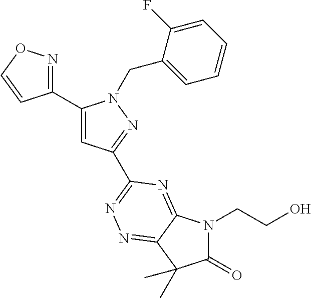 [SMILES: CC1(C)C(=O)N(CCO)c2nc(-c3cc(-c4ccon4)n(Cc4ccccc4F)n3)nnc21] | I-4 |
| 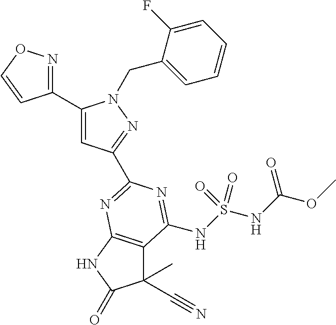 [SMILES: COC(=O)NS(=O)(=O)Nc1nc(-c2cc(-c3ccon3)n(Cc3ccccc3F)n2)nc2c1C(C)(C#N)C(=O)N2] | I-5 |
| 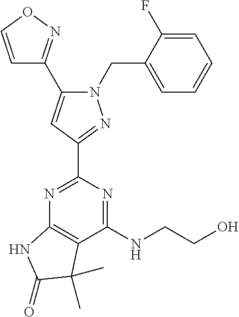 [SMILES: CC1(C)C(=O)Nc2nc(-c3cc(-c4ccon4)n(Cc4ccccc4F)n3)nc(NCCO)c21] | I-6 |
| 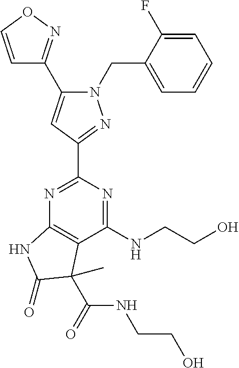 [SMILES: CC1(C(=O)NCCO)C(=O)Nc2nc(-c3cc(-c4ccon4)n(Cc4ccccc4F)n3)nc(NCCO)c21] | I-7 |
| 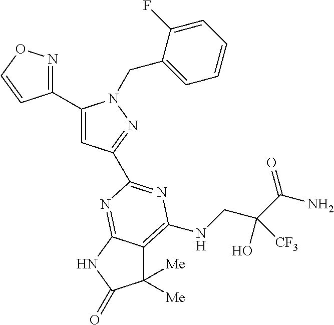 [SMILES: CC1(C)C(=O)Nc2nc(-c3cc(-c4ccon4)n(Cc4ccccc4F)n3)nc(NCC(O)(C(N)=O)C(F)(F)F)c21] | I-8 |
| 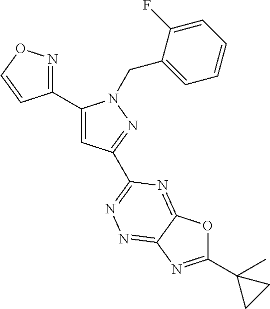 [SMILES: CC1(c2nc3nnc(-c4cc(-c5ccon5)n(Cc5ccccc5F)n4)nc3o2)CC1] | I-9 |
| 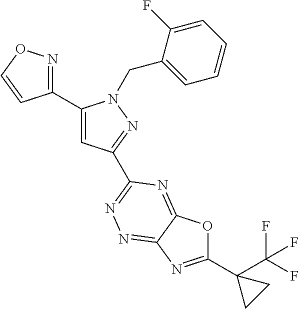 [SMILES: Fc1ccccc1Cn1nc(-c2nnc3nc(C4(C(F)(F)F)CC4)oc3n2)cc1-c1ccon1] | I-10 |
| 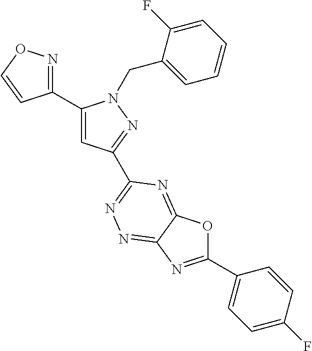 [SMILES: Fc1ccc(-c2nc3nnc(-c4cc(-c5ccon5)n(Cc5ccccc5F)n4)nc3o2)cc1] | I-11 |
| 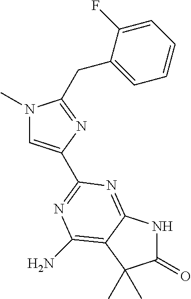 [SMILES: Cn1cc(-c2nc(N)c3c(n2)NC(=O)C3(C)C)nc1Cc1ccccc1F] | I-12 |
| 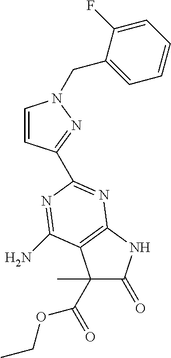 [SMILES: CCOC(=O)C1(C)C(=O)Nc2nc(-c3ccn(Cc4ccccc4F)n3)nc(N)c21] | I-13 |
| 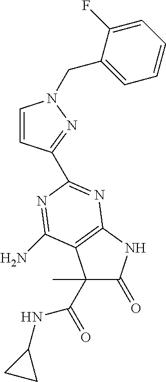 [SMILES: CC1(C(=O)NC2CC2)C(=O)Nc2nc(-c3ccn(Cc4ccccc4F)n3)nc(N)c21] | I-14 |
| 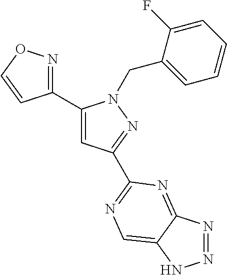 [SMILES: Fc1ccccc1Cn1nc(-c2ncc3[nH]nnc3n2)cc1-c1ccon1] | I-15 |
| 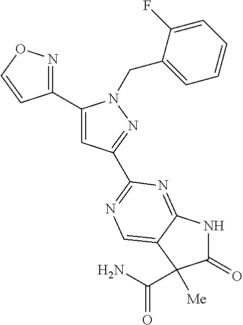 [SMILES: CC1(C(N)=O)C(=O)Nc2nc(-c3cc(-c4ccon4)n(Cc4ccccc4F)n3)ncc21] | I-16 |
| 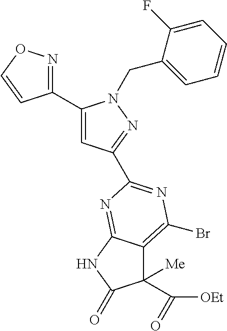 [SMILES: CCOC(=O)C1(C)C(=O)Nc2nc(-c3cc(-c4ccon4)n(Cc4ccccc4F)n3)nc(Br)c21] | I-17 |
| 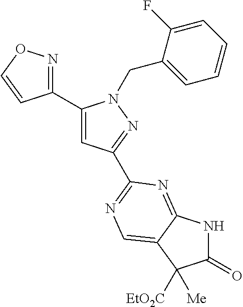 [SMILES: CCOC(=O)C1(C)C(=O)Nc2nc(-c3cc(-c4ccon4)n(Cc4ccccc4F)n3)ncc21] | I-18 |
| 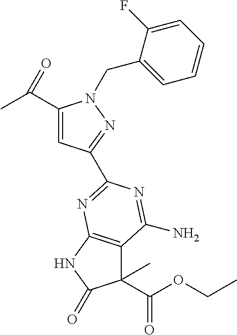 [SMILES: CCOC(=O)C1(C)C(=O)Nc2nc(-c3cc(C(C)=O)n(Cc4ccccc4F)n3)nc(N)c21] | I-19 |
| 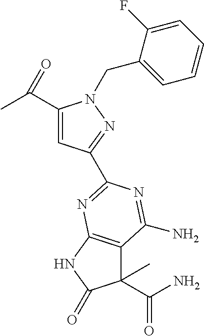 [SMILES: CC(=O)c1cc(-c2nc(N)c3c(n2)NC(=O)C3(C)C(N)=O)nn1Cc1ccccc1F] | I-20 |
| 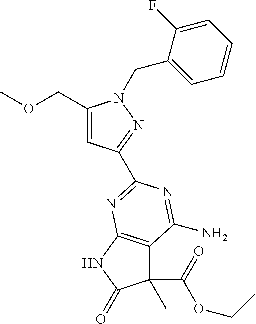 [SMILES: CCOC(=O)C1(C)C(=O)Nc2nc(-c3cc(COC)n(Cc4ccccc4F)n3)nc(N)c21] | I-21 |
| 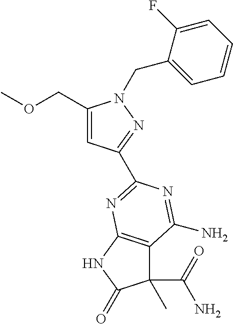 [SMILES: COCc1cc(-c2nc(N)c3c(n2)NC(=O)C3(C)C(N)=O)nn1Cc1ccccc1F] | I-22 |
| 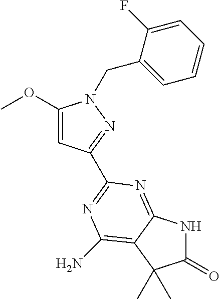 [SMILES: COc1cc(-c2nc(N)c3c(n2)NC(=O)C3(C)C)nn1Cc1ccccc1F] | I-23 |
| 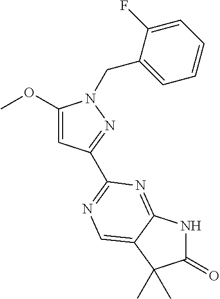 [SMILES: COc1cc(-c2ncc3c(n2)NC(=O)C3(C)C)nn1Cc1ccccc1F] | I-24 |
| 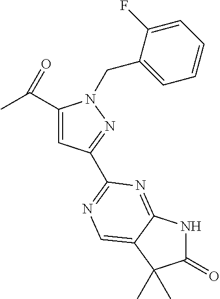 [SMILES: CC(=O)c1cc(-c2ncc3c(n2)NC(=O)C3(C)C)nn1Cc1ccccc1F] | I-25 |
| 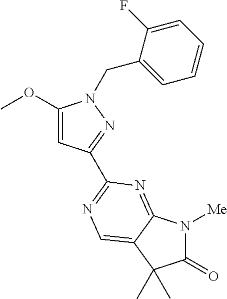 [SMILES: COc1cc(-c2ncc3c(n2)N(C)C(=O)C3(C)C)nn1Cc1ccccc1F] | I-26 |
| 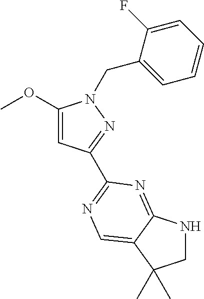 [SMILES: COc1cc(-c2ncc3c(n2)NCC3(C)C)nn1Cc1ccccc1F] | I-27 |
| 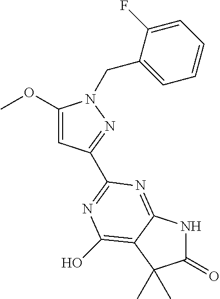 [SMILES: COc1cc(-c2nc(O)c3c(n2)NC(=O)C3(C)C)nn1Cc1ccccc1F] | I-28 |
| 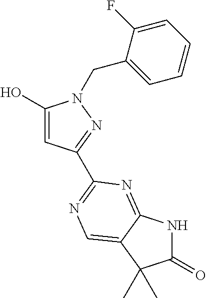 [SMILES: CC1(C)C(=O)Nc2nc(-c3cc(O)n(Cc4ccccc4F)n3)ncc21] | I-29 |
| 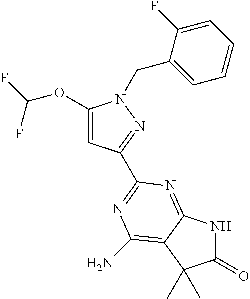 [SMILES: CC1(C)C(=O)Nc2nc(-c3cc(OC(F)F)n(Cc4ccccc4F)n3)nc(N)c21] | I-30 |
| 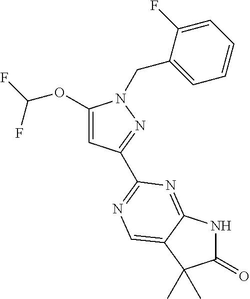 [SMILES: CC1(C)C(=O)Nc2nc(-c3cc(OC(F)F)n(Cc4ccccc4F)n3)ncc21] | I-31 |
| 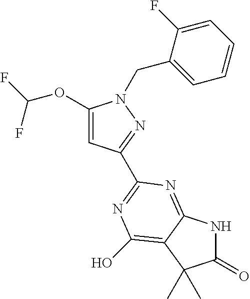 [SMILES: CC1(C)C(=O)Nc2nc(-c3cc(OC(F)F)n(Cc4ccccc4F)n3)nc(O)c21] | I-32 |
| 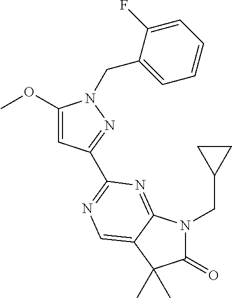 [SMILES: COc1cc(-c2ncc3c(n2)N(CC2CC2)C(=O)C3(C)C)nn1Cc1ccccc1F] | I-33 |
| 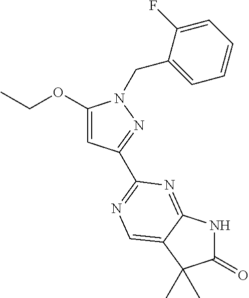 [SMILES: CCOc1cc(-c2ncc3c(n2)NC(=O)C3(C)C)nn1Cc1ccccc1F] | I-34 |
| 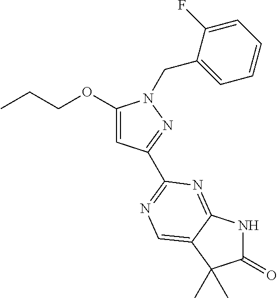 [SMILES: CCCOc1cc(-c2ncc3c(n2)NC(=O)C3(C)C)nn1Cc1ccccc1F] | I-35 |
| 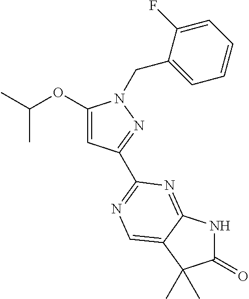 [SMILES: CC(C)Oc1cc(-c2ncc3c(n2)NC(=O)C3(C)C)nn1Cc1ccccc1F] | I-36 |
| 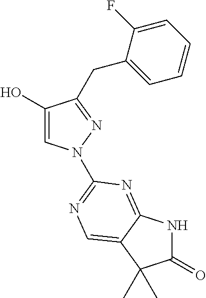 [SMILES: CC1(C)C(=O)Nc2nc(-n3cc(O)c(Cc4ccccc4F)n3)ncc21] | I-37 |
| 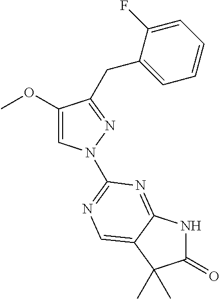 [SMILES: COc1cn(-c2ncc3c(n2)NC(=O)C3(C)C)nc1Cc1ccccc1F] | I-38 |
| 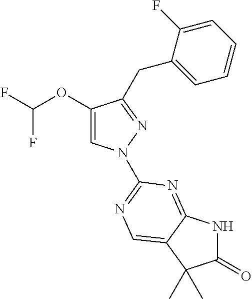 [SMILES: CC1(C)C(=O)Nc2nc(-n3cc(OC(F)F)c(Cc4ccccc4F)n3)ncc21] | I-39 |
| 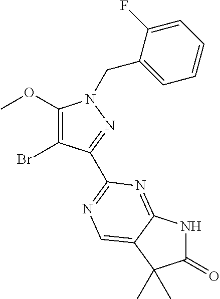 [SMILES: COc1c(Br)c(-c2ncc3c(n2)NC(=O)C3(C)C)nn1Cc1ccccc1F] | I-40 |
| 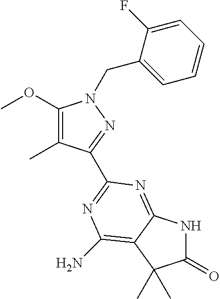 [SMILES: COc1c(C)c(-c2nc(N)c3c(n2)NC(=O)C3(C)C)nn1Cc1ccccc1F] | I-41 |
| 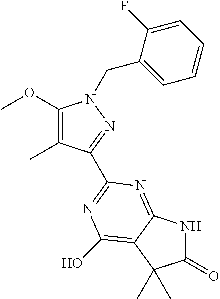 [SMILES: COc1c(C)c(-c2nc(O)c3c(n2)NC(=O)C3(C)C)nn1Cc1ccccc1F] | I-42 |
| 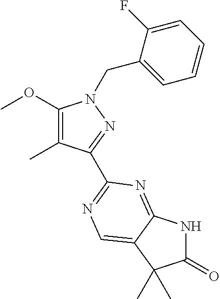 [SMILES: COc1c(C)c(-c2ncc3c(n2)NC(=O)C3(C)C)nn1Cc1ccccc1F] | I-43 |
| 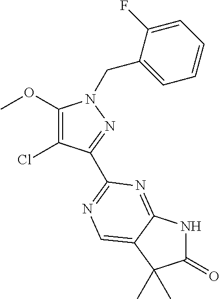 [SMILES: COc1c(Cl)c(-c2ncc3c(n2)NC(=O)C3(C)C)nn1Cc1ccccc1F] | I-44 |
| 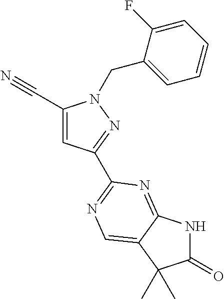 [SMILES: CC1(C)C(=O)Nc2nc(-c3cc(C#N)n(Cc4ccccc4F)n3)ncc21] | I-45 |
| 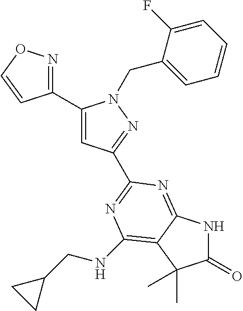 [SMILES: CC1(C)C(=O)Nc2nc(-c3cc(-c4ccon4)n(Cc4ccccc4F)n3)nc(NCC3CC3)c21] | I-46 |
| 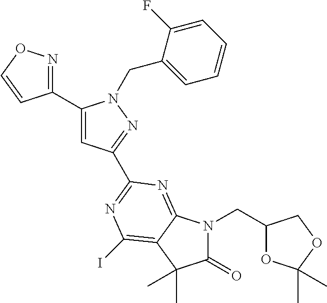 [SMILES: CC1(C)OCC(CN2C(=O)C(C)(C)c3c(I)nc(-c4cc(-c5ccon5)n(Cc5ccccc5F)n4)nc32)O1] | I-47 |
| 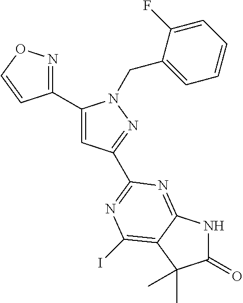 [SMILES: CC1(C)C(=O)Nc2nc(-c3cc(-c4ccon4)n(Cc4ccccc4F)n3)nc(I)c21] | I-48 |
| 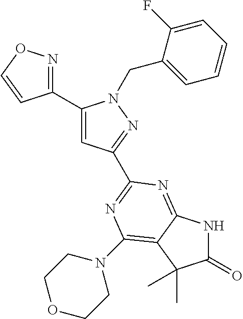 [SMILES: CC1(C)C(=O)Nc2nc(-c3cc(-c4ccon4)n(Cc4ccccc4F)n3)nc(N3CCOCC3)c21] | I-49 |
| 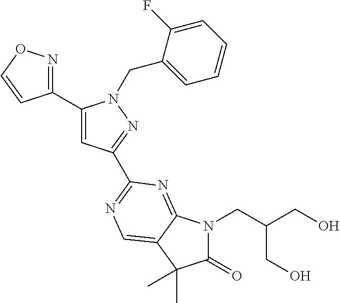 [SMILES: CC1(C)C(=O)N(CC(CO)CO)c2nc(-c3cc(-c4ccon4)n(Cc4ccccc4F)n3)ncc21] | I-50 |
| 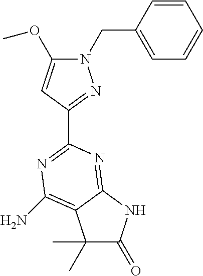 [SMILES: COc1cc(-c2nc(N)c3c(n2)NC(=O)C3(C)C)nn1Cc1ccccc1] | I-51 |
| 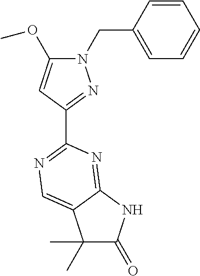 [SMILES: COc1cc(-c2ncc3c(n2)NC(=O)C3(C)C)nn1Cc1ccccc1] | I-52 |
| 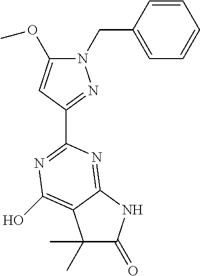 [SMILES: COc1cc(-c2nc(O)c3c(n2)NC(=O)C3(C)C)nn1Cc1ccccc1] | I-53 |
| 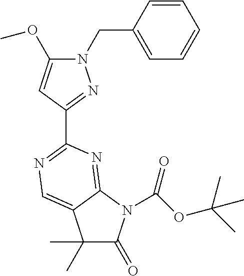 [SMILES: COc1cc(-c2ncc3c(n2)N(C(=O)OC(C)(C)C)C(=O)C3(C)C)nn1Cc1ccccc1] | I-54 |
| 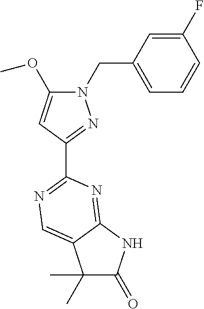 [SMILES: COc1cc(-c2ncc3c(n2)NC(=O)C3(C)C)nn1Cc1cccc(F)c1] | I-55 |
| 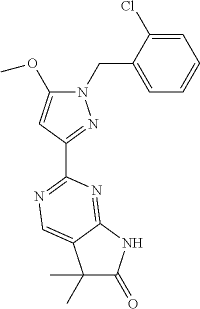 [SMILES: COc1cc(-c2ncc3c(n2)NC(=O)C3(C)C)nn1Cc1ccccc1Cl] | I-56 |
| 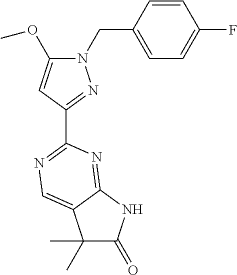 [SMILES: COc1cc(-c2ncc3c(n2)NC(=O)C3(C)C)nn1Cc1ccc(F)cc1] | I-57 |
| 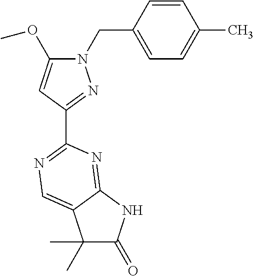 | I-58 |
| 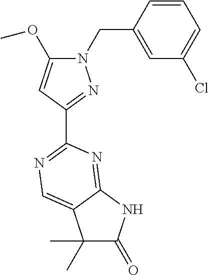 [SMILES: COc1cc(-c2ncc3c(n2)NC(=O)C3(C)C)nn1Cc1cccc(Cl)c1] | I-59 |
| 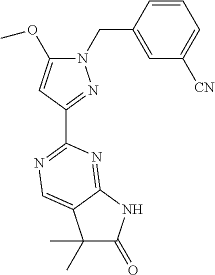 [SMILES: COc1cc(-c2ncc3c(n2)NC(=O)C3(C)C)nn1Cc1cccc(C#N)c1] | I-60 |
| 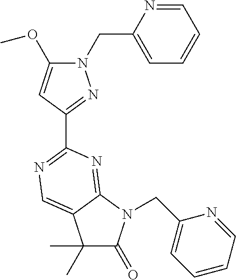 [SMILES: COc1cc(-c2ncc3c(n2)N(Cc2ccccn2)C(=O)C3(C)C)nn1Cc1ccccn1] | I-61 |
| 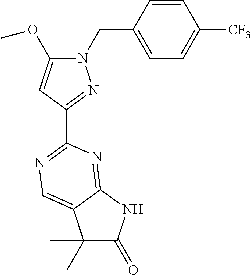 [SMILES: COc1cc(-c2ncc3c(n2)NC(=O)C3(C)C)nn1Cc1ccc(C(F)(F)F)cc1] | I-62 |
| 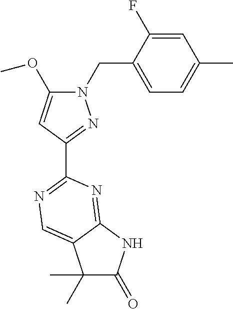 | I-63 |
| 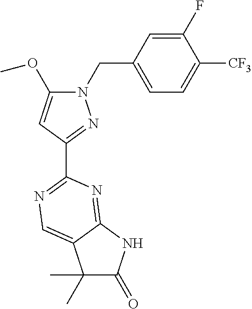 [SMILES: COc1cc(-c2ncc3c(n2)NC(=O)C3(C)C)nn1Cc1ccc(C(F)(F)F)c(F)c1] | I-64 |
| 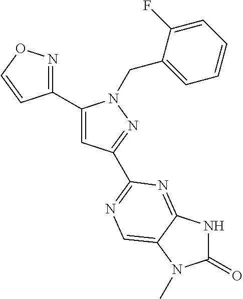 [SMILES: Cn1c(=O)[nH]c2nc(-c3cc(-c4ccon4)n(Cc4ccccc4F)n3)ncc21] | I-65 |
| 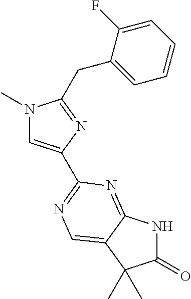 [SMILES: Cn1cc(-c2ncc3c(n2)NC(=O)C3(C)C)nc1Cc1ccccc1F] | I-66 |
| 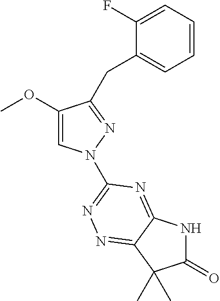 [SMILES: COc1cn(-c2nnc3c(n2)NC(=O)C3(C)C)nc1Cc1ccccc1F] | I-67 |
| 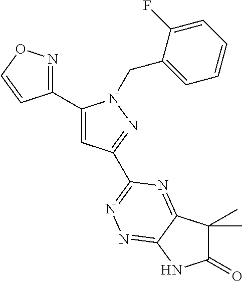 [SMILES: CC1(C)C(=O)Nc2nnc(-c3cc(-c4ccon4)n(Cc4ccccc4F)n3)nc21] | I-68 |
| 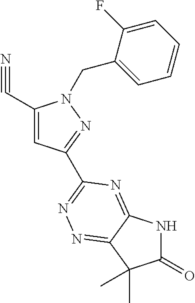 [SMILES: CC1(C)C(=O)Nc2nc(-c3cc(C#N)n(Cc4ccccc4F)n3)nnc21] | I-69 |
| 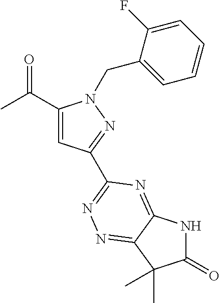 [SMILES: CC(=O)c1cc(-c2nnc3c(n2)NC(=O)C3(C)C)nn1Cc1ccccc1F] | I-70 |
| 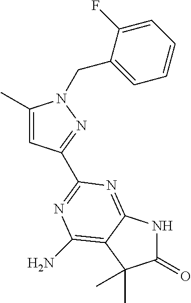 [SMILES: Cc1cc(-c2nc(N)c3c(n2)NC(=O)C3(C)C)nn1Cc1ccccc1F] | I-71 |
| 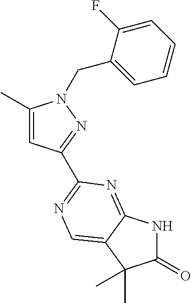 [SMILES: Cc1cc(-c2ncc3c(n2)NC(=O)C3(C)C)nn1Cc1ccccc1F] | I-72 |
| 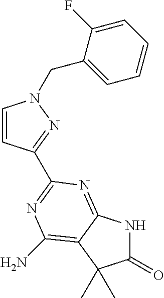 [SMILES: CC1(C)C(=O)Nc2nc(-c3ccn(Cc4ccccc4F)n3)nc(N)c21] | I-73 |
| 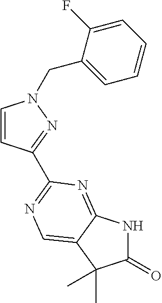 [SMILES: CC1(C)C(=O)Nc2nc(-c3ccn(Cc4ccccc4F)n3)ncc21] | I-74 |
| 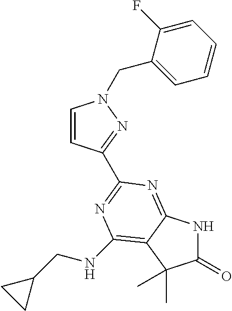 [SMILES: CC1(C)C(=O)Nc2nc(-c3ccn(Cc4ccccc4F)n3)nc(NCC3CC3)c21] | I-75 |
| 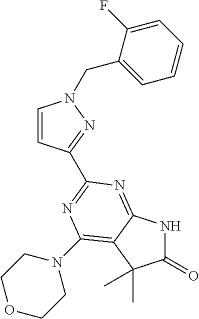 [SMILES: CC1(C)C(=O)Nc2nc(-c3ccn(Cc4ccccc4F)n3)nc(N3CCOCC3)c21] | I-76 |
| 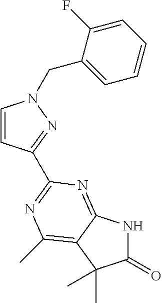 [SMILES: Cc1nc(-c2ccn(Cc3ccccc3F)n2)nc2c1C(C)(C)C(=O)N2] | I-77 |
| 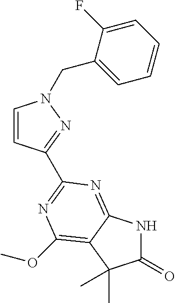 [SMILES: COc1nc(-c2ccn(Cc3ccccc3F)n2)nc2c1C(C)(C)C(=O)N2] | I-78 |
| 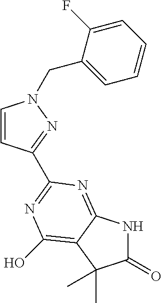 [SMILES: CC1(C)C(=O)Nc2nc(-c3ccn(Cc4ccccc4F)n3)nc(O)c21] | I-79 |
| 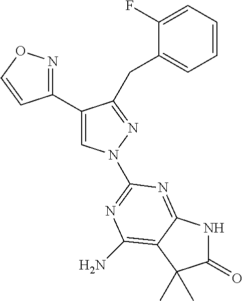 [SMILES: CC1(C)C(=O)Nc2nc(-n3cc(-c4ccon4)c(Cc4ccccc4F)n3)nc(N)c21] | I-80 |
| 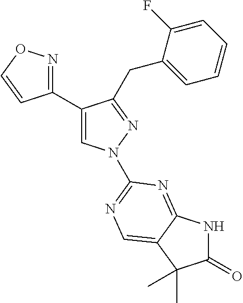 [SMILES: CC1(C)C(=O)Nc2nc(-n3cc(-c4ccon4)c(Cc4ccccc4F)n3)ncc21] | I-81 |
| 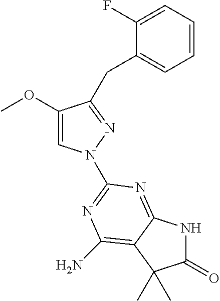 [SMILES: COc1cn(-c2nc(N)c3c(n2)NC(=O)C3(C)C)nc1Cc1ccccc1F] | I-82 |
| 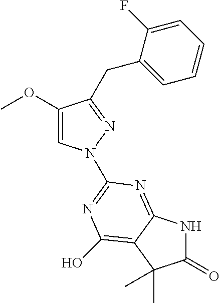 [SMILES: COc1cn(-c2nc(O)c3c(n2)NC(=O)C3(C)C)nc1Cc1ccccc1F] | I-83 |
| 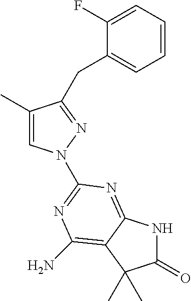 [SMILES: Cc1cn(-c2nc(N)c3c(n2)NC(=O)C3(C)C)nc1Cc1ccccc1F] | I-84 |
| 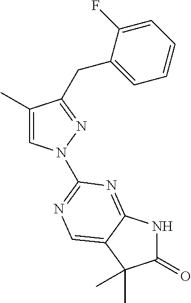 [SMILES: Cc1cn(-c2ncc3c(n2)NC(=O)C3(C)C)nc1Cc1ccccc1F] | I-85 |
| 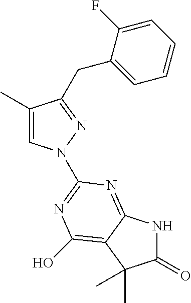 [SMILES: Cc1cn(-c2nc(O)c3c(n2)NC(=O)C3(C)C)nc1Cc1ccccc1F] | I-86 |
| 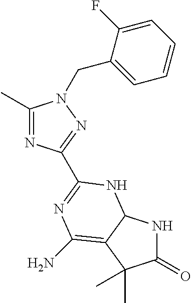 [SMILES: Cc1nc(C2=NC(N)=C3C(NC(=O)C3(C)C)N2)nn1Cc1ccccc1F] | I-87 |
| 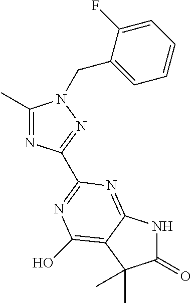 [SMILES: Cc1nc(-c2nc(O)c3c(n2)NC(=O)C3(C)C)nn1Cc1ccccc1F] | I-88 |
| 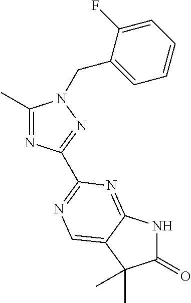 [SMILES: Cc1nc(-c2ncc3c(n2)NC(=O)C3(C)C)nn1Cc1ccccc1F] | I-89 |
| 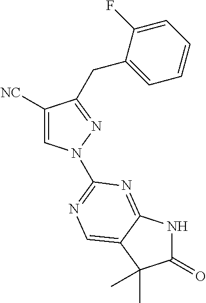 [SMILES: CC1(C)C(=O)Nc2nc(-n3cc(C#N)c(Cc4ccccc4F)n3)ncc21] | I-90 |
| 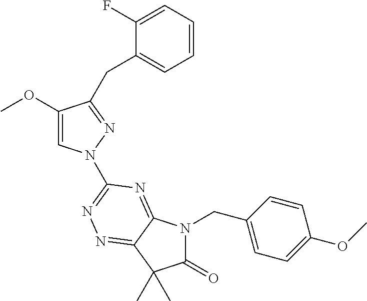 | I-91 |
| 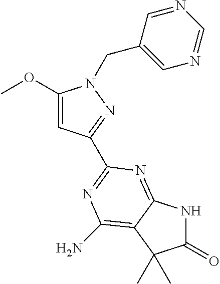 [SMILES: COc1cc(-c2nc(N)c3c(n2)NC(=O)C3(C)C)nn1Cc1cncnc1] | I-92 |
| 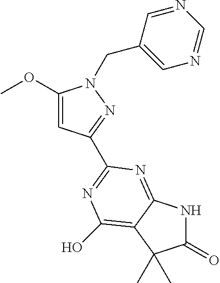 [SMILES: COc1cc(-c2nc(O)c3c(n2)NC(=O)C3(C)C)nn1Cc1cncnc1] | I-93 |
| 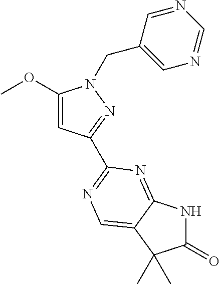 [SMILES: COc1cc(-c2ncc3c(n2)NC(=O)C3(C)C)nn1Cc1cncnc1] | I-94 |

In some of the above embodiments, the compound is one selected from Table XX, below, or a pharmaceutically acceptable salt thereof:

TABLE XX

| | |
|---|---|
| 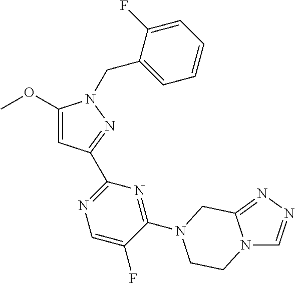 [SMILES: COc1cc(-c2ncc(F)c(N3CCn4cnnc4C3)n2)nn1Cc1ccccc1F] | XI-1 |
| 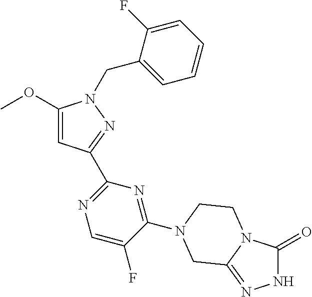 [SMILES: COc1cc(-c2ncc(F)c(N3CCn4c(n[nH]c4=O)C3)n2)nn1Cc1ccccc1F] | XI-2 |
| 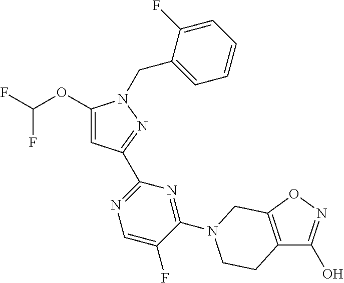 [SMILES: Oc1noc2c1CCN(c1nc(-c3cc(OC(F)F)n(Cc4ccccc4F)n3)ncc1F)C2] | XI-3 |
| 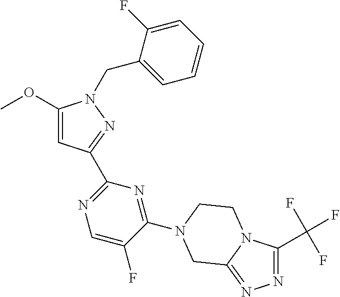 [SMILES: COc1cc(-c2ncc(F)c(N3CCn4c(nnc4C(F)(F)F)C3)n2)nn1Cc1ccccc1F] | XI-4 |
| 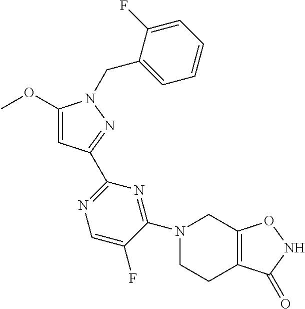 [SMILES: COc1cc(-c2ncc(F)c(N3CCc4c(o[nH]c4=O)C3)n2)nn1Cc1ccccc1F] | XI-5 |
| 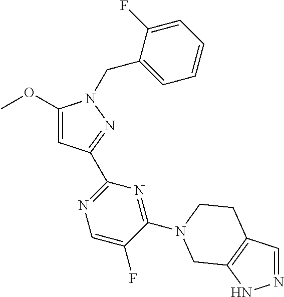 [SMILES: COc1cc(-c2ncc(F)c(N3CCc4cn[nH]c4C3)n2)nn1Cc1ccccc1F] | XI-6 |
| 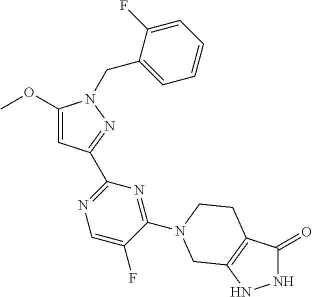 [SMILES: COc1cc(-c2ncc(F)c(N3CCc4c([nH][nH]c4=O)C3)n2)nn1Cc1ccccc1F] | XI-7 |
| 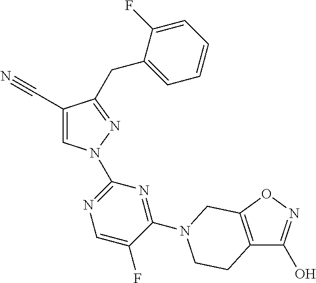 [SMILES: N#Cc1cn(-c2ncc(F)c(N3CCc4c(O)noc4C3)n2)nc1Cc1ccccc1F] | XI-8 |
| 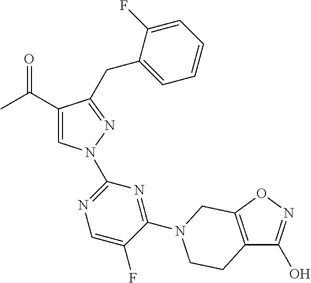 [SMILES: CC(=O)c1cn(-c2ncc(F)c(N3CCc4c(O)noc4C3)n2)nc1Cc1ccccc1F] | XI-9 |
| 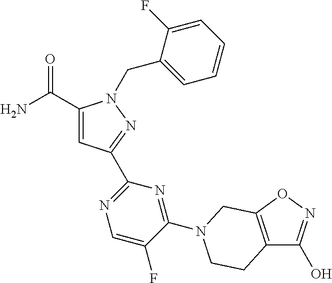 [SMILES: NC(=O)c1cc(-c2ncc(F)c(N3CCc4c(O)noc4C3)n2)nn1Cc1ccccc1F] | XI-10 |
| 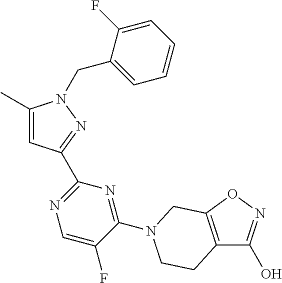 [SMILES: Cc1cc(-c2ncc(F)c(N3CCc4c(O)noc4C3)n2)nn1Cc1ccccc1F] | XI-12 |
| 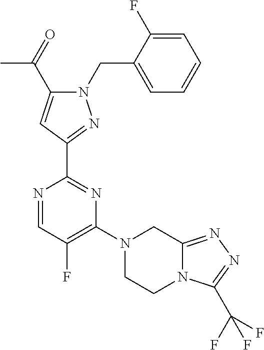 [SMILES: CC(=O)c1cc(-c2ncc(F)c(N3CCn4c(nnc4C(F)(F)F)C3)n2)nn1Cc1ccccc1F] | XI-13 |
| 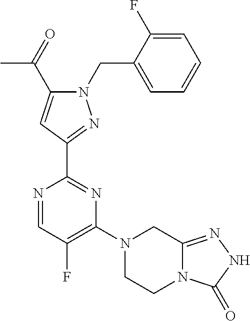 [SMILES: CC(=O)c1cc(-c2ncc(F)c(N3CCn4c(n[nH]c4=O)C3)n2)nn1Cc1ccccc1F] | XI-14 |
| 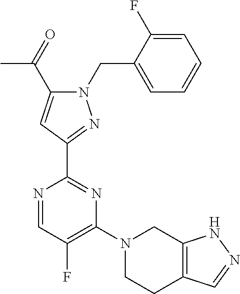 [SMILES: CC(=O)c1cc(-c2ncc(F)c(N3CCc4cn[nH]c4C3)n2)nn1Cc1ccccc1F] | XI-15 |
| 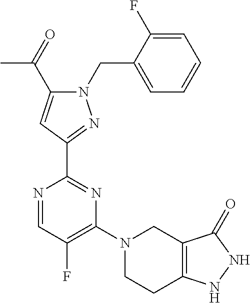 [SMILES: CC(=O)c1cc(-c2ncc(F)c(N3CCc4[nH][nH]c(=O)c4C3)n2)nn1Cc1ccccc1F] | XI-16 |
| 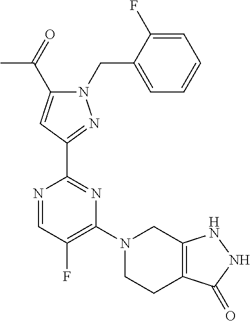 [SMILES: CC(=O)c1cc(-c2ncc(F)c(N3CCc4c([nH][nH]c4=O)C3)n2)nn1Cc1ccccc1F] | XI-17 |
| 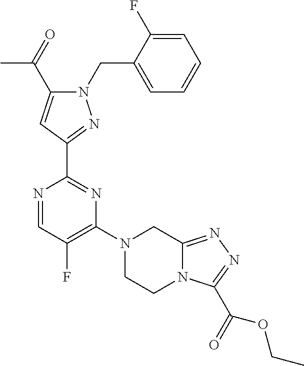 [SMILES: CCOC(=O)c1nnc2n1CCN(c1nc(-c3cc(C(C)=O)n(Cc4ccccc4F)n3)ncc1F)C2] | XI-18 |
| 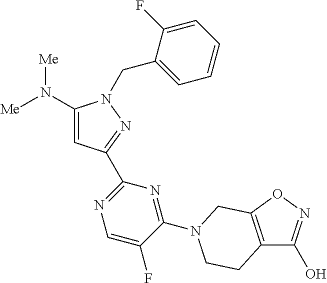 [SMILES: CN(C)c1cc(-c2ncc(F)c(N3CCc4c(O)noc4C3)n2)nn1Cc1ccccc1F] | XI-19 |
| 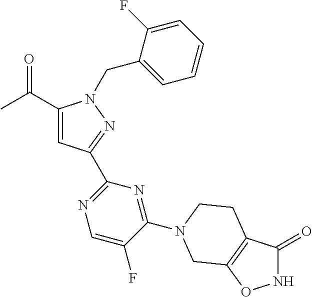 [SMILES: CC(=O)c1cc(-c2ncc(F)c(N3CCc4c(o[nH]c4=O)C3)n2)nn1Cc1ccccc1F] | XI-20 |
| 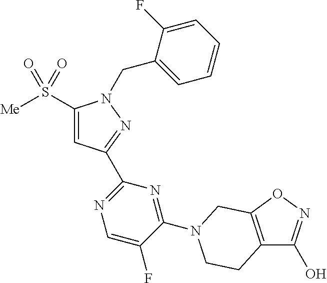 [SMILES: CS(=O)(=O)c1cc(-c2ncc(F)c(N3CCc4c(O)noc4C3)n2)nn1Cc1ccccc1F] | XI-21 |
| 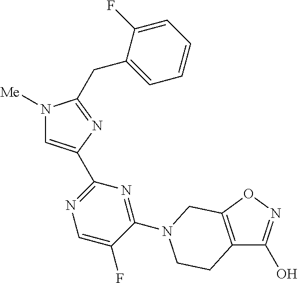 [SMILES: Cn1cc(-c2ncc(F)c(N3CCc4c(O)noc4C3)n2)nc1Cc1ccccc1F] | XI-22 |
| 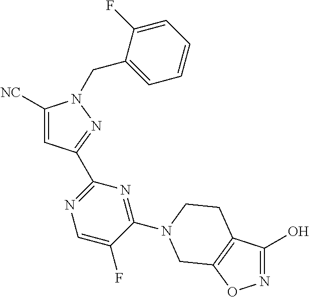 [SMILES: N#Cc1cc(-c2ncc(F)c(N3CCc4c(O)noc4C3)n2)nn1Cc1ccccc1F] | XI-11 |

In some embodiments of the invention, the compound is one selected from the Table XXX, below, or a pharmaceutically acceptable salt thereof:

TABLE XXX

| | |
|---|---|
| 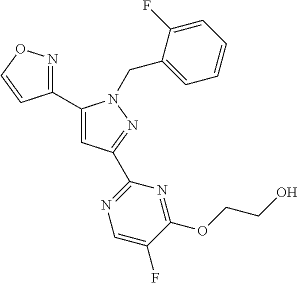 [SMILES: OCCOc1nc(-c2cc(-c3ccon3)n(Cc3ccccc3F)n2)ncc1F] | XXI- 1 |
| 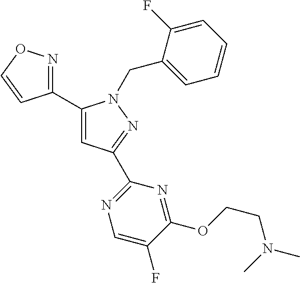 [SMILES: CN(C)CCOc1nc(-c2cc(-c3ccon3)n(Cc3ccccc3F)n2)ncc1F] | XXI- 2 |
| 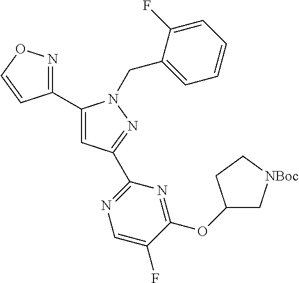 [SMILES: CC(C)(C)OC(=O)N1CCC(Oc2nc(-c3cc(-c4ccon4)n(Cc4ccccc4F)n3)ncc2F)C1] | XXI- 3 |
| 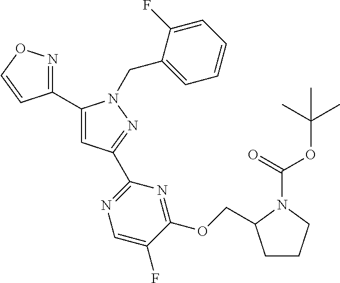 [SMILES: CC(C)(C)OC(=O)N1CCCC1COc1nc(-c2cc(-c3ccon3)n(Cc3ccccc3F)n2)ncc1F] | XXI- 4 |
| 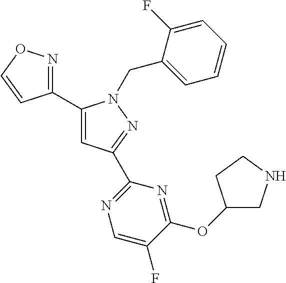 [SMILES: Fc1ccccc1Cn1nc(-c2ncc(F)c(OC3CCNC3)n2)cc1-c1ccon1] | XXI- 5 |
| 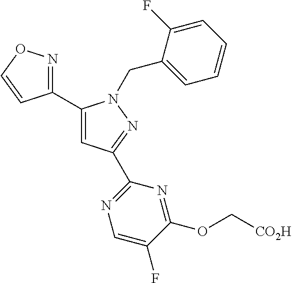 [SMILES: O=C(O)COc1nc(-c2cc(-c3ccon3)n(Cc3ccccc3F)n2)ncc1F] | XXI- 6 |
| 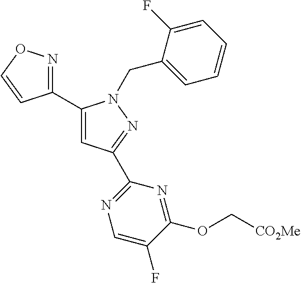 [SMILES: COC(=O)COc1nc(-c2cc(-c3ccon3)n(Cc3ccccc3F)n2)ncc1F] | XXI- 7 |
| 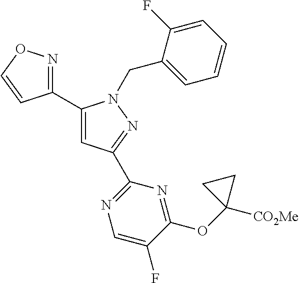 [SMILES: COC(=O)C1(Oc2nc(-c3cc(-c4ccon4)n(Cc4ccccc4F)n3)ncc2F)CC1] | XXI- 8 |
| 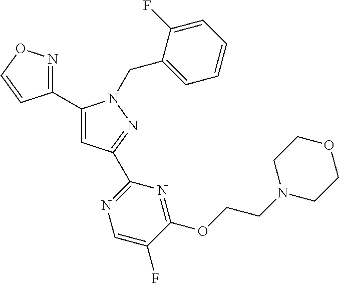 [SMILES: Fc1ccccc1Cn1nc(-c2ncc(F)c(OCCN3CCOCC3)n2)cc1-c1ccon1] | XXI- 9 |
| 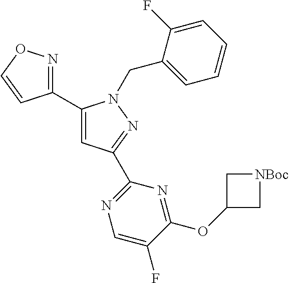 [SMILES: CC(C)(C)OC(=O)N1CC(Oc2nc(-c3cc(-c4ccon4)n(Cc4ccccc4F)n3)ncc2F)C1] | XXI- 10 |
| 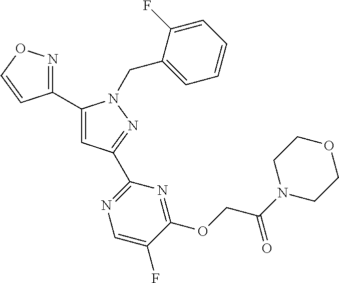 [SMILES: O=C(COc1nc(-c2cc(-c3ccon3)n(Cc3ccccc3F)n2)ncc1F)N1CCOCC1] | XXI- 11 |
| 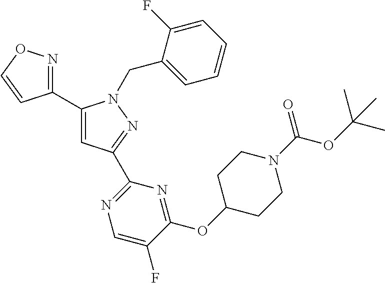 [SMILES: CC(C)(C)OC(=O)N1CCC(Oc2nc(-c3cc(-c4ccon4)n(Cc4ccccc4F)n3)ncc2F)CC1] | XXI- 12 |
| 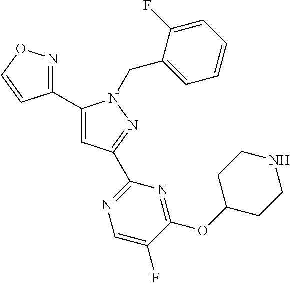 [SMILES: Fc1ccccc1Cn1nc(-c2ncc(F)c(OC3CCNCC3)n2)cc1-c1ccon1] | XXI- 13 |
| 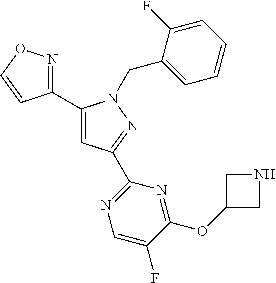 [SMILES: Fc1ccccc1Cn1nc(-c2ncc(F)c(OC3CNC3)n2)cc1-c1ccon1] | XXI- 14 |
| 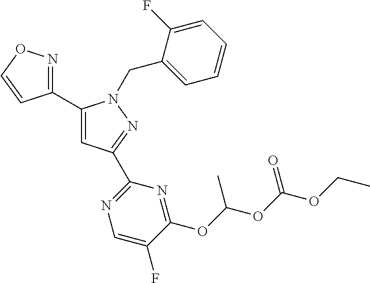 [SMILES: CCOC(=O)OC(C)Oc1nc(-c2cc(-c3ccon3)n(Cc3ccccc3F)n2)ncc1F] | XXI- 29 |
| 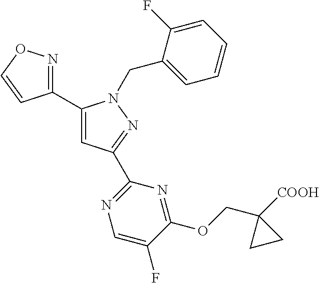 [SMILES: O=C(O)C1(COc2nc(-c3cc(-c4ccon4)n(Cc4ccccc4F)n3)ncc2F)CC1] | XXI- 31 |
| 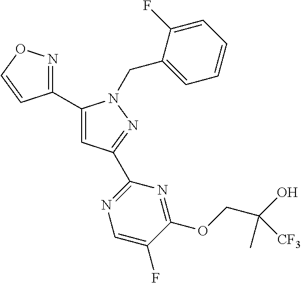 [SMILES: CC(O)(COc1nc(-c2cc(-c3ccon3)n(Cc3ccccc3F)n2)ncc1F)C(F)(F)F] | XXI- 33 |
| 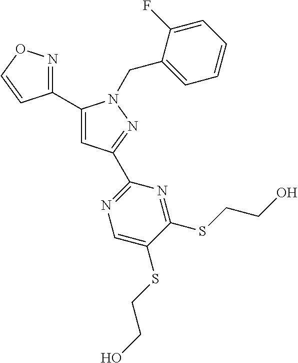 [SMILES: OCCSc1cnc(-c2cc(-c3ccon3)n(Cc3ccccc3F)n2)nc1SCCO] | XXI- 35 |
| 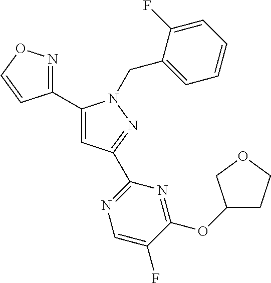 [SMILES: Fc1ccccc1Cn1nc(-c2ncc(F)c(OC3CCOC3)n2)cc1-c1ccon1] | XXI- 15 |
| 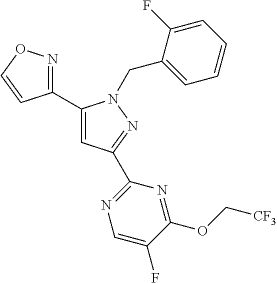 [SMILES: Fc1ccccc1Cn1nc(-c2ncc(F)c(OCC(F)(F)F)n2)cc1-c1ccon1] | XXI- 16 |
| 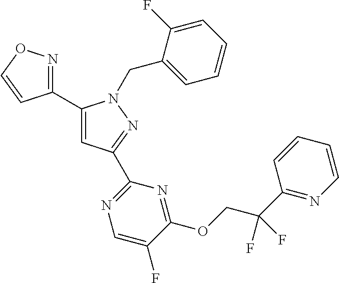 [SMILES: Fc1ccccc1Cn1nc(-c2ncc(F)c(OCC(F)(F)c3ccccn3)n2)cc1-c1ccon1] | XXI- 17 |
| 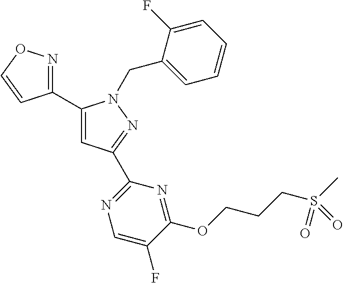 [SMILES: CS(=O)(=O)CCCOc1nc(-c2cc(-c3ccon3)n(Cc3ccccc3F)n2)ncc1F] | XXI- 18 |
| 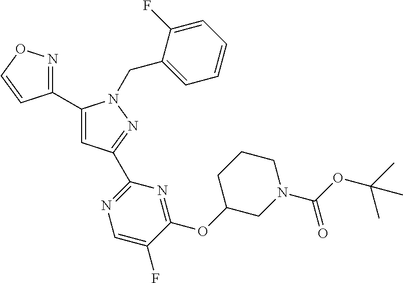 [SMILES: CC(C)(C)OC(=O)N1CCCC(Oc2nc(-c3cc(-c4ccon4)n(Cc4ccccc4F)n3)ncc2F)C1] | XXI- 19 |
| 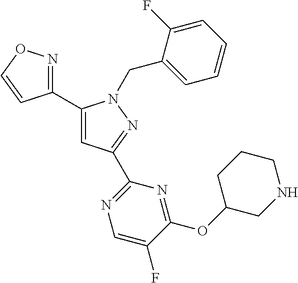 [SMILES: Fc1ccccc1Cn1nc(-c2ncc(F)c(OC3CCCNC3)n2)cc1-c1ccon1] | XXI- 20 |
| 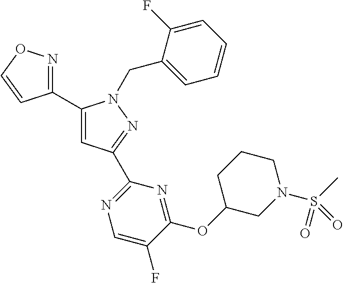 [SMILES: CS(=O)(=O)N1CCCC(Oc2nc(-c3cc(-c4ccon4)n(Cc4ccccc4F)n3)ncc2F)C1] | XXI- 21 |
| 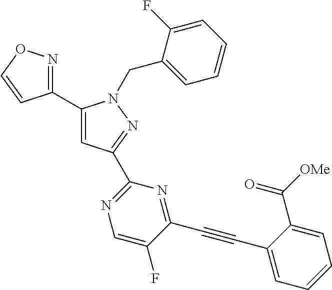 [SMILES: COC(=O)c1ccccc1C#Cc1nc(-c2cc(-c3ccon3)n(Cc3ccccc3F)n2)ncc1F] | XXI- 23 |
| 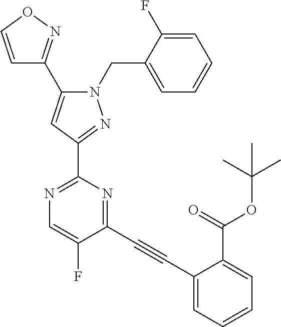 [SMILES: CC(C)(C)OC(=O)c1ccccc1C#Cc1nc(-c2cc(-c3ccon3)n(Cc3ccccc3F)n2)ncc1F] | XXI- 24 |
| 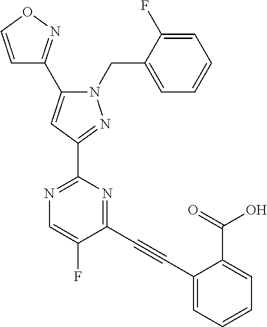 [SMILES: O=C(O)c1ccccc1C#Cc1nc(-c2cc(-c3ccon3)n(Cc3ccccc3F)n2)ncc1F] | XXI- 25 |
| 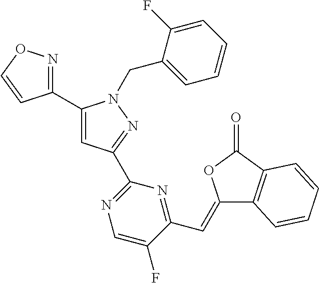 [SMILES: O=C1O/C(=C\c2nc(-c3cc(-c4ccon4)n(Cc4ccccc4F)n3)ncc2F)c2ccccc21] | XXI- 26 |
| 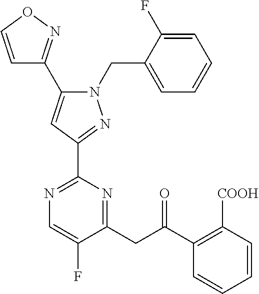 [SMILES: O=C(O)c1ccccc1C(=O)Cc1nc(-c2cc(-c3ccon3)n(Cc3ccccc3F)n2)ncc1F] | XXI- 27 |
| 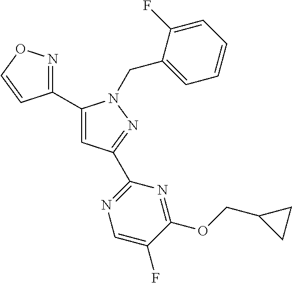 [SMILES: Fc1ccccc1Cn1nc(-c2ncc(F)c(OCC3CC3)n2)cc1-c1ccon1] | XXI- 30 |
| 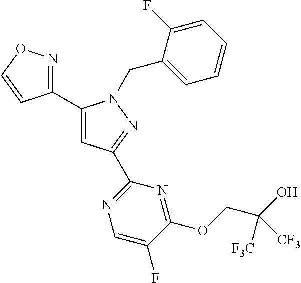 [SMILES: OC(COc1nc(-c2cc(-c3ccon3)n(Cc3ccccc3F)n2)ncc1F)(C(F)(F)F)C(F)(F)F] | XXI- 32 |
| 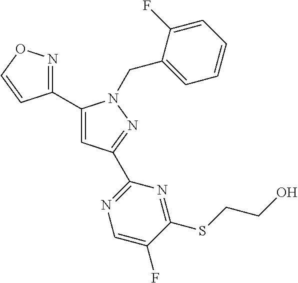 [SMILES: OCCSc1nc(-c2cc(-c3ccon3)n(Cc3ccccc3F)n2)ncc1F] | XXI- 34 |
| 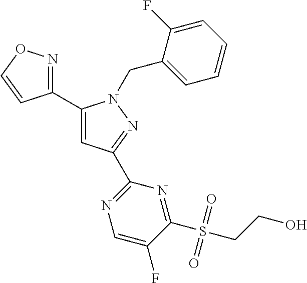 [SMILES: O=S(=O)(CCO)c1nc(-c2cc(-c3ccon3)n(Cc3ccccc3F)n2)ncc1F] | XXI- 36 |
| 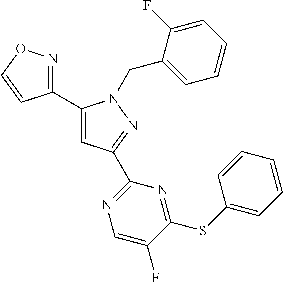 [SMILES: Fc1ccccc1Cn1nc(-c2ncc(F)c(Sc3ccccc3)n2)cc1-c1ccon1] | XXI- 37 |
| 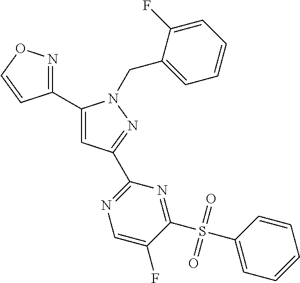 [SMILES: O=S(=O)(c1ccccc1)c1nc(-c2cc(-c3ccon3)n(Cc3ccccc3F)n2)ncc1F] | XXI- 38 |
| 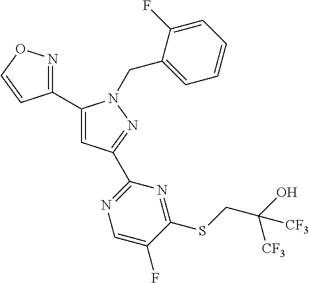 [SMILES: OC(CSc1nc(-c2cc(-c3ccon3)n(Cc3ccccc3F)n2)ncc1F)(C(F)(F)F)C(F)(F)F] | XXI- 39 |

In some embodiments of the above methods, uses, compositions and kits, the sGC stimulator is one depicted in Table IV or Table XIV, or a pharmaceutically acceptable salt thereof.

TABLE IV

| | |
|---|---|
| 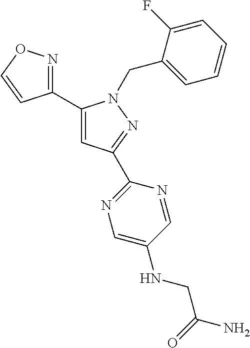 [SMILES: NC(=O)CNc1cnc(-c2cc(-c3ccon3)n(Cc3ccccc3F)n2)nc1] | 6 |
| 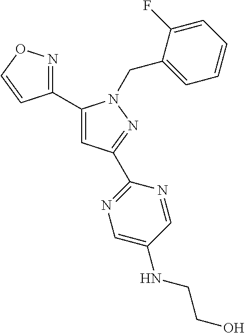 [SMILES: OCCNc1cnc(-c2cc(-c3ccon3)n(Cc3ccccc3F)n2)nc1] | 8 |
| 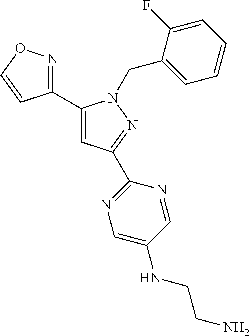 [SMILES: NCCNc1cnc(-c2cc(-c3ccon3)n(Cc3ccccc3F)n2)nc1] | 9 |
| 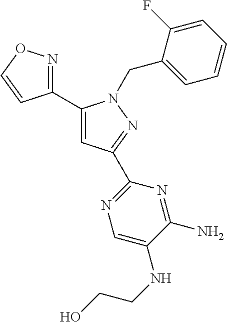 [SMILES: Nc1nc(-c2cc(-c3ccon3)n(Cc3ccccc3F)n2)ncc1NCCO] | 10 |
| 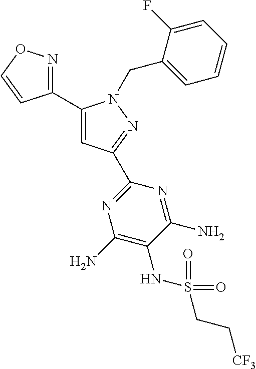 [SMILES: Nc1nc(-c2cc(-c3ccon3)n(Cc3ccccc3F)n2)nc(N)c1NS(=O)(=O)CCC(F)(F)F] | 19 |
| 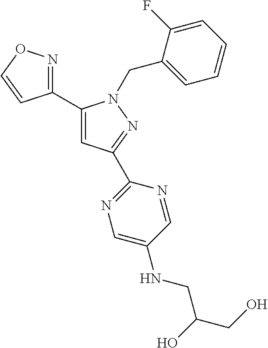 [SMILES: OCC(O)CNc1cnc(-c2cc(-c3ccon3)n(Cc3ccccc3F)n2)nc1] | 21 |
| 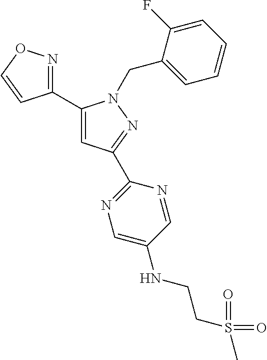 [SMILES: CS(=O)(=O)CCNc1cnc(-c2cc(-c3ccon3)n(Cc3ccccc3F)n2)nc1] | 22 |
| 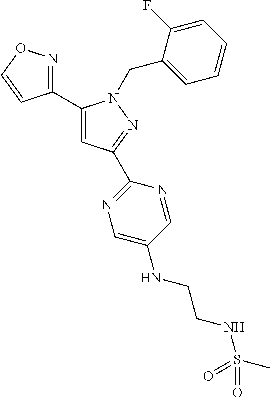 [SMILES: CS(=O)(=O)NCCNc1cnc(-c2cc(-c3ccon3)n(Cc3ccccc3F)n2)nc1] | 24 |
| 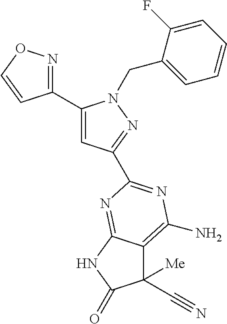 [SMILES: CC1(C#N)C(=O)Nc2nc(-c3cc(-c4ccon4)n(Cc4ccccc4F)n3)nc(N)c21] | 29 |
| 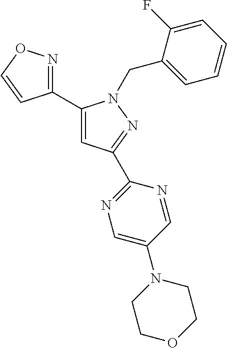 [SMILES: Fc1ccccc1Cn1nc(-c2ncc(N3CCOCC3)cn2)cc1-c1ccon1] | 37 |
| 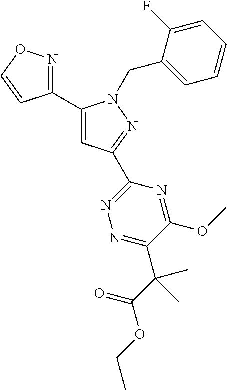 [SMILES: CCOC(=O)C(C)(C)c1nnc(-c2cc(-c3ccon3)n(Cc3ccccc3F)n2)nc1OC] | 61 |
| 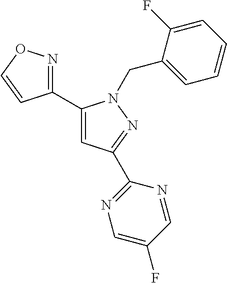 [SMILES: Fc1cnc(-c2cc(-c3ccon3)n(Cc3ccccc3F)n2)nc1] | 109 |
| 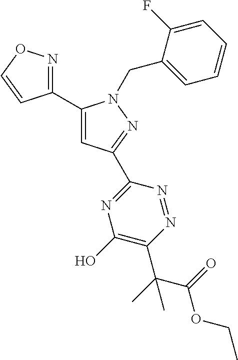 [SMILES: CCOC(=O)C(C)(C)c1nnc(-c2cc(-c3ccon3)n(Cc3ccccc3F)n2)nc1O] | 110 |
| 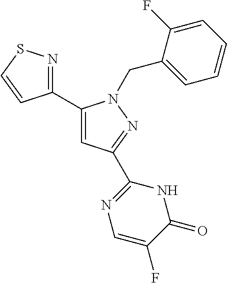 [SMILES: O=c1[nH]c(-c2cc(-c3ccsn3)n(Cc3ccccc3F)n2)ncc1F] | 111 |
| 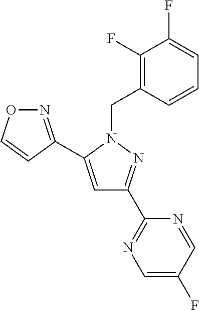 [SMILES: Fc1cnc(-c2cc(-c3ccon3)n(Cc3cccc(F)c3F)n2)nc1] | 142 |
| 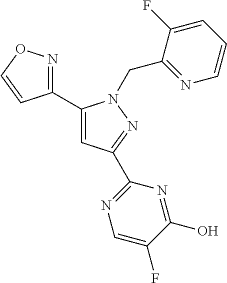 [SMILES: Oc1nc(-c2cc(-c3ccon3)n(Cc3ncccc3F)n2)ncc1F] | 143 |
| 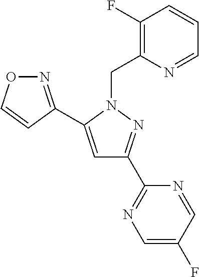 [SMILES: Fc1cnc(-c2cc(-c3ccon3)n(Cc3ncccc3F)n2)nc1] | 144 |
| 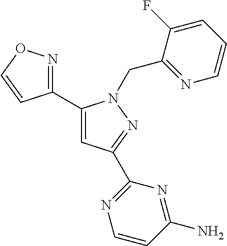 [SMILES: Nc1ccnc(-c2cc(-c3ccon3)n(Cc3ncccc3F)n2)n1] | 145 |
| 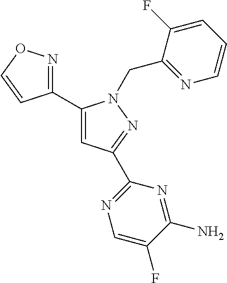 [SMILES: Nc1nc(-c2cc(-c3ccon3)n(Cc3ncccc3F)n2)ncc1F] | 146 |
| 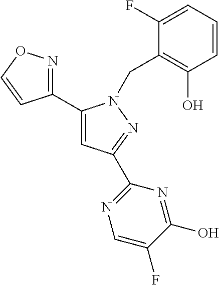 [SMILES: Oc1cccc(F)c1Cn1nc(-c2ncc(F)c(O)n2)cc1-c1ccon1] | 182 |
| 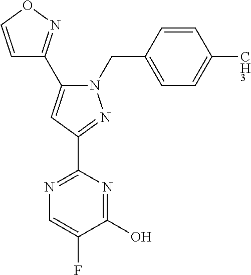 [SMILES: Cc1ccc(Cn2nc(-c3ncc(F)c(O)n3)cc2-c2ccon2)cc1] | 185 |
| 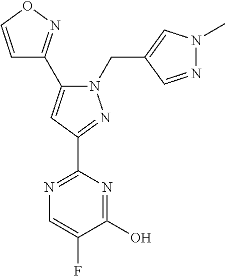 [SMILES: Cn1cc(Cn2nc(-c3ncc(F)c(O)n3)cc2-c2ccon2)cn1] | 186 |
| 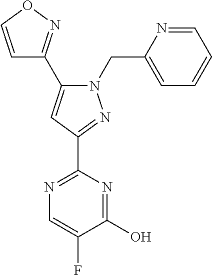 [SMILES: Oc1nc(-c2cc(-c3ccon3)n(Cc3ccccn3)n2)ncc1F] | 187 |
| 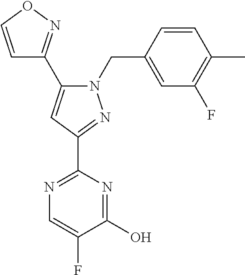 [SMILES: Cc1ccc(Cn2nc(-c3ncc(F)c(O)n3)cc2-c2ccon2)cc1F] | 188 |
| 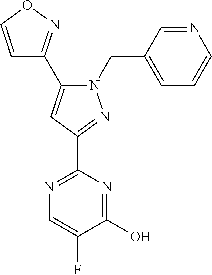 [SMILES: Oc1nc(-c2cc(-c3ccon3)n(Cc3cccnc3)n2)ncc1F] | 189 |
| 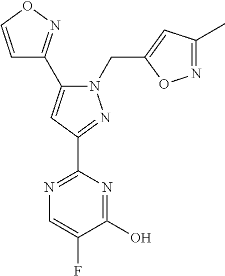 [SMILES: Cc1cc(Cn2nc(-c3ncc(F)c(O)n3)cc2-c2ccon2)on1] | 190 |
| 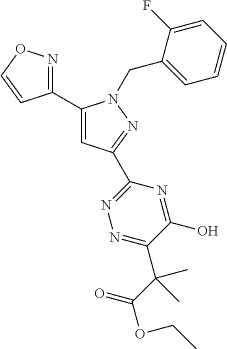 [SMILES: CCOC(=O)C(C)(C)c1nnc(-c2cc(-c3ccon3)n(Cc3ccccc3F)n2)nc1O] | 191 |
| 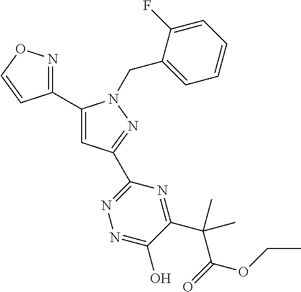 [SMILES: CCOC(=O)C(C)(C)c1nc(-c2cc(-c3ccon3)n(Cc3ccccc3F)n2)nnc1O] | 192 |
| 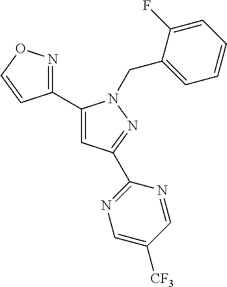 [SMILES: Fc1ccccc1Cn1nc(-c2ncc(C(F)(F)F)cn2)cc1-c1ccon1] | 205 |
| 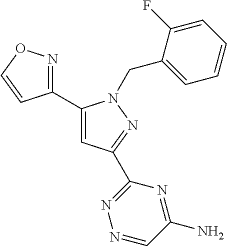 [SMILES: Nc1cnnc(-c2cc(-c3ccon3)n(Cc3ccccc3F)n2)n1] | 207 |
| 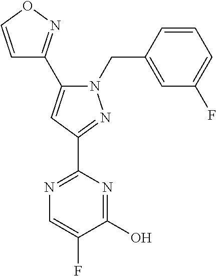 [SMILES: Oc1nc(-c2cc(-c3ccon3)n(Cc3cccc(F)c3)n2)ncc1F] | 197 |
| 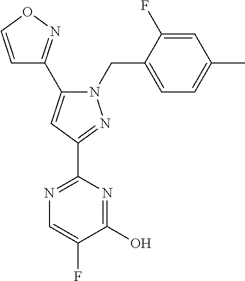 [SMILES: Cc1ccc(Cn2nc(-c3ncc(F)c(O)n3)cc2-c2ccon2)c(F)c1] | 208 |
| 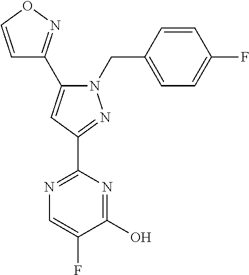 [SMILES: Oc1nc(-c2cc(-c3ccon3)n(Cc3ccc(F)cc3)n2)ncc1F] | 213 |
| 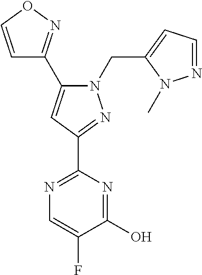 [SMILES: Cn1nccc1Cn1nc(-c2ncc(F)c(O)n2)cc1-c1ccon1] | 212 |
| 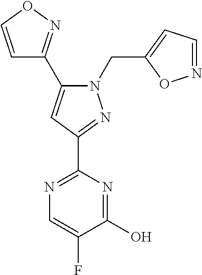 [SMILES: Oc1nc(-c2cc(-c3ccon3)n(Cc3ccno3)n2)ncc1F] | 211 |
| 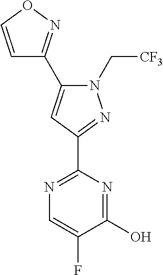 [SMILES: Oc1nc(-c2cc(-c3ccon3)n(CC(F)(F)F)n2)ncc1F] | 214 |
| 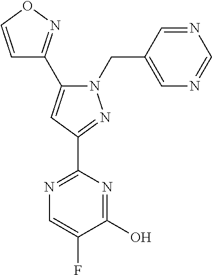 [SMILES: Oc1nc(-c2cc(-c3ccon3)n(Cc3cncnc3)n2)ncc1F] | 216 |
| 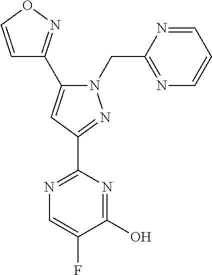 [SMILES: Oc1nc(-c2cc(-c3ccon3)n(Cc3ncccn3)n2)ncc1F] | 215 |
| 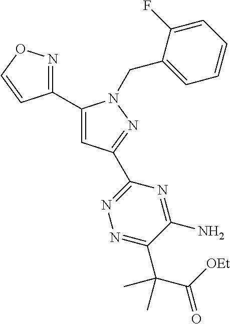 [SMILES: CCOC(=O)C(C)(C)c1nnc(-c2cc(-c3ccon3)n(Cc3ccccc3F)n2)nc1N] | 209 |

TABLE XIV

| | |
|---|---|
| 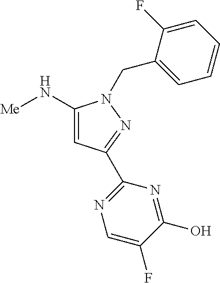 [SMILES: CNc1cc(-c2ncc(F)c(O)n2)nn1Cc1ccccc1F] | 1 |
| 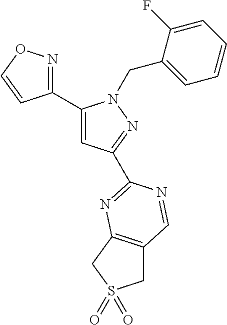 [SMILES: O=S1(=O)Cc2cnc(-c3cc(-c4ccon4)n(Cc4ccccc4F)n3)nc2C1] | 3 |
| 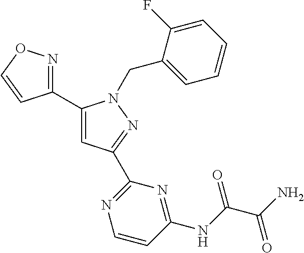 [SMILES: NC(=O)C(=O)Nc1ccnc(-c2cc(-c3ccon3)n(Cc3ccccc3F)n2)n1] | 4 |
| 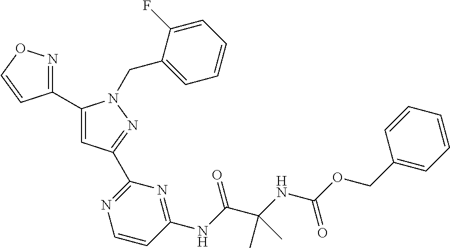 [SMILES: CC(C)(NC(=O)OCc1ccccc1)C(=O)Nc1ccnc(-c2cc(-c3ccon3)n(Cc3ccccc3F)n2)n1] | 5 |
| 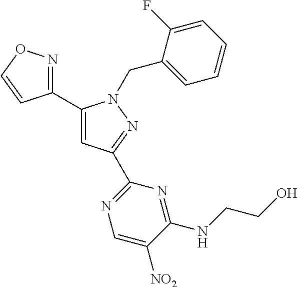 [SMILES: O=[N+]([O-])c1cnc(-c2cc(-c3ccon3)n(Cc3ccccc3F)n2)nc1NCCO] | 7 |
| 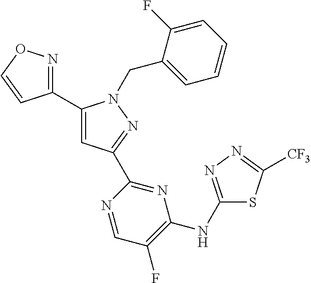 [SMILES: Fc1ccccc1Cn1nc(-c2ncc(F)c(Nc3nnc(C(F)(F)F)s3)n2)cc1-c1ccon1] | 11 |
| 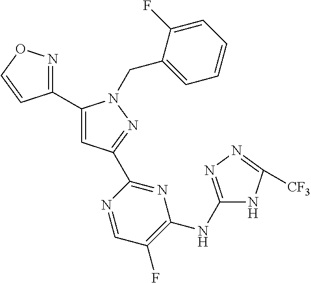 [SMILES: Fc1ccccc1Cn1nc(-c2ncc(F)c(Nc3nnc(C(F)(F)F)[nH]3)n2)cc1-c1ccon1] | 12 |
| 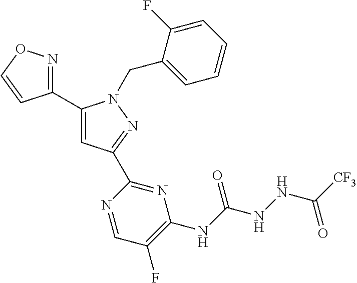 [SMILES: O=C(NNC(=O)C(F)(F)F)Nc1nc(-c2cc(-c3ccon3)n(Cc3ccccc3F)n2)ncc1F] | 13 |
| 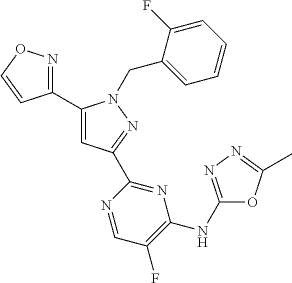 [SMILES: Cc1nnc(Nc2nc(-c3cc(-c4ccon4)n(Cc4ccccc4F)n3)ncc2F)o1] | 14 |
| 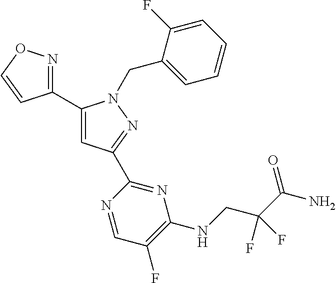 [SMILES: NC(=O)C(F)(F)CNc1nc(-c2cc(-c3ccon3)n(Cc3ccccc3F)n2)ncc1F] | 15 |
| 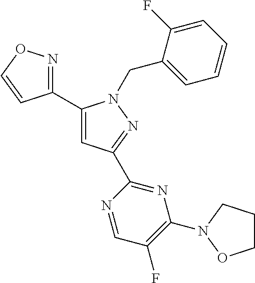 [SMILES: Fc1ccccc1Cn1nc(-c2ncc(F)c(N3CCCO3)n2)cc1-c1ccon1] | 16 |
| 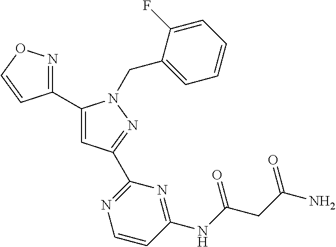 [SMILES: NC(=O)CC(=O)Nc1ccnc(-c2cc(-c3ccon3)n(Cc3ccccc3F)n2)n1] | 17 |
| 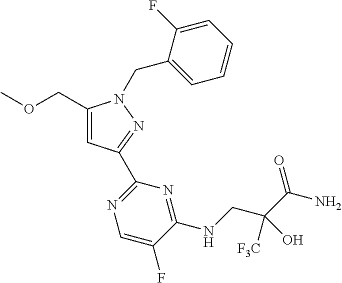 [SMILES: COCc1cc(-c2ncc(F)c(NCC(O)(C(N)=O)C(F)(F)F)n2)nn1Cc1ccccc1F] | 20 |
| 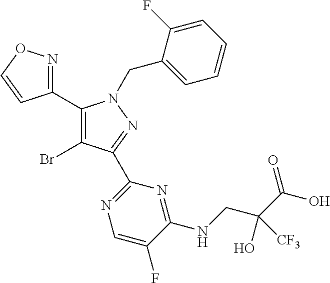 [SMILES: O=C(O)C(O)(CNc1nc(-c2nn(Cc3ccccc3F)c(-c3ccon3)c2Br)ncc1F)C(F)(F)F] | 25 |
| 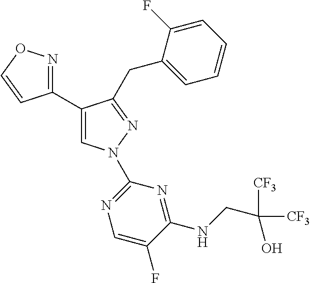 [SMILES: OC(CNc1nc(-n2cc(-c3ccon3)c(Cc3ccccc3F)n2)ncc1F)(C(F)(F)F)C(F)(F)F] | 26 |
| 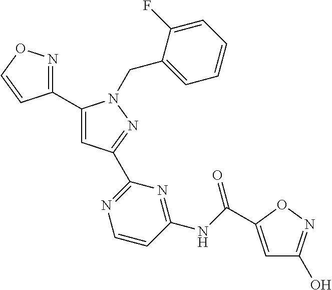 [SMILES: O=C(Nc1ccnc(-c2cc(-c3ccon3)n(Cc3ccccc3F)n2)n1)c1cc(O)no1] | 27 |
| 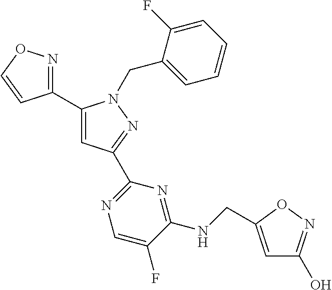 [SMILES: Oc1cc(CNc2nc(-c3cc(-c4ccon4)n(Cc4ccccc4F)n3)ncc2F)on1] | 28 |
| 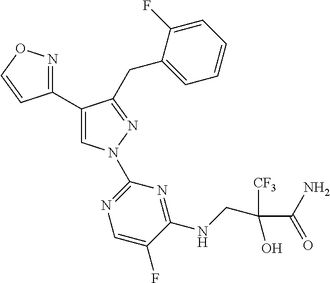 [SMILES: NC(=O)C(O)(CNc1nc(-n2cc(-c3ccon3)c(Cc3ccccc3F)n2)ncc1F)C(F)(F)F] | 30 |
| 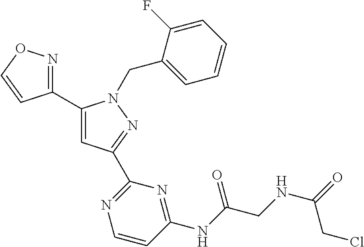 [SMILES: O=C(CCl)NCC(=O)Nc1ccnc(-c2cc(-c3ccon3)n(Cc3ccccc3F)n2)n1] | 32 |
| 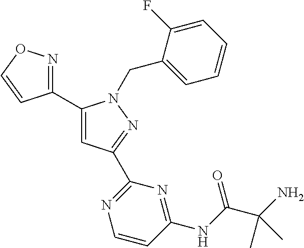 [SMILES: CC(C)(N)C(=O)Nc1ccnc(-c2cc(-c3ccon3)n(Cc3ccccc3F)n2)n1] | 33 |
| 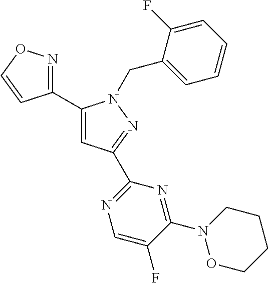 [SMILES: Fc1ccccc1Cn1nc(-c2ncc(F)c(N3CCCCO3)n2)cc1-c1ccon1] | 34 |
| 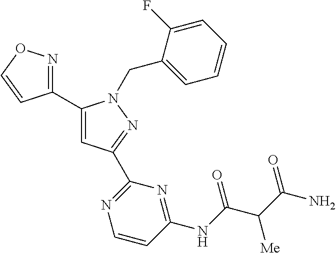 [SMILES: CC(C(N)=O)C(=O)Nc1ccnc(-c2cc(-c3ccon3)n(Cc3ccccc3F)n2)n1] | 35 |
| 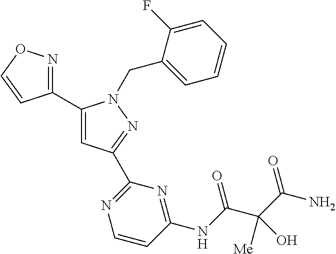 [SMILES: CC(O)(C(N)=O)C(=O)Nc1ccnc(-c2cc(-c3ccon3)n(Cc3ccccc3F)n2)n1] | 36 |
| 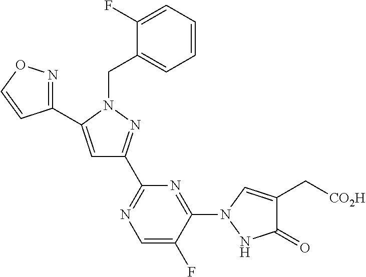 [SMILES: O=C(O)Cc1cn(-c2nc(-c3cc(-c4ccon4)n(Cc4ccccc4F)n3)ncc2F)[nH]c1=O] | 38 |
| 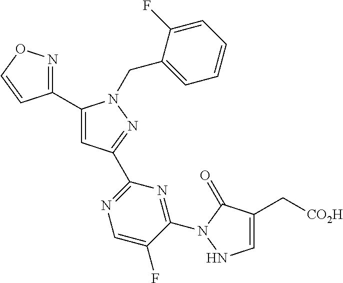 [SMILES: O=C(O)Cc1c[nH]n(-c2nc(-c3cc(-c4ccon4)n(Cc4ccccc4F)n3)ncc2F)c1=O] | 39 |
| 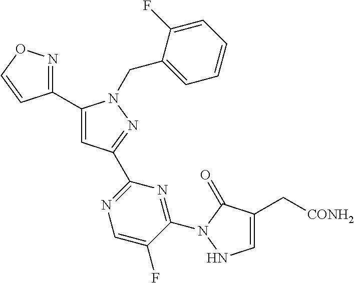 [SMILES: NC(=O)Cc1c[nH]n(-c2nc(-c3cc(-c4ccon4)n(Cc4ccccc4F)n3)ncc2F)c1=O] | 40 |
| 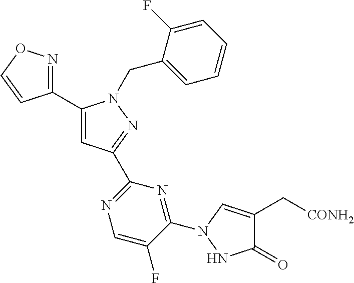 [SMILES: NC(=O)Cc1cn(-c2nc(-c3cc(-c4ccon4)n(Cc4ccccc4F)n3)ncc2F)[nH]c1=O] | 41 |
| 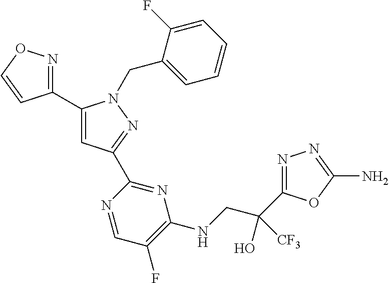 [SMILES: Nc1nnc(C(O)(CNc2nc(-c3cc(-c4ccon4)n(Cc4ccccc4F)n3)ncc2F)C(F)(F)F)o1] | 42 |
| 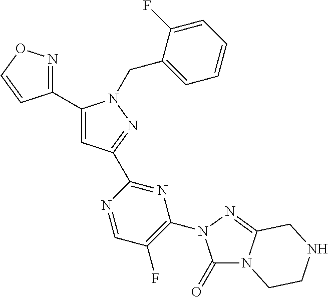 [SMILES: O=c1n(-c2nc(-c3cc(-c4ccon4)n(Cc4ccccc4F)n3)ncc2F)nc2n1CCNC2] | 43 |
| 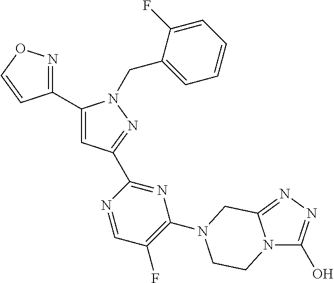 [SMILES: Oc1nnc2n1CCN(c1nc(-c3cc(-c4ccon4)n(Cc4ccccc4F)n3)ncc1F)C2] | 44 |
| 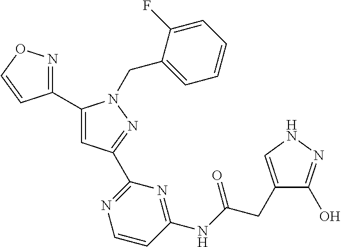 [SMILES: O=C(Cc1c[nH]nc1O)Nc1ccnc(-c2cc(-c3ccon3)n(Cc3ccccc3F)n2)n1] | 45 |
| 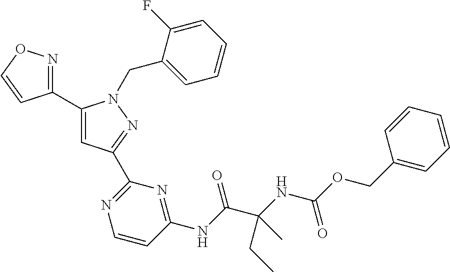 [SMILES: CCC(C)(NC(=O)OCc1ccccc1)C(=O)Nc1ccnc(-c2cc(-c3ccon3)n(Cc3ccccc3F)n2)n1] | 46 |
| 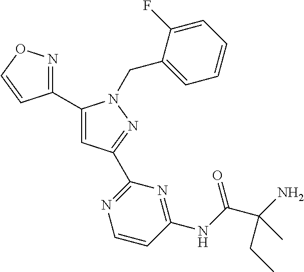 [SMILES: CCC(C)(N)C(=O)Nc1ccnc(-c2cc(-c3ccon3)n(Cc3ccccc3F)n2)n1] | 47 |
| 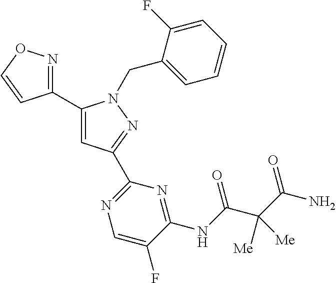 [SMILES: CC(C)(C(N)=O)C(=O)Nc1nc(-c2cc(-c3ccon3)n(Cc3ccccc3F)n2)ncc1F] | 48 |
| 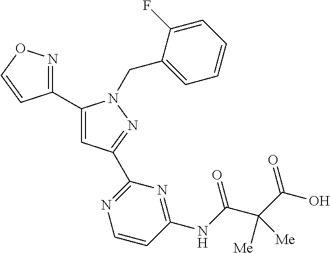 [SMILES: CC(C)(C(=O)O)C(=O)Nc1ccnc(-c2cc(-c3ccon3)n(Cc3ccccc3F)n2)n1] | 49 |
| 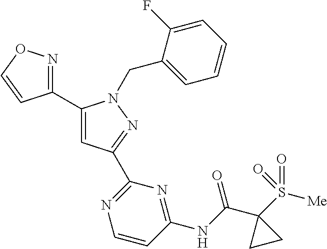 [SMILES: CS(=O)(=O)C1(C(=O)Nc2ccnc(-c3cc(-c4ccon4)n(Cc4ccccc4F)n3)n2)CC1] | 50 |
| 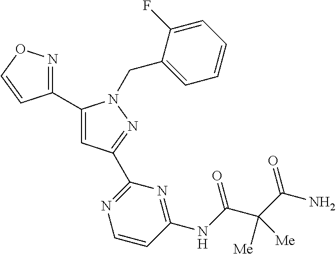 [SMILES: CC(C)(C(N)=O)C(=O)Nc1ccnc(-c2cc(-c3ccon3)n(Cc3ccccc3F)n2)n1] | 51 |
| 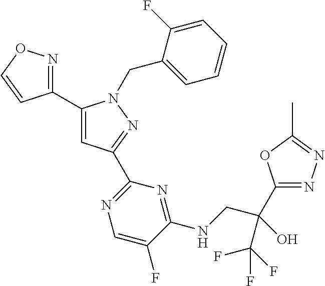 [SMILES: Cc1nnc(C(O)(CNc2nc(-c3cc(-c4ccon4)n(Cc4ccccc4F)n3)ncc2F)C(F)(F)F)o1] | 52 |
| 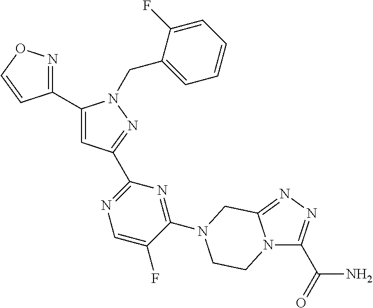 [SMILES: NC(=O)c1nnc2n1CCN(c1nc(-c3cc(-c4ccon4)n(Cc4ccccc4F)n3)ncc1F)C2] | 54 |
| 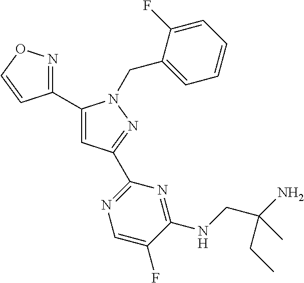 [SMILES: CCC(C)(N)CNc1nc(-c2cc(-c3ccon3)n(Cc3ccccc3F)n2)ncc1F] | 55 |
| 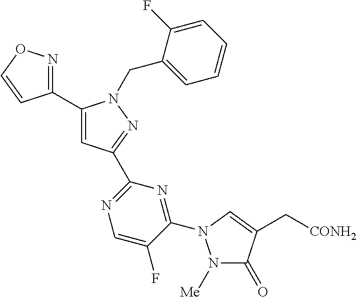 [SMILES: Cn1c(=O)c(CC(N)=O)cn1-c1nc(-c2cc(-c3ccon3)n(Cc3ccccc3F)n2)ncc1F] | 56 |
| 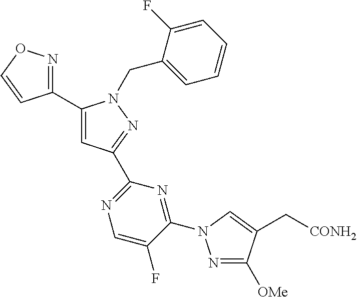 [SMILES: COc1nn(-c2nc(-c3cc(-c4ccon4)n(Cc4ccccc4F)n3)ncc2F)cc1CC(N)=O] | 57 |
| 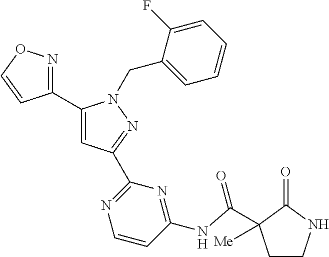 [SMILES: CC1(C(=O)Nc2ccnc(-c3cc(-c4ccon4)n(Cc4ccccc4F)n3)n2)CCNC1=O] | 59 |
| 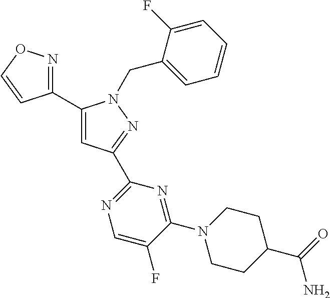 [SMILES: NC(=O)C1CCN(c2nc(-c3cc(-c4ccon4)n(Cc4ccccc4F)n3)ncc2F)CC1] | 60 |
| 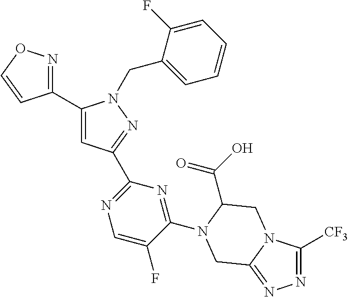 [SMILES: O=C(O)C1Cn2c(nnc2C(F)(F)F)CN1c1nc(-c2cc(-c3ccon3)n(Cc3ccccc3F)n2)ncc1F] | 62 |
| 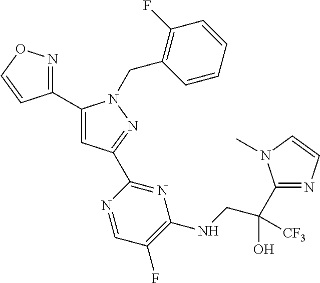 [SMILES: Cn1ccnc1C(O)(CNc1nc(-c2cc(-c3ccon3)n(Cc3ccccc3F)n2)ncc1F)C(F)(F)F] | 64 |
| 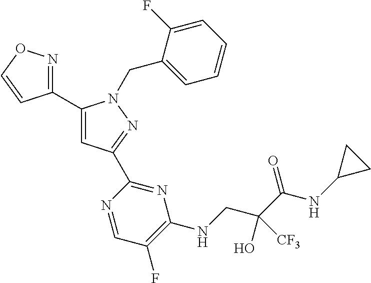 [SMILES: O=C(NC1CC1)C(O)(CNc1nc(-c2cc(-c3ccon3)n(Cc3ccccc3F)n2)ncc1F)C(F)(F)F] | 65 |
| 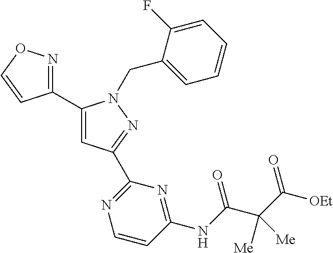 [SMILES: CCOC(=O)C(C)(C)C(=O)Nc1ccnc(-c2cc(-c3ccon3)n(Cc3ccccc3F)n2)n1] | 66 |
| 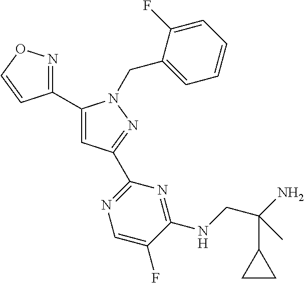 [SMILES: CC(N)(CNc1nc(-c2cc(-c3ccon3)n(Cc3ccccc3F)n2)ncc1F)C1CC1] | 67 |
| 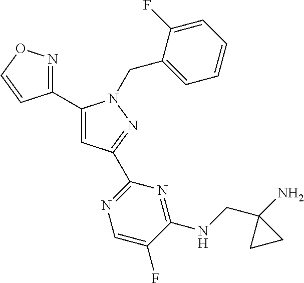 [SMILES: NC1(CNc2nc(-c3cc(-c4ccon4)n(Cc4ccccc4F)n3)ncc2F)CC1] | 68 |
| 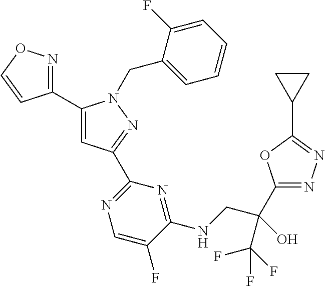 [SMILES: OC(CNc1nc(-c2cc(-c3ccon3)n(Cc3ccccc3F)n2)ncc1F)(c1nnc(C2CC2)o1)C(F)(F)F] | 69 |
| 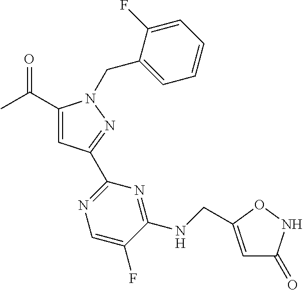 [SMILES: CC(=O)c1cc(-c2ncc(F)c(NCc3cc(=O)[nH]o3)n2)nn1Cc1ccccc1F] | 70 |
| 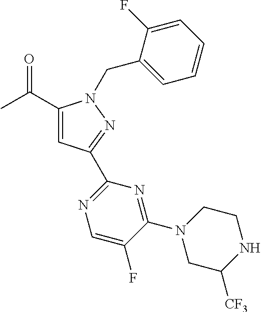 [SMILES: CC(=O)c1cc(-c2ncc(F)c(N3CCNC(C(F)(F)F)C3)n2)nn1Cc1ccccc1F] | 71 |
| 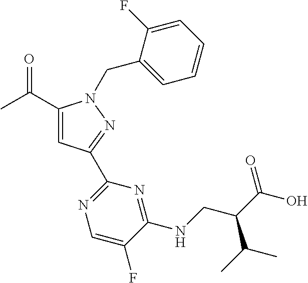 [SMILES: CC(=O)c1cc(-c2ncc(F)c(NC[C@@H](C(=O)O)C(C)C)n2)nn1Cc1ccccc1F] | 72 |
| 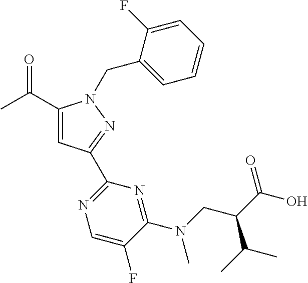 [SMILES: CC(=O)c1cc(-c2ncc(F)c(N(C)C[C@@H](C(=O)O)C(C)C)n2)nn1Cc1ccccc1F] | 73 |
| 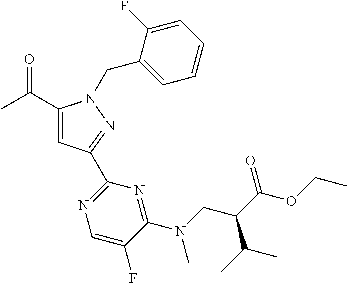 [SMILES: CCOC(=O)[C@H](CN(C)c1nc(-c2cc(C(C)=O)n(Cc3ccccc3F)n2)ncc1F)C(C)C] | 74 |
| 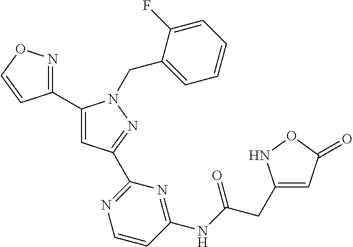 [SMILES: O=C(Cc1cc(=O)o[nH]1)Nc1ccnc(-c2cc(-c3ccon3)n(Cc3ccccc3F)n2)n1] | 75 |
| 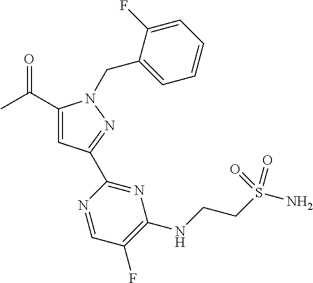 [SMILES: CC(=O)c1cc(-c2ncc(F)c(NCCS(N)(=O)=O)n2)nn1Cc1ccccc1F] | 76 |
| 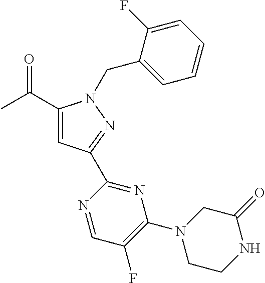 [SMILES: CC(=O)c1cc(-c2ncc(F)c(N3CCNC(=O)C3)n2)nn1Cc1ccccc1F] | 77 |
| 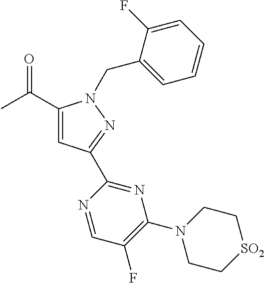 [SMILES: CC(=O)c1cc(-c2ncc(F)c(N3CCS(=O)(=O)CC3)n2)nn1Cc1ccccc1F] | 78 |
| 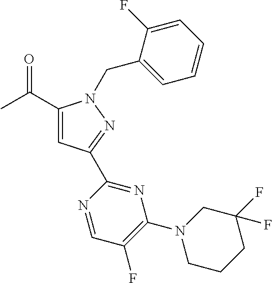 [SMILES: CC(=O)c1cc(-c2ncc(F)c(N3CCCC(F)(F)C3)n2)nn1Cc1ccccc1F] | 79 |
| 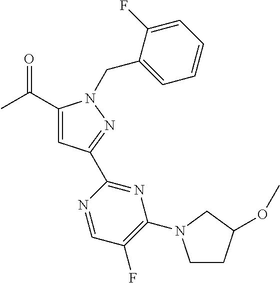 [SMILES: COC1CCN(c2nc(-c3cc(C(C)=O)n(Cc4ccccc4F)n3)ncc2F)C1] | 80 |
| 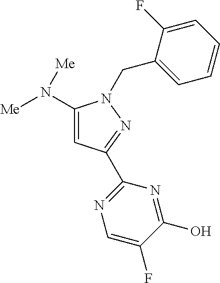 [SMILES: CN(C)c1cc(-c2ncc(F)c(O)n2)nn1Cc1ccccc1F] | 81 |
| 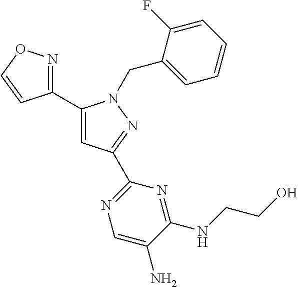 [SMILES: Nc1cnc(-c2cc(-c3ccon3)n(Cc3ccccc3F)n2)nc1NCCO] | 82 |
| 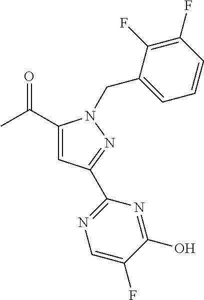 [SMILES: CC(=O)c1cc(-c2ncc(F)c(O)n2)nn1Cc1cccc(F)c1F] | 83 |
| 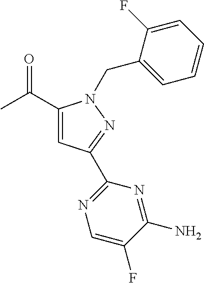 [SMILES: CC(=O)c1cc(-c2ncc(F)c(N)n2)nn1Cc1ccccc1F] | 84 |
| 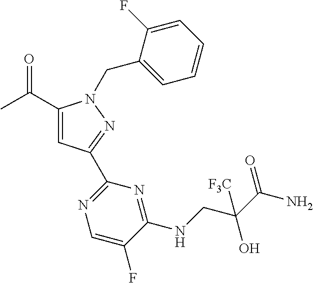 [SMILES: CC(=O)c1cc(-c2ncc(F)c(NCC(O)(C(N)=O)C(F)(F)F)n2)nn1Cc1ccccc1F] | 85 |
| 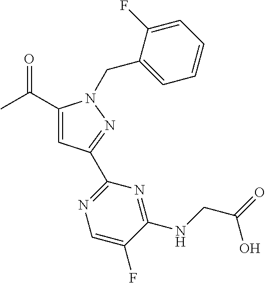 [SMILES: CC(=O)c1cc(-c2ncc(F)c(NCC(=O)O)n2)nn1Cc1ccccc1F] | 86 |
| 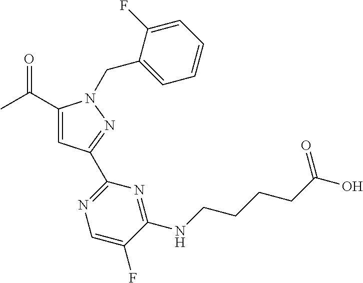 [SMILES: CC(=O)c1cc(-c2ncc(F)c(NCCCCC(=O)O)n2)nn1Cc1ccccc1F] | 87 |
| 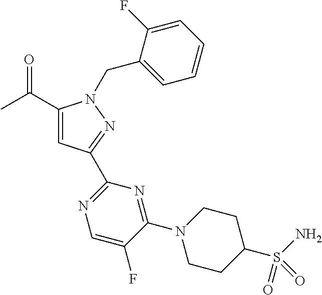 [SMILES: CC(=O)c1cc(-c2ncc(F)c(N3CCC(S(N)(=O)=O)CC3)n2)nn1Cc1ccccc1F] | 88 |
| 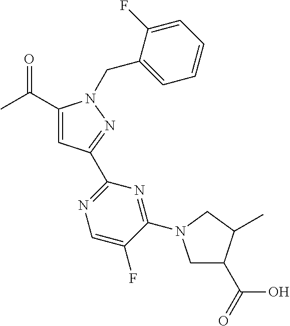 [SMILES: CC(=O)c1cc(-c2ncc(F)c(N3CC(C)C(C(=O)O)C3)n2)nn1Cc1ccccc1F] | 89 |
| 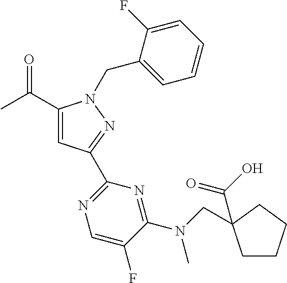 [SMILES: CC(=O)c1cc(-c2ncc(F)c(N(C)CC3(C(=O)O)CCCC3)n2)nn1Cc1ccccc1F] | 90 |
| 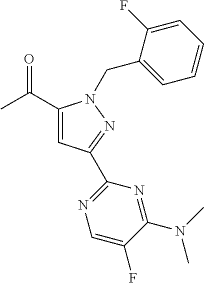 [SMILES: CC(=O)c1cc(-c2ncc(F)c(N(C)C)n2)nn1Cc1ccccc1F] | 91 |
| 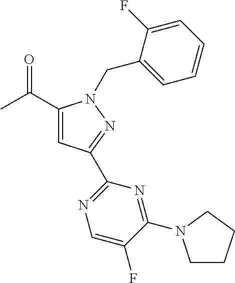 [SMILES: CC(=O)c1cc(-c2ncc(F)c(N3CCCC3)n2)nn1Cc1ccccc1F] | 92 |
| 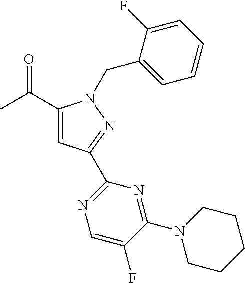 [SMILES: CC(=O)c1cc(-c2ncc(F)c(N3CCCCC3)n2)nn1Cc1ccccc1F] | 93 |
| 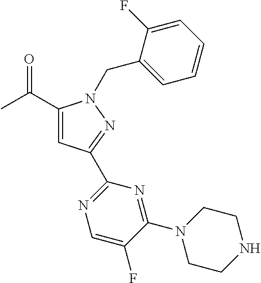 [SMILES: CC(=O)c1cc(-c2ncc(F)c(N3CCNCC3)n2)nn1Cc1ccccc1F] | 94 |
| 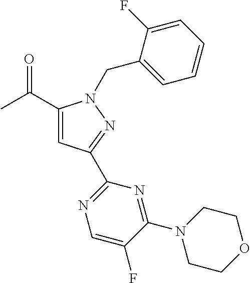 [SMILES: CC(=O)c1cc(-c2ncc(F)c(N3CCOCC3)n2)nn1Cc1ccccc1F] | 95 |
| 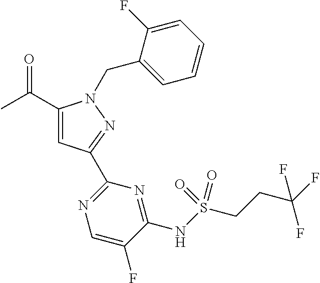 [SMILES: CC(=O)c1cc(-c2ncc(F)c(NS(=O)(=O)CCC(F)(F)F)n2)nn1Cc1ccccc1F] | 96 |
| 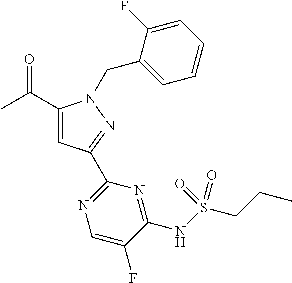 [SMILES: CCCS(=O)(=O)Nc1nc(-c2cc(C(C)=O)n(Cc3ccccc3F)n2)ncc1F] | 97 |
| 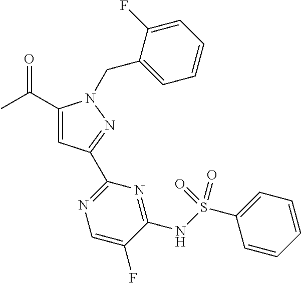 [SMILES: CC(=O)c1cc(-c2ncc(F)c(NS(=O)(=O)c3ccccc3)n2)nn1Cc1ccccc1F] | 98 |
| 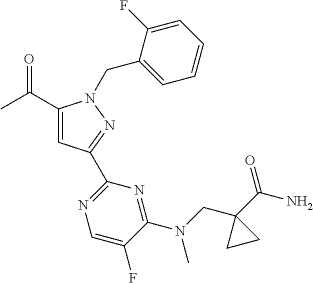 [SMILES: CC(=O)c1cc(-c2ncc(F)c(N(C)CC3(C(N)=O)CC3)n2)nn1Cc1ccccc1F] | 99 |
| 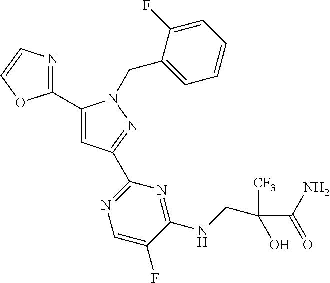 [SMILES: NC(=O)C(O)(CNc1nc(-c2cc(-c3ncco3)n(Cc3ccccc3F)n2)ncc1F)C(F)(F)F] | 102 |
| 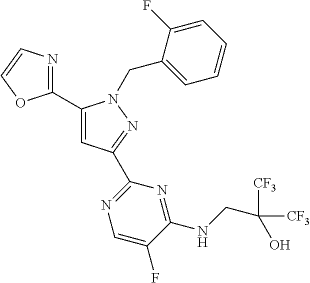 [SMILES: OC(CNc1nc(-c2cc(-c3ncco3)n(Cc3ccccc3F)n2)ncc1F)(C(F)(F)F)C(F)(F)F] | 103 |
| 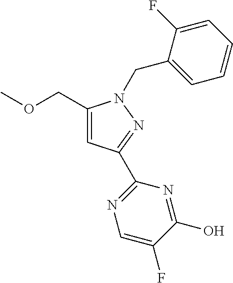 [SMILES: COCc1cc(-c2ncc(F)c(O)n2)nn1Cc1ccccc1F] | 105 |
| 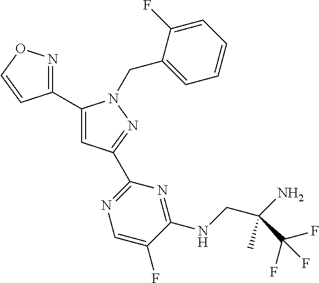 [SMILES: C[C@@](N)(CNc1nc(-c2cc(-c3ccon3)n(Cc3ccccc3F)n2)ncc1F)C(F)(F)F] | 106 |
| 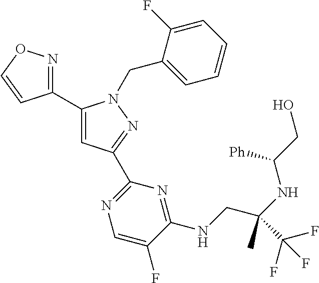 [SMILES: C[C@@](CNc1nc(-c2cc(-c3ccon3)n(Cc3ccccc3F)n2)ncc1F)(N[C@@H](CO)c1ccccc1)C(F)(F)F] | 107 |
| 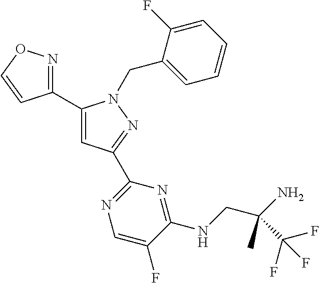 [SMILES: C[C@](N)(CNc1nc(-c2cc(-c3ccon3)n(Cc3ccccc3F)n2)ncc1F)C(F)(F)F] | 108 |
| 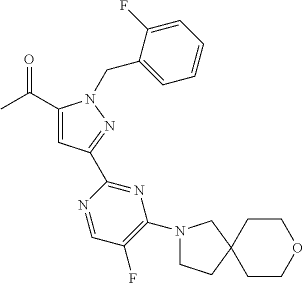 [SMILES: CC(=O)c1cc(-c2ncc(F)c(N3CCC4(CCOCC4)C3)n2)nn1Cc1ccccc1F] | 112 |
| 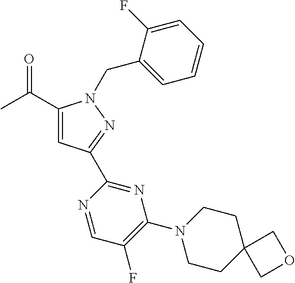 [SMILES: CC(=O)c1cc(-c2ncc(F)c(N3CCC4(CC3)COC4)n2)nn1Cc1ccccc1F] | 113 |
| 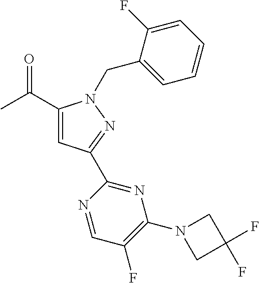 [SMILES: CC(=O)c1cc(-c2ncc(F)c(N3CC(F)(F)C3)n2)nn1Cc1ccccc1F] | 114 |
| 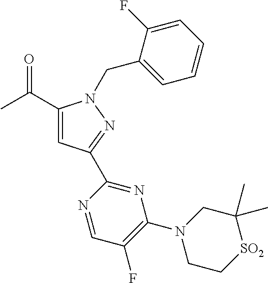 [SMILES: CC(=O)c1cc(-c2ncc(F)c(N3CCS(=O)(=O)C(C)(C)C3)n2)nn1Cc1ccccc1F] | 115 |
| 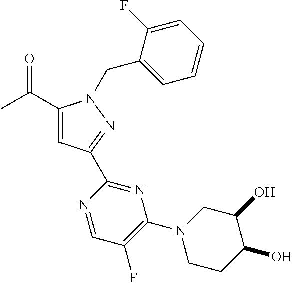 [SMILES: CC(=O)c1cc(-c2ncc(F)c(N3CC[C@H](O)[C@H](O)C3)n2)nn1Cc1ccccc1F] | 116 |
| 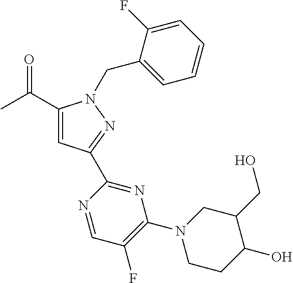 [SMILES: CC(=O)c1cc(-c2ncc(F)c(N3CCC(O)C(CO)C3)n2)nn1Cc1ccccc1F] | 117 |
| 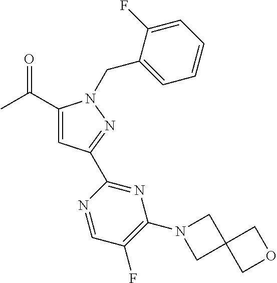 [SMILES: CC(=O)c1cc(-c2ncc(F)c(N3CC4(COC4)C3)n2)nn1Cc1ccccc1F] | 118 |
| 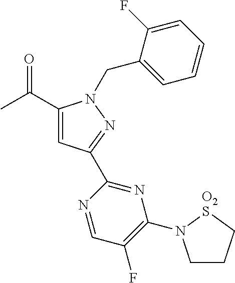 [SMILES: CC(=O)c1cc(-c2ncc(F)c(N3CCCS3(=O)=O)n2)nn1Cc1ccccc1F] | 119 |
| 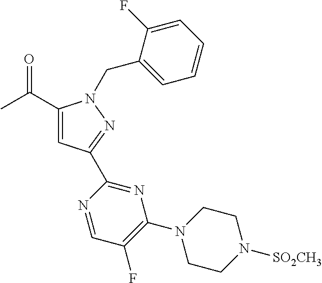 [SMILES: CC(=O)c1cc(-c2ncc(F)c(N3CCN(S(C)(=O)=O)CC3)n2)nn1Cc1ccccc1F] | 120 |
| 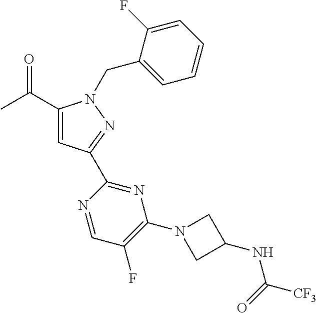 [SMILES: CC(=O)c1cc(-c2ncc(F)c(N3CC(NC(=O)C(F)(F)F)C3)n2)nn1Cc1ccccc1F] | 121 |
| 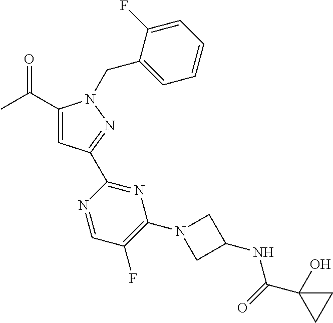 [SMILES: CC(=O)c1cc(-c2ncc(F)c(N3CC(NC(=O)C4(O)CC4)C3)n2)nn1Cc1ccccc1F] | 122 |
| 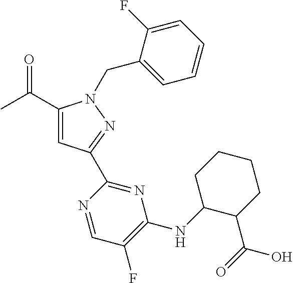 [SMILES: CC(=O)c1cc(-c2ncc(F)c(NC3CCCCC3C(=O)O)n2)nn1Cc1ccccc1F] | 123 |
| 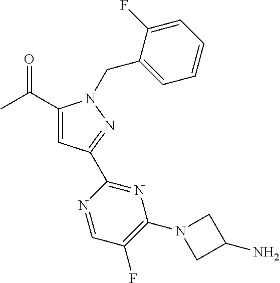 [SMILES: CC(=O)c1cc(-c2ncc(F)c(N3CC(N)C3)n2)nn1Cc1ccccc1F] | 124 |
| 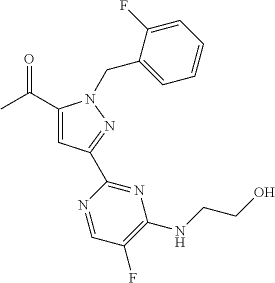 [SMILES: CC(=O)c1cc(-c2ncc(F)c(NCCO)n2)nn1Cc1ccccc1F] | 125 |
| 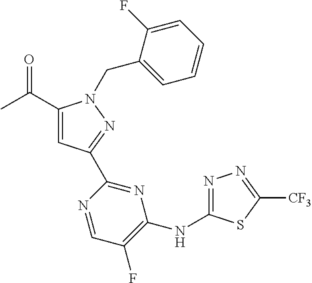 [SMILES: CC(=O)c1cc(-c2ncc(F)c(Nc3nnc(C(F)(F)F)s3)n2)nn1Cc1ccccc1F] | 126 |
| 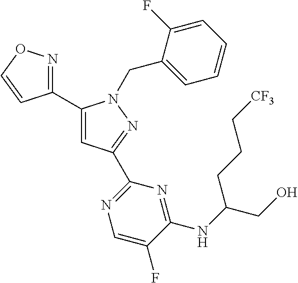 [SMILES: OCC(CCCC(F)(F)F)Nc1nc(-c2cc(-c3ccon3)n(Cc3ccccc3F)n2)ncc1F] | 127 |
| 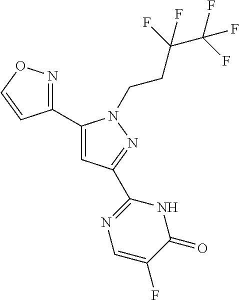 [SMILES: O=c1[nH]c(-c2cc(-c3ccon3)n(CCC(F)(F)C(F)(F)F)n2)ncc1F] | 128 |
| 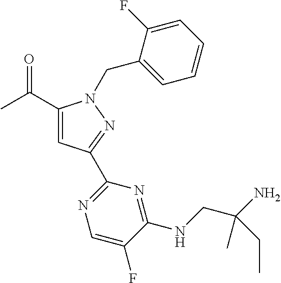 [SMILES: CCC(C)(N)CNc1nc(-c2cc(C(C)=O)n(Cc3ccccc3F)n2)ncc1F] | 129 |
| 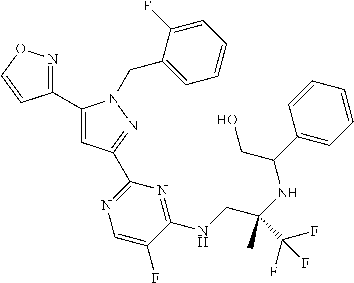 [SMILES: C[C@@](CNc1nc(-c2cc(-c3ccon3)n(Cc3ccccc3F)n2)ncc1F)(NC(CO)c1ccccc1)C(F)(F)F] | 130 |
| 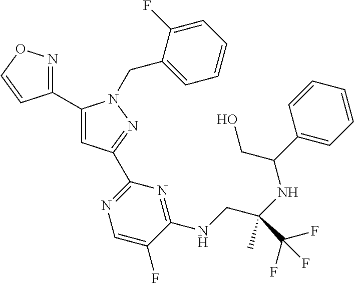 [SMILES: C[C@](CNc1nc(-c2cc(-c3ccon3)n(Cc3ccccc3F)n2)ncc1F)(NC(CO)c1ccccc1)C(F)(F)F] | 131 |
| 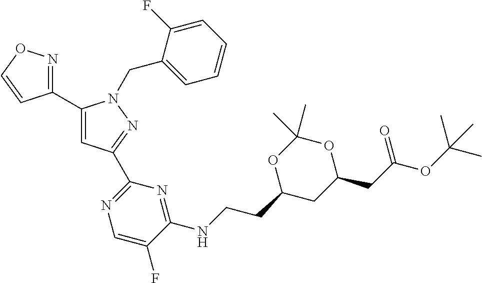 [SMILES: CC(C)(C)OC(=O)C[C@H]1C[C@@H](CCNc2nc(-c3cc(-c4ccon4)n(Cc4ccccc4F)n3)ncc2F)OC(C)(C)O1] | 132 |
| 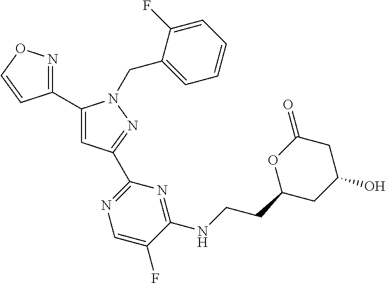 [SMILES: O=C1C[C@H](O)C[C@@H](CCNc2nc(-c3cc(-c4ccon4)n(Cc4ccccc4F)n3)ncc2F)O1] | 133 |
| 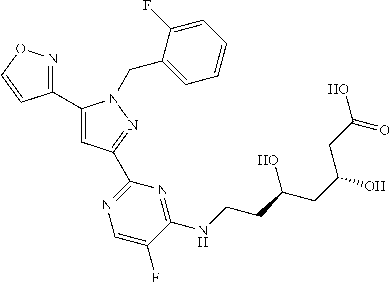 [SMILES: O=C(O)C[C@H](O)C[C@H](O)CCNc1nc(-c2cc(-c3ccon3)n(Cc3ccccc3F)n2)ncc1F] | 134 |
| 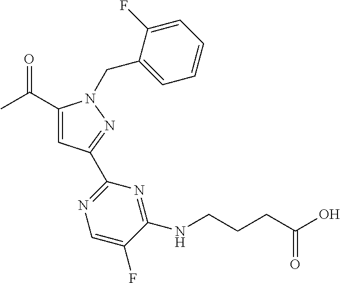 [SMILES: CC(=O)c1cc(-c2ncc(F)c(NCCCC(=O)O)n2)nn1Cc1ccccc1F] | 135 |
| 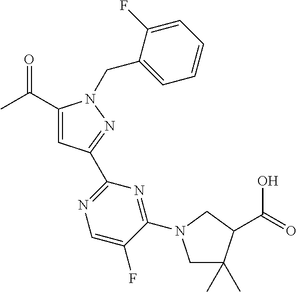 [SMILES: CC(=O)c1cc(-c2ncc(F)c(N3CC(C(=O)O)C(C)(C)C3)n2)nn1Cc1ccccc1F] | 136 |
| 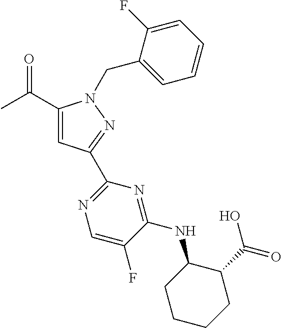 [SMILES: CC(=O)c1cc(-c2ncc(F)c(N[C@@H]3CCCC[C@H]3C(=O)O)n2)nn1Cc1ccccc1F] | 137 |
| 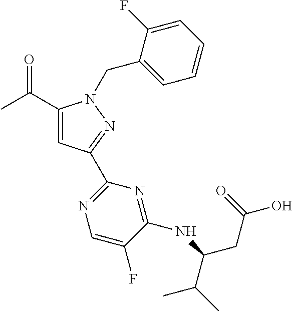 [SMILES: CC(=O)c1cc(-c2ncc(F)c(N[C@@H](CC(=O)O)C(C)C)n2)nn1Cc1ccccc1F] | 138 |
| 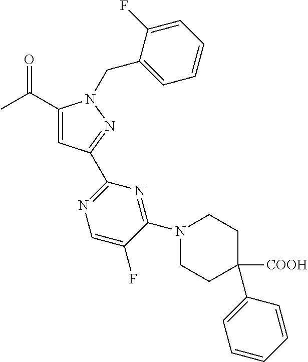 [SMILES: CC(=O)c1cc(-c2ncc(F)c(N3CCC(C(=O)O)(c4ccccc4)CC3)n2)nn1Cc1ccccc1F] | 139 |
| 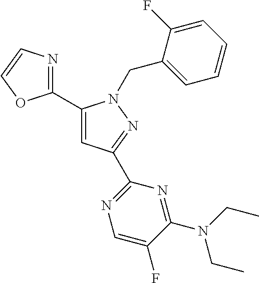 [SMILES: CCN(CC)c1nc(-c2cc(-c3ncco3)n(Cc3ccccc3F)n2)ncc1F] | 140 |
| 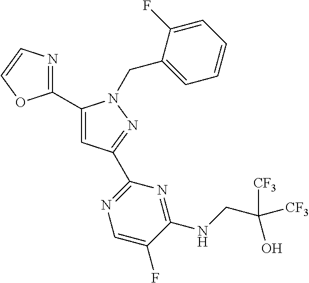 [SMILES: OC(CNc1nc(-c2cc(-c3ncco3)n(Cc3ccccc3F)n2)ncc1F)(C(F)(F)F)C(F)(F)F] | 141 |
| 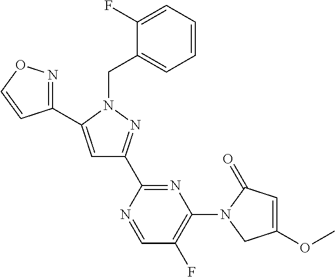 [SMILES: COC1=CC(=O)N(c2nc(-c3cc(-c4ccon4)n(Cc4ccccc4F)n3)ncc2F)C1] | 147 |
| 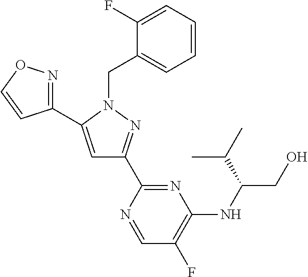 [SMILES: CC(C)[C@H](CO)Nc1nc(-c2cc(-c3ccon3)n(Cc3ccccc3F)n2)ncc1F] | 148 |
| 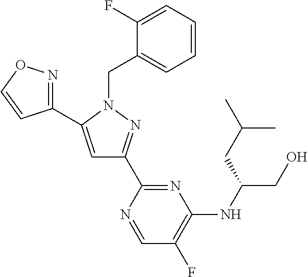 [SMILES: CC(C)C[C@H](CO)Nc1nc(-c2cc(-c3ccon3)n(Cc3ccccc3F)n2)ncc1F] | 149 |
| 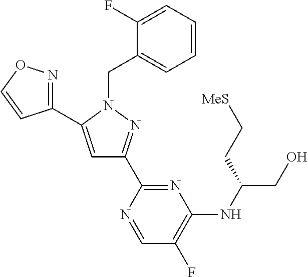 [SMILES: CSCC[C@H](CO)Nc1nc(-c2cc(-c3ccon3)n(Cc3ccccc3F)n2)ncc1F] | 150 |
| 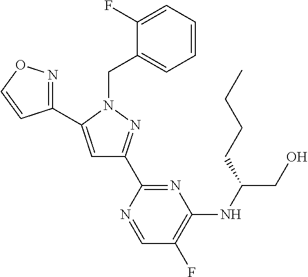 [SMILES: CCCC[C@H](CO)Nc1nc(-c2cc(-c3ccon3)n(Cc3ccccc3F)n2)ncc1F] | 151 |
| 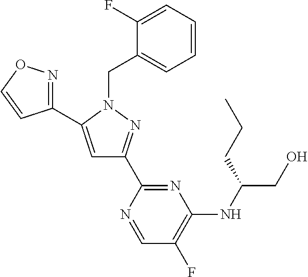 [SMILES: CCC[C@H](CO)Nc1nc(-c2cc(-c3ccon3)n(Cc3ccccc3F)n2)ncc1F] | 152 |
| 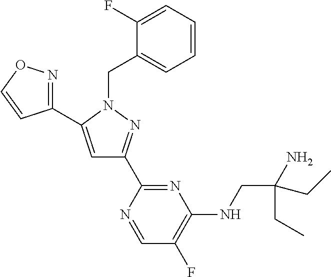 [SMILES: CCC(N)(CC)CNc1nc(-c2cc(-c3ccon3)n(Cc3ccccc3F)n2)ncc1F] | 153 |
| 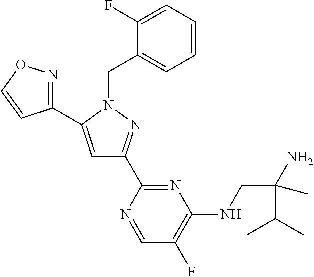 [SMILES: CC(C)C(C)(N)CNc1nc(-c2cc(-c3ccon3)n(Cc3ccccc3F)n2)ncc1F] | 154 |
| 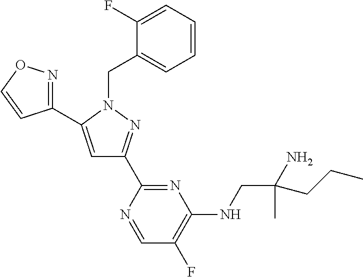 [SMILES: CCCC(C)(N)CNc1nc(-c2cc(-c3ccon3)n(Cc3ccccc3F)n2)ncc1F] | 155 |
| 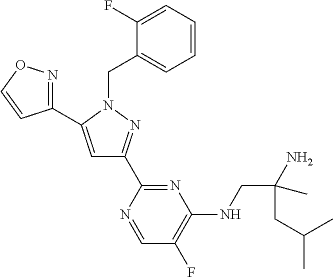 [SMILES: CC(C)CC(C)(N)CNc1nc(-c2cc(-c3ccon3)n(Cc3ccccc3F)n2)ncc1F] | 156 |
| 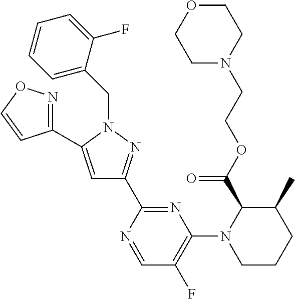 [SMILES: C[C@H]1CCCN(c2nc(-c3cc(-c4ccon4)n(Cc4ccccc4F)n3)ncc2F)[C@H]1C(=O)OCCN1CCOCC1] | 157 |
| 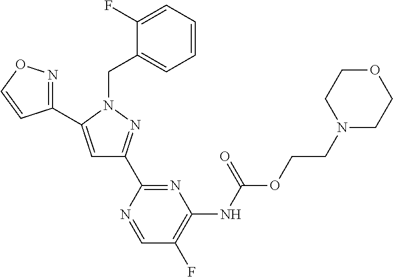 [SMILES: O=C(Nc1nc(-c2cc(-c3ccon3)n(Cc3ccccc3F)n2)ncc1F)OCCN1CCOCC1] | 158 |
| 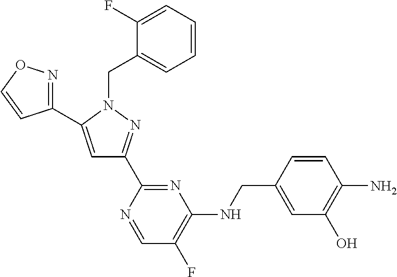 [SMILES: Nc1ccc(CNc2nc(-c3cc(-c4ccon4)n(Cc4ccccc4F)n3)ncc2F)cc1O] | 159 |
| 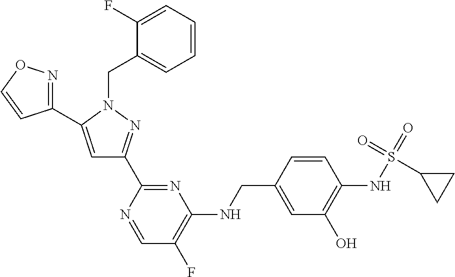 [SMILES: O=S(=O)(Nc1ccc(CNc2nc(-c3cc(-c4ccon4)n(Cc4ccccc4F)n3)ncc2F)cc1O)C1CC1] | 160 |
| 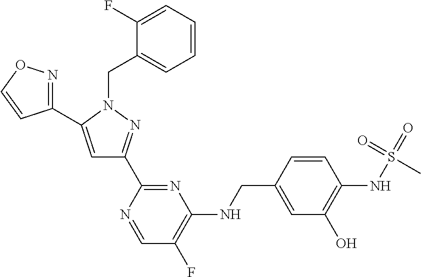 [SMILES: CS(=O)(=O)Nc1ccc(CNc2nc(-c3cc(-c4ccon4)n(Cc4ccccc4F)n3)ncc2F)cc1O] | 161 |
| 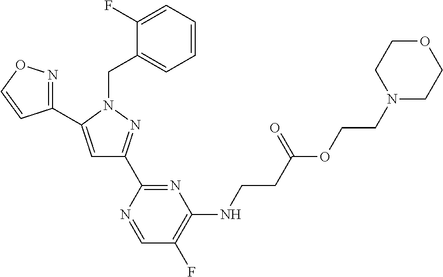 [SMILES: O=C(CCNc1nc(-c2cc(-c3ccon3)n(Cc3ccccc3F)n2)ncc1F)OCCN1CCOCC1] | 162 |
| 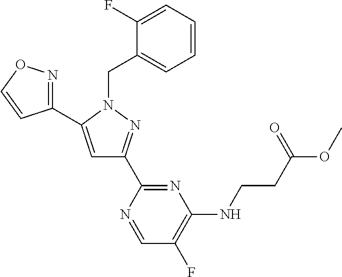 [SMILES: COC(=O)CCNc1nc(-c2cc(-c3ccon3)n(Cc3ccccc3F)n2)ncc1F] | 163 |
| 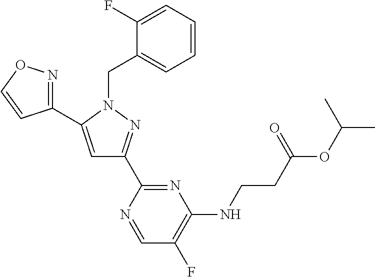 [SMILES: CC(C)OC(=O)CCNc1nc(-c2cc(-c3ccon3)n(Cc3ccccc3F)n2)ncc1F] | 164 |
| 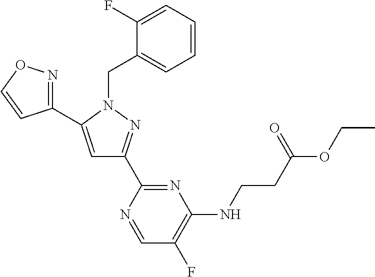 [SMILES: CCOC(=O)CCNc1nc(-c2cc(-c3ccon3)n(Cc3ccccc3F)n2)ncc1F] | 165 |
| 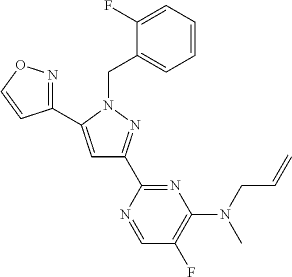 [SMILES: C=CCN(C)c1nc(-c2cc(-c3ccon3)n(Cc3ccccc3F)n2)ncc1F] | 166 |
| 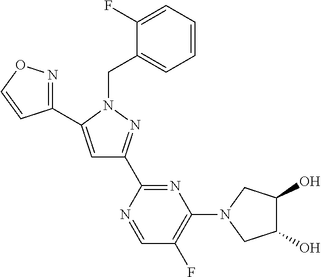 [SMILES: O[C@@H]1CN(c2nc(-c3cc(-c4ccon4)n(Cc4ccccc4F)n3)ncc2F)C[C@H]1O] | 167 |
| 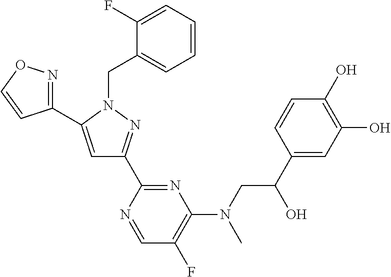 [SMILES: CN(CC(O)c1ccc(O)c(O)c1)c1nc(-c2cc(-c3ccon3)n(Cc3ccccc3F)n2)ncc1F] | 168 |
| 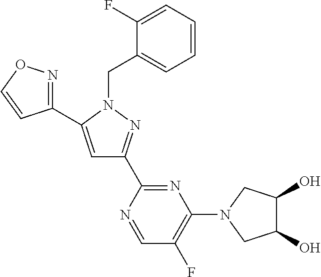 [SMILES: O[C@@H]1CN(c2nc(-c3cc(-c4ccon4)n(Cc4ccccc4F)n3)ncc2F)C[C@@H]1O] | 169 |
| 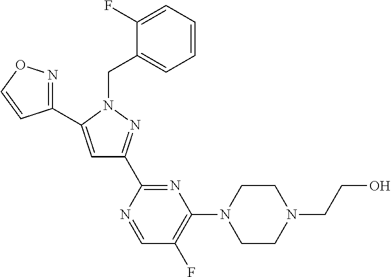 [SMILES: OCCN1CCN(c2nc(-c3cc(-c4ccon4)n(Cc4ccccc4F)n3)ncc2F)CC1] | 170 |
| 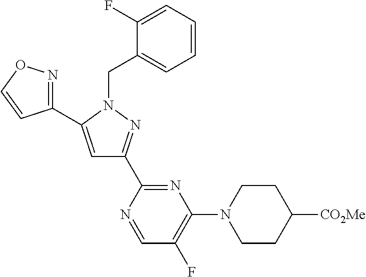 [SMILES: COC(=O)C1CCN(c2nc(-c3cc(-c4ccon4)n(Cc4ccccc4F)n3)ncc2F)CC1] | 171 |
| 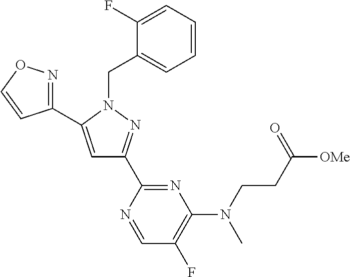 [SMILES: COC(=O)CCN(C)c1nc(-c2cc(-c3ccon3)n(Cc3ccccc3F)n2)ncc1F] | 172 |
| 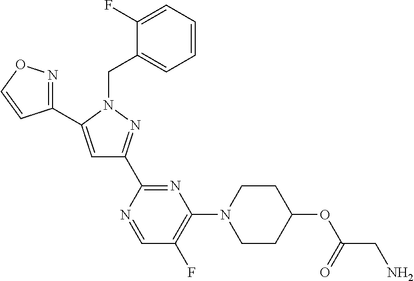 [SMILES: NCC(=O)OC1CCN(c2nc(-c3cc(-c4ccon4)n(Cc4ccccc4F)n3)ncc2F)CC1] | 173 |
| 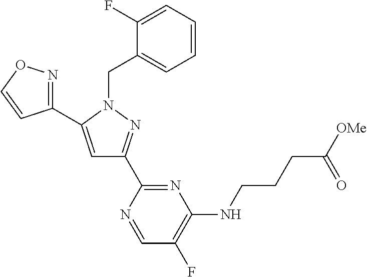 [SMILES: COC(=O)CCCNc1nc(-c2cc(-c3ccon3)n(Cc3ccccc3F)n2)ncc1F] | 174 |
| 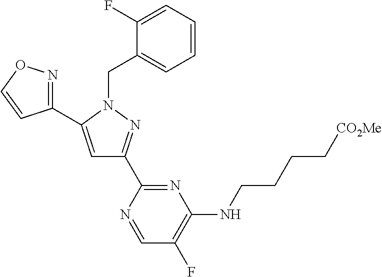 [SMILES: COC(=O)CCCCNc1nc(-c2cc(-c3ccon3)n(Cc3ccccc3F)n2)ncc1F] | 175 |
| 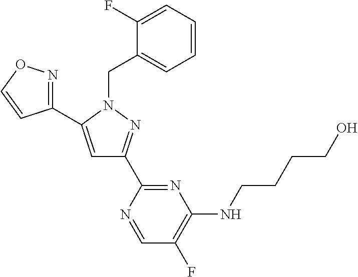 [SMILES: OCCCCNc1nc(-c2cc(-c3ccon3)n(Cc3ccccc3F)n2)ncc1F] | 176 |
| 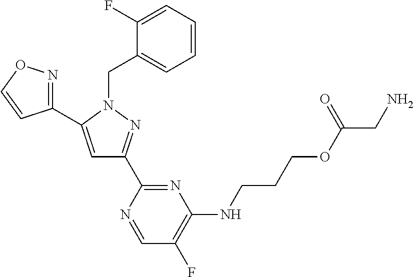 [SMILES: NCC(=O)OCCCNc1nc(-c2cc(-c3ccon3)n(Cc3ccccc3F)n2)ncc1F] | 177 |
| 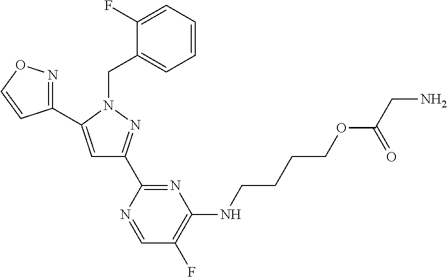 [SMILES: NCC(=O)OCCCCNc1nc(-c2cc(-c3ccon3)n(Cc3ccccc3F)n2)ncc1F] | 178 |
| 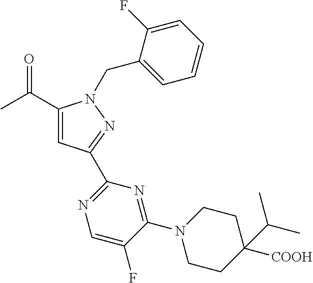 [SMILES: CC(=O)c1cc(-c2ncc(F)c(N3CCC(C(=O)O)(C(C)C)CC3)n2)nn1Cc1ccccc1F] | 179 |
| 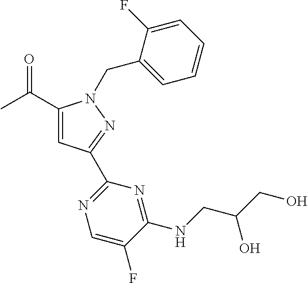 [SMILES: CC(=O)c1cc(-c2ncc(F)c(NCC(O)CO)n2)nn1Cc1ccccc1F] | 180 |
| 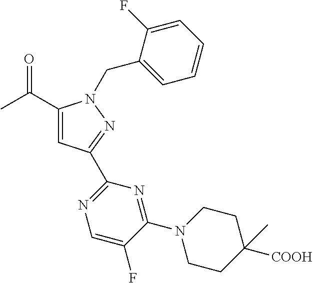 [SMILES: CC(=O)c1cc(-c2ncc(F)c(N3CCC(C)(C(=O)O)CC3)n2)nn1Cc1ccccc1F] | 181 |
| 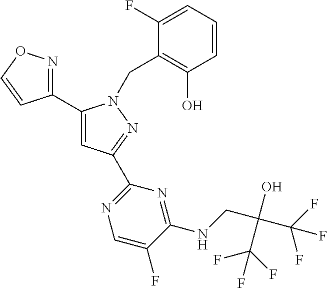 [SMILES: Oc1cccc(F)c1Cn1nc(-c2ncc(F)c(NCC(O)(C(F)(F)F)C(F)(F)F)n2)cc1-c1ccon1] | 183 |
| 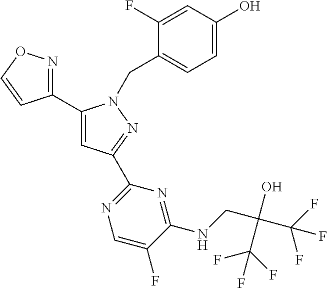 [SMILES: Oc1ccc(Cn2nc(-c3ncc(F)c(NCC(O)(C(F)(F)F)C(F)(F)F)n3)cc2-c2ccon2)c(F)c1] | 184 |
| 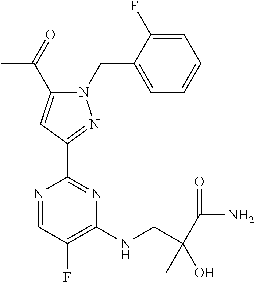 [SMILES: CC(=O)c1cc(-c2ncc(F)c(NCC(C)(O)C(N)=O)n2)nn1Cc1ccccc1F] | 193 |
| 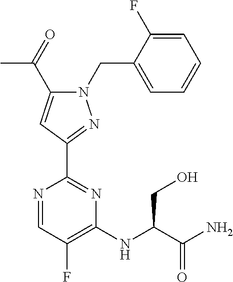 [SMILES: CC(=O)c1cc(-c2ncc(F)c(N[C@@H](CO)C(N)=O)n2)nn1Cc1ccccc1F] | 194 |
| 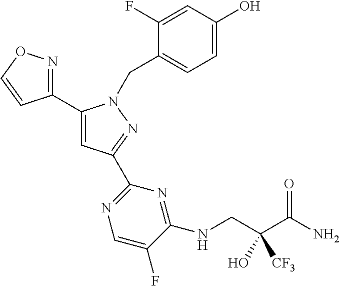 [SMILES: NC(=O)[C@](O)(CNc1nc(-c2cc(-c3ccon3)n(Cc3ccc(O)cc3F)n2)ncc1F)C(F)(F)F] | 195 |
| 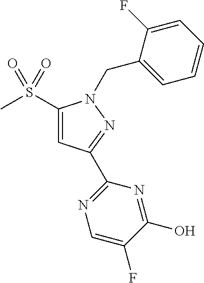 [SMILES: CS(=O)(=O)c1cc(-c2ncc(F)c(O)n2)nn1Cc1ccccc1F] | 196 |
| 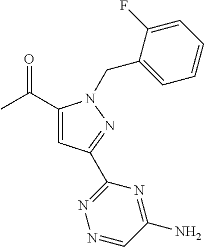 [SMILES: CC(=O)c1cc(-c2nncc(N)n2)nn1Cc1ccccc1F] | 198 |
| 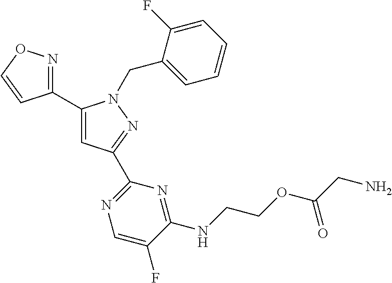 [SMILES: NCC(=O)OCCNc1nc(-c2cc(-c3ccon3)n(Cc3ccccc3F)n2)ncc1F] | 199 |
| 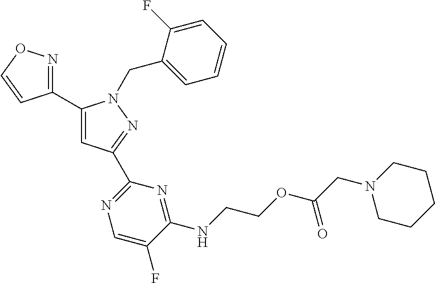 [SMILES: O=C(CN1CCCCC1)OCCNc1nc(-c2cc(-c3ccon3)n(Cc3ccccc3F)n2)ncc1F] | 200 |
| 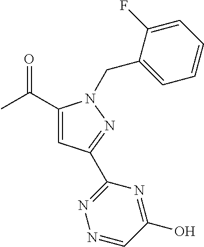 [SMILES: CC(=O)c1cc(-c2nncc(O)n2)nn1Cc1ccccc1F] | 201 |
| 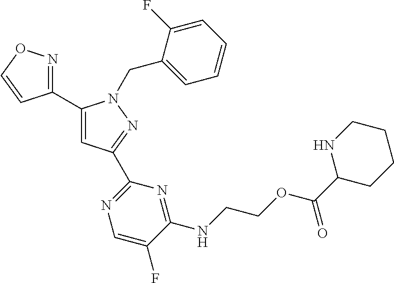 [SMILES: O=C(OCCNc1nc(-c2cc(-c3ccon3)n(Cc3ccccc3F)n2)ncc1F)C1CCCCN1] | 202 |
| 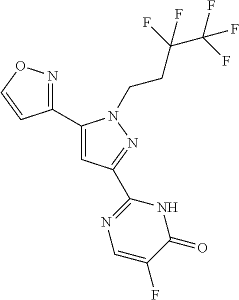 [SMILES: O=c1[nH]c(-c2cc(-c3ccon3)n(CCC(F)(F)C(F)(F)F)n2)ncc1F] | 203 |
| 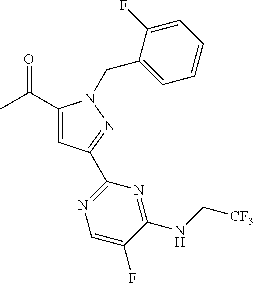 [SMILES: CC(=O)c1cc(-c2ncc(F)c(NCC(F)(F)F)n2)nn1Cc1ccccc1F] | 204 |
| 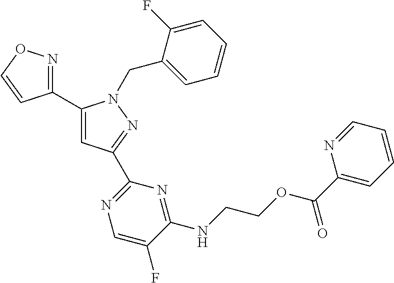 [SMILES: O=C(OCCNc1nc(-c2cc(-c3ccon3)n(Cc3ccccc3F)n2)ncc1F)c1ccccn1] | 206 |
| 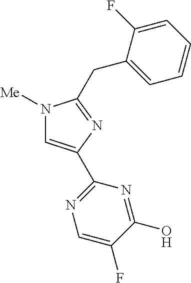 [SMILES: Cn1cc(-c2ncc(F)c(O)n2)nc1Cc1ccccc1F] | 217 |

In some embodiments of the above methods, uses, pharmaceutical compositions and kits, the sGC stimulator is a compound according to Formula IA, or pharmaceutically acceptable salts thereof,

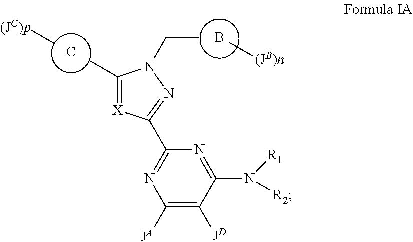

wherein:
X is selected from N, CH, C(C₁₋₄ alkyl), C(C₁₋₄ haloalkyl), CCl and CF;
ring B is a phenyl or a 6-membered heteroaryl ring containing 1 or 2 ring nitrogen atoms, or ring B is a thiophene;
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said R^{B} that is a C₁₋₆ aliphatic and each of said R^{B} that is a C₃₋₈ cycloaliphatic ring is optionally substituted with up to 3 instances of halogen;
J^{A} is selected from hydrogen, halogen, methyl, methoxy, trifluoromethyl, trifluoromethoxy or —NR^{a}R^{b}, wherein R^{a} and R^{b} are each independently selected from hydrogen, C₁₋₆ alkyl or a 3-6 cycloalkyl ring;
J^{D} is hydrogen or selected from halogen, —CN, —CF₃, methoxy, trifluoromethoxy, nitro, amino or methyl;
R¹ and R², together with the nitrogen atom to which they are attached, form a 4 to 8-membered heterocyclic ring or 5 or 6-membered heteroaryl ring; wherein said 4 to 8-membered heterocyclic ring or said 5 or 6-membered heteroaryl ring optionally contains in addition to the nitrogen atom to which R¹ and R² are attached, up to 3 ring heteroatoms independently selected from N, O or S, and is optionally substituted by up to 5 instances of R⁵; or
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, each of said C₃₋₈ cycloalkyl ring, each of said 4 to 8-membered heterocyclic ring group, each of said 5 or 6-membered heteroaryl and each of said C₁₋₆ alkyl portion of each said C₁₋₆ alkyl-R^{Y} is optionally and independently substituted with up to 5 instances of R^{5a}; provided that R¹ and R² are not simultaneously hydrogen; and provided than when X is one of CH, C(C₁₋₄ alkyl), C(C₁₋₄ haloalkyl), CCl or CF, neither of R¹ and R² is a pyridine or a pyrimidine; or
alternatively, J^{D} and one of R¹ or R² can form a 5-6 membered heterocyclic ring containing up to two heteroatoms selected from O, N and S and optionally substituted with up to 3 instances of oxo or —(Y)—R⁹;
wherein Y is either absent or is a linkage in the form of a C₁₋₆ alkyl chain optionally substituted by up to 6 instances of fluoro;
each R⁹ is independently selected from hydrogen, fluoro, —CN, —OR¹⁰, —SR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, a C₃₋₆ cycloalkyl ring, a 4-8-membered heterocyclic ring or a 5-6 membered heteroaryl ring; wherein each said 4 to 8-membered heterocyclic ring and each said 5 to 6-membered heteroaromatic ring contains up to 4 ring heteroatoms independently selected from N, O or S; and wherein each said C₃₋₆ cycloalkyl ring, each said 4 to 8-membered heterocyclic ring and each said 5 to 6-membered heteroaromatic ring is optionally substituted with up to 3 instances of R¹¹;
each R¹¹ is independently selected from halogen, C₁₋₆ alkyl, —CN, —OR¹², —SR¹², —COR¹², —OC(O)R¹², —C(O)OR¹², —C(O)N(R¹²)₂, —C(O)N(R¹²)SO₂R¹², —N(R¹²)C(O)R¹², —N(R¹²)C(O)OR¹², —N(R¹²)C(O)N(R¹²)₂, —N(R¹²)₂, —SO₂R¹², —SO₂N(R¹²)₂, —SO₂N(R¹²)COOR¹², —SO₂N(R¹²)C(O)R¹², —N(R¹²)SO₂R¹² and —N═OR¹²; wherein each of said C₁₋₆ alkyl is optionally and independently substituted by up to 3 instances of fluoro, —OH, —O(C₁₋₄ alkyl), phenyl or —O(C₁₋₄ fluoroalkyl)
wherein each R¹⁰ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo; and
wherein each R¹² is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
R^{Y} is selected from a C₃₋₈ cycloalkyl ring, a 4 to 8-membered heterocyclic ring, phenyl, or a 5 to 6-membered heteroaromatic ring; wherein each of said 4 to 8-membered heterocyclic ring and each of said 5 to 6-membered heteroaromatic ring contains up to 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaromatic ring is optionally substituted with up to 5 instances of R^{5c};
each R^{5c} is independently selected from halogen, —CN, C₁₋₆ alkyl, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each of said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
two instances of R^{5c} attached to the same or different ring atoms of R^{Y}, together with said ring atom or atoms, may form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; wherein R′ is hydrogen or a C₁₋₂ alkyl;
each R^{5a} is independently selected from halogen, —CN, C₁₋₆ alkyl, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring and each of said 4 to 7-membered heterocyclic ring contains up to 4 ring heteroatoms independently selected from N, O and S, wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; or
when one of R¹ or R² is the C₃₋₈ cycloalkyl ring, 4 to 8-membered heterocyclic ring or 5 or 6-membered heteroaryl substituted with up to 5 instances of R^{5a}, two of the instances of R^{5a} attached to the same or different ring atoms of said R¹ or R², together with said atom or atoms, may optionally form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl;
each R⁵ is independently selected from halogen, —CN, C₁₋₆ alkyl, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each said benzyl or each said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁶ is independently selected from hydrogen, a C₁₋₆ alkyl, phenyl, benzyl, a C₃₋₈ cycloalkyl ring or a 4 to 7-membered heterocyclic ring, a 5 or 6-membered heteroaryl ring; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
when R¹ and R² attached to the nitrogen atom form the 4 to 8-membered heterocyclic ring or 5 or 6-membered heteroaryl ring substituted with up to 5 instances of R⁵, two of the instances of R⁵ attached to the same or different atoms of said ring, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, said 4 to 6-membered heterocyclic ring, said phenyl or said 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen; wherein R is hydrogen or a C₁₋₂ alkyl;
p is an integer selected from 0, 1 or 2;
ring C is a monocyclic 5-membered heteroaryl ring containing up to 4 ring heteroatoms selected from N, O or S; wherein said monocyclic 5-membered heteroaryl ring is not a 1,3,5-triazinyl ring;
each J^{C} is independently selected from halogen or a C₁₋₄ aliphatic optionally and independently substituted by up to 3 instances of C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen.

In other embodiments of the above methods, uses, compositions and kits, the sGC stimulator is a compound having Formula IB

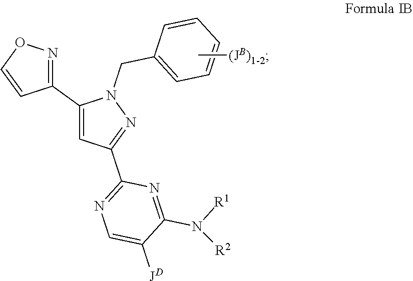

wherein J^{D} is selected from hydrogen or halogen; J^{B} is halogen and
R¹ and R², together with the nitrogen atom to which they are attached, form a 4 to 8-membered heterocyclic ring or 5-membered heteroaryl ring; wherein said 4 to 8-membered heterocyclic ring or said 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 10-membered heterocyclic ring, a 5 or 6-membered heteroaryl, phenyl or a C₁₋₆ alkyl-R^{Y}; wherein each of said 4 to 10-membered heterocyclic ring and each of said 5 or 6-membered heteroaryl ring contains up to 3 ring heteroatoms independently selected from N, O and S; and wherein each of said C₁₋₆ alkyl, each of said C₁₋₆ alkyl portion of each said C₁₋₆ alkyl-R^{Y} moiety, each of said C₃₋₈ cycloalkyl ring, each of said 4 to 10-membered heterocyclic ring group, each of said 5 or 6-membered heteroaryl, each of said phenyl is optionally and independently substituted with up to 5 instances of R^{5f}; provided that neither of R¹ or R² are pyridine or pyrimidine;
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring and each of said 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, each said C₁₋₆ alkyl, each said C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen; and
R″ is hydrogen or a C₁₋₂ alkyl.

In some embodiments of the above methods, uses, compositions and kits, the sGC stimulator is a compound of Formula IC:

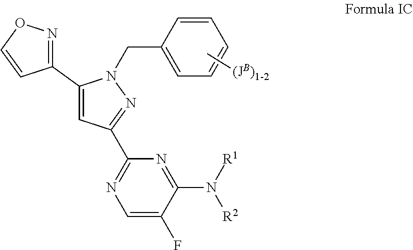

wherein J^{B} is halogen;
R¹ is hydrogen or C₁₋₆ alkyl;
R² is a C₁₋₆ alkyl group optionally and independently substituted by up to three instances of R^{5a}, wherein R^{5a} has been defined in previous paragraphs as part of the description of Formula IA.

In some embodiments of the above methods, uses, compositions and kits, the sGC stimulator is a compound selected from those depicted below, or a pharmaceutical salt thereof:

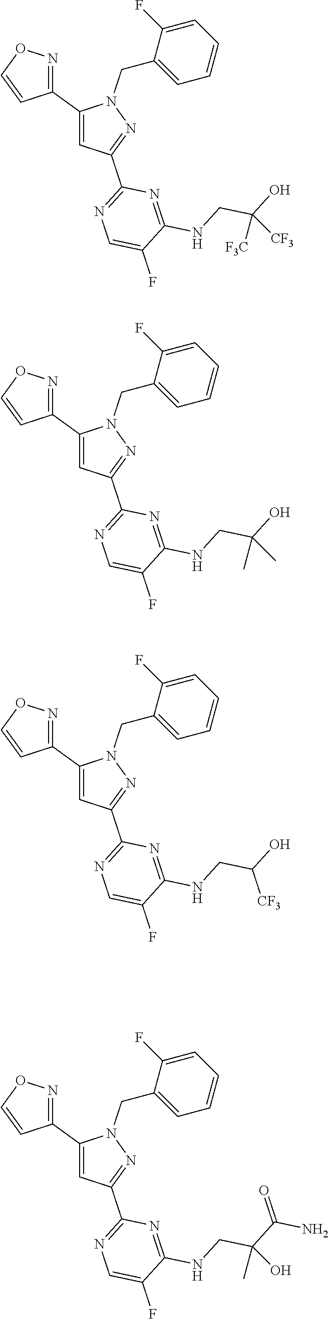 [SMILES: OC(CNc1nc(-c2cc(-c3ccon3)n(Cc3ccccc3F)n2)ncc1F)(C(F)(F)F)C(F)(F)F ; CC(C)(O)CNc1nc(-c2cc(-c3ccon3)n(Cc3ccccc3F)n2)ncc1F ; OC(CNc1nc(-c2cc(-c3ccon3)n(Cc3ccccc3F)n2)ncc1F)C(F)(F)F ; CC(O)(CNc1nc(-c2cc(-c3ccon3)n(Cc3ccccc3F)n2)ncc1F)C(N)=O]

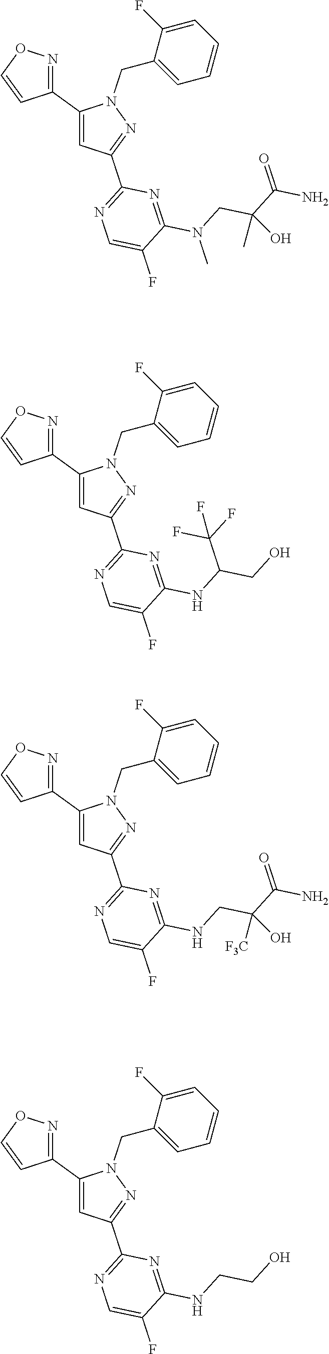 [SMILES: CN(CC(C)(O)C(N)=O)c1nc(-c2cc(-c3ccon3)n(Cc3ccccc3F)n2)ncc1F ; OCC(Nc1nc(-c2cc(-c3ccon3)n(Cc3ccccc3F)n2)ncc1F)C(F)(F)F ; NC(=O)C(O)(CNc1nc(-c2cc(-c3ccon3)n(Cc3ccccc3F)n2)ncc1F)C(F)(F)F ; OCCNc1nc(-c2cc(-c3ccon3)n(Cc3ccccc3F)n2)ncc1F]

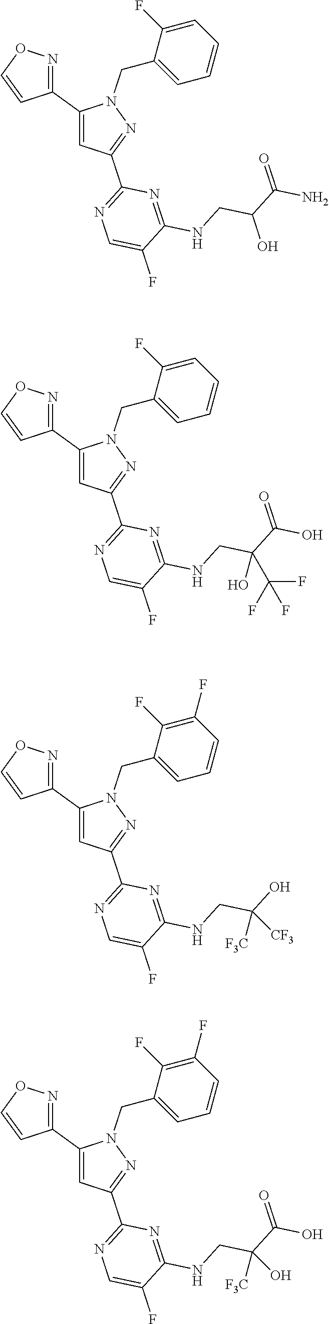 [SMILES: NC(=O)C(O)CNc1nc(-c2cc(-c3ccon3)n(Cc3ccccc3F)n2)ncc1F ; O=C(O)C(O)(CNc1nc(-c2cc(-c3ccon3)n(Cc3ccccc3F)n2)ncc1F)C(F)(F)F ; OC(CNc1nc(-c2cc(-c3ccon3)n(Cc3cccc(F)c3F)n2)ncc1F)(C(F)(F)F)C(F)(F)F ; O=C(O)C(O)(CNc1nc(-c2cc(-c3ccon3)n(Cc3cccc(F)c3F)n2)ncc1F)C(F)(F)F]

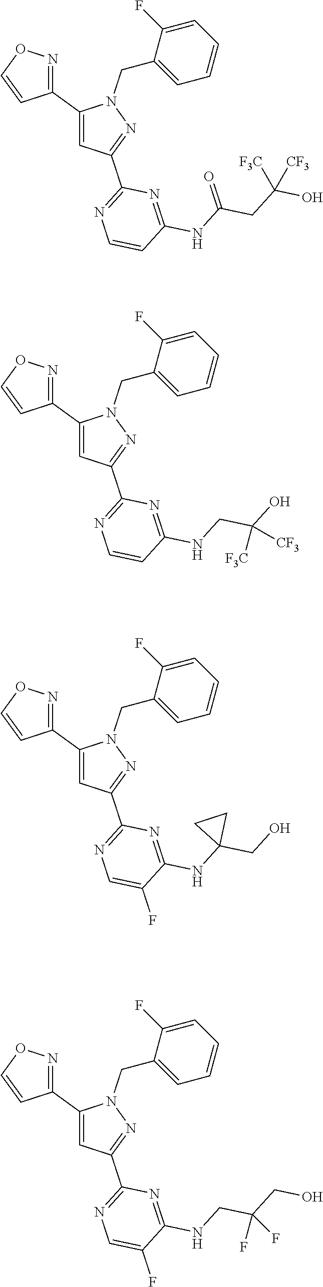 [SMILES: O=C(CC(O)(C(F)(F)F)C(F)(F)F)Nc1ccnc(-c2cc(-c3ccon3)n(Cc3ccccc3F)n2)n1 ; OC(CNc1ccnc(-c2cc(-c3ccon3)n(Cc3ccccc3F)n2)n1)(C(F)(F)F)C(F)(F)F ; OCC1(Nc2nc(-c3cc(-c4ccon4)n(Cc4ccccc4F)n3)ncc2F)CC1 ; OCC(F)(F)CNc1nc(-c2cc(-c3ccon3)n(Cc3ccccc3F)n2)ncc1F]

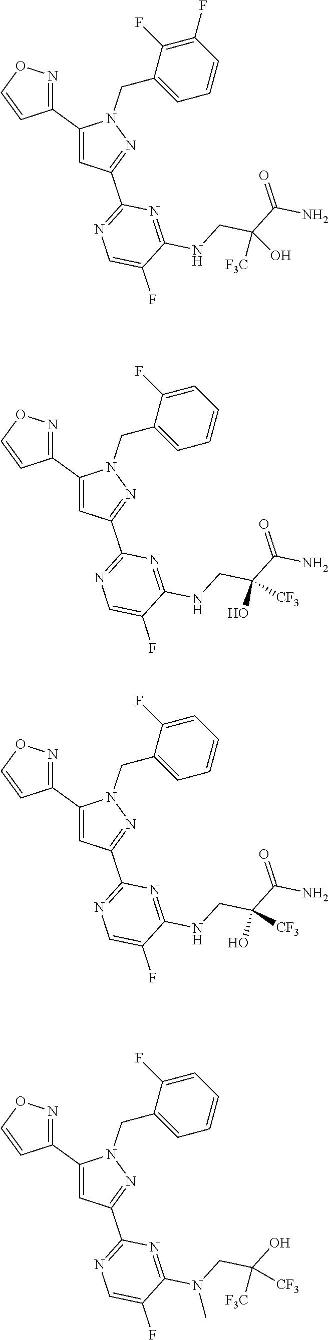 [SMILES: NC(=O)C(O)(CNc1nc(-c2cc(-c3ccon3)n(Cc3cccc(F)c3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F ; CN(CC(O)(C(F)(F)F)C(F)(F)F)c1nc(-c2cc(-c3ccon3)n(Cc3ccccc3F)n2)ncc1F]

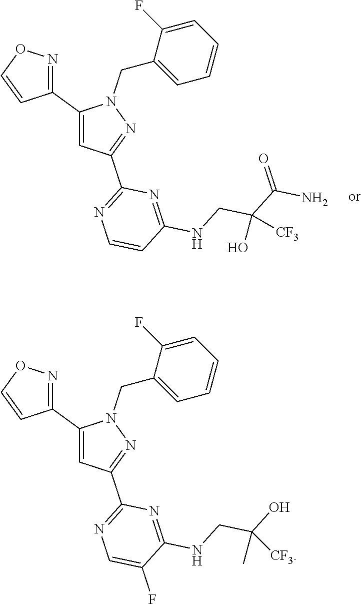 [SMILES: NC(=O)C(O)(CNc1ccnc(-c2cc(-c3ccon3)n(Cc3ccccc3F)n2)n1)C(F)(F)F ; CC(O)(CNc1nc(-c2cc(-c3ccon3)n(Cc3ccccc3F)n2)ncc1F)C(F)(F)F]

In some embodiments of the above methods, uses, compositions and kits, the sGC stimulator is a compound of Formula XZ:

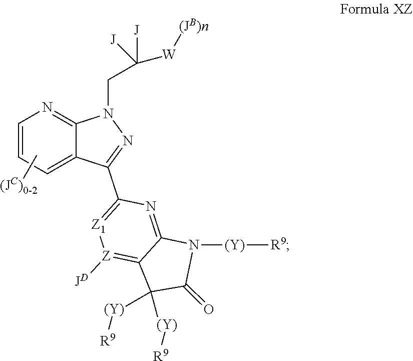

wherein:
W is either
i) absent, and J^{B} is connected directly to the carbon atom bearing two J groups; each J is independently selected from hydrogen or methyl, n is 1 and J^{B} is a C₂₋₇ alkyl chain optionally substituted by between 2 and 9 instances of fluorine; wherein, optionally, one —CH₂— unit of said C₂₋₇ alkyl chain can be replaced by —O— or —S—.
ii) a ring B selected from phenyl, a 5 or 6-membered heteroaryl ring, containing 1 or 2 ring heteroatoms independently selected from N, O or S, a C₃₋₇ cycloalkyl ring and a 4 to 7-membered heterocyclic compound, containing up to 3 heteroatoms independently selected from O, N or S;
wherein when W is ring B
   each J is hydrogen;
   n is 0 or an integer selected from 1, 2 or 3;
   each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³;
   each R^{B} is independently selected from hydrogen, a C₁₋₆ aliphatic or a C₃₋₈ cycloaliphatic; wherein each of said R^{B} that is a C₁₋₆ aliphatic and each of said R^{B} that is a C₃₋₈ cycloaliphatic ring is optionally and independently substituted with up to 3 instances of R^{3a};
   each R³ is independently selected from halogen, —CN, C₁₋₄ alkyl, C₁₋₄ haloalkyl, —O(C₁₋₄ alkyl) or —O(C₁₋₄ haloalkyl);
   each R^{3a} is independently selected from halogen, —CN, C₁₋₄ alkyl, C₁₋₄ haloalkyl, —O(C₁₋₄ alkyl) or —O(C₁₋₄ haloalkyl);
   Z¹ in ring D is selected from CH or N; Z is selected from C or N; wherein if Z¹ is CH, then Z must be C; and if Z¹ is N, then Z may be C or N;
   each J^{D} is independently selected from J^{A}, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, —N(R^{d})SO₂NHR^{D}, —N(R^{d})SO₂NHC(O)OR^{D}, —N(R^{d})SO₂NHC(O)R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocyclic ring and each said 5 to 10-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{D} moiety, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R^{5d};
J^{A} is selected from a lone pair on nitrogen, hydrogen, halogen, oxo, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
each R^{D} is independently selected from hydrogen, a C₁₋₆ aliphatic, —(C₁₋₆ aliphatic)-R^{f}, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 10-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—;
each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b}; wherein when any R^{d} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{dd})—, —CO— or —O—;
each R^{dd} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b};
each R^{f} is independently selected from a C₁₋₃ alkyl, a C₃₋₈ cycloaliphatic ring, a 4 to 10-membered heterocyclic ring, phenyl or a 5 to 6-membered heteroaryl ring; wherein each said 4 to 10-membered heterocyclic ring and each said 5 to 6-membered heteroaryl ring contains between 1 and 4 heteroatoms independently selected from O, N or S; and wherein each said C₃₋₈ cycloaliphatic ring, each said 4 to 10-membered heterocyclic ring, each said phenyl and each said 5 to 6-membered heteroaryl ring is optionally and independently substituted by up to 5 instances of R^{5c};
when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R⁵;
when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R⁵;
when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})SO₂R^{D}, the R^{D} group together with the sulfur atom attached to the R^{D} group, with the nitrogen atom attached to the R^{a} group, and with said R^{a} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
each R⁵ is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of the —(C₁₋₆ alkyl)-R⁶ moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
each R^{5a} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
each R^{5b} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5a} or two instances of R^{5b} attached to the same or different atoms of R^{D} or R^{d}, respectively, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
each R^{5c} is independently selected from halogen, —CN, C₁₋₆ alkyl, —(C₁₋₆ alkyl)-R^{6b}, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR⁶b a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R^{6b} moiety, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5c} attached to the same or different atoms of R^{f}, together with said atom or atoms to which it is attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
each R^{5d} is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl, C₁₋₆ alkyl portion of the —(C₁₋₆ alkyl)-R⁶ moiety, C₇₋₁₂ aralkyl, C₃₋₈ cycloalkyl ring, 4 to 7-membered heterocyclic ring, 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5d} attached to the same or different atoms of J^{D}, together with said atom or atoms of J^{D} to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —C(O)NH₂, —OH or halogen; wherein R is hydrogen or a C₁₋₂ alkyl;
each R⁶ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6b} linked to a nitrogen atom of R^{5c}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
Y is either absent or is a C₁₋₆ alkyl chain, optionally substituted by up to 6 instances of fluoro; and wherein in said Y that is a C₁₋₆ alkyl chain, up to 3 methylene units of this alkyl chain, can be replaced by a group selected from —O—, —C(O)— or —N((Y¹)—R⁹⁰)—, wherein
Y¹ is either absent or is a C₁₋₆ alkyl chain, optionally substituted by up to 6 instances of fluoro; and:
when Y¹ is absent, each R⁹⁰ is independently selected from hydrogen, —COR¹⁰, —C(O)OR, —C(O)N(R¹⁰)₂, —C(O)N(R¹⁰)SO₂ R¹⁰, —SO₂R¹⁰, —SO₂N(R¹⁰)₂, —SO₂N(R¹⁰)COOR¹⁰, —SO₂N(R¹⁰)C(O)R¹⁰, —(C═O)NHOR¹⁰ a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹; and
when Y¹ is present, each R⁹⁰ is independently selected from hydrogen, halogen, —CN, —OR, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R⁹ is independently selected from hydrogen, halogen, a C₁₋₆ alkyl, —CN, —OR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₁₋₆ alkyl, each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R¹⁰ is independently selected from hydrogen, a C₁₋₆ alkyl, —(C₁₋₆ alkyl)-R¹³, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R¹³ moiety, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11a};
each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11b};
each R¹¹ is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R^{11a} is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹; and
each R^{11b} is independently selected from halogen, C₁₋₆ alkyl, oxo, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
each R¹² is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
each R¹²¹ is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo; and
each J^{C} is independently selected from hydrogen or a C₁₋₆ alkyl.

In some embodiments of the above methods, uses, compositions and kits, the sGC stimulator is a compound of Formula XY:

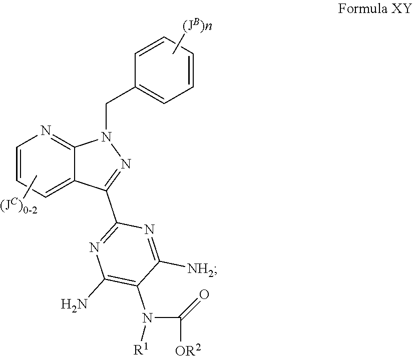

wherein
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said R^{B} that is a C₁₋₆ aliphatic and each of said R^{B} that is a C₃₋₈ cycloaliphatic ring is optionally substituted with up to 3 instances of halogen;
each J^{C}, if present, is independently selected from halogen;
R¹ is hydrogen or C₁₋₆ alkyl; and
R² is a C₁₋₆ alkyl.

In some embodiments of Formula XY, n is 1 or 2. In some embodiments, n is 1.

In some embodiments of Formula XY, each J^{B} is a halogen. In some of these embodiments, each J^{B} is fluoro. In some embodiments of Formula XY, n is 1 and J^{B} is fluoro.

In some embodiments of Formula XY, one or two instances of J^{C} are present. In other embodiments, only one instance of J^{C} is present. In some of these embodiments, J^{C} is fluoro.

In some embodiments of Formula XY, R¹ is selected from hydrogen, methyl or ethyl. In other embodiments, R¹ is hydrogen. In still other embodiments, R¹ is methyl.

In some embodiments of Formula XY, R² is methyl or ethyl. In still other embodiments, R² is methyl.

In some embodiments of Formula XY, the compound is vericiguat or riociguat, depicted supra.

In some embodiments of the above methods, uses, compositions and kits, the sGC stimulator is a compound of Formula IZ, or pharmaceutically acceptable salts thereof,

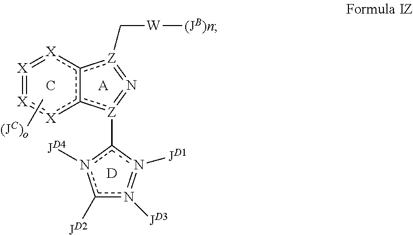

wherein:
rings A and C constitute the core of the molecule; rings A and D are heteroaryl rings; ring C may be a phenyl or a heteroaryl ring; each bond in these rings is either a single or a double bond depending on the substituents, so that each of said rings has aromatic character;
one instance of Z on ring A is N and the other instance of Z is C;
each instance of X on ring C is independently selected from C or N; wherein 0, 1 or 2 instances of X can simultaneously be N;
o is an integer selected from 2, 3 or 4;
each J^{C} is a substituent on a carbon atom independently selected from hydrogen, halogen, —CN, C₁₋₄ aliphatic, C₁₋₄ haloalkyl or C₁₋₄ alkoxy;
W is either:
   i) absent, and J^{B} is connected directly to the methylene group linked to the core; n is 1; and J^{B} is a C₁₋₇ alkyl chain optionally substituted by up to 9 instances of fluorine; or
   ii) a ring B selected from phenyl or a 5 or 6-membered heteroaryl ring, containing 1 or 2 ring heteroatoms independently selected from N, O or S; wherein when W is ring B, n is 0 or an integer selected from 1, 2 or 3;
each J^{B} is independently selected from halogen, —CN, a C₁₋₆ aliphatic, —OR^{B} or a C₃₋₈ cycloaliphatic ring; wherein each said C₁₋₆ aliphatic and each said C₃₋₈ cycloaliphatic ring is optionally and independently substituted with up to 3 instances of R³;
each R^{B} is independently selected from a methyl, propyl, butyl, isopropyl, isobutyl or a C₃₋₈ cycloaliphatic ring; wherein each of said R^{B} is optionally and independently substituted with up to 3 instances of R^{3a};
each R³ and each R^{3a} is independently selected in each instance from halogen, —CN, C₁₋₄ alkyl, C₁₋₄ haloalkyl, —O(C₁₋₄ alkyl) or —O(C₁₋₄ haloalkyl);
J^{D1} and J^{D4} are independently selected from a lone pair on the nitrogen atom to which they are attached or hydrogen, wherein J^{D1} and J^{D4} are not both simultaneously hydrogen or both simultaneously a lone pair;
J^{D3} is either a lone pair on the nitrogen atom to which it is attached, hydrogen, or a substituent selected from —C(O)R^{D}, a C₁₋₆ aliphatic, —(C₁₋₆ aliphatic)-R^{D}, a C₃₋₈ cycloaliphatic ring, a phenyl ring, a 4 to 8-membered heterocyclic ring or a 5 or 6-membered heteroaryl ring; wherein said 4 to 8-membered heterocyclic ring and said 5 or 6-membered heteroaryl ring contains between 1 and 3 heteroatoms independently selected from O, N or S; and wherein said C₁₋₆ aliphatic, said C₁₋₆ aliphatic portion of the —(C₁₋₆ aliphatic)-R^{D} moiety, said C₃₋₈ cycloaliphatic ring, said 4 to 8-membered heterocyclic ring, and said 5 or 6-membered heteroaryl ring is optionally and independently substituted with up to 5 instances of R⁵; and wherein said phenyl ring is optionally and independently substituted with up to 5 instances of R^{5a};
J^{D1} and J^{D3} cannot both simultaneously be hydrogen;
J^{D2} is hydrogen, or a substituent selected from halogen, —CN, —NO₂, —OR^{D1}, —C(O)R^{D}, —C(O)N(R^{D})₂, —N(R^{D})₂, —N(R^{D})C(O)R^{D}, —N(R^{D})C(O)OR^{D}, —N(R^{D})C(O)N(R^{D})₂, —OC(O)N(R^{D})₂, a C₁₋₆ aliphatic, —(C₁₋₆ aliphatic)-R^{D}, a C₃₋₈ cycloaliphatic ring, a phenyl ring, a 4 to 8-membered heterocyclic ring or a 5 or 6-membered heteroaryl ring; wherein said 4 to 8-membered heterocyclic ring and said 5 or 6-membered heteroaryl ring contains between 1 and 3 heteroatoms independently selected from O, N or S; and wherein said C₁₋₆ aliphatic, said C₁₋₆ aliphatic portion of the —(C₁₋₆ aliphatic)-R^{D} moiety, said C₃₋₈ cycloaliphatic ring, said 4 to 8-membered heterocyclic ring and said 5 or 6-membered heteroaryl ring is optionally and independently substituted with up to 5 instances of R⁵; and wherein said phenyl ring is optionally and independently substituted with up to 5 instances of R^{5a};
each R^{D} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring and each said 5 to 6-membered heteroaryl ring is optionally and independently substituted with up to 5 instances of R⁵; and wherein each said phenyl ring is optionally and independently substituted with up to 5 instances of R^{5a};
R^{D1} is selected from a C₁₋₆ aliphatic, —(C₁₋₆ aliphatic)-R^{f}, a C₃₋₈ cycloaliphatic ring, a 4 to 8-membered heterocyclic ring, a phenyl ring or a 5 to 6-membered heteroaryl ring; wherein said 4 to 8-membered heterocyclic ring and said 5 to 6-membered heteroaryl ring contains between 1 and 3 heteroatoms independently selected from O, N or S; and wherein said C₁₋₆ aliphatic, said C₁₋₆ aliphatic portion of the —(C₁₋₆ aliphatic)-R^{f} moiety, said C₃₋₈ cycloaliphatic ring, said 4 to 8-membered heterocyclic ring and said 5 to 6-membered heteroaryl ring is optionally and independently substituted with up to 5 instances of R⁵; wherein said phenyl ring is optionally and independently substituted with up to 5 instances of R^{5a};
each R^{f} is independently selected from a C₃₋₈ cycloaliphatic ring, a 4 to 8-membered heterocyclic ring, a phenyl ring or a 5 to 6-membered heteroaryl ring; wherein each said 4 to 8-membered heterocyclic ring and each said 5 to 6-membered heteroaryl ring contains between 1 and 3 heteroatoms independently selected from O, N or S; and wherein each said C₃₋₈ cycloaliphatic ring, each said 4 to 8-membered heterocyclic ring and each said 5 to 6-membered heteroaryl ring is optionally and independently substituted by up to 5 instances of R⁵; and wherein each said phenyl is optionally and independently substituted by up to 5 instances of R^{5a};
each R⁵ is independently selected from halogen, —CN, C₁₋₆ aliphatic, —(C₁₋₆ alkyl)-R⁶, —OR⁶, —COR⁶, —C(O)N(R⁶)₂, —N(R⁶)C(O)R⁶, —N(R⁶)C(O)OR⁶, —N(R⁶)C(O)N(R⁶)₂, —N(R⁶)₂, a C₃₋₈ cycloalkyl ring, a 4 to 8-membered heterocyclic ring, a 5 or 6-membered heteroaryl ring, phenyl, benzyl or an oxo group; wherein if two instances of R⁵ are oxo and —OH or oxo and —OR⁶, they are not substituents on the same carbon atom; wherein each of said 5 or 6-membered heteroaryl ring or 4 to 8-membered heterocyclic ring contains up to 3 ring heteroatoms independently selected from N, O and S; and wherein each of said C₁₋₆ aliphatic, each said C₁₋₆ alkyl portion of the —(C₁₋₆ alkyl)-R⁶ moiety, each said C₃₋₈ cycloalkyl ring, each said 5 or 6-membered heteroaryl ring and each said 4 to 8-membered heterocyclic ring, is optionally and independently substituted with up to 3 instances of halogen, C₁₋₄ alkyl, —OH, —NH₂, —NH(C₁₋₄ alkyl), —N(C₁₋₄ alkyl)₂, —CN, —CONH₂, —O(C₁₋₄ alkyl), —O(C₁₋₄ haloalkyl) or oxo; wherein if two instances of a substituent on R⁵ are a) oxo and —OH or b) oxo and —O(C₁₋₄ alkyl) or c) oxo and —O(C₁₋₄ haloalkyl), they are not substituents on the same carbon atom; wherein each said benzyl or phenyl is optionally and independently substituted with up to 3 instances of halogen, C₁₋₄ alkyl, —NH₂, —NH(C₁₋₄ alkyl), —N(C₁₋₄ alkyl)₂, —CN, —CONH₂, —O(C₁₋₄ alkyl), —O(C₁₋₄ haloalkyl);
each R^{5a} is independently selected from halogen, —CN, C₁₋₆ aliphatic, —(C₁₋₆ alkyl)-R⁶, —OR^{6a}, —COR⁶, —C(O)N(R⁶)₂, —N(R⁶)C(O)R⁶, —N(R⁶)C(O)OR⁶, —N(R⁶)C(O)N(R⁶)₂, —N(R⁶)₂, a C₃₋₈ cycloalkyl ring, a 4 to 8-membered heterocyclic ring, a 5 or 6-membered heteroaryl ring, phenyl, benzyl or an oxo group; wherein each of said 5 or 6-membered heteroaryl ring and each of said 4 to 8-membered heterocyclic ring contains up to 3 ring heteroatoms independently selected from N, O and S; and wherein each of said C₁₋₆ aliphatic, each of said C₁₋₆ alkyl portion of the —(C₁₋₆ alkyl)-R⁶ moiety, each of said C₃₋₈ cycloalkyl ring, each of said 4 to 8-membered heterocyclic ring and each of said 5 or 6-membered heteroaryl ring is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —CONH₂, —O(C₁₋₄ alkyl), —O(C₁₋₄ haloalkyl) or oxo; wherein if two instances of a substituent on R^{5a} are a) oxo and —OH or b) oxo and —O(C₁₋₄ alkyl) or c) oxo and —O(C₁₋₄ haloalkyl), they are not substituents on the same carbon atom; and wherein each of said benzyl and each of said phenyl is optionally and independently substituted with up to 3 instances of halogen, C₁₋₄ alkyl, C₁₋₄ haloalkyl —NH₂, —NH(C₁₋₄ alkyl), —N(C₁₋₄ alkyl)₂, —CN, —CONH₂, —O(C₁₋₄ alkyl) or —O(C₁₋₄ haloalkyl);
each R⁶ is independently selected from hydrogen, a C₁₋₆ aliphatic, phenyl, benzyl, a C₃₋₈ cycloalkyl ring, a 4 to 8-membered heterocyclic ring or a 5 or 6-membered heteroaryl ring; wherein each of said 5 or 6-membered heteroaryl ring or 4 to 8-membered heterocyclic ring contains up to 3 ring heteroatoms independently selected from N, O and S; wherein each of said C₁₋₆ aliphatic, each of said C₃₋₈ cycloalkyl ring, each of said 4 to 8-membered heterocyclic ring and each of said 5 or 6-membered heteroaryl ring is optionally and independently substituted with up to 3 instances of halogen, C₁₋₄ alkyl, C₁₋₄ haloalkyl —OH, —NH₂, —NH(C₁₋₄ alkyl), —N(C₁₋₄ alkyl)₂, —CN, —C(O)NH₂, —O(C₁₋₄ alkyl), —O(C₁₋₄ haloalkyl) or oxo; wherein if two instances of a substituent on R⁶ are a) oxo and —OH or b) oxo and —O(C₁₋₄ alkyl) or c) oxo and —O(C₁₋₄ haloalkyl), they are not substituents on the same carbon atom; wherein each of said phenyl and each of said benzyl is optionally and independently substituted with up to 3 instances of halogen, C₁₋₄ alkyl, C₁₋₄ haloalkyl, —NH₂, —NH(C₁₋₄ alkyl), —N(C₁₋₄ alkyl)₂, —CN, —C(O)NH₂, —O(C₁₋₄ alkyl), —O(C₁₋₄ haloalkyl) or oxo;
each R^{6a} is independently selected from a C₁₋₆ aliphatic, phenyl, benzyl, a C₃₋₈ cycloalkyl ring, a 4 to 8-membered heterocyclic ring or a 5 or 6-membered heteroaryl ring; wherein each of said 5 or 6-membered heteroaryl ring and each of said 4 to 8-membered heterocyclic ring contains up to 3 ring heteroatoms independently selected from N, O and S; wherein each of said C₁₋₆ aliphatic, each of said C₃₋₈ cycloalkyl ring, each of said 4 to 8-membered heterocyclic ring and each of said 5 or 6-membered heteroaryl ring is optionally and independently substituted with up to 3 instances of halogen, C₁₋₄ alkyl, C₁₋₄ haloalkyl —OH, —NH₂, —NH(C₁₋₄ alkyl), —N(C₁₋₄ alkyl)₂, —CN, —C(O)NH₂, —O(C₁₋₄ alkyl), —O(C₁₋₄ haloalkyl) or oxo; wherein if two instances of R^{6a} are a) oxo and —OH or b) oxo and —O(C₁₋₄ alkyl) or c) oxo and —O(C₁₋₄ haloalkyl), they are not substituents on the same carbon atom; wherein each of said phenyl and each of said benzyl is optionally and independently substituted with up to 3 instances of halogen, C₁₋₄ alkyl, C₁₋₄ haloalkyl, —NH₂, —NH(C₁₋₄ alkyl), —N(C₁₋₄ alkyl)₂, —CN, —C(O)NH₂, —O(C₁₋₄ alkyl), —O(C₁₋₄ haloalkyl) or oxo;
alternatively, J^{D2} and J^{D3}, together with the atoms to which they are attached, form a 5 or 6-membered heteroaryl ring or a 5 to 8-membered heterocyclic ring; wherein said heteroaryl ring or heterocyclic ring contains between 1 and 3 heteroatoms independently selected from N, O or S, including the N to which J^{D3} is attached; wherein said heterocyclic or heteroaryl ring can be substituted by up to three instances of J^{E}; and
J^{E} is selected from halogen, C₁₋₄ alkyl, C₁₋₄ haloalkyl or oxo.

In some embodiments of Formula IZ, the compound is one of Formula IIZA, Formula IIZB or Formula IIZC, or a pharmaceutically acceptable salt thereof:

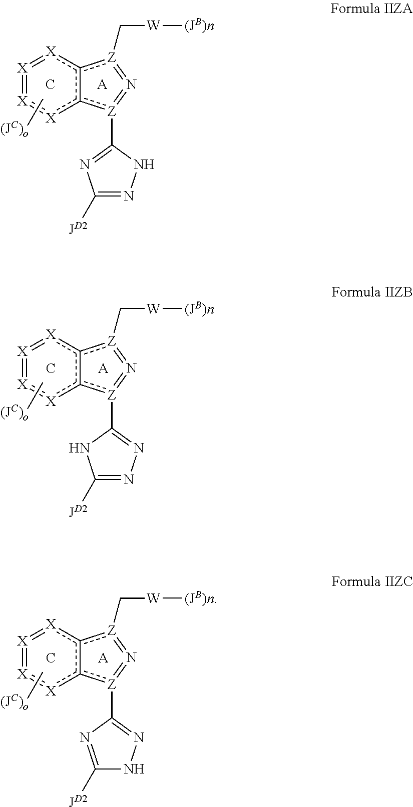

In some embodiments of Formula IZ, Formula IIZA, Formula IIZB or Formula IIZC, J^{D2} is selected from: hydrogen, halogen, —CN, —OR^{D1}, —C(O)R^{D}, —C(O)N(R^{D})₂, —N(R^{D})₂, —N(R^{D})C(O)R^{D}, a C₁₋₆ aliphatic, —(C₁₋₆ aliphatic)-R^{D}, a C₃₋₈ cycloaliphatic ring, a phenyl ring, and a 4 to 8-membered heterocyclic ring containing between 1 and 3 heteroatoms independently selected from O, N or S. In some embodiments, the C₁₋₆ aliphatic, C₁₋₆ aliphatic portion of the —(C₁₋₆ aliphatic)-R^{D} moiety, C₃₋ₛ cycloaliphatic ring, 4 to 8-membered heterocyclic ring, or 5 or 6-membered heteroaryl ring may be substituted with up to 5 instances of R⁵, and each instance of R⁵ may be the same or different. In some of these embodiments, R⁵ is selected in each instance from halogen, C₁₋₆ haloalkyl, —OH, —OCH₃, —C(O)CF₃, —NHC(O)O(C₁₋₆ aliphatic), —NH₂, phenyl, —CH₂— heteroaryl, —N(CH₃)₂, C₁₋₆ aliphatic, —NHC(O)R⁶, or oxo. In other embodiments, the phenyl ring may be substituted with up to 5 instances of R^{5a}, and each instance of R^{5a} may be the same or different. In some of these embodiments, R^{5a} is selected in each instance from halogen, C₁₋₆ haloalkyl, —OH, —OCH₃, —C(O)CF₃, —NHC(O)O(C₁₋₆ aliphatic), —NH₂, phenyl, —CH₂-heteroaryl, —N(CH₃)₂, C₁₋₆ aliphatic, —NHC(O)R⁶, or oxo.

In some embodiments of Formula IZ, Formula IIZA, Formula IIZB or Formula IIZC, J^{D3} is hydrogen or a lone pair of electrons on the nitrogen to which it is attached.

In some embodiments of Formula IZ, Formula IIZA, Formula IIZB or Formula IIZC, the compound is one of Formula IIIZ, or a pharmaceutically acceptable salt thereof:

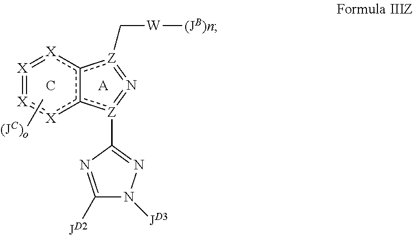

wherein J^{D3} is not hydrogen or a lone pair on the N atom to which it is attached.

In some embodiments of Formula IZ or Formula IIIZ, J^{D2} and J^{D3}, together with the atoms to which they are attached, form a 5 or 6-membered heteroaryl ring or a 5 to 8-membered heterocyclic ring; wherein said heteroaryl ring or heterocyclic ring contains between 1 and 3 heteroatoms independently selected from N, O or S, including the N to which J^{D3} is attached. In some of these embodiments, the heterocyclic or heteroaryl ring can be substituted by up to three instances of J^{E}. In some of these embodiments, J^{E} is selected from halogen, C₁₋₄ alkyl, C₁₋₄ haloalkyl or oxo. In other embodiments, J^{D2} and J^{D3}, together with the atoms to which they are attached, form a ring selected from pyrrole, pyridine, oxazine, pyrimidine, diazepine, pyrazine, pyridazine, and imidazole. In these embodiments, the ring is partially or fully saturated and is optionally substituted by up to three instances of J^{E}.

In some embodiments of Formula IZ, Formula IIZA, Formula IIZB, Formula IIZC and Formula IIIZ, J^{D2} is selected from hydrogen, halogen, —NH₂, —CF₃, —CH₃, and —CH₂OH.

In some embodiments of Formula IZ or Formula IIIZ, J^{D3} is a C₁₋₆ aliphatic. In some of these embodiments, the C₁₋₆ aliphatic may be substituted with up to 5 instances of R⁵, and each instance of R⁵ may be the same or different.

In some embodiments of Formula IZ or Formula IIIZ, J^{D2} is selected from hydrogen, halogen, —NH₂, —CF₃, —CH₃, and —CH₂OH; and J^{D3} is a C₁₋₆ aliphatic. In some of these embodiments, the C₁₋₆ aliphatic may be substituted with up to 5 instances of R⁵, and each instance of R⁵ may be the same or different. In some of these embodiments, each R⁵ is independently selected from halogen, —CN, —OR⁶, —C(O)N(R⁶)₂, a 4 to 8-membered heterocyclic ring (containing up to 3 ring heteroatoms independently selected from N, O and S), or phenyl. In some embodiments, the 4 to 8-membered heterocyclic ring is optionally and independently substituted with up to 3 instances of halogen, —O(C₁₋₄ alkyl), or oxo. In some embodiments, the phenyl is optionally and independently substituted with up to 3 instances of halogen. In some of these embodiments, J^{D3} is selected from —C₁₋₄ alkyl, —CH₂CF₃, —(CH₂)₂OH, —CH₂C(O)NH₂, —CH₂CN, —CH₂C(OH)CF₃, —(CH₂)₂ pyrrolidin-2-one, or benzyl optionally substituted with methoxy or halogen.

In some embodiments of Formula IZ, Formula IIZA, Formula IIZB, Formula IIZC or Formula IIIZ, W is absent, and J^{B} is connected directly to the methylene group linked to the core; n is 1; and J^{B} is a C₁₋₇ alkyl chain optionally substituted by up to 9 instances of fluorine.

In some embodiments of Formula IZ, Formula IIZA, Formula IIZB, Formula IIZC or Formula IIIZ, W is a ring B selected from phenyl or a 5 or 6-membered heteroaryl ring, and the compound is one of Formula IVZ, or a pharmaceutically acceptable salt thereof:

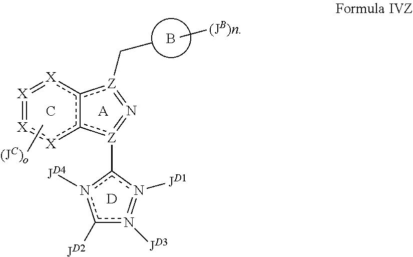

In other embodiments of Formula IZ, Formula IIZA, Formula IIZB, Formula IIZC, Formula IIIZ or Formula IVZ, ring B is selected from phenyl, pyridine, pyridazine, pyrazine, and pyrimidine. In still other embodiments, ring B is phenyl. In yet other embodiments, ring B is pyridine or pyrimidine

In some embodiments of Formula IZ, Formula IIZA, Formula IIZB, Formula IIZC, Formula IIIZ or Formula IVZ, n is 1. In other embodiments of Formula IZ, Formula IIZA, Formula IIZB, Formula IIZC, Formula IIIZ or Formula IVZ, n is 2. In still other embodiments of Formula IZ, Formula IIZA, Formula IIZB, Formula IIZC, Formula IIIZ or Formula IVZ, n is 0. In some embodiments of Formula IZ, Formula IIZA, Formula IIZB, Formula IIZC, Formula IIIZ or Formula IVZ, n is 3.

In some embodiments of Formula IZ, Formula IIZA, Formula IIZB, Formula IIZC, Formula IIIZ or Formula IVZ, each J^{B} is independently selected from halogen and a C₁₋₆ aliphatic. In other embodiments, each J^{B} is independently selected from halogen atoms. In still other embodiments, each J^{B} is independently selected from fluoro or chloro. In yet other embodiments, each J^{B} is fluoro. In some embodiments, each J^{B} is a C₁₋₆ aliphatic. In other embodiments, each J^{B} is methyl.

In some embodiments of Formula IZ, Formula IIZA, Formula IIZB, Formula IIZC, Formula IIIZ or Formula IVZ, wherein ring B is present, at least one J^{B} is ortho to the attachment of the methylene linker between ring B and ring A. In some embodiments, one J^{B} is ortho to the attachment of the methylene linker between rings B and Ring A and is fluoro.

In some embodiments of Formula IZ, Formula IIZA, Formula IIZB, Formula IIZC, Formula IIIZ or Formula IVZ, the core formed by rings C and A is selected from:

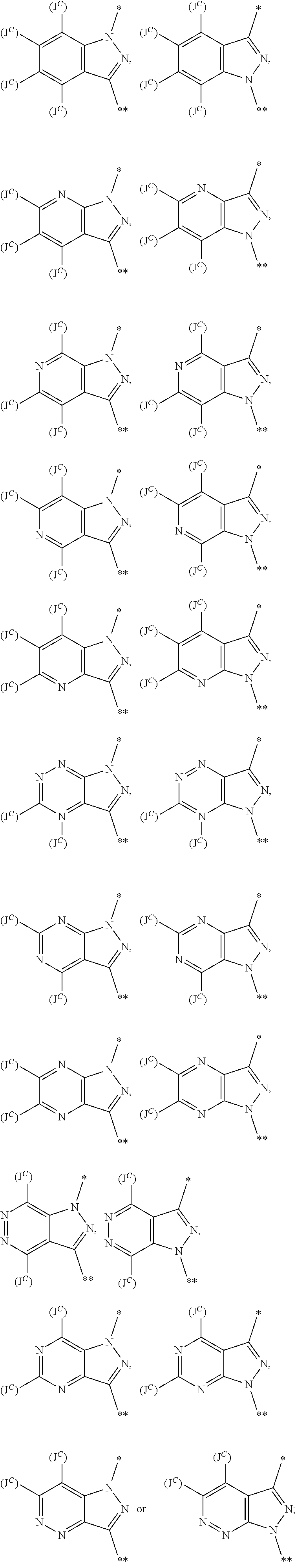

wherein the atom with a symbol * represents the attachment point to the methylene linker to W-(J^{B})ₙ; and the atom with a symbol ** represents the point of attachment to ring D. In other embodiments, the core formed by rings C and A is selected from:

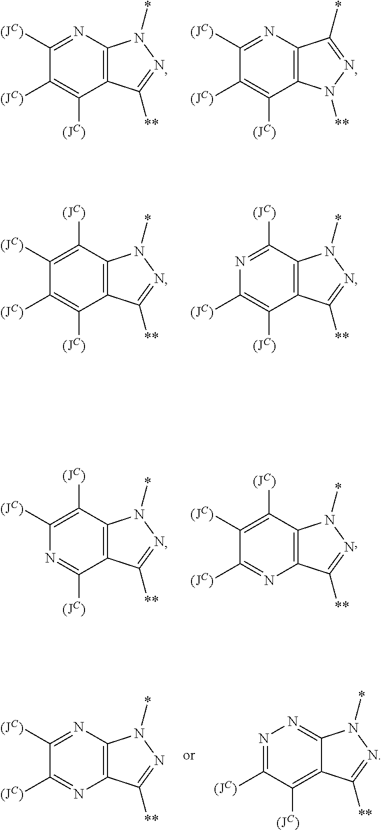

In still other embodiments, the core formed by rings C and A is selected from:

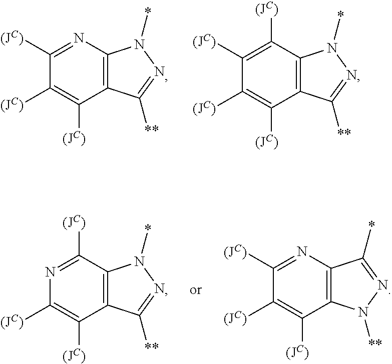

In some embodiments of Formula IZ, Formula IIZA, Formula IIZB, Formula IIZC, Formula IIIZ or Formula IVZ, the core formed by rings C and A is selected from:

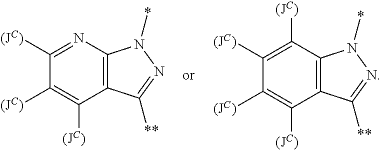

In other embodiments of Formula IZ, Formula IIZA, Formula IIZB, Formula IIZC, Formula IIIZ or Formula IVZ, the core formed by rings C and A is selected from:

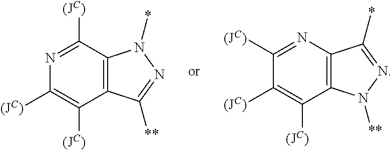

In some embodiments of Formula IZ, Formula IIZA, Formula IIZB, Formula IIZC, Formula IIIZ or Formula IVZ, each J^{C} is independently selected from hydrogen, halogen, or C₁₋₄ aliphatic. In other embodiments, each J^{C} is independently selected from hydrogen, fluoro, chloro, or methyl.

In some embodiments, the compounds of Formula IZ are selected from those listed in Table IZA, or a pharmaceutically acceptable salt thereof.

| | |
|---|---|
| 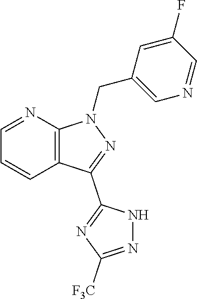 [SMILES: Fc1cncc(Cn2nc(-c3nc(C(F)(F)F)n[nH]3)c3cccnc32)c1] | IZA-1 |
| 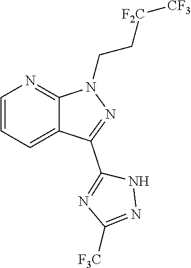 [SMILES: FC(F)(F)c1n[nH]c(-c2nn(CCC(F)(F)C(F)(F)F)c3ncccc23)n1] | IZA-2 |
| 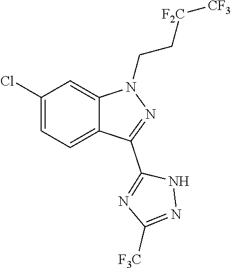 [SMILES: FC(F)(F)c1n[nH]c(-c2nn(CCC(F)(F)C(F)(F)F)c3cc(Cl)ccc23)n1] | IZA-3 |
| 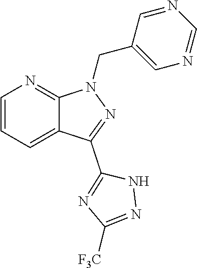 [SMILES: FC(F)(F)c1n[nH]c(-c2nn(Cc3cncnc3)c3ncccc23)n1] | IZA-4 |
| 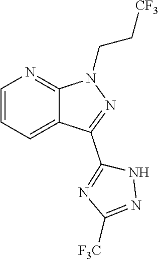 [SMILES: FC(F)(F)CCn1nc(-c2nc(C(F)(F)F)n[nH]2)c2cccnc21] | IZA-7 |
| 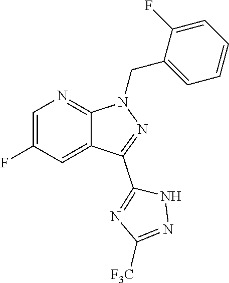 [SMILES: Fc1cnc2c(c1)c(-c1nc(C(F)(F)F)n[nH]1)nn2Cc1ccccc1F] | IZA-8 |
| 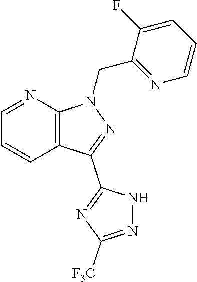 [SMILES: Fc1cccnc1Cn1nc(-c2nc(C(F)(F)F)n[nH]2)c2cccnc21] | IZA-13 |
| 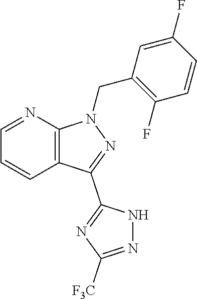 [SMILES: Fc1ccc(F)c(Cn2nc(-c3nc(C(F)(F)F)n[nH]3)c3cccnc32)c1] | IZA-14 |
| 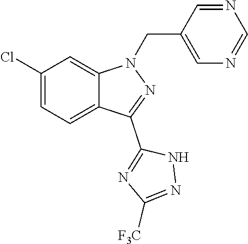 [SMILES: FC(F)(F)c1n[nH]c(-c2nn(Cc3cncnc3)c3cc(Cl)ccc23)n1] | IZA-16 |
| 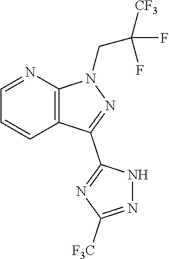 [SMILES: FC(F)(F)c1n[nH]c(-c2nn(CC(F)(F)C(F)(F)F)c3ncccc23)n1] | IZA-19 |
| 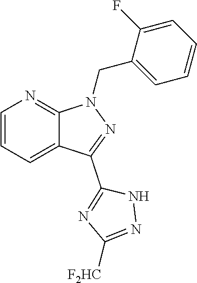 [SMILES: Fc1ccccc1Cn1nc(-c2nc(C(F)F)n[nH]2)c2cccnc21] | IZA-20 |
| 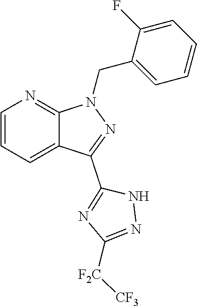 [SMILES: Fc1ccccc1Cn1nc(-c2nc(C(F)(F)C(F)(F)F)n[nH]2)c2cccnc21] | IZA-21 |
| 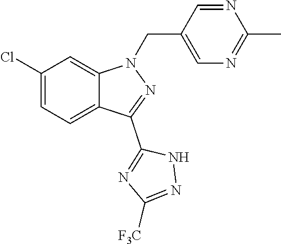 [SMILES: Cc1ncc(Cn2nc(-c3nc(C(F)(F)F)n[nH]3)c3ccc(Cl)cc32)cn1] | IZA-22 |
| 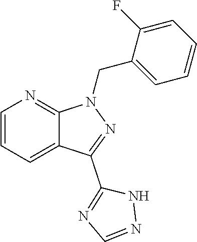 [SMILES: Fc1ccccc1Cn1nc(-c2ncn[nH]2)c2cccnc21] | IZA-25 |
| 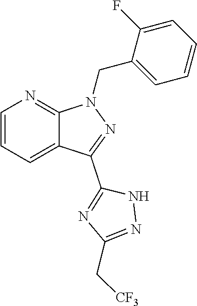 [SMILES: Fc1ccccc1Cn1nc(-c2nc(CC(F)(F)F)n[nH]2)c2cccnc21] | IZA-26 |
| 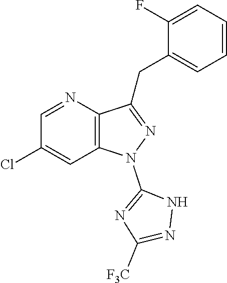 [SMILES: Fc1ccccc1Cc1nn(-c2nc(C(F)(F)F)n[nH]2)c2cc(Cl)cnc12] | IZA-30 |
| 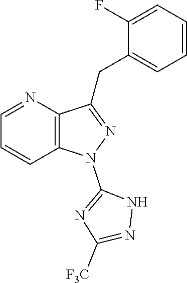 [SMILES: Fc1ccccc1Cc1nn(-c2nc(C(F)(F)F)n[nH]2)c2cccnc12] | IZA-31 |
| 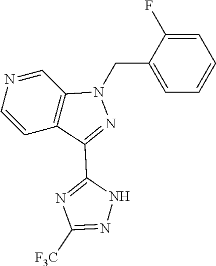 [SMILES: Fc1ccccc1Cn1nc(-c2nc(C(F)(F)F)n[nH]2)c2ccncc21] | IZA-32 |
| 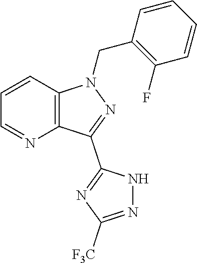 [SMILES: Fc1ccccc1Cn1nc(-c2nc(C(F)(F)F)n[nH]2)c2ncccc21] | IZA-35 |
| 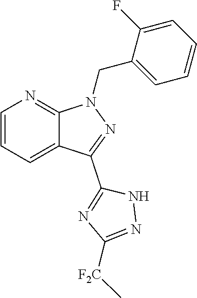 [SMILES: CC(F)(F)c1n[nH]c(-c2nn(Cc3ccccc3F)c3ncccc23)n1] | IZA-36 |
| 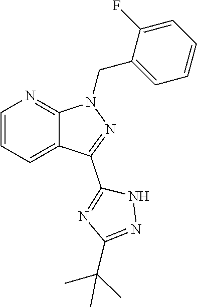 [SMILES: CC(C)(C)c1n[nH]c(-c2nn(Cc3ccccc3F)c3ncccc23)n1] | IZA-37 |
| 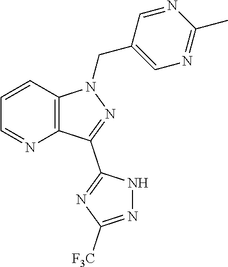 [SMILES: Cc1ncc(Cn2nc(-c3nc(C(F)(F)F)n[nH]3)c3ncccc32)cn1] | IZA-38 |
| 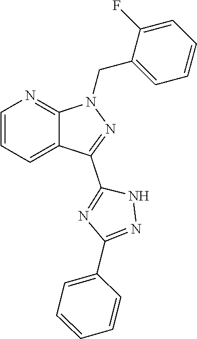 [SMILES: Fc1ccccc1Cn1nc(-c2nc(-c3ccccc3)n[nH]2)c2cccnc21] | IZA-39 |
| 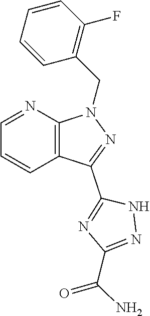 [SMILES: NC(=O)c1n[nH]c(-c2nn(Cc3ccccc3F)c3ncccc23)n1] | IZA-40 |
| 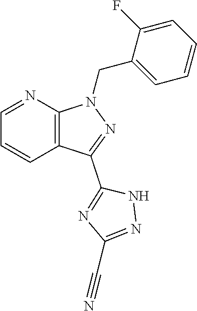 [SMILES: N#Cc1n[nH]c(-c2nn(Cc3ccccc3F)c3ncccc23)n1] | IZA-41 |
| 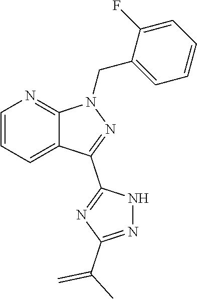 [SMILES: C=C(C)c1n[nH]c(-c2nn(Cc3ccccc3F)c3ncccc23)n1] | IZA-55 |
| 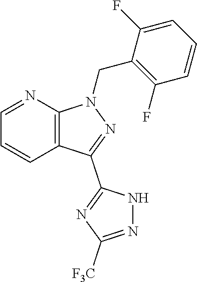 [SMILES: Fc1cccc(F)c1Cn1nc(-c2nc(C(F)(F)F)n[nH]2)c2cccnc21] | IZA-42 |
| 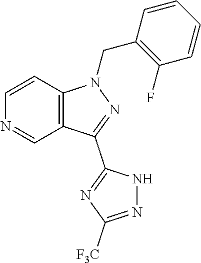 [SMILES: Fc1ccccc1Cn1nc(-c2nc(C(F)(F)F)n[nH]2)c2cnccc21] | IZA-43 |
| 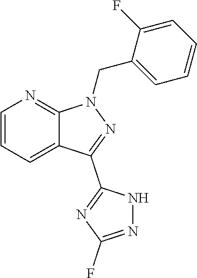 [SMILES: Fc1n[nH]c(-c2nn(Cc3ccccc3F)c3ncccc23)n1] | IZA-45 |
| 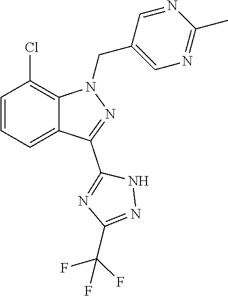 [SMILES: Cc1ncc(Cn2nc(-c3nc(C(F)(F)F)n[nH]3)c3cccc(Cl)c32)cn1] | IZA-46 |
| 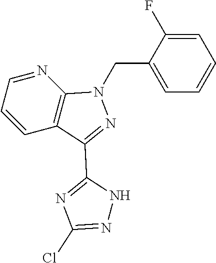 [SMILES: Fc1ccccc1Cn1nc(-c2nc(Cl)n[nH]2)c2cccnc21] | IZA-47 |
| 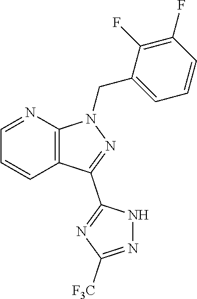 [SMILES: Fc1cccc(Cn2nc(-c3nc(C(F)(F)F)n[nH]3)c3cccnc32)c1F] | IZA-48 |
| 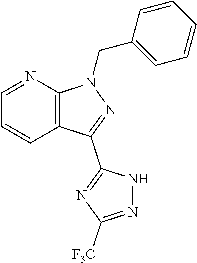 [SMILES: FC(F)(F)c1n[nH]c(-c2nn(Cc3ccccc3)c3ncccc23)n1] | IZA-49 |
| 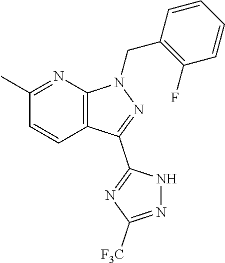 [SMILES: Cc1ccc2c(-c3nc(C(F)(F)F)n[nH]3)nn(Cc3ccccc3F)c2n1] | IZA-50 |
| 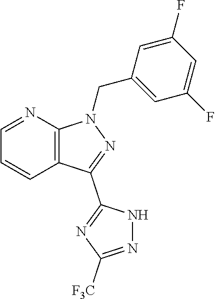 [SMILES: Fc1cc(F)cc(Cn2nc(-c3nc(C(F)(F)F)n[nH]3)c3cccnc32)c1] | IZA-51 |
| 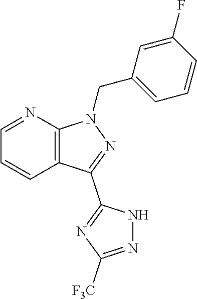 [SMILES: Fc1cccc(Cn2nc(-c3nc(C(F)(F)F)n[nH]3)c3cccnc32)c1] | IZA-52 |
| 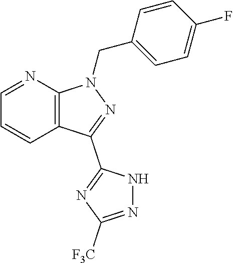 [SMILES: Fc1ccc(Cn2nc(-c3nc(C(F)(F)F)n[nH]3)c3cccnc32)cc1] | IZA-53 |
| 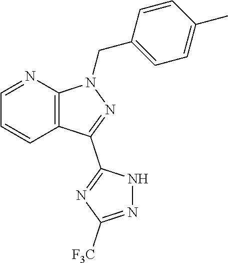 [SMILES: Cc1ccc(Cn2nc(-c3nc(C(F)(F)F)n[nH]3)c3cccnc32)cc1] | IZA-54 |
| 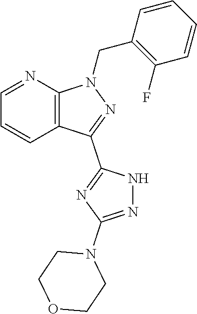 [SMILES: Fc1ccccc1Cn1nc(-c2nc(N3CCOCC3)n[nH]2)c2cccnc21] | IZA-57 |
| 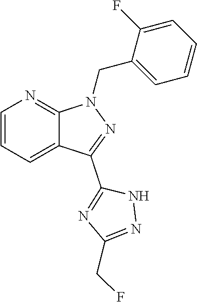 [SMILES: FCc1n[nH]c(-c2nn(Cc3ccccc3F)c3ncccc23)n1] | IZA-58 |
| 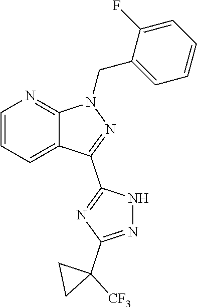 [SMILES: Fc1ccccc1Cn1nc(-c2nc(C3(C(F)(F)F)CC3)n[nH]2)c2cccnc21] | IZA-59 |
| 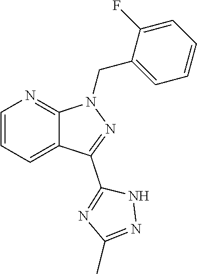 [SMILES: Cc1n[nH]c(-c2nn(Cc3ccccc3F)c3ncccc23)n1] | IZA-60 |
| 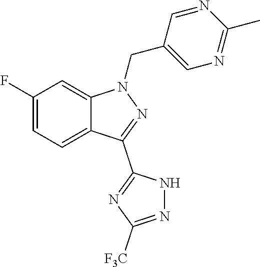 [SMILES: Cc1ncc(Cn2nc(-c3nc(C(F)(F)F)n[nH]3)c3ccc(F)cc32)cn1] | IZA-61 |
| 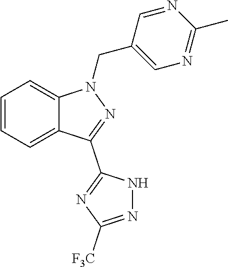 [SMILES: Cc1ncc(Cn2nc(-c3nc(C(F)(F)F)n[nH]3)c3ccccc32)cn1] | IZA-62 |
| 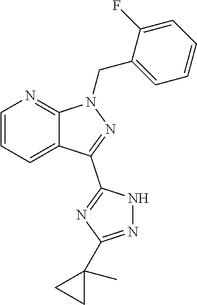 [SMILES: CC1(c2n[nH]c(-c3nn(Cc4ccccc4F)c4ncccc34)n2)CC1] | IZA-63 |
| 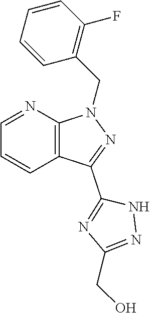 [SMILES: OCc1n[nH]c(-c2nn(Cc3ccccc3F)c3ncccc23)n1] | IZA-64 |
| 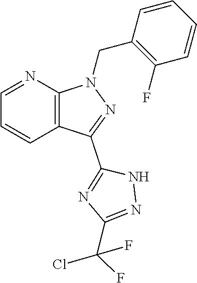 [SMILES: Fc1ccccc1Cn1nc(-c2nc(C(F)(F)Cl)n[nH]2)c2cccnc21] | IZA-65 |
| 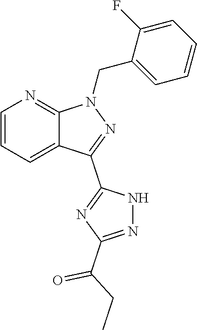 [SMILES: CCC(=O)c1n[nH]c(-c2nn(Cc3ccccc3F)c3ncccc23)n1] | IZA-66 |
| 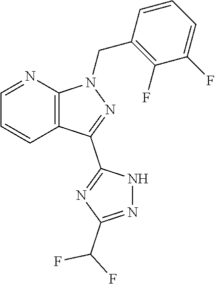 [SMILES: Fc1cccc(Cn2nc(-c3nc(C(F)F)n[nH]3)c3cccnc32)c1F] | IZA-67 |
| 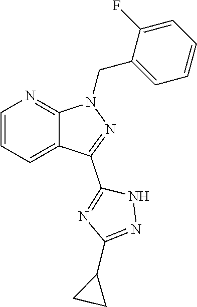 [SMILES: Fc1ccccc1Cn1nc(-c2nc(C3CC3)n[nH]2)c2cccnc21] | IZA-68 |
| 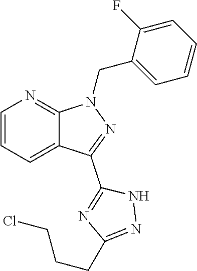 [SMILES: Fc1ccccc1Cn1nc(-c2nc(CCCCl)n[nH]2)c2cccnc21] | IZA-69 |
| 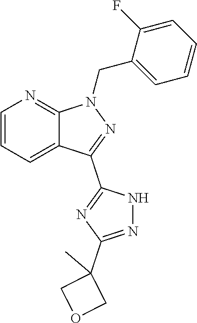 [SMILES: CC1(c2n[nH]c(-c3nn(Cc4ccccc4F)c4ncccc34)n2)COC1] | IZA-70 |
| 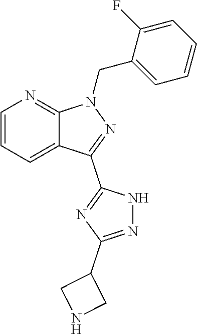 [SMILES: Fc1ccccc1Cn1nc(-c2nc(C3CNC3)n[nH]2)c2cccnc21] | IZA-73 |
| 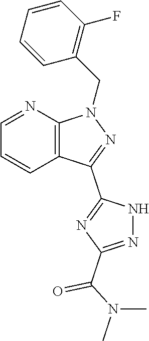 [SMILES: CN(C)C(=O)c1n[nH]c(-c2nn(Cc3ccccc3F)c3ncccc23)n1] | IZA-74 |
| 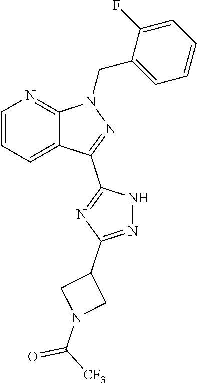 [SMILES: O=C(N1CC(c2n[nH]c(-c3nn(Cc4ccccc4F)c4ncccc34)n2)C1)C(F)(F)F] | IZA-75 |
| 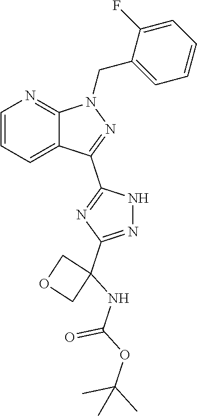 [SMILES: CC(C)(C)OC(=O)NC1(c2n[nH]c(-c3nn(Cc4ccccc4F)c4ncccc34)n2)COC1] | IZA-76 |
| 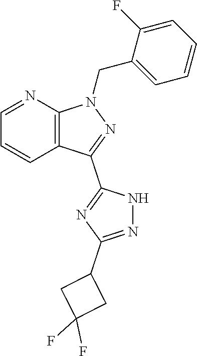 [SMILES: Fc1ccccc1Cn1nc(-c2nc(C3CC(F)(F)C3)n[nH]2)c2cccnc21] | IZA-77 |
| 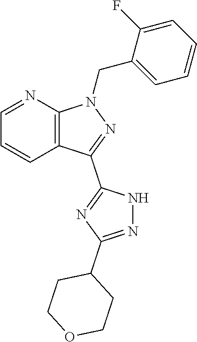 [SMILES: Fc1ccccc1Cn1nc(-c2nc(C3CCOCC3)n[nH]2)c2cccnc21] | IZA-78 |
| 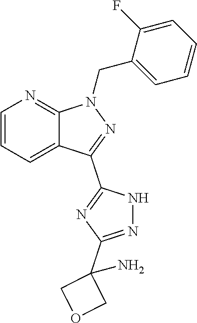 [SMILES: NC1(c2n[nH]c(-c3nn(Cc4ccccc4F)c4ncccc34)n2)COC1] | IZA-79 |
| 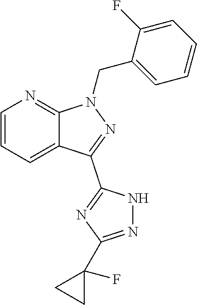 [SMILES: Fc1ccccc1Cn1nc(-c2nc(C3(F)CC3)n[nH]2)c2cccnc21] | IZA-80 |
| 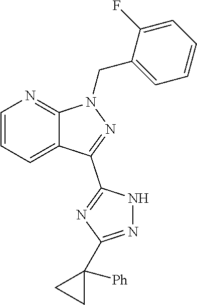 [SMILES: Fc1ccccc1Cn1nc(-c2nc(C3(c4ccccc4)CC3)n[nH]2)c2cccnc21] | IZA-81 |
| 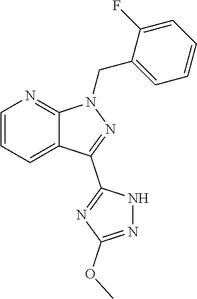 [SMILES: COc1n[nH]c(-c2nn(Cc3ccccc3F)c3ncccc23)n1] | IZA-82 |
| 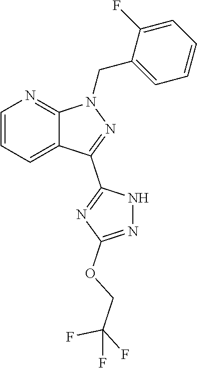 [SMILES: Fc1ccccc1Cn1nc(-c2nc(OCC(F)(F)F)n[nH]2)c2cccnc21] | IZA-83 |
| 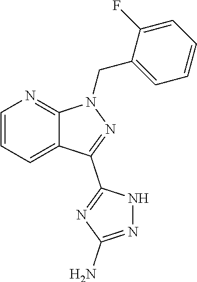 [SMILES: Nc1n[nH]c(-c2nn(Cc3ccccc3F)c3ncccc23)n1] | IZA-84 |
| 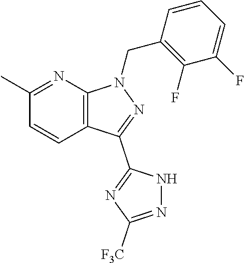 [SMILES: Cc1ccc2c(-c3nc(C(F)(F)F)n[nH]3)nn(Cc3cccc(F)c3F)c2n1] | IZA-85 |
| 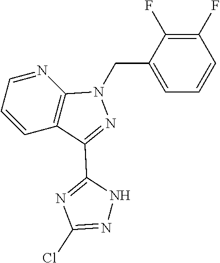 [SMILES: Fc1cccc(Cn2nc(-c3nc(Cl)n[nH]3)c3cccnc32)c1F] | IZA-86 |
| 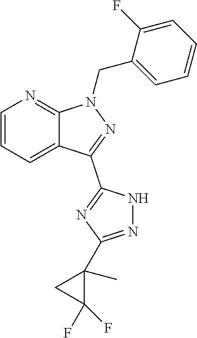 [SMILES: CC1(c2n[nH]c(-c3nn(Cc4ccccc4F)c4ncccc34)n2)CC1(F)F] | IZA-87 |
| 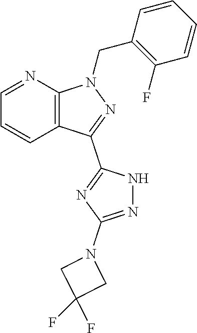 [SMILES: Fc1ccccc1Cn1nc(-c2nc(N3CC(F)(F)C3)n[nH]2)c2cccnc21] | IZA-88 |
| 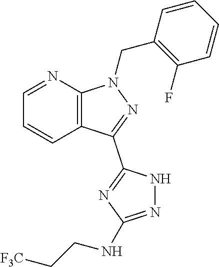 [SMILES: Fc1ccccc1Cn1nc(-c2nc(NCCC(F)(F)F)n[nH]2)c2cccnc21] | IZA-89 |
| 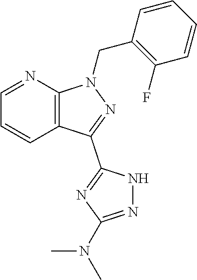 [SMILES: CN(C)c1n[nH]c(-c2nn(Cc3ccccc3F)c3ncccc23)n1] | IZA-90 |
| 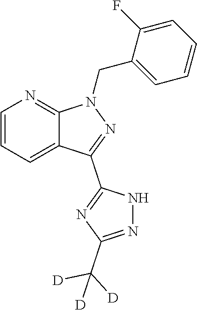 [SMILES: [2H]C([2H])([2H])c1n[nH]c(-c2nn(Cc3ccccc3F)c3ncccc23)n1] | IZA-91 |
| 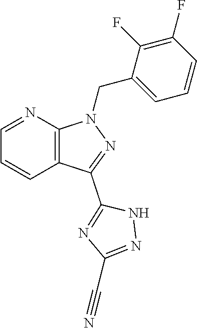 [SMILES: N#Cc1n[nH]c(-c2nn(Cc3cccc(F)c3F)c3ncccc23)n1] | IZA-92 |
| 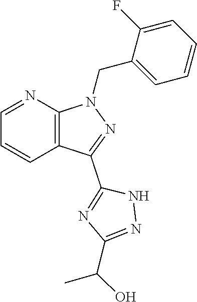 [SMILES: CC(O)c1n[nH]c(-c2nn(Cc3ccccc3F)c3ncccc23)n1] | IZA-107 |
| 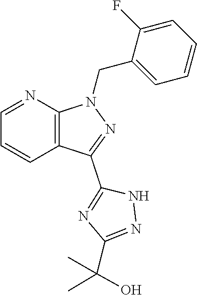 [SMILES: CC(C)(O)c1n[nH]c(-c2nn(Cc3ccccc3F)c3ncccc23)n1] | IZA-94 |
| 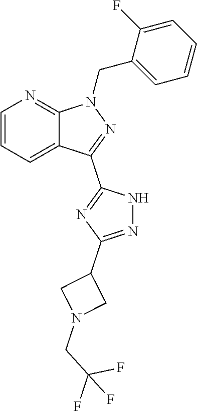 [SMILES: Fc1ccccc1Cn1nc(-c2nc(C3CN(CC(F)(F)F)C3)n[nH]2)c2cccnc21] | IZA-95 |
| 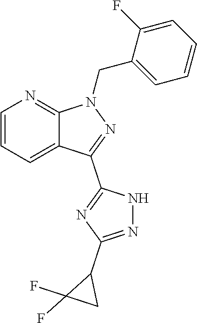 [SMILES: Fc1ccccc1Cn1nc(-c2nc(C3CC3(F)F)n[nH]2)c2cccnc21] | IZA-96 |
| 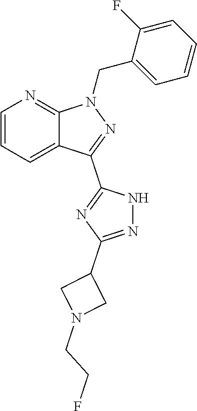 [SMILES: FCCN1CC(c2n[nH]c(-c3nn(Cc4ccccc4F)c4ncccc34)n2)C1] | IZA-97 |
| 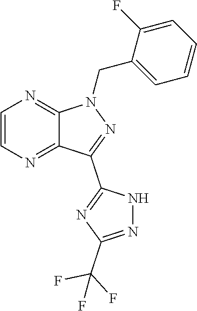 [SMILES: Fc1ccccc1Cn1nc(-c2nc(C(F)(F)F)n[nH]2)c2nccnc21] | IZA-98 |
| 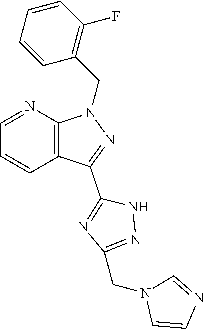 [SMILES: Fc1ccccc1Cn1nc(-c2nc(Cn3ccnc3)n[nH]2)c2cccnc21] | IZA-99 |
| 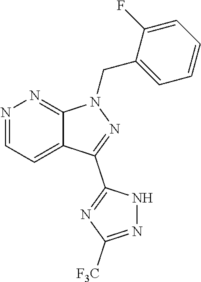 [SMILES: Fc1ccccc1Cn1nc(-c2nc(C(F)(F)F)n[nH]2)c2ccnnc21] | IZA-100 |
| 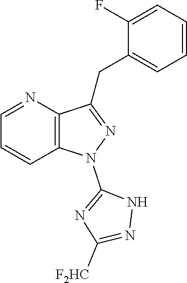 [SMILES: Fc1ccccc1Cc1nn(-c2nc(C(F)F)n[nH]2)c2cccnc12] | IZA-101 |
| 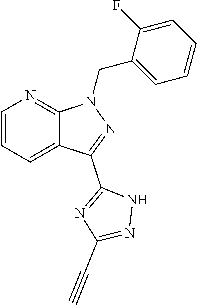 [SMILES: C#Cc1n[nH]c(-c2nn(Cc3ccccc3F)c3ncccc23)n1] | IZA-102 |
| 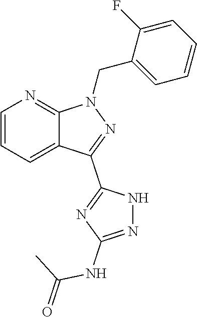 [SMILES: CC(=O)Nc1n[nH]c(-c2nn(Cc3ccccc3F)c3ncccc23)n1] | IZA-103 |
| 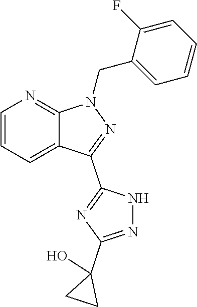 [SMILES: OC1(c2n[nH]c(-c3nn(Cc4ccccc4F)c4ncccc34)n2)CC1] | IZA-104 |
| 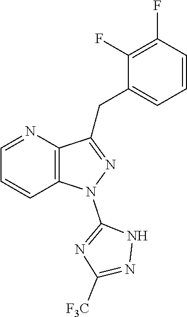 [SMILES: Fc1cccc(Cc2nn(-c3nc(C(F)(F)F)n[nH]3)c3cccnc23)c1F] | IZA-105 |
| 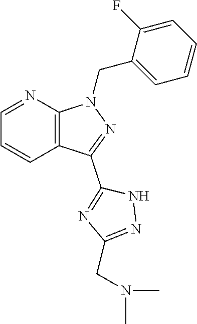 [SMILES: CN(C)Cc1n[nH]c(-c2nn(Cc3ccccc3F)c3ncccc23)n1] | IZA-106 |
| 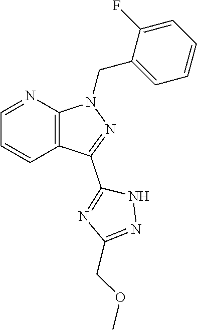 [SMILES: COCc1n[nH]c(-c2nn(Cc3ccccc3F)c3ncccc23)n1] | IZA-112 |
| 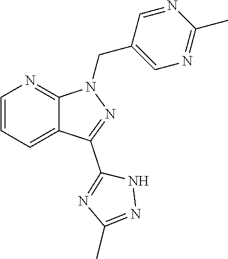 [SMILES: Cc1ncc(Cn2nc(-c3nc(C)n[nH]3)c3cccnc32)cn1] | IZA-113 |
| 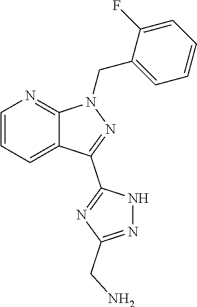 [SMILES: NCc1n[nH]c(-c2nn(Cc3ccccc3F)c3ncccc23)n1] | IZA-115 |
| 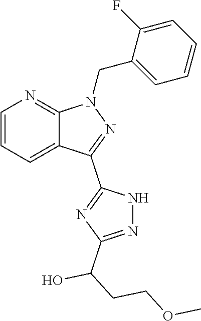 [SMILES: COCCC(O)c1n[nH]c(-c2nn(Cc3ccccc3F)c3ncccc23)n1] | IZA-116 |
| 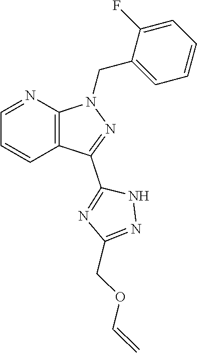 [SMILES: C=COCc1n[nH]c(-c2nn(Cc3ccccc3F)c3ncccc23)n1] | IZA-117 |
| 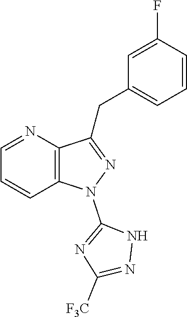 [SMILES: Fc1cccc(Cc2nn(-c3nc(C(F)(F)F)n[nH]3)c3cccnc23)c1] | IZA-120 |
| 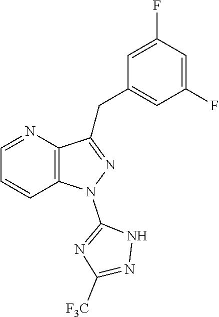 [SMILES: Fc1cc(F)cc(Cc2nn(-c3nc(C(F)(F)F)n[nH]3)c3cccnc23)c1] | IZA-121 |
| 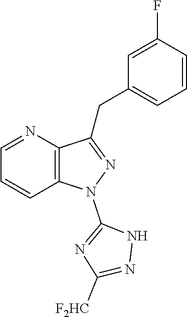 [SMILES: Fc1cccc(Cc2nn(-c3nc(C(F)F)n[nH]3)c3cccnc23)c1] | IZA-122 |
| 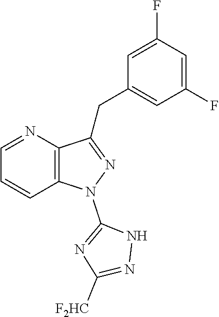 [SMILES: Fc1cc(F)cc(Cc2nn(-c3nc(C(F)F)n[nH]3)c3cccnc23)c1] | IZA-123 |
| 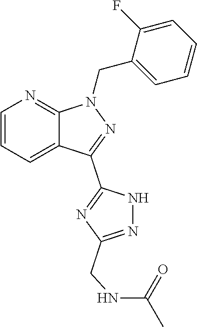 [SMILES: CC(=O)NCc1n[nH]c(-c2nn(Cc3ccccc3F)c3ncccc23)n1] | IZA-124 |
| 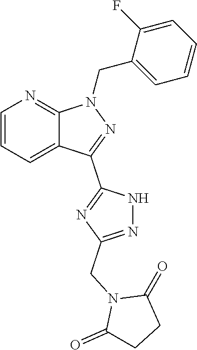 [SMILES: O=C1CCC(=O)N1Cc1n[nH]c(-c2nn(Cc3ccccc3F)c3ncccc23)n1] | IZA-125 |
| 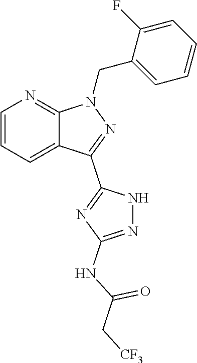 [SMILES: O=C(CC(F)(F)F)Nc1n[nH]c(-c2nn(Cc3ccccc3F)c3ncccc23)n1] | IZA-126 |
| 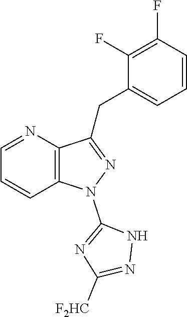 [SMILES: Fc1cccc(Cc2nn(-c3nc(C(F)F)n[nH]3)c3cccnc23)c1F] | IZA-127 |
| 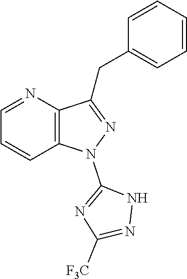 [SMILES: FC(F)(F)c1n[nH]c(-n2nc(Cc3ccccc3)c3ncccc32)n1] | IZA-128 |
| 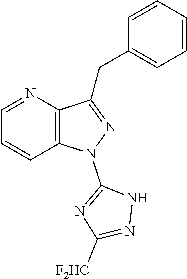 [SMILES: FC(F)c1n[nH]c(-n2nc(Cc3ccccc3)c3ncccc32)n1] | IZA-129 |
| 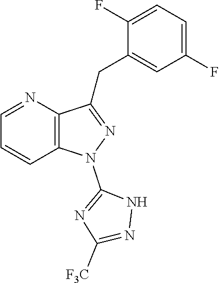 [SMILES: Fc1ccc(F)c(Cc2nn(-c3nc(C(F)(F)F)n[nH]3)c3cccnc23)c1] | IZA-130 |
| 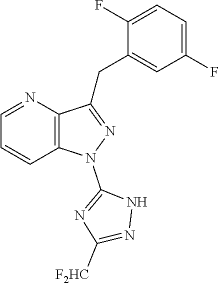 [SMILES: Fc1ccc(F)c(Cc2nn(-c3nc(C(F)F)n[nH]3)c3cccnc23)c1] | IZA-131 |
| 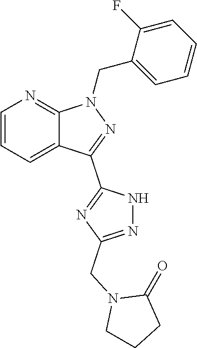 [SMILES: O=C1CCCN1Cc1n[nH]c(-c2nn(Cc3ccccc3F)c3ncccc23)n1] | IZA-132 |
| 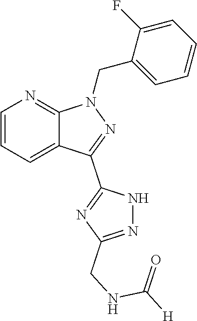 [SMILES: O=CNCc1n[nH]c(-c2nn(Cc3ccccc3F)c3ncccc23)n1] | IZA-133 |
| 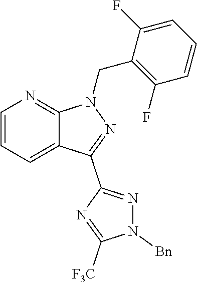 [SMILES: Fc1cccc(F)c1Cn1nc(-c2nc(C(F)(F)F)n(Cc3ccccc3)n2)c2cccnc21] | IZA-134 |
| 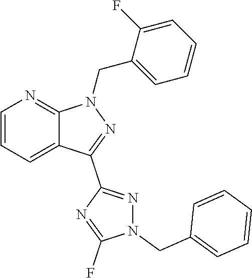 [SMILES: Fc1ccccc1Cn1nc(-c2nc(F)n(Cc3ccccc3)n2)c2cccnc21] | IZA-135 |

In some embodiments of the above methods, uses, compositions and kits, the sGC stimulator is a compound of Table IZB:

| | |
|---|---|
| 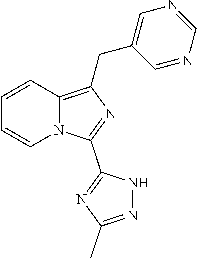 [SMILES: Cc1n[nH]c(-c2nc(Cc3cncnc3)c3ccccn23)n1] | IZB-5 |
| 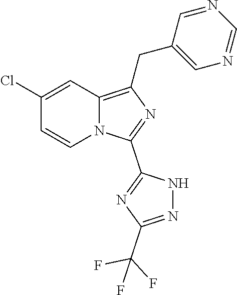 [SMILES: FC(F)(F)c1n[nH]c(-c2nc(Cc3cncnc3)c3cc(Cl)ccn23)n1] | IZB-6 |
| 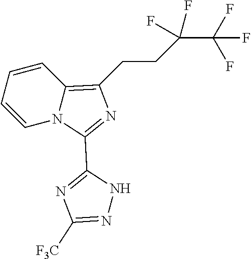 [SMILES: FC(F)(F)c1n[nH]c(-c2nc(CCC(F)(F)C(F)(F)F)c3ccccn23)n1] | IZB-9 |
| 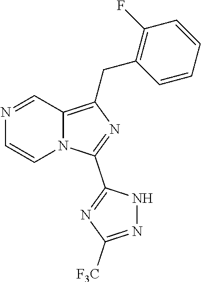 [SMILES: Fc1ccccc1Cc1nc(-c2nc(C(F)(F)F)n[nH]2)n2ccncc12] | IZB-44 |
| 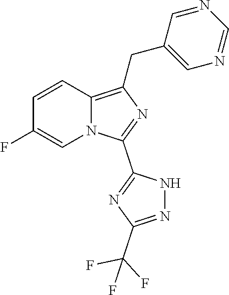 [SMILES: Fc1ccc2c(Cc3cncnc3)nc(-c3nc(C(F)(F)F)n[nH]3)n2c1] | IZB-12 |
| 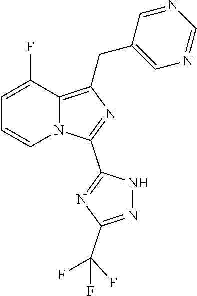 [SMILES: Fc1cccn2c(-c3nc(C(F)(F)F)n[nH]3)nc(Cc3cncnc3)c12] | IZB-15 |
| 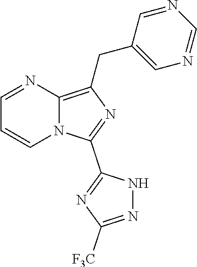 [SMILES: FC(F)(F)c1n[nH]c(-c2nc(Cc3cncnc3)c3ncccn23)n1] | IZB-17 |
| 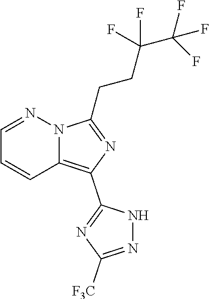 [SMILES: FC(F)(F)c1n[nH]c(-c2nc(CCC(F)(F)C(F)(F)F)n3ncccc23)n1] | IZB-18 |
| 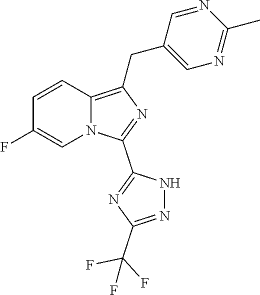 [SMILES: Cc1ncc(Cc2nc(-c3nc(C(F)(F)F)n[nH]3)n3cc(F)ccc23)cn1] | IZB-23 |
| 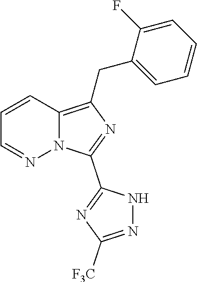 [SMILES: Fc1ccccc1Cc1nc(-c2nc(C(F)(F)F)n[nH]2)n2ncccc12] | IZB-24 |
| 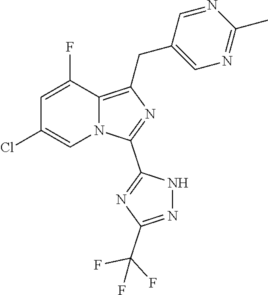 [SMILES: Cc1ncc(Cc2nc(-c3nc(C(F)(F)F)n[nH]3)n3cc(Cl)cc(F)c23)cn1] | IZB-27 |
| 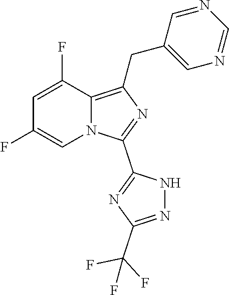 [SMILES: Fc1cc(F)c2c(Cc3cncnc3)nc(-c3nc(C(F)(F)F)n[nH]3)n2c1] | IZB-28 |
| 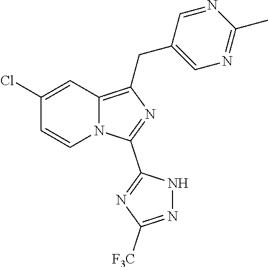 [SMILES: Cc1ncc(Cc2nc(-c3nc(C(F)(F)F)n[nH]3)n3ccc(Cl)cc23)cn1] | IZB-29 |
| 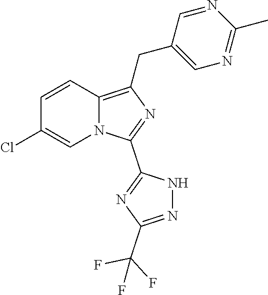 [SMILES: Cc1ncc(Cc2nc(-c3nc(C(F)(F)F)n[nH]3)n3cc(Cl)ccc23)cn1] | IZB-34 |

In some embodiments of the above methods, uses, compositions and kits, the sGC stimulator is a compound selected from Table IZC:

Structure

| | |
|---|---|
| 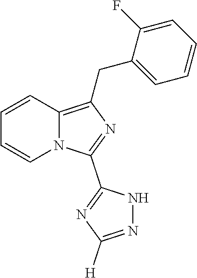 [SMILES: Fc1ccccc1Cc1nc(-c2ncn[nH]2)n2ccccc12] | IZC-8 |
| 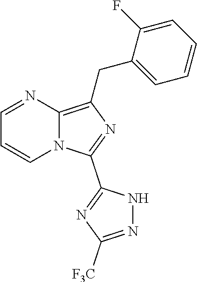 [SMILES: Fc1ccccc1Cc1nc(-c2nc(C(F)(F)F)n[nH]2)n2cccnc12] | IZC-7 |
| 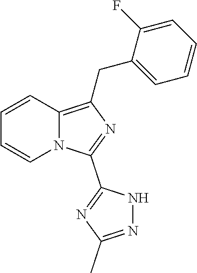 [SMILES: Cc1n[nH]c(-c2nc(Cc3ccccc3F)c3ccccn23)n1] | IZC-9 |
| 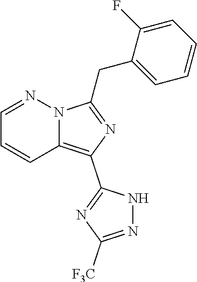 [SMILES: Fc1ccccc1Cc1nc(-c2nc(C(F)(F)F)n[nH]2)c2cccnn12] | IZC-6 |
| 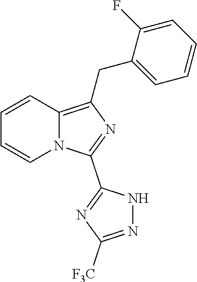 [SMILES: Fc1ccccc1Cc1nc(-c2nc(C(F)(F)F)n[nH]2)n2ccccc12] | IZC-3 |
| 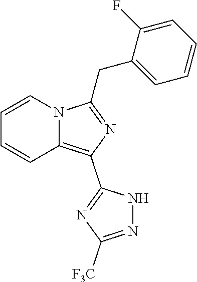 [SMILES: Fc1ccccc1Cc1nc(-c2nc(C(F)(F)F)n[nH]2)c2ccccn12] | IZC-10 |
| 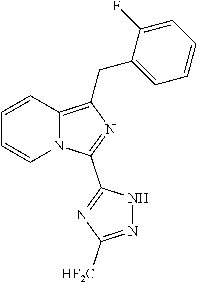 | IZC-11 |
| 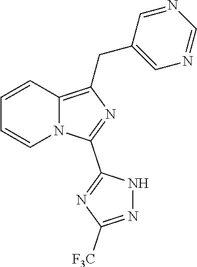 [SMILES: FC(F)(F)c1n[nH]c(-c2nc(Cc3cncnc3)c3ccccn23)n1] | IZC-5 |
| 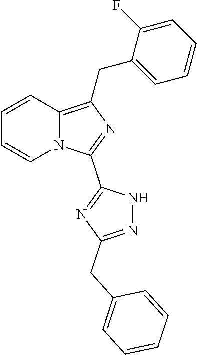 [SMILES: Fc1ccccc1Cc1nc(-c2nc(Cc3ccccc3)n[nH]2)n2ccccc12] | IZC-12 |
| 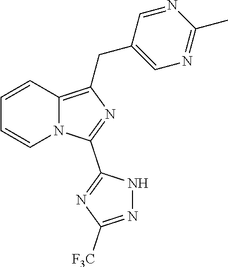 [SMILES: Cc1ncc(Cc2nc(-c3nc(C(F)(F)F)n[nH]3)n3ccccc23)cn1] | IZC-4 |
| 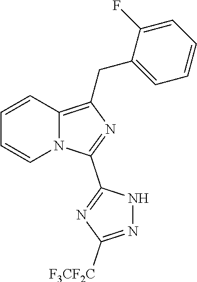 | IZC-13 |
| 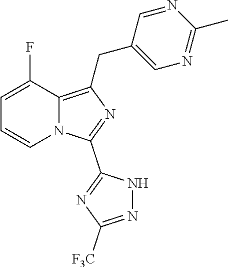 [SMILES: Cc1ncc(Cc2nc(-c3nc(C(F)(F)F)n[nH]3)n3cccc(F)c23)cn1] | IZC-16 |
| 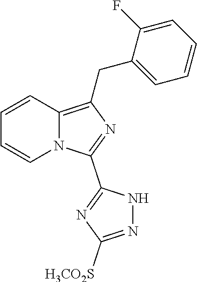 | IZC-14 |
| 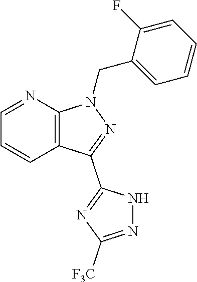 [SMILES: Fc1ccccc1Cn1nc(-c2nc(C(F)(F)F)n[nH]2)c2cccnc21] | IZC-2 |
| 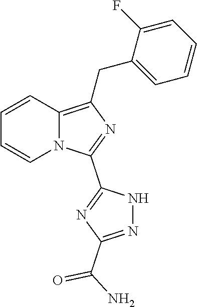 [SMILES: NC(=O)c1n[nH]c(-c2nc(Cc3ccccc3F)c3ccccn23)n1] | IZC-15 |
| 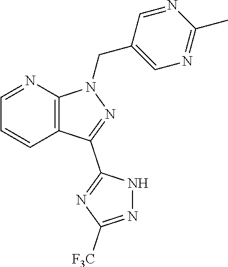 [SMILES: Cc1ncc(Cn2nc(-c3nc(C(F)(F)F)n[nH]3)c3cccnc32)cn1] | IZC-1 |

### Pharmaceutically Acceptable Salts

In some embodiments of the methods, uses, pharmaceutical compositions and kits, the sGC stimulator may be provided as (i) the compound itself (e.g., as the free base); (ii) a pharmaceutically acceptable salt of the compound; or (iii) part of a pharmaceutical composition. In some embodiments of the above methods, uses, pharmaceutical compositions and kits, the additional therapeutic agent may be provided as (i) the compound itself (e.g., as the free base); (ii) a pharmaceutically acceptable salt of the compound; (iii) or part of a pharmaceutical composition.

The phrase “pharmaceutically acceptable salt,” as used herein, refers to pharmaceutically acceptable organic or inorganic salts of a compound described herein. For use in medicine, the salts of the compounds described herein will be pharmaceutically acceptable salts. Other salts may, however, be useful in the preparation of the compounds described herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound described herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N, N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound described herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety. Compounds, compositions and kits of the invention are also useful for veterinary treatment of companion animals, exotic animals and farm animals, including, without limitation, dogs, cats, mice, rats, hamsters, gerbils, guinea pigs, rabbits, horses, pigs and cattle.

### Methods of Administration and Co-Administration

In some embodiments of the above methods and uses, the sGC stimulator is administered before a symptom of achalasia fully develops in said patient. In other embodiments of the above methods and uses, the sGC stimulator is administered after one or more symptoms of achalasia develops in said patient.

As used herein, the terms “in combination” or “co-administration” can be used interchangeably to refer to the use of more than one therapy (e.g., an sGC stimulator and one or more additional therapeutic agents). The use of the terms does not restrict the order in which therapies (e.g., the sGC stimulator and the additional therapeutic agents) are administered to a subject.

In some embodiments, the sGC stimulator is administered prior to, at the same time or after the initiation of treatment with another therapeutic agent.

In some embodiments of the above methods and uses, the additional therapeutic agent and the sGC stimulator are administered simultaneously. In other embodiments of the above methods and uses, the additional therapeutic agent and the sGC stimulator are administered sequentially or separately.

In some embodiments, the above pharmaceutical compositions or kits comprise (a) an sGC stimulator as discussed above or a pharmaceutically acceptable salt thereof, and (b) a pharmaceutically acceptable carrier, vehicle or adjuvant. In some embodiments, the pharmaceutical composition or kit comprises (a) one or more additional therapeutic agents as discussed above, or a pharmaceutically acceptable salt thereof, and (b) a pharmaceutically acceptable carrier, vehicle or adjuvant. In some embodiments, the pharmaceutical composition comprises (i) an sGC stimulator as discussed above, or a pharmaceutically acceptable salt thereof, (ii) one or more additional therapeutic agents as discussed above, or a pharmaceutically acceptable salt thereof, and (iii) a pharmaceutically acceptable carrier, vehicle or adjuvant.

The sGC stimulators and pharmaceutical compositions described herein can be used in combination therapy with one or more additional therapeutic agents. For combination treatment with more than one active agent, the additional active agents may be in the same dosage form or in separate dosage forms. Wherein the additional active agents are present in separate dosage forms, the active agents may be administered separately or in conjunction with the sGC stimulator. In addition, the administration of one agent may be prior to, concurrent to, or subsequent to the administration of the other agent.

When co-administered with other agents, e.g., when co-administered with another sGC stimulator, arginine, etc, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When “combination therapy” is employed, an effective amount can be achieved using a first amount of an sGC stimulator or a pharmaceutically acceptable salt thereof and a second amount of an additional suitable therapeutic agent (e.g., another sGC stimulator, arginine, a NO modulator, a cGMP modulator, a therapeutic that increases the function of nitric oxide synthase, etc.).

In one embodiment of this invention, the sGC stimulator and the additional therapeutic agent are each administered in an effective amount (i.e., each in an amount which would be therapeutically effective if administered alone). In another embodiment, the sGC stimulator and the additional therapeutic agent are each administered in an amount which alone does not provide a therapeutic effect (“a sub-therapeutic dose”). In yet another embodiment, the sGC stimulator can be administered in an effective amount, while the additional therapeutic agent is administered in a sub-therapeutic dose. In still another embodiment, the sGC stimulator can be administered in a sub-therapeutic dose, while the additional therapeutic agent, for example, a suitable anti-inflammatory agent is administered in an effective amount.

“Co-administration” encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, co-administration also encompasses use of each compound in a sequential manner in either order. When co-administration involves the separate administration of the first amount of an sGC stimulator and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, an sGC stimulator and the second therapeutic agent can be administered in any order within about 24 hours of each other, within about 16 hours of each other, within about 8 hours of each other, within about 4 hours of each other, within about 1 hour of each other or within about 30 minutes of each other, within about 5 minutes of each other, etc.

More, specifically, a first therapy (e.g., a prophylactic or therapeutically used sGC stimulator) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., an additional therapeutic agent or prophylactic agent described herein) to a subject.

### Combination Therapies

In some embodiments of the above methods, uses, compositions and kits, the additional therapeutic agent or agents may be selected from one or more of the following:
(1) Endothelium-derived releasing factor (EDRF) or NO gas.
(2) NO donors such as a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide (Dilatrate®-SR, Imdur®, Ismo®, Isordil®, Isordil®, Titradose®, Monoket®), 3-morpholinosydnonimine; linsidomine chlorohydrate (“SIN-1”); S-nitroso-N-acetylpenicillamine (“SNAP”); S-nitrosoglutathione (GSNO), sodium nitroprusside, S-nitrosoglutathione mono-ethyl-ester (GSNO-ester), 6-(2-hydroxy-1-methyl-nitrosohydrazino)-N-methyl-1-hexanamine or diethylamine NONOate.
(3) Other substances that enhance cGMP concentrations such as protoporphyrin IX, arachidonic acid and phenyl hydrazine derivatives.
(4) Nitric Oxide Synthase substrates: for example, n-hydroxyguanidine based analogs, such as N[G]-hydroxy-L-arginine (NOHA), 1-(3, 4-dimethoxy-2-chlorobenzylideneamino)-3-hydroxyguanidine, and PR5 (1-(3, 4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine.
(5) Compounds which enhance eNOS transcription.
(6) NO independent heme-independent sGC activators, including, but not limited to:

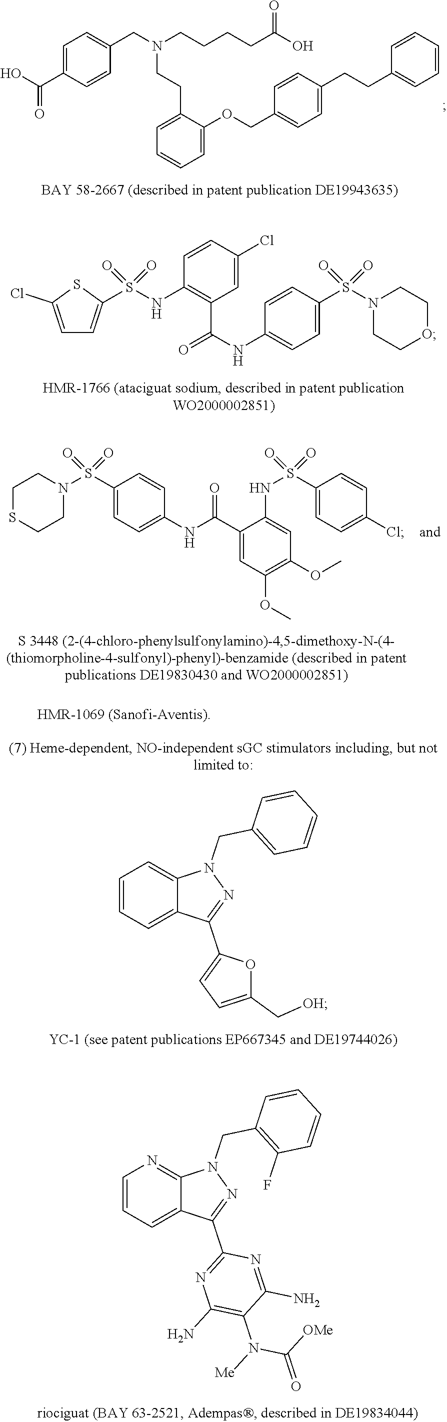 [SMILES: O=C(O)CCCCN(CCc1ccccc1OCc1ccc(CCc2ccccc2)cc1)Cc1ccc(C(=O)O)cc1 ; O=C(Nc1ccc(S(=O)(=O)N2CCOCC2)cc1)c1cc(Cl)ccc1NS(=O)(=O)c1ccc(Cl)s1 ; COc1cc(NS(=O)(=O)c2ccc(Cl)cc2)c(C(=O)Nc2ccc(S(=O)(=O)N3CCSCC3)cc2)cc1OC ; OCc1ccc(-c2nn(Cc3ccccc3)c3ccccc23)o1 ; COC(=O)N(C)c1c(N)nc(-c2nn(Cc3ccccc3F)c3ncccc23)nc1N]

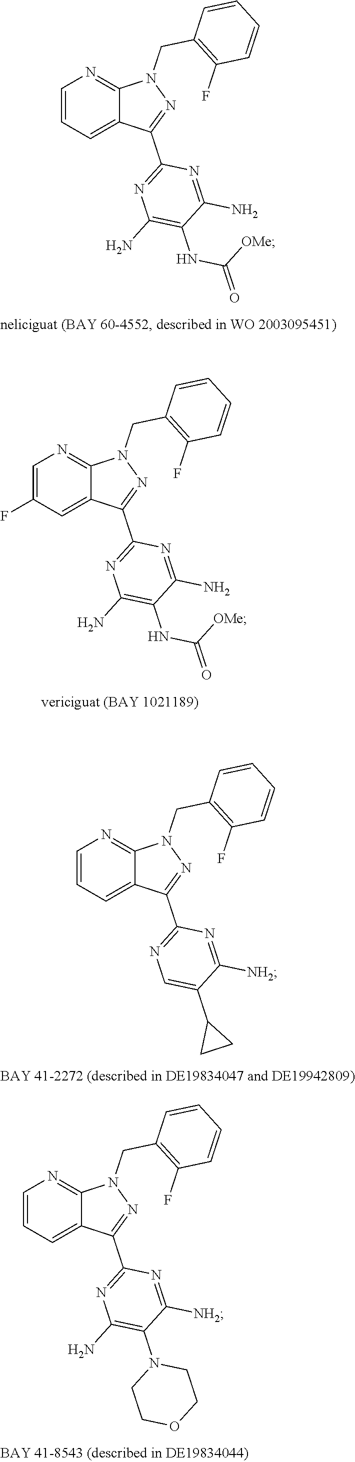 [SMILES: COC(=O)Nc1c(N)nc(-c2nn(Cc3ccccc3F)c3ncccc23)nc1N ; COC(=O)Nc1c(N)nc(-c2nn(Cc3ccccc3F)c3ncc(F)cc23)nc1N ; Nc1nc(-c2nn(Cc3ccccc3F)c3ncccc23)ncc1C1CC1 ; Nc1nc(-c2nn(Cc3ccccc3F)c3ncccc23)nc(N)c1N1CCOCC1]

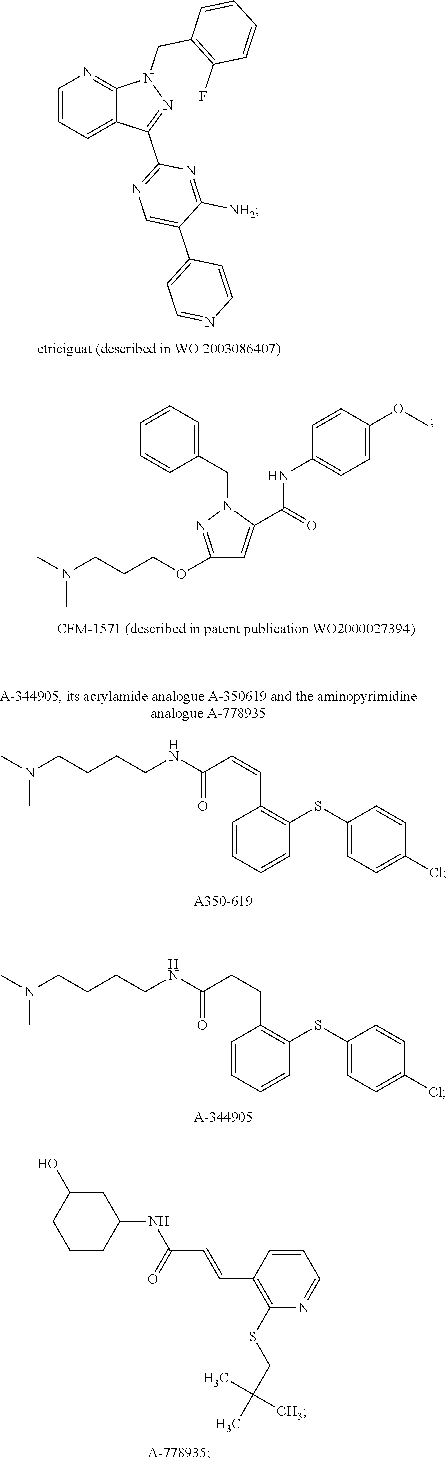

and other sGC stimulators described in one of publications US20090209556, U.S. Pat. No. 8,455,638, US20110118282 (WO2009032249), US20100292192, US20110201621, U.S. Pat. No. 7,947,664, U.S. Pat. No. 8,053,455 (WO2009094242), US20100216764, U.S. Pat. No. 8,507,512, (WO2010099054) US20110218202 (WO2010065275), US20130012511 (WO2011119518), US20130072492 (WO2011149921), US20130210798 (WO2012058132) and other compounds described in Tetrahedron Letters (2003), 44(48): 8661-8663.
(8) Compounds that inhibit the degradation of cGMP, such as:
   PDE5 inhibitors, such as, for example, sildenafil (Viagra®) and related agents such as avanafil, lodenafil, mirodenafil, sildenafil citrate (Revatio®), tadalafil (Cialis® or Adcirca®), vardenafil (Levitra®) and udenafil; alprostadil; dipyridamole and PF-00489791; and
   PDE9 inhibitors, such as, for example, PF-04447943.
(9) Calcium channel blockers of the following types: dihydropyridine calcium channel blockers such asamlodipine (Norvasc®), aranidipine (Sapresta®), azelnidipine (Calblock®), barnidipine (HypoCa®), benidipine (Coniel®), cilnidipine (Atelec®, Cinalong®, Siscard®), clevidipine (Cleviprex®), diltiazem, efonidipine (Landel®), felodipine (Plendil®), lacidipine (Motens®, Lacipil®), lercanidipine (Zanidip®), manidipine (Calslot®, Madipine®), nicardipine (Cardene®, Carden SR®), nifedipine (Procardia®, Adalat®), nilvadipine (Nivadil®), nimodipine (Nimotop®), nisoldipine (Baymycard®, Sular®, Syscor®), nitrendipine (Cardif®, Nitrepin®, Baylotensin®), pranidipine (Acalas®), isradipine (Lomir®);

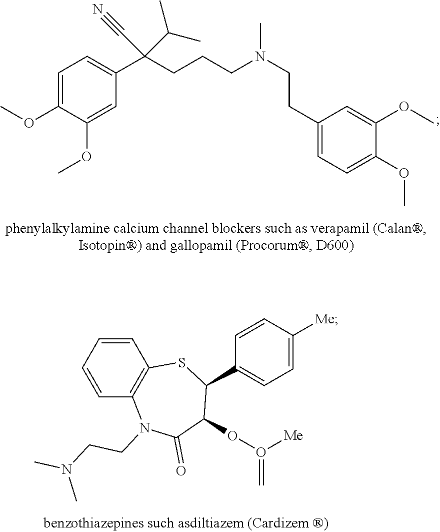

and nonselective calcium channel inhibitors such as mibefradil, bepridil, fluspirilene, and fendiline.
(10) Endothelin receptor antagonists (ERAs) such as the dual (ET_{A} and ET_{B}) endothelin receptor antagonist bosentan (Tracleer®), sitaxentan (Thelin®) or ambrisentan (Letairis®).
(11) Prostacyclin derivatives or analogues, such asprostacyclin (prostaglandin I₂), epoprostenol (synthetic prostacyclin, Flolan®), treprostinil (Remodulin®), iloprost (Ilomedin®), iloprost (Ventavis®); and oral and inhaled forms of RemodulinR under development.
(12) Antihyperlipidemics such as the following types:
bile acid sequestrants like cholestyramine, colestipol, colestilan, colesevelam or sevelamer;
statins like atorvastatin, simvastatin, lovastatin, fluvastatin, pitavastatin, rosuvastatin and pravastatin;
cholesterol absorption inhibitors such as ezetimibe;
other lipid lowering agents such as icosapent ethyl ester, omega-3-acid ethyl esters, reducol;
fibric acid derivatives such as clofibrate, bezafibrate, clinofibrate, gemfibrozil, ronifibrate, binifibrate, fenofibrate, ciprofibrate, choline fenofibrate;
nicotinic acid derivatives such as acipimox and niacin;
combinations of statins, niacin and intestinal cholesterol absorption-inhibiting supplements (ezetimibe and others) and fibrates; and
antiplatelet therapies such as clopidogrel bisulfate.
(13) Anticoagulants, such as the following types:
coumarines (Vitamin K antagonists) such as warfarin (Coumadin®), cenocoumarol, phenprocoumon and phenindione;
heparin and derivatives such as low molecular weight heparin, fondaparinux and idraparinux;
direct thrombin inhibitors such as argatroban, lepirudin, bivalirudin, dabigatran and ximelagatran (Exanta®); and
tissue-plasminogen activators, used to dissolve clots and unblock arteries, such as alteplase.
(14) Antiplatelet drugs such as, for instance, topidogrel, ticlopidine, dipyridamoleand aspirin.
(15) ACE inhibitors, for example the following types:
sulfhydryl-containing agents such as captopril (Capoten®) and zofenopril;
dicarboxylate-containing agents such as enalapril (Vasotec/Renitec®), ramipril (Altace®/Tritace®/Ramace®/Ramiwin®), quinapril (Accupril®), perindopril (Coversyl®/Aceon®), lisinopril (Lisodur®/Lopril®/Novatec®/Prinivil®/Zestril®) and benazepril (Lotensin®);
phosphonate-containing agents such as fosinopril;
naturally occurring ACE inhibitors such as casokinins and lactokinins, which are breakdown products of casein and whey that occur naturally after ingestion of milk products, especially cultured milk;
the lactotripeptides Val-Pro-Pro and Ile-Pro-Pro produced by the probiotic Lactobacillus helveticus or derived from casein also having ACE-inhibiting and antihypertensive functions;
other ACE inhibitors such as alacepril, delapril, cilazapril, imidapril, trandolapril, temocapril, moexipril and pirapril.
(16) Supplemental oxygen therapy.
(17) Beta blockers, such as the following types:
non-selective agents such as alprenolol, bucindolol, carteolol, carvedilol, labetalol, nadolol, penbutolol, pindolol, oxprenonol, acebutolol, sotalol, mepindolol, celiprolol, arotinolol, tertatolol, amosulalol, nipradilol, propranolol and timolol;
β₁-Selective agents such as cebutolol, atenolol, betaxolol, bisoprolol, celiprolol, dobutamine hydrochloride, irsogladine maleate, carvedilol, talinolol, esmolol, metoprolol and nebivolol; and
β₂-Selective agents such as butaxamine.
(18) Antiarrhythmic agents such as the following types:
Type I (sodium channel blockers) such as quinidine, lidocaine, phenytoin, propafenone;
Type III (potassium channel blockers) such as amiodarone, dofetilide and sotalol; and
Type V such as adenosine and digoxin.
(19) Diuretics such as thiazide diuretics, for example chlorothiazide, chlorthalidone and hydrochlorothiazide, bendroflumethiazide, cyclopenthiazide, methyclothiazide, polythiazide, quinethazone, xipamide, metolazone, indapamide, cicletanine; loop diuretics, such as furosemide and toresamide; potassium-sparing diuretics such as amiloride, spironolactone, canrenoate potassium, eplerenone and triamterene; combinations of these agents; other diuretics such as acetazolamid and carperitide.
(20) Direct-acting vasodilators such as hydralazine hydrochloride, diazoxide, sodium nitroprusside, cadralazine; other vasodilators such as isosorbide dinitrate and isosorbide 5-mononitrate.
(21) Exogenous vasodilators such as Adenocard® and alpha blockers.
(22) Alpha-1-adrenoceptor antagonists such as prazosin, indoramin, urapidil, bunazosin, terazosin and doxazosin; atrial natriuretic peptide (ANP), ethanol, histamine-inducers, tetrahydrocannabinol (THC) and papaverine.
(23) Bronchodilators of the following types:
short acting β₂ agonists, such as albutamol or albuterol (Ventolin®) and terbutaline;
long acting β₂ agonists (LABAs) such as salmeterol and formoterol;
anticholinergics such as pratropium and tiotropium; and
theophylline, a bronchodilator and phosphodiesterase inhibitor.
(24) Corticosteroids such as beclomethasone, methylprednisolone, betamethasone, prednisone, prednisolone, triamcinolone, dexamethasone, fluticasone, flunisolide, hydrocortisone, and corticosteroid analogs such as budesonide.
(25) Dietary supplements such as, for example omega-3 oils; folic acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; vitamin C, Vitamin E, Vitamin K2; testosterone supplements, testosterone transdermal patch; zoraxel, naltrexone, bremelanotide and melanotan II.
(26) PGD2 receptor antagonists.
(27) Immunosuppressants such as cyclosporine (cyclosporine A, Sandimmune®, Neoral®), tacrolimus (FK-506, Prograf®), rapamycin (Sirolimus®, Rapamune®) and other FK-506 type immunosuppressants, mycophenolate, e.g., mycophenolate mofetil (CellCept®).
(28) Non-steroidal anti-asthmatics such as β2-agonists like terbutaline, metaproterenol, fenoterol, isoetharine, albuterol, salmeterol, bitolterol and pirbuterol; β2-agonist-corticosteroid combinations such as salmeterol-fluticasone (Advair®), formoterol-budesonide (Symbicort®), theophylline, cromolyn, cromolyn sodium, nedocromil, atropine, ipratropium, ipratropium bromide and leukotriene biosynthesis inhibitors (zileuton, BAY1005).
(29) Non-steroidal anti-inflammatory agents (NSAIDs) such as propionic acid derivatives like alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen); acetic acid derivatives such as indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac; fenamic acid derivatives such as flufenamic acid, meclofenamic acid, mefenamic acid, niflumic acid and tolfenamic acid; biphenylcarboxylic acid derivatives such as diflunisal and flufenisal; oxicams such as isoxicam, piroxicam, sudoxicam and tenoxican; salicylates such as acetyl salicylic acid and sulfasalazine; and the pyrazolones such as apazone, bezpiperylon, feprazone, mofebutazone, oxyphenbutazone and phenylbutazone.
(30) Cyclooxygenase-2 (COX-2) inhibitors such as celecoxib (Celebrex®), rofecoxib (Vioxx®), valdecoxib, etoricoxib, parecoxib and lumiracoxib; opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine;
(31) Anti-diabetic agents such as insulin and insulin mimetics; sulfonylureas such as glyburide, glybenclamide, glipizide, gliclazide, gliquidone, glimepiride, meglinatide, tolbutamide, chlorpropamide, acetohexamide and olazamide; biguanides such as metformin (Glucophage®); α-glucosidase inhibitors such as acarbose, epalrestat, voglibose, miglitol; thiazolidinone compounds such as rosiglitazone (Avandia®), troglitazone (Rezulin®), ciglitazone, pioglitazone (Actos®) and englitazone; insulin sensitizers such as pioglitazone and rosiglitazone; insulin secretagogues such as repaglinide, nateglinide and mitiglinide; incretin mimetics such as exanatide and liraglutide; amylin analogues such as pramlintide; glucose lowering agents such as chromium picolinate, optionally combined with biotin; dipeptidyl peptidase IV inhibitors such as sitagliptin, vildagliptin, saxagliptin, alogliptin and linagliptin.
(32) HDL cholesterol-increasing agents such as anacetrapib and dalcetrapib.
(33) Antiobesity drugs such as methamphetamine hydrochloride, amfepramone hydrochloride (Tenuate®), phentermine (Ionamin®), benzfetamine hydrochloride (Didrex®), phendimetrazine tartrate (Bontril®, Prelu-2 ®, Plegine®), mazindol (Sanorex®), orlistat (Xenical®), sibutramine hydrochloride monohydrate (Meridia®, Reductil®), rimonabant (Acomplia®), amfepramone, chromium picolinate; combination such as phentermine/topiramate, bupropion/naltrexone, sibutramine/metformin, bupropion SR/zonisamide SR, salmeterol, xinafoate/fluticasone propionate; lorcaserin hydrochloride, phentermine/topiramate, cetilistat, exenatide, liraglutide, metformin hydrochloride, sibutramine/metformin, bupropion SR/zonisamide SR, CORT-108297, canagliflozin, chromium picolinate, GSK-1521498, LY-377604, metreleptin, obinepitide, P-57AS3, PSN-821, salmeterol xinafoate/fluticasone propionate, sodium tungstate, somatropin (recombinant), tesamorelin, tesofensine, velneperit, zonisamide, beloranib hemioxalate, insulinotropin, resveratrol, sobetirome, tetrahydrocannabivarin and beta-lapachone.
(34) Angiotensin receptor blockers such as losartan, valsartan, candesartan, cilexetil, eprosaran, irbesartan, telmisartan, olmesartran, medoxomil, azilsartan and medoxomil.
(35) Renin inhibitors such as aliskiren hemifumirate.
(36) Centrally acting alpha-2-adrenoceptor agonists such as methyldopa, clonidine and guanfacine.
(37) Adrenergic neuron blockers such as guanethidine and guanadrel.
(38) Imidazoline I-1 receptor agonists such as rimenidine dihydrogen phosphate and moxonidine hydrochloride hydrate.
(39) Aldosterone antagonists such as spironolactone and eplerenone.
(40) Potassium channel activators such as pinacidil.
(41) Dopamine D1 agonists such as fenoldopam mesilate; other dopamine agonists such as ibopamine, dopexamine and docarpamine.
(42) 5-HT2 antagonists such as ketanserin.
(43) Vasopressin antagonists such as tolvaptan.
(44) Calcium channel sensitizers such as levosimendan or activators such as nicorandil.
(45) PDE-3 inhibitors such as amrinone, milrinone, enoximone, vesnarinone, pimobendan, and olprinone.
(46) Adenylate cyclase activators such as colforsin dapropate hydrochloride.
(47) Positive inotropic agents such as digoxin and metildigoxin; metabolic cardiotonic agents such as ubidecarenone; brain natriuretic peptides such as nesiritide.
(48) Drugs used for the treatment of erectile dysfunction such as alprostadil, aviptadil, and phentolamine mesilate.
(49) Drugs used in the treatment of obesity, including but not limited to, methamphetamine hydrochloride (Desoxyn®), amfepramone hydrochloride (Tenuate®), phentermine (Ionamin®), benzfetamine hydrochloride (Didrex®), phendimetrazine hydrochloride (Bontril®, Prelu-2®, Plegine®), mazindol (Sanorex®) and orlistat (Xenical®).
(50) Drugs used for the treatment of Alzheimer's disease and dementias such as the following types:
acetyl cholinesterase inhibitors including galantamine (Razadyne®), rivastigmine (Exelong®), donepezil (Aricept®) and tacrine (Cognex®);
NMDA receptor antagonists such as memantine (Namenda®); and
oxidoreductase inhibitors such as idebenone.
(51) Psychiatric medications such as the following types:
ziprasidone (Geodon™), risperidone (Risperdal™), olanzapine (Zyprexa™), valproate;
dopamine D4 receptor antagonists such as clozapine;
dopamine D2 receptor antagonists such as nemonapride;
mixed dopamine D1/D2 receptor antagonists such as zuclopenthixol;
GABA A receptor modulators such as carbamazepine;
sodium channel inhibitors such as lamotrigine;
monoamine oxidase inhibitors such as moclobemide and indeloxazine;
primavanserin, perospirone; and
PDE4 inhibitors such as rolumilast.
(52) Drugs used for the treatment of movement disorders or symptoms such as the following types:
catechol-O-methyl transferase inhibitors such as entacapone;
monoamine oxidase B inhibitors such as selegiline;
dopamine receptor modulators such as levodopa;
dopamine D3 receptor agonists such as pramipexole;
decarboxylase inhibitors such as carbidopa;
other dopamine receptor agonists such as pergolide, ropinirole, cabergoline;
ritigonide, istradefylline, talipexole; zonisamide and safinamide; and
synaptic vesicular amine transporter inhibitors such as tetrabenazine.
(53) Drugs used for the treatment of mood or affective disorders or OCD such as the following types tricyclic antidepressants such as amitriptyline (Elavil®), desipramine (Norpramin®), imipramine (Tofranil®), amoxapine (Asendin®), nortriptyline and clomipramine;
selective serotonin reuptake inhibitors (SSRIs) such as paroxetine (Paxil®), fluoxetine (Prozac®), sertraline (Zoloft®), and citralopram (Celexa®);
doxepin (Sinequan®), trazodone (Desyrel®) and agomelatine;
selective norepinephrine reuptake inhibitors (SNRIs) such as venlafaxine, reboxetine and atomoxetine; dopaminergic antidepressants such as bupropion and amineptine.
(54) Drugs for the enhancement of synaptic plasticity such as the following types:
nicotinic receptor antagonists such as mecamylamine; and
mixed 5-HT, dopamine and norepinephrine receptor agonists such as lurasidone.
(55) Drugs used for the treatment of ADHD such as amphetamine; 5-HT receptor modulators such as vortioxetine and alpha-2 adrenoceptor agonists such as clonidine.
(56) Neutral endopeptidase (NEP) inhibitors such as sacubitril, omapatrilat; and
(57) Methylene blue (MB).
   Pharmaceutical Compositions and their Routes of Administration

The compounds herein disclosed, and their pharmaceutically acceptable salts, thereof may be formulated as pharmaceutical compositions or “formulations”.

A typical formulation is prepared by mixing a compound described herein, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound described herein is being formulated. Solvents are generally selected based on solvents recognized by persons skilled in the art as safe (e.g., one described in the GRAS (Generally Recognized as Safe) database) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations may be prepared using conventional dissolution and mixing procedures. For example, the bulk drug substance (i.e., one or more of the compounds described herein, a pharmaceutically acceptable salt thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

A compound described herein or a pharmaceutically acceptable salt thereof is typically formulated into pharmaceutical dosage forms to provide an easily controllable dosage of the drug and to enable patient compliance with the prescribed regimen. Pharmaceutical formulations of compounds described herein, or a pharmaceutically acceptable salt thereof, may be prepared for various routes and types of administration. Various dosage forms may exist for the same compound. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The pharmaceutical compositions described herein will be formulated, dosed, and administered in a fashion, i.e., amounts, concentrations, schedules, course, vehicles, and route of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms develop further.

In some embodiments, a prophylactically effective amount of an sGC stimulator is one that prevents or delays the occurrence, progression or reoccurrence of muscle wasting, muscle necrosis, muscle weakness or muscle ischemia. In further embodiments, a prophylactically effective amount of an sGC stimulator is one that prevents or delays the occurrence or reoccurrence of muscle wasting, muscle necrosis, muscle weakness or muscle ischemia in a subject suffering from a Muscular Dystrophy. In further embodiments, a prophylactically effective amount of an sGC stimulator is one that prevents or delays the progression of muscle wasting, muscle necrosis, muscle weakness or muscle ischemia in a subject suffering from a Muscular Dystrophy. In other embodiments, a prophylactically effective amount of an sGC stimulator is one that prevents or delays the occurrence or reoccurrence of muscle wasting, muscle necrosis, muscle weakness or muscle ischemia in a subject suffering with one of Duchenne or Becker Muscular Dystrophy. In other embodiments, a prophylactically effective amount of an sGC stimulator is one that prevents or delays the progression of muscle wasting, muscle necrosis, muscle weakness or muscle ischemia in a subject suffering with one of Duchenne or Becker Muscular Dystrophy. In other embodiments, a prophylactically effective amount of an sGC stimulator is one that prevents or delays the progression of muscle wasting, muscle necrosis, muscle weakness or muscle ischemia in a subject suffering with one of the other known types of Muscular Dystrophy.

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release.

Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Gastroretentive formulations” are preparations designed to have increased retention in the stomach cavity. In some cases, they are used where a drug is preferentially or primarily absorbed via the stomach, is designed to treat the stomach directly, or where drug dissolution or absorption is aided drug absorption is aided by prolonged exposure to gastric acids. Examples of gastroretentive formulations include but are not limited to, high-density formulations, where the density of the formulation is higher than gastric fluid; floating formulations, which can float on top of gastric fluids due to increased buoyancy or lower density of the formulation; temporarily expandable formulations that are temporarily larger than the gastric opening; muco- and bio-adhesive formulations; swellable gel formulations; and in situ gel forming formulations. (See, e.g., Bhardwaj, L. et al. African J. of Basic & Appl. Sci. 4(6): 300-312 (2011)).

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

The terms “administer”, “administering” or “administration”, in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques.

In particular embodiments, the compositions are administered orally, intraperitoneally or intravenously.

In other embodiments, the compositions are administered rectally.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound described herein that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water-soluble carrier such as polyethylene glycol or an oil medium, for example, peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending a compound described herein in a vegetable oil, for example arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable drug-depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Drug-depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl ester wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, between 0.075% and 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds described herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds described herein include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration may have a mean particle size in the range of, for example, 0.1 to 500 microns (including particles with a mean particle size in the range between 0.1 and 500 microns in increments such as 0.5, 1, 30, 35 microns, etc.), which may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient. In another aspect, a compound described herein or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Kits

The pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) another therapeutic agent and a pharmaceutically acceptable carrier, vehicle or diluent in one or more containers or separate packaging. The kits may optionally comprise instructions describing a method of using the pharmaceutical compositions in one or more of the methods described herein (e.g. preventing or treating one or more of the diseases and disorders described herein). The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening. It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or other compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

### Example 1. Non-Clinical Studies

In Vivo Mouse Models:

A transgenic rat model (Pvr13-Cre) of achalasia has recently been developed and described (“Megaesophagus in a line of transgenic rats: a model of achalasia”; Pang J; Borjeson T M; Muthupalani S; Ducore R M; Carr C A; Feng Y; Sullivan M P; Cristofaro V; Luo J; Lindstrom J M; Fox J G; Veterinary pathology, 51(6): 1187-200, 2014). These rats present with an abnormal enlargement of the esophagus at 3 to 4 months of age and a reduced number of myenteric neurons leading to symptomology similar to human disease. The utility of an sGC stimulator to treat achalasia could be assessed in a study utilizing these rats. 4-week-old Pvr13-Cre mice would be divided into groups of 10-12 rats per treatment group and would receive sGC stimulator over the course of 7 weeks. Rats would be dosed with an sGC stimulator by oral gavage (ranging from 1 to 10 mg/kg/day, qd or bid) or by administration of an equivalent dose in food. One group would serve as a vehicle control. Relevant endpoints would be body weight, assessment of the esophagus and lower esophageal sphincter by contrast radiography and fluoroscopy, and histological assessment of the esophagus including the number of myenteric neurons. An sGC stimulator would be expected to preserve body weight, normalize enlargement of the esophagus, and normalize esophageal function.

Ex Vivo Models:

The effect of sGC stimulators on muscle contractility would be measured in ex vivo studies on lower esophageal sphincter tissue isolated from rats. The lower esophageal sphincter would be isolated from the esophagus of a rat and strips of circular smooth muscle tissue would be prepared. The tissue strip would be suspended under tension in an organ bath and the mechanical force of the tissue would be determined using an isometric force transducer. Simultaneous measurement of multiple isolated tissues from the same sphincter from the same donor would be conducted over the course of the study. The tissue would be subjected to a steady and consistent tension and then treated with carbachol to induce a contraction. The ability of an sGC stimulator to induce relaxation of carbachol-induced contraction would be determined as follows:

Vehicle

DETA-NO, a nitric oxide donor (cumulative concentrations)

sGC stimulator (cumulative concentrations ranging from 1 nM to 100 uM)

Sub-threshold concentration of DETA_NO+sGC stimulator (1 nM to 10 uM)

Both NO donors and sGC stimulators would be expected to relax esophageal smooth muscle and act together in an additive or synergistic fashion.

Ex-Vivo Study with Human LES Tissues

The objective of this study was to determine the relaxation effects of two sGC stimulators, Compound A and Compound B (depicted below) on human lower esophageal sphincter (LES) tissues ex vivo. Human lower esophageal sphincter muscle strips were mounted in a wire myograph apparatus and pre-contracted with carbachol (Cch). A cumulative concentration response curve (CCRC) of Compound A and Compound B was then performed on the tissues. The concentration response was performed with the compounds alone, and/or with the test compounds in the presence of a fixed concentration of the NO donor DETA-NONOate.

### Materials

### Test Substances, Reference Substances, and Vehicle

Compound A and Compound B were generated by Ironwood Pharmaceuticals.

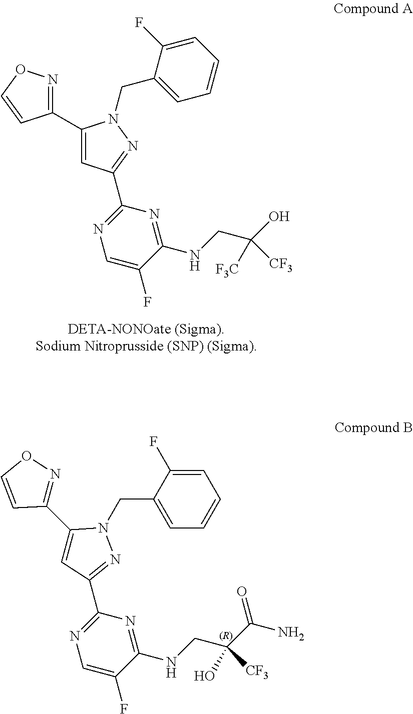

### Storage and Formulation

Compound A and Compound B were stored at room temperature. On each experimental day, 10 mM stock of the test compound was prepared in DMSO. Further dilutions up to 0.1 μM were made serially in ethanol, and subsequently in distilled water, depending on solubility of the compounds in those solvents. Compound A and Compound B Vehicle solutions were made by following the same serial dilution methods

DETA-NONOate was stored at −20° C. On each experimental day, a stock solution of 100 mM was prepared in Phosphate Buffered Saline (PBS), pH 8 (Boston Bioproducts). A 1:10 dilution from the stock was made in PBS pH 8, to obtain a concentration of 10 mM.

SNP was stored at −20° C. On each experimental day, a stock solution of 100 mM was prepared in distilled water. 25 μl of the 100 mM stock was added to each bath for a final concentration of 100 μM in bath.

### Methods

### Study Tissue or Subjects

Tissues were obtained postmortem. Only macroscopically normal tissue that was obtained from donors with no known recent history of gastrointestinal disease.

### Study Design

### Dissection, Mounting, and Equilibration

Human LES mucosa-free muscle strips, of approximately 15 mm in length and 2-3 mm width were dissected free from surrounding tissue and mounted on tissue posts in 25 mL organ baths (Panlab 16 channel automated organ bath and thermostatic controller) containing physiological saline solution (PSS; composition: 119.0 mM NaCl, 4.70 mM KCl, 1.20 mM MgSO₄, 24.9 mM NaHCO₃, 1.20 mM KH₂PO₄, 2.50 mM CaCl₂, 11.1 mM glucose), aerated with 95% O₂/5% CO₂, warmed and maintained at approximately 37° C. The PSS solution was supplemented with 1 μM indomethacin. The LES strips were mounted on the tissue posts and allowed to equilibrate for approximately 30 minutes.

### Tension Application

The LES strips were set to a tension of 2.0 g+0.2 g. Baths were washed approximately every 15 minutes over a 60-minute period, with re-tensioning to 2.0±0.2 g if tension dropped below 1.0 g.

### Check of the Sphincter Muscle Function

The viability of the LES muscle strips was tested by application of 80 mM KCl, looking for maximum contractility response. Upon plateau all baths were washed three times with PSS and the tension was allowed to return to baseline levels. The strips that responded to the KCl functional check were used for CCh-induced contractions.

### Assay Conditions

For each one of compounds A and B, experiments were conducted with LES tissues from two human donors on two separate days.

Upon stabilization of baseline tension, each of the following experiments was allocated to one bath such that 9 or more muscle strips were set up for each compound on each experimental day.

Compound A Experiments with tissues from Donor 1:
1. CCRC to DETA-NO vehicle
2. CCRC to Compound A vehicle
3. CCRC to DETA-NO
4. CCRC to DETA-NO
5. CCRC to Compound A
6. CCRC to Compound A
7. Since DETA-NO relaxed LES, a CCRC to Compound A was performed in the presence of DETA-NO at a sub-threshold concentration (1 μM, determined in conditions 3 and 4)
8. CCRC to Compound A in the presence of DETA-NO (1 M)
9. CCRC to Compound A vehicle in the presence of DETA-NO (1 M).

Compound A Experiments with tissues from Donor 2:
1. CCRC to Compound A vehicle
2. CCRC to Compound A vehicle
3. CCRC to Compound A vehicle in the presence of 10 μM DETA-NO
4. CCRC to Compound A vehicle in the presence of 30 μM DETA-NO
5. CCRC to Compound A
6. CCRC to Compound A
7. CCRC to Compound A in the presence of 10 μM DETA-NO
8. CCRC to Compound A in the presence of 10 μM DETA-NO
9. CCRC to Compound A in the presence of 10 μM DETA-NO
10. CCRC to Compound A in the presence of 10 μM DETA-NO
11. CCRC to Compound A in the presence of 30 μM DETA-NO
12. CCRC to Compound A in the presence of 30 μM DETA-NO
13. CCRC to Compound A in the presence of 30 μM DETA-NO

All of the LES strips were pre-contracted with 1 μM CCh prior to the CCRCs. Application of vehicle controls, DETA-NO or Compound A followed stabilization of the CCh-induced contraction. Application of the sub-threshold concentration of DETA-NO (conditions 7, 8 and 9, Donor 1) was immediately prior to CCRC to Compound A. Application of PBS or DETA-NO was immediately prior to the CCRC to vehicle or Compound A. The incubation time at each concentration was approximately 15-20 minutes or time to plateau of the relaxation effect.

The Compound A, Donor 1 CCRCs consisted of 5 concentrations:

DETA-NO: 0.01 μM, 0.1 μM, 1 μM, and 10 μM and 100 μM

Compound A: 0.001 μM and 0.01 μM, 0.1 μM, 1 μM and 10 μM

The Compound A, Donor 2 CCRCs consisted of 4 concentrations of Compound A: 0.01 μM, 0.1 μM, 1 μM and 10 μM

In all cases, 1000-fold dilutions of test article solution into the bathing solution were performed in order to reach the desired batch concentration.

Compound B Experiments:
1. CCRC to Compound B vehicle in the presence of PBS
2. CCRC to Compound B in the presence of PBS
3. CCRC to Compound B in the presence of PBS
4. CCRC to Compound B in the presence of PBS
5. CCRC to Compound B in the presence of PBS
6. CCRC to Compound B vehicle in the presence of DETA-NO (30 μM)
7. CCRC to Compound B vehicle in the presence of DETA-NO (30 μM)
8. CCRC to Compound B in the presence of DETA-NO (30 μM)
9. CCRC to Compound B in the presence of DETA-NO (30 μM)
10. CCRC to Compound B in the presence of DETA-NO (30 μM)
11. CCRC to Compound B in the presence of DETA-NO (30 μM).

All of the LES strips were pre-contracted with 1 μM CCh prior to the CCRCs. Application of 30 μM DETA-NO or PBS followed stabilization of the CCh-induced contraction. Application of Compound B or vehicle followed addition of 30 μM DETA-NO or PBS. The incubation time at each concentration was approximately 20 minutes or time to plateau of the relaxation effect. Incubation times in the CCRC did not exceed 70 min.

The Compound B CCRCs consisted of 4 concentrations of Compound B: 0.01 μM, 0.1 μM, 1 μM, and 10 μM. 1000-fold dilutions of test article solution into the bathing solution were performed in order to reach the desired bath concentration.

Following the Compound B or vehicle CCRCs, 100 μM SNP was added to all baths to determine the maximal achievable relaxation by a high NO concentration. 1 μM isoprenaline was added to all baths to test the LES function at the end of each experiment, and also was used as reference for 100% relaxation.

Experiments with Compounds C, D and E

Experiments paralleling those described above for compounds A and B were performed with three additional compounds C, D and E (IZC-2):

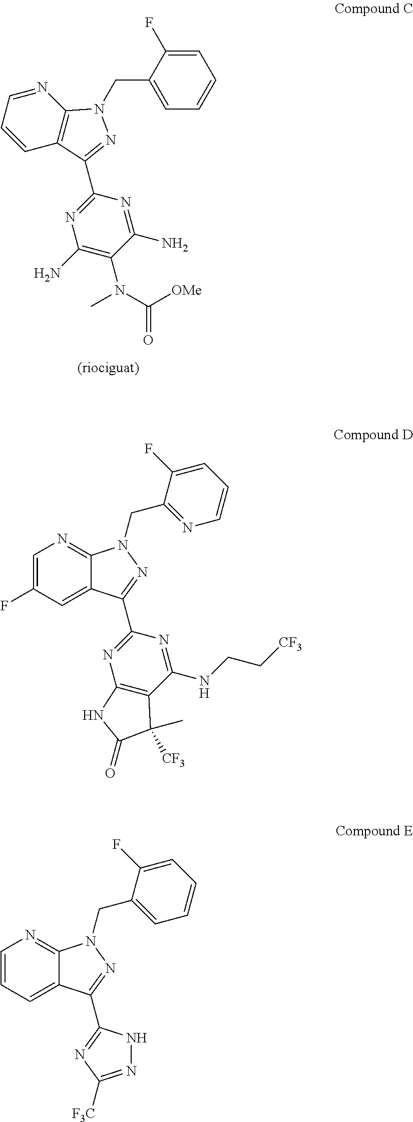 [SMILES: COC(=O)N(C)c1c(N)nc(-c2nn(Cc3ccccc3F)c3ncccc23)nc1N ; C[C@@]1(C(F)(F)F)C(=O)Nc2nc(-c3nn(Cc4ncccc4F)c4ncc(F)cc34)nc(NCCC(F)(F)F)c21 ; Fc1ccccc1Cn1nc(-c2nc(C(F)(F)F)n[nH]2)c2cccnc21]

LES strips were isolated from two human donors. All of the LES strips were pre-contracted with 1 μM CCh prior to the CCRCs. All of the LES strips were pre-treated with 30 uM DETA-NO. For Compounds C, D and E, CCRC included the concentrations: 0.01 μM, 0.1 μM, 1 μM and 10 μM
Vehicle CCRC (n=5)
Cmpd C CCRC (n=6)
Cmpd D CCRC (n=6)
Cmpd E CCRC (n=6)

Following the Compound C, D, or E or vehicle CCRCs, 10 μM sildenafil was added to all baths to determine the achievable relaxation by a PDE5 inhibitor. 1 μM isoprenaline was added to all baths to test the LES function at the end of each experiment, and also was used as reference for 100% relaxation.

### Data Analysis

Analysis was conducted in units of force or normalized to % relaxation of compound response to CCh, relative to isoprenaline control, according to the equation: % relaxation=100*(tensionₓ−tension_{Cch})/(tension_{Iso}−tension_{Cch}), where tensionₓ=tension at a given concentration of test article, tension_{Cch}=tension following CCh contraction and stabilization, and tension_{Iso}=tension following isoprenaline relaxation. EC₅₀ values were determined in GraphPad Prism using nonlinear regression fitting data points from all tissues to a 4 parameter logistic regression curve, with the top constrained to 100% relaxation and Hill slope constrained to 1.0. Analyzed data, with non-linear regression and EC₅₀ values were displayed graphically using GraphPad Prism, where appropriate.

Where more than one replicate was obtained from one tissue, the mean response was calculated.

### Results

In the absence of NO, 10 μM Compound A induced a maximum relaxation of 49% (N=2) in the first donor LES and a maximum relaxation of 31% (N=2) in the second donor. In the second donor, 10 μM DETA-NO induced a mean percentage relaxation of 8% (N=5), while 30 μM DETA-NO induced a mean percentage relaxation of 13% (N=4).

For donor 1, in the presence of 1 μM DETA-NO, 10 μM Compound A induced a relaxation of 16%. In donor 2, in the presence of 10 μM DETA-NO, 10 μM Compound A induced a maximum relaxation of 38% (N=4), and in the presence of 30 μM DETA-NO, Compound A induced a maximum relaxation of 51% (N=3).

In the absence of NO, 10 μM Compound B induced a maximum relaxation of 47% (N=8) relative to isoprenaline in the lower esophageal sphincter. 30 μM DETA-NO induced a mean relaxation of 7.6% (N=12), relative to isoprenaline.

In the presence of 30 μM DETA-NO, 10 μM Compound B induced a maximum relaxation of 77% (N=8) relative to isoprenaline. In the presence of 30 μM DETA-NO, Compound B relaxed LES with an EC₅₀ of 1.7 μM.

In the presence of 30 μM DETA-NO, 10 μM Compound C induced a maximum relaxation of 90% (N=6) relative to isoprenaline. In the presence of 30 μM DETA-NO, Compound C relaxed LES with an EC₅₀ of 0.50 μM.

In the presence of 30 μM DETA-NO, 10 μM Compound D induced a maximum relaxation of 80% (N=6) relative to isoprenaline. In the presence of 30 μM DETA-NO, Compound D relaxed LES with an EC₅₀ of 1.6 μM.

In the presence of 30 μM DETA-NO, 10 μM Compound E induced a maximum relaxation of 89% (N=6) relative to isoprenaline. In the presence of 30 μM DETA-NO, Compound E relaxed LES with an EC₅₀ of 0.53 μM.

In conclusion, it was demonstrated that five sGC stimulators induced a concentration-dependent relaxation of human LES.

### Example 2

### Clinical Studies-A

The effect of sGC stimulators will be determined clinically in human patients with idiopathic achalasia by manometry—a measure of the esophageal pressure gradient in response to swallowing. PDE5 inhibitors, such as sildenafil, which similarly result in increased levels of cGMP, have been used off label in achalasia patients and have shown some limited utility (“Effects of sildenafil on esophageal motility of patients with idiopathic achalasia”; Bortolotti M; Mari C; Lopilato C; Porrazzo G; Miglioli M; Gastroenterology, 118(2): 253-7, 2000). Achalasia patients would be fasted overnight and then prepped in the morning with a manometric pressure probe. sGC stimulators would be administered p.o. Patients would then be asked to perform dry swallows at approximately 30-60-second intervals for the entire recording period while manometric pressure would be measured. An sGC stimulator would be expected to reduce esophageal pressure, induce relaxation of the lower esophageal sphincter, and restore esophageal peristalsis.

### Clinical Studies-B

A multicenter, randomized, double-blind, placebo-controlled, parallel-group, single-dose study will randomize approximately 20 patients to receive an sGC stimulator of the invention (15 patients to sGC stimulator and 5 to matching placebo). The study will randomize patients diagnosed with primary Type II achalasia with an integrated relaxation pressure (IRP) >15 mm Hg by baseline High Resolution Impedance Manometry (HRIM).

Test product (an sGC stimulator described above) will be administered orally as 1 mg tablets; the dose will be a total of 5 mg (5 tablets). Placebo will match the sGC stimulator oral tablets. Patients will begin a liquid diet on Day −1 and then will fast overnight. To confirm eligibility, patients will undergo a baseline protocol-specific HRIM procedure that includes 2 swallowing sequences recorded 1 hour (+15 minutes) apart.

After the second recording, the HRIM catheter will be removed, and patients will complete a baseline symptom assessment. Patients who meet all eligibility criteria in addition to having confirmed Type II achalasia and IRP >15 mm Hg will be randomized to receive a single 5-mg dose of the sGC stimulator or matching placebo, together with 8 oz. of water. Following study drug administration, the HRIM catheter will be reinserted for the postdose HRIM procedure. The HRIM catheter will be removed after the final recording, and patients will complete a postdose symptom assessment.

Various embodiments of the invention can be described in the text below. As explained supra, it is to be understood that pharmaceutically acceptable salts are included in these embodiments, even though the phrase “pharmaceutically acceptable salt” is not written.

[1]. A method of treating achalasia in a patient in need thereof, comprising administering to said patient a therapeutically effective amount of an sGC stimulator or a pharmaceutically acceptable salt thereof.
[2]. A method of [1] above, or according to other embodiments of the invention, wherein the achalasia is primary achalasia.
[3]. A method of [1] above, or according to other embodiments of the invention, wherein the achalasia is secondary achalasia.
[4]. A method of [3] above, or according to other embodiments of the invention, wherein the achalasia is secondary achalasia associated with Chagas disease.
[5]. A method of [3] above, or according to other embodiments of the invention, wherein the achalasia is secondary achalasia associated with esophageal cancer.
[6]. A method of [1], [2], [3], [4], [5], or [6] above, or according to other embodiments of the invention, wherein said sGC stimulator or pharmaceutically acceptable salt thereof is administered as a monotherapy.
[7]. A method of [1], [2], [3], [4], [5], or [6] above, or according to other embodiments of the invention, wherein said sGC stimulator or pharmaceutically acceptable salt thereof is administered in combination with a therapeutically or prophylactically effective amount of one or more additional therapeutic agents.
[8]. A method of [7] above, or according to other embodiments of the invention, wherein the additional therapeutic agent is a calcium channel blocker.
[9]. A method of [8] above, or according to other embodiments of the invention, wherein the additional therapeutic agent is nifedipine.
[10]. A method of [9] above, or according to other embodiments of the invention, wherein the nifedipine is administered sublingually.
[11]. A method of [7] above, or according to other embodiments of the invention, wherein the additional therapeutic agent is a botox injection.
[12]. A method of [7] above, or according to other embodiments of the invention, wherein the additional therapeutic agent is a compound known to up-regulate the NO-pathway.
[13]. A method of [12] above, or according to other embodiments of the invention, wherein said compound is selected from nitric oxide, a NO-donor, an sGC stimulator, a sGC activator or a PDE5 inhibitor.
[14]. A method of [13] above, or according to other embodiments of the invention, wherein the compound is an NO-donor.
[15]. A method of [14] above, or according to other embodiments of the invention, wherein the NO-donor is selected from a nitrate, a nitrite, a NONOate or a nitrosothiol.
[16]. A method of [13] above, or according to other embodiments of the invention, wherein the sGC stimulator is selected from riociguat or vericiguat.
[17]. A method of [13] above, or according to other embodiments of the invention, wherein the sGC activator is ataciguat or cinaciguat.
[18]. A method of any one of [1] to [17] above, or according to other embodiments of the invention, wherein the patient in need thereof is an adult.
[19]. A method of any one of [1] to [17] above, or according to other embodiments of the invention, wherein the patient in need thereof is a child.
[20]. A method of [18] or [19] above, or according to other embodiments of the invention, wherein the patient in need thereof is a person that has been diagnosed with achalasia.
[21]. A method of [18] or [19] above, or according to other embodiments of the invention, wherein the patient in need thereof is a person who displays the symptoms associated with achalasia.
[22]. A method of [18], [19], [20], or [21] above, or according to other embodiments of the invention, wherein the patient in need thereof displays an elevated level of LES pressure measured by manometry.
[23]. A method of [22] above, or according to other embodiments of the invention, wherein the patient in need thereof displays a LES pressure higher than 50 mm Hg a measured by manometry.
[24]. A method of [23] above, or according to other embodiments of the invention, wherein the patient in need thereof displays a LES pressure higher than 75 mm Hg as measured by manometry.
[25]. A method of [24] above, or according to other embodiments of the invention, wherein the patient in need thereof displays a LES pressure higher than 100 mm Hg as measured by manometry.
[26]. A method of [18], [19], [20], or [21] above, or according to other embodiments of the invention, wherein the patient in need thereof displays a manometry pattern consistent with failure of the esophagus to relax appropriately after swallowing.
[27]. A method of [26] above, or according to other embodiments of the invention, wherein the patient in need thereof displays a manometry pattern consistent with less than 75% relaxation of the esophagus after swallowing.
[28]. A method of any one of [1] to [27] above, or according to other embodiments of the invention, wherein the administration of an sGC stimulator or pharmaceutically acceptable salt thereof, alone or in combination with another therapeutic agent, results in an observable or measurable decrease in the degree of failure of the esophageal smooth muscle to relax after swallowing.
[29]. A method of any one of [1] to [27] above, or according to other embodiments of the invention, wherein the administration of an sGC stimulator or pharmaceutically acceptable salt thereof, alone or in combination with another therapeutic agent, results in an observable or measurable decrease in the degree of failure of the LES to relax after swallowing.
[30]. A method of any one of [1] to [27] above, or according to other embodiments of the invention, wherein the administration of an sGC stimulator or pharmaceutically acceptable salt thereof, alone or in combination with another therapeutic agent, results in an observable or measurable decrease in the degree of aperistalsis of the esophageal body in response to swallowing.
[31]. A method of any one of [1] to [27] above, or according to other embodiments of the invention, wherein the administration of an sGC stimulator or pharmaceutically acceptable salt thereof, alone or in combination with another therapeutic agent, results in an observable or measurable decrease in the degree of dysphagia.
[32]. A method of any one of [1] to [27] above, or according to other embodiments of the invention, wherein the administration of an sGC stimulator or pharmaceutically acceptable salt thereof, alone or in combination with another therapeutic agent, results in an observable or measurable reduction in regurgitation of undigested food.
[33]. A method of any one of [1] to [27] above, or according to other embodiments of the invention, wherein the administration of an sGC stimulator or pharmaceutically acceptable salt thereof, alone or in combination with another therapeutic agent, results in an observable or measurable decrease in the progression of esophageal fibrosis.
[34]. A method of any one of [1] to [27] above, or according to other embodiments of the invention, wherein the administration of an sGC stimulator or pharmaceutically acceptable salt thereof, alone or in combination with another therapeutic agent, results in an observable or measurable reduction in inflammation around the myenteric plexus.
[35]. A method of any one of [1] to [27] above, or according to other embodiments of the invention, wherein the administration of an sGC stimulator or pharmaceutically acceptable salt thereof, alone or in combination with another therapeutic agent, results in an observable or measurable reduction in heartburn.
[36]. A method of any one of [1] to [27] above, or according to other embodiments of the invention, wherein the administration of an sGC stimulator or pharmaceutically acceptable salt thereof, alone or in combination with another therapeutic agent, results in a measurable or observable reduction in chest pain.
[37]. A method of any one of [1] to [27] above, or according to other embodiments of the invention, wherein the administration of an sGC stimulator or pharmaceutically acceptable salt thereof, alone or in combination with another therapeutic agent, results in an observable or measurable reduction of a symptom selected from wheezing, hoarseness, sore throat, coughing when lying in a horizontal position, degree of retention of food in the esophagus, aspiration of food into the lungs or cardiospasm.
[38]. A method of any one of [1] to [27] above, or according to other embodiments of the invention, wherein the administration of an sGC stimulator or pharmaceutically acceptable salt thereof, alone or in combination with another therapeutic agent, results in an observable or measurable inhibition of weight loss.
[39]. A method of any one of [1] to [27] above, or according to other embodiments of the invention, wherein the administration of an sGC stimulator or pharmaceutically acceptable salt thereof, alone or in combination with another therapeutic agent, results in an observable or measurable improvement in the ability of esophageal smooth muscles fibers to relax after swallowing.
[40]. A method of any one of [1] to [27] above, or according to other embodiments of the invention, wherein the administration of an sGC stimulator or pharmaceutically acceptable salt thereof, alone or in combination with another therapeutic agent, results in an observable or measurable improvement in peristalsis of the esophagus.
[41]. A method of any one of [1] to [27] above, or according to other embodiments of the invention, wherein the administration of an sGC stimulator or pharmaceutically acceptable salt thereof, alone or in combination with another therapeutic agent, results in an observable or measurable improvement in the ability to swallow liquids or solids.
[42]. A method of any one of [1] to [27] above, or according to other embodiments of the invention, wherein the administration of an sGC stimulator or pharmaceutically acceptable salt thereof, alone or in combination with another therapeutic agent, results in an observable or measurable improvement in chest pain.
[43]. A method of any one of [1] to [27] above, or according to other embodiments of the invention, wherein the administration of an sGC stimulator or pharmaceutically acceptable salt thereof, alone or in combination with another therapeutic agent, results in an observable or measurable improvement in heartburn.
[44]. A method of any one of [1] to [27] above, or according to other embodiments of the invention, wherein the administration of an sGC stimulator or pharmaceutically acceptable salt thereof, alone or in combination with another therapeutic agent, results in a measurable reduction in the LES pressure after swallowing as measured by manometry.
[45]. A method of any one of [1] to [27] above, or according to other embodiments of the invention, wherein the administration of an sGC stimulator or pharmaceutically acceptable salt thereof, alone or in combination with another therapeutic agent, results in a measurable increase in the percentage of relaxation of the LES after swallowing as measured by manometry.
[46]. A method of any one of [1] to [27] above, or according to other embodiments of the invention, wherein the administration of an sGC stimulator or pharmaceutically acceptable salt thereof, alone or in combination with another therapeutic agent, results in a measurable decrease in intra-esophageal pressure compared to intragastric pressure after swallowing as measured by manometry.
[47]. A method of any one of [1] to [27] above, or according to other embodiments of the invention, wherein the administration of an sGC stimulator or pharmaceutically acceptable salt thereof, alone or in combination with another therapeutic agent, results in the improvement or reduction, or slowing down in the development of one or more symptoms selected from: dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus and aspiration of food into the lungs.
[48]. A method of any one of [1] to [27] above, or according to other embodiments of the invention, wherein the administration of an sGC stimulator or pharmaceutically acceptable salt thereof, alone or in combination with another therapeutic agent, is aimed at treating one or more symptoms selected from: dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus and aspiration of food into the lungs.
[49]. A method of any one of [1] to [48] above, or according to other embodiments of the invention, wherein the sGC stimulator is administered prior to, at the same time as, or after the initiation of treatment with another therapeutic agent.
[50]. A method of any one of [1] to [48] above, or according to other embodiments of the invention, wherein the sGC stimulator is selected from riociguat, neliciguat, vericiguat, BAY-41-2272, BAY 41-8543 or etriciguat.
[51]. A method of any one of [1] to [48] above, or according to other embodiments of the invention, wherein the sGC stimulator is one of Formula IA, or a pharmaceutically acceptable salt thereof,

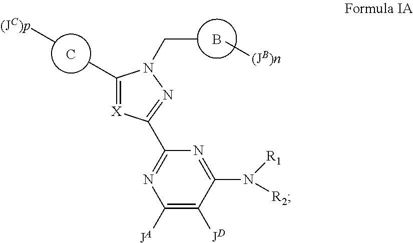

wherein:
X is selected from N, CH, C(C₁₋₄ alkyl), C(C₁₋₄ haloalkyl), CCl and CF;
ring B is a phenyl or a 6-membered heteroaryl ring containing 1 or 2 ring nitrogen atoms, or ring B is a thiophene;
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said R^{B} that is a C₁₋₆ aliphatic and each of said R^{B} that is a C₃₋₈ cycloaliphatic ring is optionally substituted with up to 3 instances of halogen;
J^{A} is selected from hydrogen, halogen, methyl, methoxy, trifluoromethyl, trifluoromethoxy or —NR^{a}R^{b}, wherein R^{a} and R^{b} are each independently selected from hydrogen, C₁₋₆ alkyl or a 3-6 cycloalkyl ring;
J^{D} is absent or selected from halogen, —CN, —CF₃, methoxy, trifluoromethoxy, nitro, amino or methyl;
R¹ and R², together with the nitrogen atom to which they are attached, form a 4 to 8-membered heterocyclic ring or 5 or 6-membered heteroaryl ring; wherein said 4 to 8-membered heterocyclic ring or 5 or 6-membered heteroaryl ring optionally contains in addition to the nitrogen atom up to 3 ring heteroatoms independently selected from N, O or S, and is optionally substituted by up to 5 instances of R⁵; or
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₃₋₈ cycloalkyl ring, 4 to 8-membered heterocyclic ring group, 5 or 6-membered heteroaryl and the C₁₋₆ alkyl portion of said C₁₋₆ alkyl-R^{Y} is optionally and independently substituted with up to 5 instances of R^{5a}; provided that R¹ and R² are never simultaneously hydrogen; and provided than when X is one of CH, C(C₁₋₄ alkyl), C(C₁₋₄ haloalkyl), CCl or CF, one of R¹ and R² is not a pyridine or a pyrimidine; or
alternatively, J^{D} and one of R¹ or R² can form a 5-6 membered heterocyclic ring containing up to two heteroatoms selected from O, N and S and optionally substituted with up to 3 instances of oxo or —(Y)—R⁹;
wherein Y is either absent or is a linkage in the form of a C₁₋₆ alkyl chain, optionally substituted by up to 6 instances of fluoro;
each R⁹ is independently selected from hydrogen, fluoro, —CN, —OR¹⁰, —SR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, a C₃₋₆ cycloalkyl ring, a 4-8-membered heterocyclic ring or a 5-6 membered heteroaryl ring; wherein each said 4 to 8-membered heterocyclic ring or 5 to 6-membered heteroaromatic ring contains up to 4 ring heteroatoms independently selected from N, O or S; and wherein each of said C₃₋₆ cycloalkyl rings, each of said 4 to 8-membered heterocyclic rings and each of said 5 to 6-membered heteroaromatic rings is optionally substituted with up to 3 instances of R¹¹;
each R¹¹ is independently selected from halogen, C₁₋₆ alkyl, —CN, —OR¹², —SR¹², —COR¹², —OC(O)R¹², —C(O)OR¹², —C(O)N(R¹²)₂, —C(O)N(R¹²)SO₂R¹², —N(R¹²)C(O)R¹², —N(R¹²)C(O)OR¹², —N(R¹²)C(O)N(R¹²)₂, —N(R¹²)₂, —SO₂R¹², —SO₂N(R¹²)₂, —SO₂N(R¹²)COOR¹², —SO₂N(R¹²)C(O)R¹², —N(R¹²)SO₂R¹² and —N═OR¹²; wherein each of said C₁₋₆ alkyl is optionally and independently substituted by up to 3 instances of fluoro, —OH, —O(C₁₋₄ alkyl), phenyl and —O(C₁₋₄ fluoroalkyl)
wherein each R¹⁰ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo; and
wherein each R¹² is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
R^{Y} is selected from a C₃₋₈ cycloalkyl ring, a 4 to 8-membered heterocyclic ring, phenyl, or a 5 to 6-membered heteroaromatic ring; wherein each of said 4 to 8-membered heterocyclic ring or 5 to 6-membered heteroaromatic ring contains up to 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaromatic ring is optionally substituted with up to 5 instances of R^{5c};
each R^{5c} is independently selected from halogen, —CN, C₁₋₆ alkyl, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
two instances of R^{5c} attached to the same or different ring atoms of R^{Y}, together with said ring atom or atoms, may form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; wherein R¹¹ is hydrogen or a C₁₋₂ alkyl;
each R^{5a} is independently selected from halogen, —CN, C₁₋₆ alkyl, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; or
when one of R¹ or R² is the C₃₋₈ cycloalkyl ring, 4 to 8-membered heterocyclic ring or 5 or 6-membered heteroaryl substituted with up to 5 instances of R^{5a}, two of the instances of R^{5a} attached to the same or different ring atoms of said R¹ or R², together with said atom or atoms, may optionally form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl;
each R⁵ is independently selected from halogen, —CN, C₁₋₆ alkyl, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁶ is independently selected from hydrogen, a C₁₋₆ alkyl, phenyl, benzyl, a C₃₋₈ cycloalkyl ring or a 4 to 7-membered heterocyclic ring, a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
when R¹ and R² attached to the nitrogen atom form the 4 to 8-membered heterocyclic ring or 5 or 6-membered heteroaryl ring substituted with up to 5 instances of R⁵, two of the instances of R⁵ attached to the same or different atoms of said ring, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen; wherein R is hydrogen or a C₁₋₂ alkyl;
p is an integer selected from 0, 1 or 2;
ring C is a monocyclic 5-membered heteroaryl ring containing up to 4 ring heteroatoms selected from N, O or S; wherein said monocyclic 5-membered heteroaryl ring is not a 1,3,5-triazinyl ring;
each J^{C} is independently selected from halogen or a C₁₋₄ aliphatic optionally and independently substituted by up to 3 instances of C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen.
   [52]. A method of [51] above, or according to other embodiments of the invention, wherein the sGC stimulator is one of Formula IB

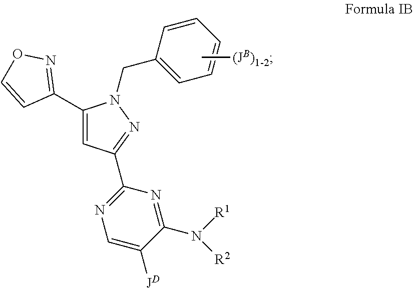

wherein J^{D} is selected from hydrogen or halogen; J^{B} is halogen and
R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 10-membered heterocyclic ring, a 5 or 6-membered heteroaryl, phenyl or a C₁₋₆ alkyl-R^{Y}; wherein each of said 4 to 10-membered heterocyclic ring and each of said 5 or 6-membered heteroaryl ring contains up to 3 ring heteroatoms independently selected from N, O and S; and wherein each of said C₁₋₆ alkyl, C₁₋₆ alkyl portion of each said C₁₋₆ alkyl-R^{Y} moiety, C₃₋₈ cycloalkyl ring, 4 to 10-membered heterocyclic ring group, 5 or 6-membered heteroaryl, phenyl and C₁₋₆ alkyl-R^{Y} is optionally and independently substituted with up to 5 instances of R^{5f}; provided that one of R¹ or R² may not be pyridine or pyrimidine;
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, C₁₋₆ alkyl, C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R′ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen; and
R″ is hydrogen or a C₁₋₂ alkyl.
   [53]. A method of [52] above, or according to other embodiments of the invention, wherein the sGC stimulator is one of Formula IC,

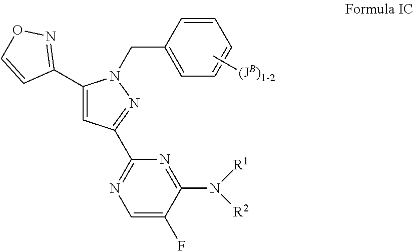

wherein J^{B} is halogen;
R¹ is hydrogen or C₁₋₆ alkyl; and
R² is a C₁₋₆ alkyl group optionally and independently substituted by up to three instances of R^{5a}.
   [54]. A method of [53] above, or according to other embodiments of the invention, wherein the sGC stimulator is selected from one depicted below:

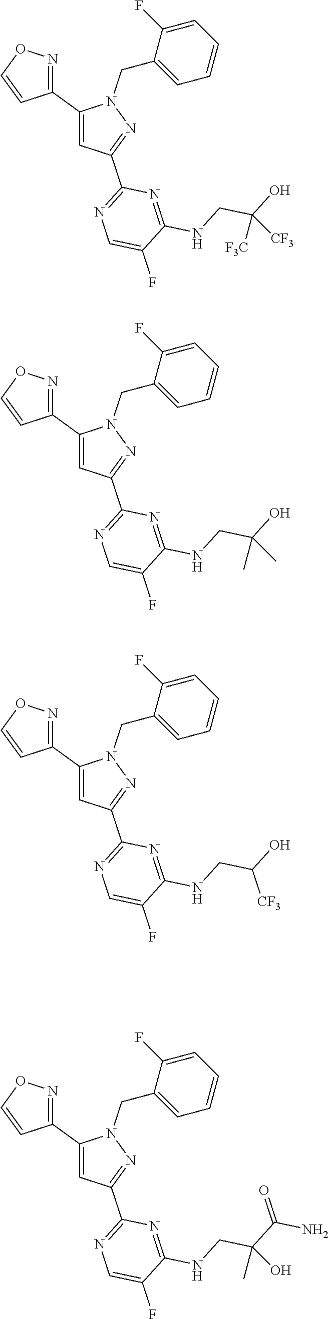 [SMILES: OC(CNc1nc(-c2cc(-c3ccon3)n(Cc3ccccc3F)n2)ncc1F)(C(F)(F)F)C(F)(F)F ; CC(C)(O)CNc1nc(-c2cc(-c3ccon3)n(Cc3ccccc3F)n2)ncc1F ; OC(CNc1nc(-c2cc(-c3ccon3)n(Cc3ccccc3F)n2)ncc1F)C(F)(F)F ; CC(O)(CNc1nc(-c2cc(-c3ccon3)n(Cc3ccccc3F)n2)ncc1F)C(N)=O]

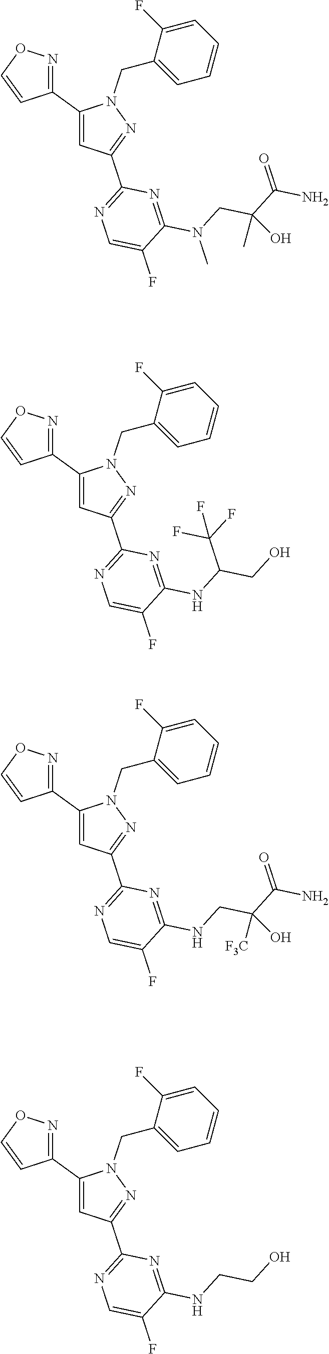 [SMILES: CN(CC(C)(O)C(N)=O)c1nc(-c2cc(-c3ccon3)n(Cc3ccccc3F)n2)ncc1F ; OCC(Nc1nc(-c2cc(-c3ccon3)n(Cc3ccccc3F)n2)ncc1F)C(F)(F)F ; NC(=O)C(O)(CNc1nc(-c2cc(-c3ccon3)n(Cc3ccccc3F)n2)ncc1F)C(F)(F)F ; OCCNc1nc(-c2cc(-c3ccon3)n(Cc3ccccc3F)n2)ncc1F]

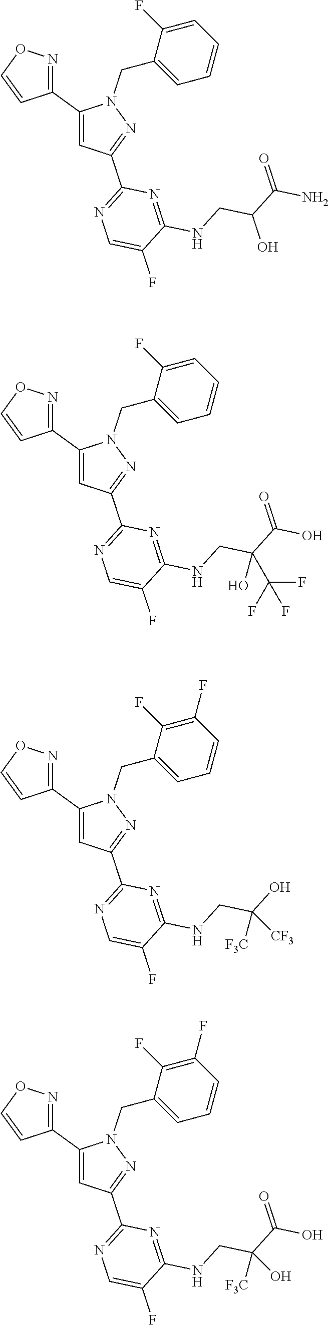 [SMILES: NC(=O)C(O)CNc1nc(-c2cc(-c3ccon3)n(Cc3ccccc3F)n2)ncc1F ; O=C(O)C(O)(CNc1nc(-c2cc(-c3ccon3)n(Cc3ccccc3F)n2)ncc1F)C(F)(F)F ; OC(CNc1nc(-c2cc(-c3ccon3)n(Cc3cccc(F)c3F)n2)ncc1F)(C(F)(F)F)C(F)(F)F ; O=C(O)C(O)(CNc1nc(-c2cc(-c3ccon3)n(Cc3cccc(F)c3F)n2)ncc1F)C(F)(F)F]

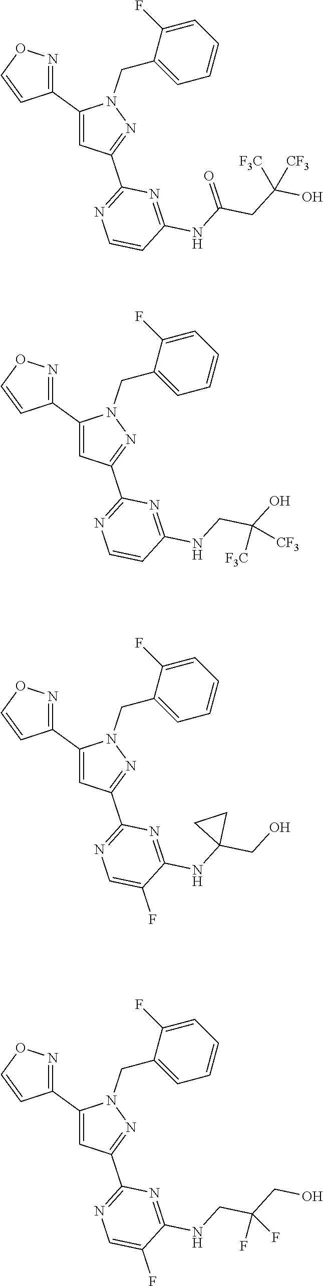 [SMILES: O=C(CC(O)(C(F)(F)F)C(F)(F)F)Nc1ccnc(-c2cc(-c3ccon3)n(Cc3ccccc3F)n2)n1 ; OC(CNc1ccnc(-c2cc(-c3ccon3)n(Cc3ccccc3F)n2)n1)(C(F)(F)F)C(F)(F)F ; OCC1(Nc2nc(-c3cc(-c4ccon4)n(Cc4ccccc4F)n3)ncc2F)CC1 ; OCC(F)(F)CNc1nc(-c2cc(-c3ccon3)n(Cc3ccccc3F)n2)ncc1F]

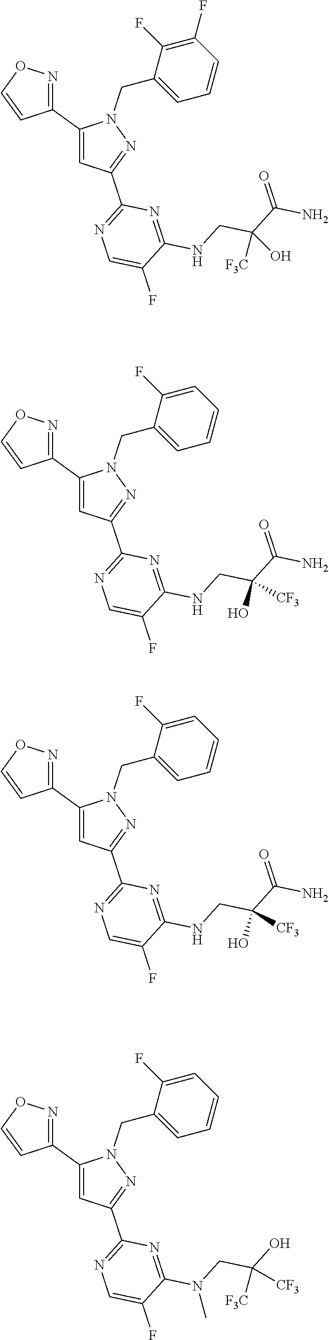 [SMILES: NC(=O)C(O)(CNc1nc(-c2cc(-c3ccon3)n(Cc3cccc(F)c3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F ; CN(CC(O)(C(F)(F)F)C(F)(F)F)c1nc(-c2cc(-c3ccon3)n(Cc3ccccc3F)n2)ncc1F]

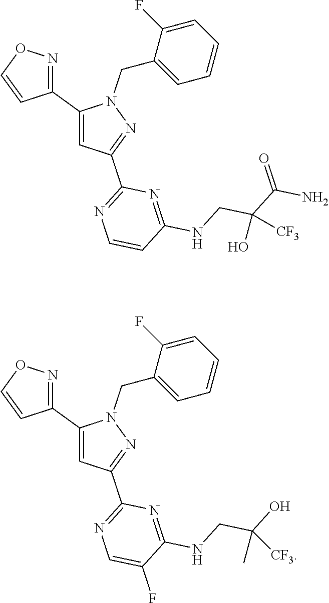 [SMILES: NC(=O)C(O)(CNc1ccnc(-c2cc(-c3ccon3)n(Cc3ccccc3F)n2)n1)C(F)(F)F ; CC(O)(CNc1nc(-c2cc(-c3ccon3)n(Cc3ccccc3F)n2)ncc1F)C(F)(F)F]

[55]. A method of any one of [1] to [48] above, or according to other embodiments of the invention, wherein the sGC stimulator is selected from one depicted in any one of Tables X, XX, XXX, IV, or XIV.
[56]. A method of any one of [1] to [48] above, or according to other embodiments of the invention, wherein the sGC stimulator is selected from one depicted in any one of Tables IZA, IZB or IZC.
[57]. A kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is an sGC stimulator, a pharmaceutically acceptable salt thereof, or a pharmaceutical composition comprising an sGC stimulator or a pharmaceutically acceptable salt thereof, for use in the treatment of achalasia in a patient in need thereof.
[58]. A kit of [57] above, or according to other embodiments of the invention, wherein the sGC stimulator is selected from one depicted in any one of [50] to [55] above, or according to other embodiments of the invention.
[59]. A kit of [57] above, or according to other embodiments of the invention, wherein the sGC stimulator is selected from one depicted in [56] above, or according to other embodiments of the invention.
[60] Use of an sGC stimulator, or a pharmaceutically acceptable salt thereof, in the manufacture of a medicament for the treatment of achalasia in a patient in need thereof.
[61]. A use of [60] above, or according to other embodiments of the invention, wherein the sGC stimulator is selected from one depicted in any one of [50] to [55] above, or according to other embodiments of the invention.
[62]. A use of [60] above, or according to other embodiments of the invention, wherein the sGC stimulator is selected from one depicted in [56] above, or according to other embodiments of the invention.
[63]. A pharmaceutical composition comprising an sGC stimulator, or a pharmaceutically acceptable salt thereof, for use in the treatment of achalasia in a patient in need thereof.
[64]. A pharmaceutical composition of [63] above, or according to other embodiments of the invention, wherein the sGC stimulator is selected from one depicted in any one of [50] to [55] above, or according to other embodiments of the invention.
[65]. A pharmaceutical composition of [63] above, or according to other embodiments of the invention, wherein the sGC stimulator is selected from one depicted in [56] above, or according to other embodiments of the invention.
[66]. A pharmaceutical composition comprising an sGC stimulator, or a pharmaceutically acceptable salt thereof, and one or more additional therapeutic agents, for use in the treatment of achalasia in a patient in need thereof.
[67]. A pharmaceutical composition of [66] above, or according to other embodiments of the invention, wherein the sGC stimulator is selected from one depicted in any one of [50] to [55] above, or according to other embodiments of the invention.
[68]. A pharmaceutical composition of [66] above, or according to other embodiments of the invention, wherein the sGC stimulator is selected from one depicted in [56] above, or according to other embodiments of the invention.

### OTHER EMBODIMENTS

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims. A number of embodiments have been described. Nevertheless, it will be understood that various modifications may be made without departing from the spirit and scope of the invention.

1. A method of treating esophageal achalasia in a patient in need thereof, comprising administering to said patient a therapeutically effective amount of an sGC stimulator or a pharmaceutically acceptable salt thereof.

2-8. (canceled)

9. The method of claim 1, wherein the achalasia is primary esophageal achalasia.

10. The method of claim 1, wherein the achalasia is secondary esophageal achalasia.

11. The method of claim 10, wherein the achalasia is a secondary achalasia associated with Chagas disease.

12. The method of claim 10, wherein the achalasia is secondary esophageal achalasia associated with esophageal cancer.

13. (canceled)

14. The method of claim 10, wherein the esophageal achalasia is a secondary esophageal achalasia associated with diabetes, systemic sclerosis, Chagas disease, a neurodegenerative or neurological disease, brain, head or neck injury or trauma or a paraneoplastic syndrome.

15. The method of claim 1, wherein said sGC stimulator or pharmaceutically acceptable salt thereof is administered as a monotherapy.

16. The method of claim 1, wherein said sGC stimulator or pharmaceutically acceptable salt thereof is administered in combination with a therapeutically or prophylactically effective amount of one or more additional therapeutic agents.

17. The method of claim 16, wherein the additional therapeutic agent is a calcium channel blocker.

18. The method of claim 17, wherein the additional therapeutic agent is nifedipine.

19. The method of claim 18, wherein the nifedipine is administered sublingually.

20. The method of claim 16, wherein the additional therapeutic agent is a botox injection.

21. The method of claim 16, wherein the additional therapeutic agent is a compound known to up-regulate the NO-pathway.

22. The method of claim 21, wherein the additional therapeutic agent is selected from nitric oxide, a NO-donor, an sGC stimulator, a sGC activator or a PDE5 inhibitor.

23. The method of claim 22, wherein the additional therapeutic agent is an NO-donor.

24. The method of claim 23, wherein the NO-donor is selected from a nitrate, a nitrite, a NONOate or a nitrosothiol.

25-26. (canceled)

27. The method of claim 1, wherein the patient in need thereof is an adult.

28. The method of claim 1, wherein the patient in need thereof is a child.

29. (canceled)

30. The method of claim 1, wherein the sGC stimulator is selected from riociguat, neliciguat, vericiguat, BAY-41-2272, BAY 41-8543 or etriciguat.

31. The method of claim 1, wherein the sGC stimulator is one of Formula IA, or a pharmaceutically acceptable salt thereof, 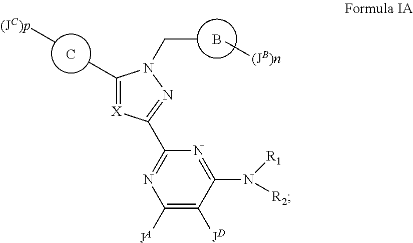
wherein:
X is selected from N, CH, C(C₁₋₄ alkyl), C(C₁₋₄ haloalkyl), CCl and CF;
ring B is a phenyl or a 6-membered heteroaryl ring containing 1 or 2 ring nitrogen atoms, or ring B is a thiophene;
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said R^{B} that is a C₁₋₆ aliphatic and each of said R^{B} that is a C₃₋₈ cycloaliphatic ring is optionally substituted with up to 3 instances of halogen;
J^{A} is selected from hydrogen, halogen, methyl, methoxy, trifluoromethyl, trifluoromethoxy or —NR^{a}R^{b}, wherein R^{a} and R^{b} are each independently selected from hydrogen, C₁₋₆ alkyl or a 3-6 cycloalkyl ring;
J^{D} is hydrogen or selected from halogen, —CN, —CF₃, methoxy, trifluoromethoxy, nitro, amino or methyl;
R¹ and R², together with the nitrogen atom to which they are attached, form a 4 to 8-membered heterocyclic ring or 5 or 6-membered heteroaryl ring; wherein said 4 to 8-membered heterocyclic ring or said 5 or 6-membered heteroaryl ring optionally contains in addition to the nitrogen atom to which R¹ and R² are attached, up to 3 ring heteroatoms independently selected from N, O or S, and is optionally substituted by up to 5 instances of R⁵; or
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, each of said C₃₋₈ cycloalkyl ring, each of said 4 to 8-membered heterocyclic ring group, each of said 5 or 6-membered heteroaryl and each of said C₁₋₆ alkyl portion of each said C₁₋₆ alkyl-R^{Y} is optionally and independently substituted with up to 5 instances of R^{5a}; provided that R¹ and R² are not simultaneously hydrogen; and provided than when X is one of CH, C(C₁₋₄ alkyl), C(C₁₋₄ haloalkyl), CCl or CF, neither of R¹ and R² is a pyridine or a pyrimidine; or
alternatively, J^{D} and one of R¹ or R² can form a 5-6 membered heterocyclic ring containing up to two heteroatoms selected from O, N and S and optionally substituted with up to 3 instances of oxo or —(Y)—R⁹;
wherein Y is either absent or is a linkage in the form of a C₁₋₆ alkyl chain optionally substituted by up to 6 instances of fluoro;
each R⁹ is independently selected from hydrogen, fluoro, —CN, —OR¹⁰, —SR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, a C₃₋₆ cycloalkyl ring, a 4-8-membered heterocyclic ring or a 5-6 membered heteroaryl ring; wherein each said 4 to 8-membered heterocyclic ring and each said 5 to 6-membered heteroaromatic ring contains up to 4 ring heteroatoms independently selected from N, O or S; and wherein each said C₃₋₆ cycloalkyl ring, each said 4 to 8-membered heterocyclic ring and each said 5 to 6-membered heteroaromatic ring is optionally substituted with up to 3 instances of R¹¹;
each R¹¹ is independently selected from halogen, C₁₋₆ alkyl, —CN, —OR¹², —SR¹², —COR¹², —OC(O)R¹², —C(O)OR¹², —C(O)N(R¹²)₂, —C(O)N(R¹²)SO₂R¹², —N(R¹²)C(O)R¹², —N(R¹²)C(O)OR¹², —N(R¹²)C(O)N(R¹²)₂, —N(R¹²)₂, —SO₂R¹², —SO₂N(R¹²)₂, —SO₂N(R¹²)COOR¹², —SO₂N(R¹²)C(O)R¹², —N(R¹²)SO₂R¹² and —N═OR¹²; wherein each of said C₁₋₆ alkyl is optionally and independently substituted by up to 3 instances of fluoro, —OH, —O(C₁₋₄ alkyl), phenyl or —O(C₁₋₄ fluoroalkyl)
wherein each R¹⁰ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo; and
wherein each R¹² is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
R^{Y} is selected from a C₃₋₈ cycloalkyl ring, a 4 to 8-membered heterocyclic ring, phenyl, or a 5 to 6-membered heteroaromatic ring; wherein each of said 4 to 8-membered heterocyclic ring and each of said 5 to 6-membered heteroaromatic ring contains up to 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaromatic ring is optionally substituted with up to 5 instances of R^{5c};
each R^{5c} is independently selected from halogen, —CN, C₁₋₆ alkyl, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring and each of said 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
two instances of R^{5c} attached to the same or different ring atoms of R^{Y}, together with said ring atom or atoms, may form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; wherein R¹¹ is hydrogen or a C₁₋₂ alkyl;
each R^{5a} is independently selected from halogen, —CN, C₁₋₆ alkyl, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring and each of said 4 to 7-membered heterocyclic ring contains up to 4 ring heteroatoms independently selected from N, O and S, wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; or
when one of R¹ or R² is the C₃₋₈ cycloalkyl ring, 4 to 8-membered heterocyclic ring or 5 or 6-membered heteroaryl substituted with up to 5 instances of R^{5a}, two of the instances of R^{5a} attached to the same or different ring atoms of said R¹ or R², together with said atom or atoms, may optionally form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl;
each R⁵ is independently selected from halogen, —CN, C₁₋₆ alkyl, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each said benzyl or each said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁶ is independently selected from hydrogen, a C₁₋₆ alkyl, phenyl, benzyl, a C₃₋₈ cycloalkyl ring or a 4 to 7-membered heterocyclic ring, a 5 or 6-membered heteroaryl ring; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
when R¹ and R² attached to the nitrogen atom form the 4 to 8-membered heterocyclic ring or 5 or 6-membered heteroaryl ring substituted with up to 5 instances of R⁵, two of the instances of R⁵ attached to the same or different atoms of said ring, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, said 4 to 6-membered heterocyclic ring, said phenyl or said 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen; wherein R is hydrogen or a C₁₋₂ alkyl;
p is an integer selected from 0, 1 or 2;
ring C is a monocyclic 5-membered heteroaryl ring containing up to 4 ring heteroatoms selected from N, O or S; wherein said monocyclic 5-membered heteroaryl ring is not a 1,3,5-triazinyl ring;
each J^{C} is independently selected from halogen or a C₁₋₄ aliphatic optionally and independently substituted by up to 3 instances of C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen.

32. The method of claim 31, wherein the sGC stimulator is one of Formula IB, or a pharmaceutically acceptable salt thereof, 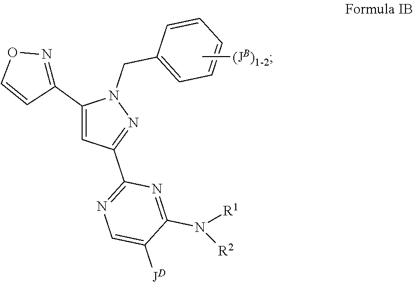
wherein J^{D} is selected from hydrogen or halogen; J^{B} is halogen and
R¹ and R², together with the nitrogen atom to which they are attached, form a 4 to 8-membered heterocyclic ring or 5-membered heteroaryl ring; wherein said 4 to 8-membered heterocyclic ring or said 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 10-membered heterocyclic ring, a 5 or 6-membered heteroaryl, phenyl or a C₁₋₆ alkyl-R^{Y}; wherein each of said 4 to 10-membered heterocyclic ring and each of said 5 or 6-membered heteroaryl ring contains up to 3 ring heteroatoms independently selected from N, O and S; and wherein each of said C₁₋₆ alkyl, each of said C₁₋₆ alkyl portion of each said C₁₋₆ alkyl-R^{Y} moiety, each of said C₃₋₈ cycloalkyl ring, each of said 4 to 10-membered heterocyclic ring group, each of said 5 or 6-membered heteroaryl, each of said phenyl is optionally and independently substituted with up to 5 instances of R^{5f}; provided that neither of R¹ or R² are pyridine or pyrimidine;
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring and each of said 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, each said C₁₋₆ alkyl, each said C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen; and
R″ is hydrogen or a C₁₋₂ alkyl.

33. The method of claim 32, wherein the sGC stimulator is one of Formula IC, or a pharmaceutically acceptable salt thereof, 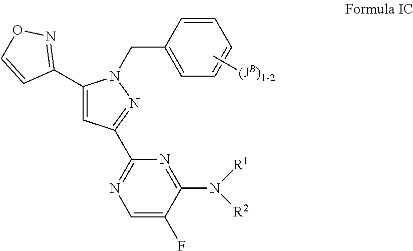
wherein J^{B} is halogen;
R¹ is hydrogen or C₁₋₆ alkyl;
R² is a C₁₋₆ alkyl group optionally and independently substituted by up to three instances of R^{5a}.

34. The method of claim 33, wherein the sGC stimulator is selected from one depicted below, or a pharmaceutically acceptable salt thereof:

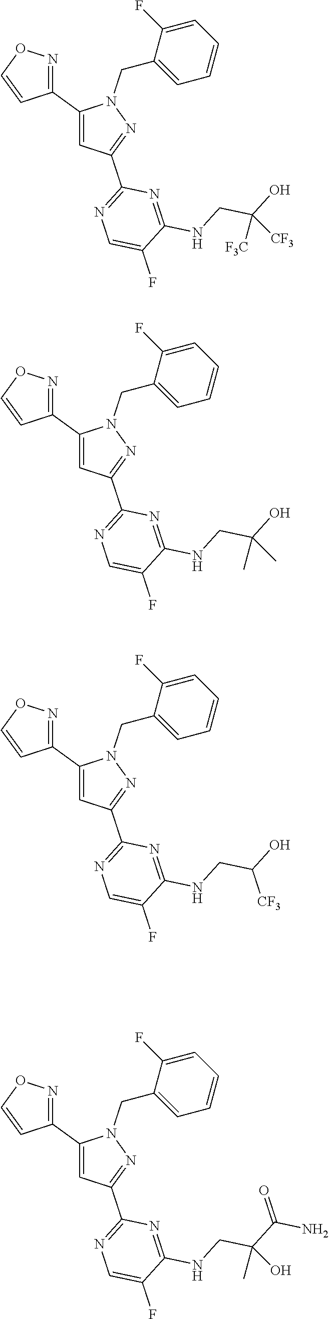 [SMILES: OC(CNc1nc(-c2cc(-c3ccon3)n(Cc3ccccc3F)n2)ncc1F)(C(F)(F)F)C(F)(F)F ; CC(C)(O)CNc1nc(-c2cc(-c3ccon3)n(Cc3ccccc3F)n2)ncc1F ; OC(CNc1nc(-c2cc(-c3ccon3)n(Cc3ccccc3F)n2)ncc1F)C(F)(F)F ; CC(O)(CNc1nc(-c2cc(-c3ccon3)n(Cc3ccccc3F)n2)ncc1F)C(N)=O]

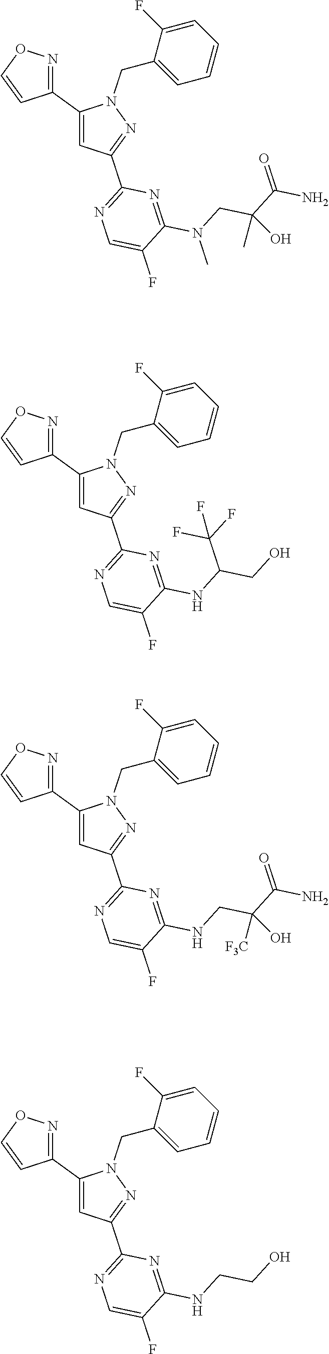 [SMILES: CN(CC(C)(O)C(N)=O)c1nc(-c2cc(-c3ccon3)n(Cc3ccccc3F)n2)ncc1F ; OCC(Nc1nc(-c2cc(-c3ccon3)n(Cc3ccccc3F)n2)ncc1F)C(F)(F)F ; NC(=O)C(O)(CNc1nc(-c2cc(-c3ccon3)n(Cc3ccccc3F)n2)ncc1F)C(F)(F)F ; OCCNc1nc(-c2cc(-c3ccon3)n(Cc3ccccc3F)n2)ncc1F]

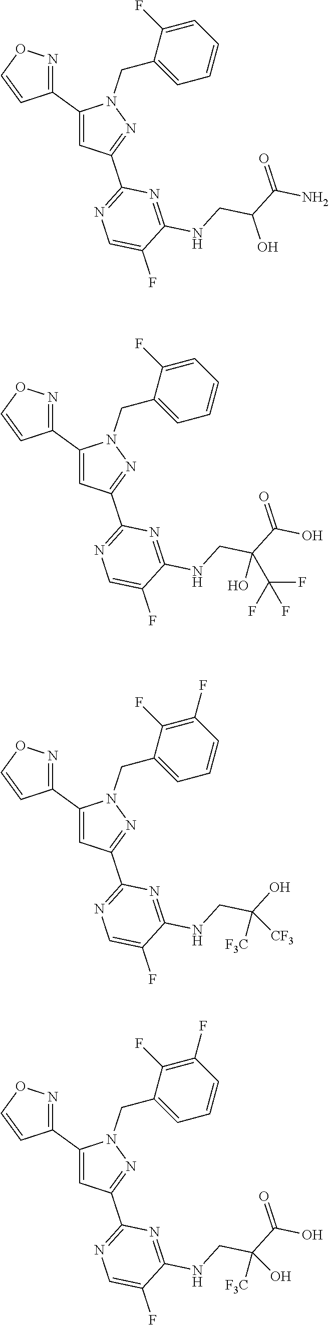 [SMILES: NC(=O)C(O)CNc1nc(-c2cc(-c3ccon3)n(Cc3ccccc3F)n2)ncc1F ; O=C(O)C(O)(CNc1nc(-c2cc(-c3ccon3)n(Cc3ccccc3F)n2)ncc1F)C(F)(F)F ; OC(CNc1nc(-c2cc(-c3ccon3)n(Cc3cccc(F)c3F)n2)ncc1F)(C(F)(F)F)C(F)(F)F ; O=C(O)C(O)(CNc1nc(-c2cc(-c3ccon3)n(Cc3cccc(F)c3F)n2)ncc1F)C(F)(F)F]

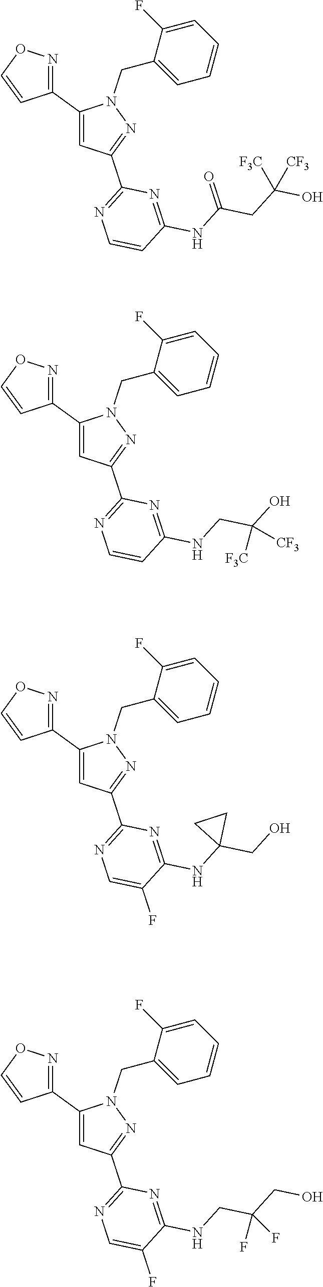 [SMILES: O=C(CC(O)(C(F)(F)F)C(F)(F)F)Nc1ccnc(-c2cc(-c3ccon3)n(Cc3ccccc3F)n2)n1 ; OC(CNc1ccnc(-c2cc(-c3ccon3)n(Cc3ccccc3F)n2)n1)(C(F)(F)F)C(F)(F)F ; OCC1(Nc2nc(-c3cc(-c4ccon4)n(Cc4ccccc4F)n3)ncc2F)CC1 ; OCC(F)(F)CNc1nc(-c2cc(-c3ccon3)n(Cc3ccccc3F)n2)ncc1F]

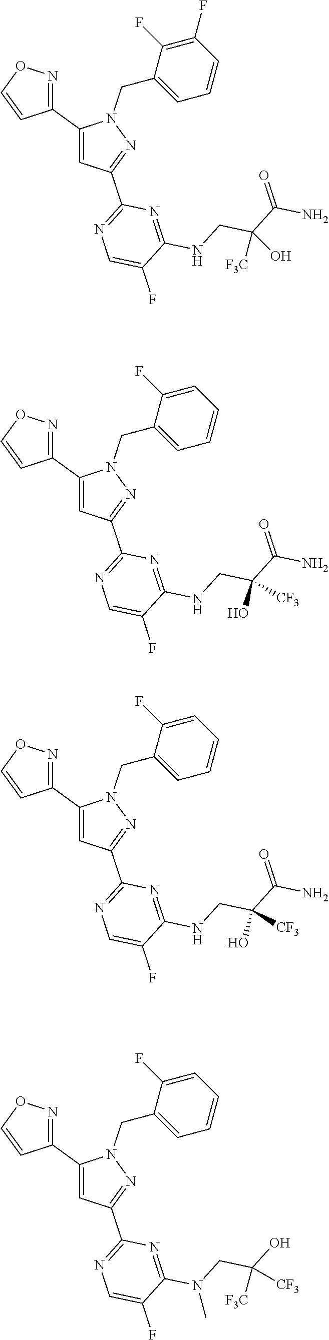 [SMILES: NC(=O)C(O)(CNc1nc(-c2cc(-c3ccon3)n(Cc3cccc(F)c3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F ; CN(CC(O)(C(F)(F)F)C(F)(F)F)c1nc(-c2cc(-c3ccon3)n(Cc3ccccc3F)n2)ncc1F]

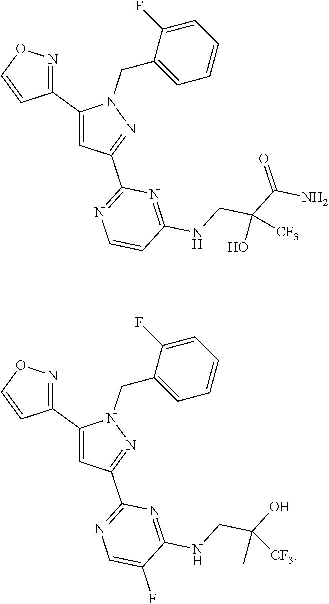 [SMILES: NC(=O)C(O)(CNc1ccnc(-c2cc(-c3ccon3)n(Cc3ccccc3F)n2)n1)C(F)(F)F ; CC(O)(CNc1nc(-c2cc(-c3ccon3)n(Cc3ccccc3F)n2)ncc1F)C(F)(F)F]

35-40. (canceled)
